(19) United States
(12) Patent Application Publication
(10) Pub. No.: US 2010/0056515 A1
(43) Pub. Date: Mar. 4, 2010

(54) BENZIMIDAZOLE COMPOUNDS

(76) Inventors: Kazuyoshi Aso, Osaka-shi (JP); Michiyo Mochizuki, Osaka-shi (JP); Takuto Kojima, Osaka-shi (JP); Katsumi Kobayashi, Osaka-shi (JP); Scott Alan Pratt, Longmont, CO (US); Albert Charles Gyorkos, Westminster, CO (US); Christopher Peter Corrette, Arvada, CO (US); Suk Young Cho, Gyeonggi-Do (KR)

Correspondence Address: WENDEROTH, LIND & PONACK, L.L.P., 1030 15th Street, N.W.,, Suite 400 East, Washington, DC, 20005-1503, US

(21) Appl. No.: 12/312,087

(22) PCT Filed: Oct. 24, 2007
(86) PCT No.: PCT/US07/22472

§ 371 Date: Jul. 17, 2009

Related U.S. Application Data
(60) Provisional application No. 60/854,062, filed on Oct. 25, 2006.

(51) Int. Cl. A61K 31/5377; A61K 31/4412; A61K 31/4166; C07D 413/10; C07D 401/12; C07D 235/26; A61P 25/24; A61P 25/22

(52) U.S. Cl. 514/234.5; 514/338; 514/395; 544/139; 546/273.7; 548/306.4

(57) ABSTRACT

There is provided a compound of the formula (I):

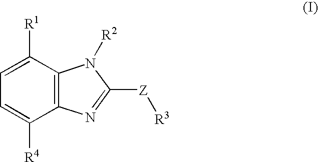

wherein R¹ is an optionally substituted C₁₋₁₀ alkyl; R² is H, or a C₁₋₆ alkyl which may be substituted with 1 to 3 substituents; R³ is a 5- or 6-membered aromatic group which may be substituted with 1 to 5 substituents, wherein the 5- or 6-membered aromatic group may be fused with a 5- or 6-membered ring which may be substituted with 1 to 3 C₁₋₆ alkyls; R⁴ is a hydrogen, a halogen, a hydroxy, a cyano, a C₁₋₆ alkyl or a C₁₋₆ alkoxy; Z is —O—, —S—, —SO—, —SO₂—, or —NR⁵— wherein R⁵ is a hydrogen or a C₁₋₆ alkyl; or a salt thereof or a prodrug thereof, which have CRF receptor antagonist activity and use thereof.

### TECHNICAL FIELD

The present invention relates to novel benzimidazole compounds having corticotropin releasing factor antagonistic activity and pharmaceutical compositions containing them.

### BACKGROUND OF INVENTION

Corticotropin-releasing factor (hereinafter, abbreviated as “CRF”) is a neuropeptide composed of 41 amino acids, and was isolated and purified as a peptide promoting release of adrenocorticotropic hormone (ACTH) from pituitary gland. First, the structure thereof was determined from sheep hypothalamus and, thereafter, the presence thereof was confirmed also in a rat or a human, and the structure thereof was determined [Science, 213, 1394 (1981); Proc. Natl. Acad. Sci. USA, 80, 4851 (1983); EMBO J. 5, 775 (1983)]. An amino acid sequence is the same in a human and a rat, but differed in 7 amino acids in ovine. CRF is synthesized as a carboxy-terminal of prepro CRF, cut and secreted. The CRF peptide and an mRNA thereof are present at the largest amount in hypothalamus and pituitary gland, and are widely distributed in a brain such as cerebral cortex, cerebellum, hippocampus and corpus amygdaloideum. In addition, in peripheral tissues, the existence has been confirmed in placenta, adrenal gland, lung, liver, pancreas, skin and digestive tract [J. Clin. Endocrinol. Metab., 65, 176 (1987); J. Clin. Endocrinol. Metab., 67, 768 (1988); Regul. Pept., 18, 173 (1987), Peptides, 5 (Suppl. 1), 71 (1984)]. A CRF receptor is a 7-transmembrane G protein-coupled receptor, and two subtypes of CRF1 and CRF2 are present. It is reported that CRF1 is present mainly in cerebral cortex, cerebellum, olfactory bulb, pituitary gland and tonsil nucleus. On the other hand, the CRF2 receptor has two subtypes of CRF2α and CRF2β. It was made clear that the CRF2α receptor is distributed much in hypothalamus, septal area and choroids plexus, and the CRF2β receptor is present mainly in peripheral tissues such as skeletal muscle and is distributed in a blood vessel in a brain [J. Neurosci. 15, 6340 (1995); Endocrinology, 137, 72 (1996); Biochim. Biophys. Acta, 1352, 129 (1997)]. Since each receptor differs in distribution in a living body, it is suggested that a role thereof is also different [Trends. Pharmacol. Sci. 23, 71(2002)].

As a physiological action of CRF, the action on the endocrine system is known in which CRF is produced and secreted in response to stress in hypothalamus and acts on pituitary gland to promote the release of ACTH [Recent Prog. Horm. Res., 39, 245 (1983)]. In addition to the action on the endocrine system, CRF acts as a neurotransmitter or a neuroregulating factor in a brain, and integrates electrophysiology, autonomic nerve and conducts to stress [Brain Res. Rev., 15, 71 (1990); Pharmacol. Rev., 43, 425 (1991)]. When CRF is administered in a cerebral ventricle of experimental animal such as a rat, anxiety conduct is observed, and much more anxiety conduct is observed in a CRF-overexpressing mouse as compared with a normal animal [Brain Res., 574, 70 (1992); J. Neurosci., 10, 176 (1992); J. Neurosci., 14, 2579 (1994)]. In addition, α-helical CRF (9-41) of a peptidergic CRF receptor antagonist exerts an anti-anxiety action in an animal model [Brain Res., 509, 80 (1990); J. Neurosci., 14, 2579 (1994)]. A blood pressure, a heart rate and a body temperature of a rat are increased by stress or CRF administration, but the α-helical CRF (9-41) of a peptidergic CRF antagonist inhibits the increase in a blood pressure, a heart rate and a body temperature due to stress [J. Physiol., 460, 221 (1993)]. The α-helical CRF (9-41) of a peptidergic CRF receptor antagonist inhibits abnormal conducts due to withdrawal of a dependent drug such as an alcohol and a cocaine [Psychopharmacology, 103, 227 (1991); Pharmacol. Rev. 53, 209 (2001)]. In addition, it has been reported that learning and memory are promoted by CRF administration in a rat [Nature, 375, 284 (1995); Neuroendocrinology, 57, 1071 (1993); Eur. J. Pharmacol., 405, 225 (2000)].

Since CRF is associated with stress response in a living body, there are clinical reports regarding stress-associated depression or anxiety. The CRF concentration in a cerebrospinal fluid of a depression patient is higher as compared with that of a normal person [Am. J. Psychiatry, 144, 873 (1987)], and the mRNA level of CRF in hypothalamus of a depression patient is increased as compared with that of a normal person [Am. J. Psychiatry, 152, 1372 (1995)]. A CRF binding site of cerebral cortex of a patient who suicided by depression is decreased [Arch. Gen. Psychiatry, 45, 577 (1988)]. The increase in the plasma ACTH concentration due to CRF administration is small in a depression patient [N. Engl. J. Med., 314, 1329 (1986)]. In a patient with panic disorder, the increase of plasma ACTH concentration due to CRF administration is small [Am. J. Psychiatry, 143, 896 (1986)]. The CRF concentration in a cerebrospinal fluid of a patient with anxiety induced by stress such as obsessive-compulsive neurosis, post-psychic trauma stress disorder, Tourette's syndrome and the like is higher as compared with that of a normal person [Arch. Gen. Psychiatry, 51, 794 (1994); Am. J. Psychiatry, 154, 624 (1997); Biol. Psychiatry, 39, 776 (1996)]. The CRF concentration in a cerebrospinal fluid of schizophrenics is higher as compared with that of a normal person [Brain Res., 437, 355 (1987); Neurology, 37, 905 (1987)]. Thus, it has been reported that there is abnormality in the living body response system via CRF in stress-associated mental disease.

The action of CRF on the endocrine system can be presumed by the characteristics of CRF gene-introduced animal and actions in an experimental animal. In a CRF-overexpressing mouse, excessive secretions of ACTH and adrenal cortex steroid occur, and abnormalities analogous to Cushing's syndrome such as atrophy of muscle, alopecia, infertility and the like are observed [Endocrinology, 130, 3378 (1992)]. CRF inhibits ingestion in an experimental animal such as a rat [Life Sci., 31, 363 (1982); Neurophamacology, 22, 337 (1983)]. In addition, α-helical CRF (9-41) of a peptidergic CRF antagonist inhibited decrease of ingestion due to stress loading in an experimental model [Brain Res. Bull., 17, 285 (1986)]. CRF inhibited weight gain in a hereditary obesity animal [Physiol. Behav., 45, 565 (1989)]. In a nervous orexia inactivity patient, the increase of ACTH in plasma upon CRF administration is small [J. Clin. Endocrinol. Metab., 62, 319 (1986)]. It has been suggested that a low CRF value is associated with obesity syndrome [Endocrinology, 130, 1931 (1992)]. There has been suggested a possibility that ingestion inhibition and weight loss action of a serotonin reuptake inhibiting agent are exerted via release of CRF [Pharmacol. Rev., 43, 425 (1991)].

CRF is centrally or peripherally associated with the digestive tract movement involved in stress or inflammation [Am. J. Physiol. Gastrointest. Liver Physiol. 280, G315 (2001)]. CRF acts centrally or peripherally, weakens the shrinkablity of stomach, and decreases the gastric excreting ability [Regulatory Peptides, 21, 173 (1988); Am. J. Physiol., 253, G241 (1987)]. In addition, α-helical CRF (9-41) of a peptidergic CRF antagonist has a restoring action for hypofunction of stomach by abdominal operation [Am. J. Physiol., 258, G152 (1990)]. CRF inhibits secretion of a bicarbonate ion in stomach, decreases gastric acid secretion and inhibits ulcer due to cold restriction stress [Am. J. Physiol., 258, G152 (1990)]. Furthermore, α-helical CRF (9-41) of a peptidergic CRF antagonist shows the inhibitory action on gastric acid secretion decrease, gastric excretion decrease, small intestinal transport decrease and large intestinal transport enhancement due to restriction stress [Gastroenterology, 95, 1510 (1988)]. In a healthy person, mental stress increases a gas and abdominal pain due to anxiety and intestine dilation, and CRF decreases a threshold of discomfort [Gastroenterology, 109, 1772 (1995); Neurogastroenterol. Mot., 8, 9 [1996]. In an irritable bowel syndrome patient, large intestinal movement is excessively enhanced by CRF administration as compared with a healthy person [Gut, 42, 845 (1998)].

It has been reported from studies on experimental animals and clinical studies that CRF is induced by inflammation and is involved in an inflammatory reaction. In an inflammatory site of an experimental animal and in a joint fluid of a rheumatoid arthritis patient, production of CRF is topically increased [Science, 254, 421 (1991); J. Clin. Invest., 90, 2555 (1992); J. Immunol., 151, 1587 (1993)]. CRF induces degranulation of a mast cell and enhances the blood vessel permeability [Endocrinology, 139, 403 (1998); J. Pharmacol. Exp. Ther., 288, 1349 (1999)]. CRF can be detected also in a thyroid gland of autoimmune thyroiditis patient [Am. J. Pathol. 145, 1159 (1994)]. When CRF is administered to an experimental autoimmune cerebrospinal meningitis rat, the progression of symptom such as paralysis was remarkably inhibited [J. Immunil., 158, 5751 (1997)]. In a rat, the immune response activities such as T-lymphocyte proliferation and the natural killer cell activity are reduced by CRF administration or stress loading [Endocrinology, 128, 1329 (1991)].

From the above-mentioned reports, it is expected that the CRF receptor antagonistic compound would exert an excellent effect for treating or preventing various diseases in which CRF is associated.

As a CRF antagonist, for example, peptide CRF receptor antagonists are reported in which a part of an amino acid sequence of CRF or associated peptides of a human or other mammals is altered or deleted, and they are reported to show a pharmacological action such as ACTH release-inhibiting action and anti-anxiety action [Science, 224, 889 (1984); J. Pharmacol. Exp. Ther., 269, 564 (1994); Brain Res. Rev., 15, 71 (1990)]. However, from a pharmacokinetic point of view such as chemical stability and absorbability for oral administration in a living body, bioavailability and intracerebral transferability, peptide derivatives have a low utility value as a medicine.

As a CRF antagonistic compound, for example, nitrogen-containing fused heterocyclic compounds are reported in WO 2005/44793, WO2005/099688 and WO2006/116412.

### DISCLOSURE OF INVENTION

According to the present invention, there is provided:

[1] A compound represented by the formula (I):

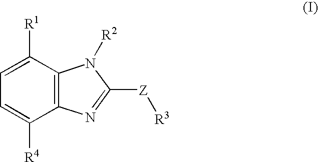

wherein R¹ is an optionally substituted C₁₋₁₀ alkyl;
R² is a hydrogen, or a C₁₋₆ alkyl which may be substituted with 1 to 3 substituents selected from the group consisting of a hydroxy, a cyano, an amino, a mono- or di-C₁₋₆ alkylamino, a C₁₋₆ alkoxy and an optionally substituted benzyloxy;
R³ is a 5- or 6-membered aromatic group which may be substituted with 1 to 5 substituents selected from the group consisting of (i) halogen, (ii) cyano, (iii) C₁₋₆ alkyl which may be substituted with 1 to 3 substituents selected from the group consisting of (i′) a mono- or di-C₁₋₆ alkylamino, (ii′) a halogen and (iii′) 5- or 6-membered heterocyclic group which may be substituted with 1 to 3 C₁₋₆ alkyls, (iv) amino which may be substituted with a C₁₋₆ alkyl, (v) C₁₋₆ alkoxy which may be substituted with 1 to 3 halogens, (vi) C₁₋₆ alkyl-carbonyl, (vii) carbamoyl, (viii) 5- or 6-membered heterocyclic group which may be substituted with 1 to 3 C₁₋₆ alkyls which may be substituted with 1 to 3 halogens, (ix) C₁₋₆ alkylthio, (x) C₁₋₆ alkylsulfinyl, (xi) C₁₋₆ alkylsulfonyl, (xii) sulfamoyl, and (xiii) tri-C₁₋₆ alkylsilyl, wherein the 5- or 6-membered aromatic group may be fused with a 5- or 6-membered ring which may be substituted with 1 to 3 C₁₋₆ alkyls;
R⁴ is a hydrogen, a halogen, a hydroxy, a cyano, a C₁₋₆ alkyl or a C₁₋₆ alkoxy;
Z is —O—, —S—, —SO—, —SO₂—, or —NR⁵— wherein R⁵ is a hydrogen or a C₁₋₆ alkyl; or a salt thereof (hereinafter, sometimes abbreviated as “Compound (I)”;
[2] A prodrug of the compound according to the above-mentioned [1];
[3] The compound according to the above-mentioned [1] wherein R¹ is a C₃₋₁₀ alkyl which may be substituted with 1 or 2 substituents selected from the group consisting of an amino and a mono- or di-C₁₋₆ alkylamino;
[4] The compound according to the above-mentioned [1] wherein R² is a hydrogen, or a C₁₋₆ alkyl which may be substituted with 1 to 3 substituents selected from the group consisting of a hydroxy and di-C₁₋₆ alkylamino;
[5] The compound according to the above-mentioned [1] wherein R³ is (i) 2,4,6-trisubstituted phenyl, (ii) 2,4-disubstituted phenyl, (iii) 2,4,6-trisubstituted 3-pyridyl, (iv) 2,6- or 4,6-disubstituted 3-pyridyl, (v) 3,5-disubstituted 2-pyridyl, or (vi) 1,4,5-trisubstituted 3-pyrazolyl;
[6] The compound according to the above-mentioned [1] wherein R⁴ is a hydrogen, a halogen, or a C₁₋₆ alkoxy;
[7] The compound according to the above-mentioned [1] wherein Z is —O— or —NH—;
[8] The compound according to the above-mentioned [1] wherein the compound is
(1) 2-[2-chloro-4-(trifluoromethoxy)phenoxy]-7-(1-ethylpropyl)-4-methoxy-1-methyl-1H-benzimidazole,
(2) 3,5-dichloro-4-{[7-(1-ethylpropyl)-4-methoxy-1-methyl-1H-benzimidazol-2-yl]oxy}-N,N-dimethylaniline,
(3) 3-chloro-4-{[7-(1-ethylpropyl)-4-methoxy-1-methyl-1H-benzimidazol-2-yl]oxy}-N,N-dimethylaniline,
(4) 5-chloro-2-{[7-(1-ethylpropyl)-4-methoxy-1-methyl-1H-benzimidazol-2-yl]oxy}-N,N-dimethylaniline,
(5) 3-{[7-(1-ethylpropyl)-4-methoxy-1-methyl-1H-benzimidazol-2-yl]oxy}-N,N,6-trimethylpyridin-2-amine,
(6) 1-(4-{[7-(1-ethylpropyl)-4-methoxy-1-methyl-1H-benzimidazol-2-yl]oxy}-3-methylphenyl)ethanone,
(7) 3,5-dichloro-2-{[7-(1-ethylpropyl)-4-methoxy-1-methyl-1H-benzimidazol-2-yl]oxy}-N,N-dimethylaniline,
(8) 2-[(5-bromo-3-methylpyridin-2-yl)oxy]-7-(1-ethylpropyl)-4-methoxy-1-methyl-1H-benzimidazole,
(9) 1-(5-chloro-2-{[7-(1-ethylpropyl)-4-methoxy-1-methyl-1H-benzimidazol-2-yl]oxy}phenyl)-N,N-dimethylmethanamine,
(10) 2-[2-bromo-4-(trifluoromethoxy)phenoxy]-7-(1-ethylpropyl)-4-methoxy-1-methyl-1H-benzimidazole,
(11) 7-(1-ethylpropyl)-4-methoxy-1-methyl-2-{[6-methyl-2-(trifluoromethyl)pyridin-3-yl]oxy}-1H-benzimidazole,
(12) 2-{[7-(1-ethylpropyl)-4-methoxy-1-methyl-1H-benzimidazol-2-yl]oxy}-N,N-dimethyl-5-(trifluoromethyl)aniline,
(13) 1-[2-{[7-(1-ethylpropyl)-4-methoxy-1-methyl-1H-benzimidazol-2-yl]oxy}-5-(trifluoromethoxy)phenyl]-N,N-dimethylmethanamine,
(14) 7-(1-ethylpropyl)-4-methoxy-1-methyl-2-[(1,4,5-trimethyl-1H-pyrazol-3-yl)oxy]-1H-benzimidazole,
(15) 1-[2-{[7-(1-ethylpropyl)-4-methoxy-1-methyl-1H-benzimidazol-2-yl]oxy}-3-methyl-5-(trifluoromethoxy)phenyl]-N,N-dimethylmethanamine,
(16) N-[2-chloro-4-(trifluoromethyl)phenyl]-7-(1-ethylpropyl)-4-methoxy-1-methyl-1H-benzimidazol-2-amine,
(17) N-[4-chloro-2-(trifluoromethyl)phenyl]-7-(1-ethylpropyl)-4-methoxy-1-methyl-1H-benzimidazol-2-amine,
(18) N-[2-chloro-4-(trifluoromethoxy)phenyl]-7-(1-ethylpropyl)-4-methoxy-1-methyl-1H-benzimidazol-2-amine,
(19) N-(2-bromo-4-chlorophenyl)-7-(1-ethylpropyl)-4-methoxy-1-methyl-1H-benzimidazol-2-amine,
(20) N-(2,4-dichlorophenyl)-7-(1-ethylpropyl)-4-methoxy-1-methyl-1H-benzimidazol-2-amine,
(21) 4-chloro-N¹-[7-(1-ethylpropyl)-4-methoxy-1-methyl-1H-benzimidazol-2-yl]-N²,N²-dimethylbenzene-1,2-diamine,
(22) 5-chloro-2-{[7-(1-ethylpropyl)-4-methoxy-1-methyl-1H-benzimidazol-2-yl]oxy}benzenesulfonamide
(23) 2-(2-bromo-4,6-dichlorophenoxy)-4-chloro-7-(1-ethylpropyl)-1-methyl-1H-benzimidazole,
(24) 3,5-dichloro-2-{[4-chloro-7-(1-ethylpropyl)-1-methyl-1H-benzimidazol-2-yl]oxy}-N,N-dimethylaniline,
(25) 4-chloro-2-(2,4-dichloro-6-morpholin-4-ylphenoxy)-7-(1-ethylpropyl)-1-methyl-1H-benzimidazole,
(26) 4-chloro-2-[2,4-dichloro-6-(4-methylpiperazin-1-yl)phenoxy]-7-(1-ethylpropyl)-1-methyl-1H-benzimidazole,
(27) 4-chloro-2-[2,4-dichloro-6-(1H-imidazol-1-yl)phenoxy]-7-(1-ethylpropyl)-1-methyl-1H-benzimidazole,
(28) 3,5-dichloro-2-{[4-chloro-7-(1-ethylpropyl)-1-methyl-1H-benzimidazol-2-yl]oxy}-N-methylaniline,
(29) 4-chloro-2-(2,4-dichloro-6-pyrrolidin-1-ylphenoxy)-7-(1-ethylpropyl)-1-methyl-1H-benzimidazole,
(30) 2-{[7-(1-ethylpropyl)-4-methoxy-1-methyl-1H-benzimidazol-2-yl]oxy}-N,N-dimethyl-5-(trifluoromethoxy)aniline,
(31) 3,5-dichloro-2-{[4-chloro-7-(1-ethylpropyl)-1-methyl-1H-benzimidazol-2-yl]oxy}benzonitrile,
(32) 3,5-dichloro-2-{[4-chloro-7-(1-ethylpropyl)-1-methyl-1H-benzimidazol-2-yl]oxy}benzenesulfonamide,
(33) 2-(4-bromo-2-chloro-6-methylphenoxy)-4-chloro-7-(1-ethylpropyl)-1-methyl-1H-benzimidazole,
(34) 3-chloro-4-{[4-chloro-7-(1-ethylpropyl)-1-methyl-1H-benzimidazol-2-yl]oxy}-5-methylbenzonitrile,
(35) 1-(3-chloro-4-{[4-chloro-7-(1-ethylpropyl)-1-methyl-1H-benzimidazol-2-yl]oxy}-5-methylphenyl)ethanone,
(36) 4-chloro-2-[2-chloro-6-methyl-4-(methylthio)phenoxy]-7-(1-ethylpropyl)-1-methyl-1H-benzimidazole,
(37) 4-chloro-2-[2-chloro-6-methyl-4-(methylsulfinyl)phenoxy]-7-(1-ethylpropyl)-1-methyl-1H-benzimidazole,
(38) 4-chloro-2-[2-chloro-6-methyl-4-(methylsulfonyl)phenoxy]-7-(1-ethylpropyl)-1-methyl-1H-benzimidazole,
(39) 3-chloro-4-{[4-chloro-7-(1-ethylpropyl)-1-methyl-1H-benzimidazol-2-yl]oxy}-5-methylbenzamide,
(40) 3-chloro-4-{[4-chloro-7-(1-ethylpropyl)-1-methyl-1H-benzimidazol-2-yl]oxy}-5-methylbenzenesulfonamide,
(41) 2-[2-bromo-6-chloro-4-(trifluoromethoxy)phenoxy]-4-chloro-7-(1-ethylpropyl)-1-methyl-1H-benzimidazole,
(42) 4-chloro-2-[2-chloro-6-(methylthio)-4-(trifluoromethoxy)phenoxy]-7-(1-ethylpropyl)-1-methyl-1H-benzimidazole,
(43) 4-chloro-2-[2-chloro-6-(methylsulfinyl)-4-(trifluoromethoxy)phenoxy]-7-(1-ethylpropyl)-1-methyl-1H-benzimidazole,
(44) 4-chloro-2-[2-chloro-6-(methylsulfonyl)-4-(trifluoromethoxy)phenoxy]-7-(1-ethylpropyl)-1-methyl-1H-benzimidazole,
(45) 4-chloro-7-(1-ethylpropyl)-N-[2-methoxy-6-methyl-4-(methylthio)phenyl]-1-methyl-1H-benzimidazol-2-amine,
(46) 4-chloro-7-(1-ethylpropyl)-N-[2-methoxy-6-methyl-4-(methylsulfinyl)phenyl]-1-methyl-1H-benzimidazol-2-amine,
(47) 4-chloro-7-(1-ethylpropyl)-N-[2-methoxy-6-methyl-4-(methylsulfonyl)phenyl]-1-methyl-1H-benzimidazol-2-amine,
(48) 4-{[4-chloro-7-(1-ethylpropyl)-1-methyl-1H-benzimidazol-2-yl]amino}-3-methoxy-5-methylbenzamide,
(49) N-(2-bromo-4-chloro-6-methylphenyl)-4-chloro-7-(1-ethylpropyl)-1-methyl-1H-benzimidazol-2-amine,
(50) 4-chloro-N-[4-chloro-2-methyl-6-(methylthio)phenyl]-7-(1-ethylpropyl)-1-methyl-1H-benzimidazol-2-amine,
(51) 4-chloro-N-[4-chloro-2-methyl-6-(methylsulfinyl)phenyl]-7-(1-ethylpropyl)-1-methyl-1H-benzimidazol-2-amine,
(52) 3-{[4-chloro-7-(1-ethylpropyl)-1-methyl-1H-benzimidazol-2-yl]oxy}-4-methoxy-N,N,6-trimethylpyridin-2-amine,
(53) 4-chloro-7-(1-ethylpropyl)-1-methyl-2-[(2-methyl-2,4,5,6-tetrahydrocyclopenta[c]pyrazol-3-yl)oxy]-1H-benzimidazole,
(54) 4-chloro-7-(1-ethylpropyl)-1-methyl-2-[(2-methyl-2,3-dihydro-1H-isoindol-4-yl)oxy]-1H-benzimidazole,
(55) 4-chloro-2-[(5,7-dichloro-2-methyl-2,3-dihydro-1H-isoindol-4-yl)oxy]-7-(1-ethylpropyl)-1-methyl-1H-benzimidazole,
(56) 4-chloro-7-(1-ethylpropyl)-2-{[4-methoxy-2-methyl-6-(trifluoromethyl)pyridin-3-yl]oxy}-1-methyl-1H-benzimidazole,
(57) 4-chloro-7-(1-ethylpropyl)-N-[2-methoxy-6-methyl-4-(1H-pyrazol-1-yl)phenyl]-1-methyl-1H-benzimidazol-2-amine,
(58) 4-chloro-7-(1-ethylpropyl)-N-{2-methoxy-6-methyl-4-[3-(trifluoromethyl)-1H-pyrazol-1-yl]phenyl}-1-methyl-1H-benzimidazol-2-amine,
(59) 4-chloro-2-[2-chloro-6-methyl-4-(trimethylsilyl)phenoxy]-7-(1-ethylpropyl)-1-methyl-1H-benzimidazole,
(60) 4-chloro-2-[2,4-dichloro-6-(trimethylsilyl)phenoxy]-7-(1-ethylpropyl)-1-methyl-1H-benzimidazole,
(61) N-(4-chloro-2-methoxy-6-methylphenyl)-1-methyl-7-[1-(methylamino)propyl]-1H-benzimidazol-2-amine,
(62) N-(4-chloro-2-methoxy-6-methylphenyl)-7-[1-(dimethylamino)propyl]-1-methyl-1H-benzimidazol-2-amine,
(63) 2-[4-bromo-2-(2,4-dichloro-6-methylphenoxy)-7-(1-ethylpropyl)-1H-benzimidazol-1-yl]-N,N-dimethylethanamine,
(64) 4-chloro-2-(2,4-dichloro-6-methylphenoxy)-7-(1-ethylpropyl)-1H-benzimidazole,
(65) 4-chloro-2-[2,6-dichloro-4-(trifluoromethoxy)phenoxy]-7-(1-ethylpropyl)-1H-benzimidazole,
(66) 4-chloro-N-(2,4-dichloro-6-methylphenyl)-7-(1-ethylpropyl)-1H-benzimidazol-2-amine,
(67) 4-chloro-N-(4-chloro-2-methoxy-6-methylphenyl)-7-(1-ethylpropyl)-1H-benzimidazol-2-amine,
(68) 4-chloro-2-[2,6-dichloro-4-(trifluoromethoxy)phenoxy]-7-(1-ethylpropyl)-1-{2-[(4-methoxybenzyl)oxy]ethyl}-1H-benzimidazole,
(69) 2-[4-chloro-2-[2,6-dichloro-4-(trifluoromethoxy)phenoxy]-7-(1-ethylpropyl)-1H-benzimidazol-1-yl]ethanol,
(70) 4-bromo-2-(2,4-dichloro-6-methylphenoxy)-7-(1-ethylpropyl)-1-methyl-1H-benzimidazole,
(71) 2-(2,4-dichloro-6-methylphenoxy)-4-ethyl-7-(1-ethylpropyl)-1-methyl-1H-benzimidazole,
(72) 2-(2-bromo-4-chlorophenoxy)-4-ethoxy-7-(1-ethylpropyl)-1-methyl-1H-benzimidazole,
(73) 2-(2,4-dichloro-6-methylphenoxy)-4-ethoxy-7-(1-ethylpropyl)-1-methyl-1H-benzimidazole,
(74) 4-chloro-2-[(4,6-dibromo-2-methylpyridin-3-yl)oxy]-7-(1-ethylpropyl)-1-methyl-1H-benzimidazole,
(75) 2-{[4-bromo-2-methyl-6-(trifluoromethyl)pyridin-3-yl]oxy}-4-chloro-7-(1-ethylpropyl)-1-methyl-1H-benzimidazole,
(76) 4-chloro-7-(1-ethylpropyl)-1-methyl-2-{[2-methyl-4,6-bis(trifluoromethyl)pyridin-3-yl]oxy}-1H-benzimidazole,
(77) 1-(3,5-dichloro-2-{[7-(1-ethylpropyl)-4-methoxy-1-methyl-1H-benzimidazol-2-yl]oxy}phenyl)-N,N-dimethylmethanamine,
(78) 4-chloro-2-[2,4-dichloro-6-(pyrrolidin-1-ylmethyl)phenoxy]-7-(1-ethylpropyl)-1-methyl-1H-benzimidazole,
(79) 2-[2,4-dichloro-6-(pyrrolidin-1-ylmethyl)phenoxy]-7-(1-ethylpropyl)-4-methoxy-1-methyl-1H-benzimidazole,
(80) 2-{2,4-dichloro-6-[(2-methylpyrrolidin-1-yl)methyl]phenoxy}-7-(1-ethylpropyl)-4-methoxy-1-methyl-1H-benzimidazole,
(81) 2-(4-bromo-2-chloro-6-fluorophenoxy)-7-(1-ethylpropyl)-4-methoxy-1-methyl-1H-benzimidazole,
(82) 7-(1-ethylpropyl)-2-[2-(1H-imidazol-1-yl)-4-(trifluoromethoxy)phenoxy]-4-methoxy-1-methyl-1H-benzimidazole,
(83) 3-chloro-4-{[4-chloro-7-(1-ethylpropyl)-1-methyl-1H-benzimidazol-2-yl]oxy}-N,N-dimethylaniline,
(84) 1-[2-{[4-chloro-7-(1-ethylpropyl)-1-methyl-1H-benzimidazol-2-yl]oxy}-3-methyl-5-(trifluoromethoxy)phenyl]-N,N-dimethylmethanamine,
(85) 1-(3,5-dichloro-2-{([4-chloro-7-(1-ethylpropyl)-1-methyl-1H-benzimidazol-2-yl]oxy}phenyl)-N,N-dimethylmethanamine,
(86) 3-chloro-4-{[7-(1-ethylpropyl)-4-methoxy-1-methyl-1H-benzimidazol-2-yl]oxy}-N,N-dimethyl-5-(pyrrolidin-1-ylmethyl)aniline,
(87) N-(2,4-dichloro-6-methylphenyl)-7-(1-ethylpropyl)-4-methoxy-1-methyl-1H-benzimidazol-2-amine,
(88) N-[2,6-dichloro-4-(trifluoromethoxy)phenyl]-7-(1-ethylpropyl)-4-methoxy-1-methyl-1H-benzimidazol-2-amine,
(89) 2-[(2,4-dichloro-6-methylphenyl)amino]-7-(1-ethylpropyl)-1-methyl-1H-benzimidazol-4-ol,
(90) 2-(2,4-dichloro-6-methylphenoxy)-7-(1-ethylpropyl)-1-methyl-1H-benzimidazole,
(91) 1-[4-chloro-2-(2,4-dichloro-6-methylphenoxy)-1-methyl-1H-benzimidazol-7-yl]propan-1-one,
(92) 4-chloro-2-(2,4-dichloro-6-methylphenoxy)-1-methyl-7-(1-methylenepropyl)-1H-benzimidazole,
(93) 2-[4-chloro-2-(2,4-dichloro-6-methylphenoxy)-1-methyl-1H-benzimidazol-7-yl]butan-1-ol,
(94) 2-[4-chloro-2-(2,4-dichloro-6-methylphenoxy)-1-methyl-1H-benzimidazol-7-yl]butanal,
(95) 4-chloro-2-(2,4-dichloro-6-methylphenoxy)-7-(1-ethylprop-2-en-1-yl)-1-methyl-1H-benzimidazole,
(96) 3-[4-chloro-2-(2,4-dichloro-6-methylphenoxy)-1-methyl-1H-benzimidazol-7-yl]pentan-1-ol,
(97) 3-[4-chloro-2-(2,4-dichloro-6-methylphenoxy)-1-methyl-1H-benzimidazol-7-yl]pentanal,
(98) 3-[4-chloro-2-(2,4-dichloro-6-methylphenoxy)-1-methyl-1H-benzimidazol-7-yl]pentanoic acid,
(99) 2-[4-chloro-2-[(2,4-dichlorophenyl)amino]-7-(1-ethylpropyl)-1H-benzimidazol-1-yl]ethanol,
(100) 2-[4-chloro-2-({4-chloro-2-[(dimethylamino)methyl]phenyl}amino)-7-(1-ethylpropyl)-1H-benzimidazol-1-yl]ethanol or
(101) 1-{2-[2,6-dichloro-4-(trifluoromethoxy)phenoxy]-1-methyl-1H-benzimidazol-7-yl}-N,N-dimethylpropan-1-amine;
   [9] A pharmaceutical which comprises the compound according to the above-mentioned [1] or its prodrug;
   [10] The pharmaceutical according to the above-mentioned [9] which is for treating or preventing a disease associated with the functions of a CRF receptor;
   [11] The pharmaceutical according to the above-mentioned [10] wherein the disease is affective disorder, depression or anxiety;
   [12] A method for treating or preventing a disease associated with the functions of a CRF receptor, which comprises administering to a subject in need thereof an effective amount of the compound according to the above-mentioned [1] or its prodrug;
   [13] The method according to the above-mentioned [12] wherein the disease is affective disorder, depression or anxiety;
   [14] Use of the compound according to the above-mentioned [1] or its prodrug for manufacturing an agent of treating or preventing a disease associated with the functions of a CRF receptor;
   [15] The use according to claim 14 wherein the disease is affective disorder, depression or anxiety; and the like.

Each symbol in the above formula is hereinafter described in more detail.

R¹ in the formula (I) is an optionally substituted C₁₋₁₀ alkyl.

The “C₁₋₁₀ alkyl” of the “optionally substituted C₁₋₁₀ alkyl” for R¹ includes, for example, methyl, ethyl, n-propyl, isopropyl, n-butyl, isobutyl, sec-butyl, tert-butyl, n-pentyl, 1-methylbuthyl, 1-ethylpropyl, 1,2-dimethylpropyl, n-hexyl, 2-ethylbuthyl, n-heptyl, 1-propylpenthyl, 1-buthylpenthyl, 1-buthylhexyl, etc.

The substituents of the “optionally substituted C₁₋₁₀ alkyl” for R¹ include, for example, amino, mono- or di-C₁₋₆ alkylamino (e.g., mono-C₁₋₆ alkylamino such as methylamino, ethylamino, propylamino, isopropylamino, butylamino etc; di-C₁₋₆ alkylamino such as dimethylamino, diethylamino, ethylmethylamino, dipropylamino, diisopropylamino, dibutylamino, etc.), halogen atom (e.g., fluoro, chloro, bromo, iodo), nitro, cyano, C₂₋₆ alkenyl (e.g., vinyl, allyl, etc.), C₂₋₆ alkynyl (e.g., ethynyl, etc.), C₃₋₇ cycloalkyl (e.g., cyclopropyl, cyclobutyl, cyclopentyl, cyclohexyl, etc.), C₆₋₁₄ aryl (e.g., phenyl, 1-naphthyl, 2-naphthyl, 2-biphenyl, 3-biphenyl, 4-biphenyl, etc.), 5- or 6-membered aromatic heterocyclic group (e.g., furyl, thienyl, 1- or 3-pyrrolyl, 2-oxazolyl, 3-isoxazolyl, thiazolyl, 3-isothiazolyl, 1- or 2-imidazolyl, 1-pyrazolyl, 1,2,3-oxadiazolyl, 1,2,4-oxadiazolyl, 1,3,4-oxadiazolyl, furazanyl, 1,2,3-thiadiazolyl, 1,2,4-thiadiazolyl, 1,3,4-thiadiazolyl, 1,2,3-triazolyl, 1,2,4-triazolyl, tetrazolyl, 2-, 3- or 4-pyridyl, 2- or 4-pyrimidinyl, 3-pyridazinyl, pyrazinyl, triazinyl, etc.), 3- to 6-membered non-aromatic heterocyclic group (e.g., oxiranyl, azetidinyl, oxetanyl, thietanyl, 1-, 2- or 3-pyrrolidinyl, tetrahydrofuryl, thiolanyl, piperidyl, tetrahydropyranyl, morpholinyl, thiomorpholinyl, piperazinyl, etc.), 8- to 12-membered bicyclic or tricyclic fused heterocyclic group (e.g., benzofuranyl, isobenzofuranyl, benzothienyl, indolyl, isoindolyl, 1H-indazolyl, benzindazolyl, benzoxazolyl, 1,2-benzoisooxazolyl, benzothiazolyl, benzopyranyl, 1,2-benzoisothiazolyl, 1H-benzotriazolyl, quinolyl, isoquinolyl, cinnolinyl, quinazolinyl, quinoxalinyl, phthalazinyl, naphthyridinyl, purinyl, pteridinyl, carbazolyl, α-carbolinyl, β-carbolinyl, γ-carbolinyl, acridinyl, phenoxazinyl, phenothiazinyl, phenazinyl, phenoxathinyl, thianthrenyl, phenanthridinyl, phenanthrolinyl, indolizinyl, pyrrolo[1,2-b]pyridazinyl, pyrazolo[1,5-a]pyridyl, imidazo[1,2-a]pyridyl, imidazo[1,5-a]pyridyl, imidazo[1,2-b]pyridazinyl, imidazo[1,2-a]pyrimidinyl, 1,2,4-triazolo[4,3-a]pyridyl, 1,2,4-triazolo[4,3-b]pyridazinyl, 1,2,5,6-tetrahydro-4H-pyrrolo[3,2,1-ij]quinolinyl, etc.), optionally halogenated C₁₋₆ alkoxy (e.g., methoxy, ethoxy, propoxy, tert-butoxy, trifluoromethoxy, etc.), hydroxy, C₆₋₁₄ aryloxy (e.g., phenyloxy, 1-naphthyloxy, etc.), C₇₋₁₆ aralkyloxy (e.g., benzyloxy, phenetyloxy, etc.), hydroxyamino, mono-C₆₋₁₄ arylamino (e.g., phenylamino, 1-naphthylamino, etc.), di-C₆₋₁₄ arylamino (e.g., diphenylamino, etc.), C₁₋₆ alkyl-carbonylamino (e.g., acetylamino, propionylamino, etc.), C₁₋₆ alkoxy-carbonylamino (e.g., methoxycarbonylamino, ethoxycarbonylamino, etc.), 5- to 7-membered saturated cyclic amino (e.g., pyrrolidin-1-yl, piperidino, piperazin-1-yl, morpholino, thiomorpholino, homo piperazin-1-yl, etc.), formyl, carboxy, C₁₋₆ alkyl-carbonyl (e.g., acetyl, propionyl, etc.), C₃₋₆ cycloalkyl-carbonyl (e.g., cyclopropyl carbonyl, cyclobutyl carbonyl, cyclopentyl carbonyl, cyclohexyl carbonyl, etc.), C₁₋₆ alkoxy-carbonyl (e.g., methoxycarbonyl, ethoxycarbonyl, etc.), C₆₋₁₄ aryl-carbonyl (e.g., benzoyl, etc.), C₇₋₁₆ aralkyl-carbonyl (e.g., phenylacetyl, 3-phenylpropionyl, etc.), C₆₋₁₄ aryloxy-carbonyl (e.g., phenoxycarbonyl, etc.), C₇₋₁₆ aralkyloxy-carbonyl (e.g., benzyloxycarbonyl, etc.), 5- or 6-membered heterocyclic-carbonyl (e.g., nicotinoyl, isonicotinoyl, thenoyl, furoyl, morpholinocarbonyl, thiomorpholinocarbonyl, piperazin-1-ylcarbonyl, pyrrolidin-1-ylcarbonyl, etc.), carbamoyl, mono-C₁₋₆ alkyl-carbamoyl (e.g., methylcarbamoyl, ethylcarbamoyl, etc.), di-C₁₋₆ alkyl-carbamoyl (e.g., dimethylcarbamoyl, diethylcarbamoyl, ethylmethylcarbamoyl, etc.), C₆₋₁₄ aryl-carbamoyl (e.g., phenylcarbamoyl, 1-naphthylcarbamoyl, etc.), C₁₋₆ alkoxy-carbamoyl (e.g., methoxycarbamoyl, ethoxycarbamoyl, etc.), 5- or 6-membered heterocyclic carbamoyl (e.g., 2-pyridylcarbamoyl, 3-pyridylcarbamoyl, 4-pyridylcarbamoyl, 2-thienylcarbamoyl, 3-thienylcarbamoyl, etc.), mercapto, sulfo, C₁₋₆ alkylsulfinyl (e.g., methylsulfinyl, ethylsulfinyl, propylsulfinyl, butylsulfinyl etc.), C₆₋₁₄ arylsulfinyl (e.g., phenylsulfinyl, etc.), C₁₋₆ alkylsulfonyl (e.g., methylsulfonyl, ethylsulfonyl, propylsulfoyl, butylsulfonyl, etc.), C₆₋₁₄ arylsulfinyl (e.g., phenylsulfinyl, etc.), formylamino, C₁₋₆ alkyl-carbonylamino (e.g., acetylamino, etc.), C₆₋₁₄ aryl-carbonylamino (e.g., benzoylamino, etc.), C₁₋₆ alkoxy-carbonylamino (e.g., methoxycarbonylamino, ethoxycarbonylamino, etc.), C₁₋₆ alkylsulfinylamino (e.g., methylsulfinylamino, ethylsulfinylamino, propylsulfinylamino, butylsulfinylamino etc.), C₁₋₆ alkylsulfonylamino (e.g., methylsulfonylamino, ethylsulfonylamino, propylsulfoylamino, butylsulfonylamino, etc.), C₁₋₆ alkyl-carbonyloxy (e.g., acetoxy, propionyloxy, etc.), C₆₋₁₄ aryl-carbonyloxy (e.g., benzoyloxy, etc.), C₁₋₆ alkoxy-carbonyloxy (e.g., methoxycarbonyloxy, ethoxycarbonyloxy, etc.), mono-C₁₋₆ alkyl-carbamoyloxy (e.g., methylcarbamoyloxy, ethylcarbamoyloxy, etc.), di-C₁₋₆ alkyl-carbamoyloxy (e.g., dimethylcarbamoyloxy, diethylcarbamoyloxy, etc.), C₆₋₁₄ aryl-carbamoyloxy (e.g., phenylcarbamoyloxy, etc.), 5- or 6-membered heterocyclic-carbonyloxy (e.g., nicotinoyloxy, isonicotinoyloxy, etc.), oxo, imino, C₁₋₆ alkylimino (e.g., methylimino, ethylimino, etc.), C₁₋₆ alkylidene (e.g., methylidene, ethylidene, propylidene, etc), and so forth. Among others, preferred is amino, mono-C₁₋₆ alkylamino, di-C₁₋₆ alkylamino, etc.

The “C₁₋₁₀ alkyl” may have 1 to 5, preferably 1 to 2, substituents as mentioned above at possible positions and, when the number of substituents is two or more, those substituents may be the same as or different from one another.

R¹ is preferably a C₃₋₁₀ alkyl (e.g., n-propyl, isopropyl, n-butyl, isobutyl, sec-butyl, tert-butyl, n-pentyl, 1-methylbuthyl, 1-ethylpropyl, 1,2-dimethylpropyl, n-hexyl, 2-ethylbuthyl, n-heptyl, 1-propylbuthyl, 1-propylpenthyl, 1-buthylpenthyl, 1-buthylhexyl, etc.) which may be substituted with 1 or 2 substituents selected from the group consisting of an amino and a mono- or di-C₁₋₆ alkylamino

R² in the formula (I) is a hydrogen, or a C₁₋₆ alkyl which may be substituted with 1 to 3 substituents selected from the group consisting of a hydroxy, a cyano, an amino, a mono- or di-C₁₋₆ alkylamino (e.g., mono-C₁₋₆ alkylamino such as methylamino, ethylamino, propylamino, isopropylamino, butylamino etc; di-C₁₋₆ alkylamino such as dimethylamino, diethylamino, ethylmethylamino, dipropylamino, diisopropylamino, dibutylamino, etc.), a C₁₋₆ alkoxy (e.g., methoxy, ethoxy, propoxy, tert-butoxy, etc.) and an optionally substituted benzyloxy.

The “C₁₋₆ alkyl” of the “C₁₋₆ alkyl which may be substituted with 1 to 3 substituents selected from the group consisting of a hydroxy, a cyano, an amino, mono- or di-C₁₋₆ alkylamino, a C₁₋₆ alkoxy and an optionally substituted benzyloxy” for R² includes, for example, methyl, ethyl, propyl, isopropyl, butyl, isobutyl, sec-butyl, tert-butyl, n-pentyl, 1-methylbuthyl, 2-methylbuthyl, 2,2-dimethylpropyl, etc.

The substituents of the “optionally substituted benzyloxy” include, for example, halogen atom (e.g., fluoro, chloro, bromo, iodo), nitro, cyano, optionally halogenated C₁₋₆ alkyl (e.g., methyl, ethyl, propyl, isopropyl, butyl, isobutyl, tert-butyl, trifluoromethyl, etc.), C₁₋₆ alkoxy-C₁₋₆ alkyl (e.g., methoxyethyl, ethoxymethyl, etc.), amino-C₁₋₆ alkyl (e.g., aminomethyl, etc.), mono- or di-C₁₋₆ alkylamino-C₁₋₆ alkyl (e.g. methylaminomethyl, dimethylaminoethyl, etc.), C₂₋₆ alkenyl (e.g., vinyl, allyl, etc.), C₂₋₆ alkynyl (e.g., ethynyl, etc.), C₃₋₇ cycloalkyl (e.g., cyclopropyl, cyclobutyl, cyclopentyl, cyclohexyl, etc.), C₆₋₁₄ aryl (e.g., phenyl, 1-naphthyl, 2-naphthyl, 2-biphenyl, 3-biphenyl, 4-biphenyl, etc.), C₇₋₁₉ aralkyl (e.g., benzyl, etc.), 5- or 6-membered aromatic heterocyclic group (e.g., furyl, thienyl, 1- or 3-pyrrolyl, 2-oxazolyl, 3-isoxazolyl, thiazolyl, 3-isothiazolyl, 1- or 2-imidazolyl, 1-pyrazolyl, 1,2,3-oxadiazolyl, 1,2,4-oxadiazolyl, 1,3,4-oxadiazolyl, furazanyl, 1,2,3-thiadiazolyl, 1,2,4-thiadiazolyl, 1,3,4-thiadiazolyl, 1,2,3-triazolyl, 1,2,4-triazolyl, tetrazolyl, 2-, 3- or 4-pyridyl, 2- or 4-pyrimidinyl, 3-pyridazinyl, pyrazinyl, triazinyl, etc.), 3- to 6-membered non-aromatic heterocyclic group (e.g., oxiranyl, azetidinyl, oxetanyl, thietanyl, 1-, 2- or 3-pyrrolidinyl, tetrahydrofuryl, thiolanyl, piperidyl, tetrahydropyranyl, morpholinyl, thiomorpholinyl, piperazinyl, etc.), 8- to 12-membered bicyclic or tricyclic fused heterocyclic group (e.g., benzofuranyl, isobenzofuranyl, benzothienyl, indolyl, isoindolyl, 1H-indazolyl, benzindazolyl, benzoxazolyl, 1,2-benzoisooxazolyl, benzothiazolyl, benzopyranyl, 1,2-benzoisothiazolyl, 1H-benzotriazolyl, quinolyl, isoquinolyl, cinnolinyl, quinazolinyl, quinoxalinyl, phthalazinyl, naphthyridinyl, purinyl, pteridinyl, carbazolyl, α-carbolinyl, β-carbolinyl, γ-carbolinyl, acridinyl, phenoxazinyl, phenothiazinyl, phenazinyl, phenoxathinyl, thianthrenyl, phenanthridinyl, phenanthrolinyl, indolizinyl, pyrrolo[1,2-b]pyridazinyl, pyrazolo[1,5-a]pyridyl, imidazo[1,2-a]pyridyl, imidazo[1,5-a]pyridyl, imidazo[1,2-b]pyridazinyl, imidazo[1,2-a]pyrimidinyl, 1,2,4-triazolo[4,3-a]pyridyl, 1,2,4-triazolo[4,3-b]pyridazinyl, 1,2,5,6-tetrahydro-4H-pyrrolo[3,2,1-ij]quinolinyl, etc.), optionally halogenated C₁₋₆ alkoxy (e.g., methoxy, ethoxy, propoxy, tert-butoxy, trifluoromethoxy, etc.), hydroxy, C₆₋₁₄ aryloxy (e.g., phenyloxy, 1-naphthyloxy, etc.), C₇₋₁₆ aralkyloxy (e.g., benzyloxy, phenetyloxy, etc.), amino, hydroxyamino, mono-C₁₋₆ alkylamino (e.g., methylamino, ethylamino, etc.), mono-C₆₋₁₄ arylamino (e.g., phenylamino, 1-naphthylamino, etc.), di-C₁₋₆ alkylamino (e.g., dimethylamino, diethylamino, ethylmethylamino, etc.), di-C₆₋₁₄ arylamino (e.g., diphenylamino, etc.), C₁₋₆ alkyl-carbonylamino (e.g., acetylamino, propionylamino, etc.), C₁₋₆ alkoxy-carbonylamino (e.g., methoxycarbonylamino, ethoxycarbonylamino, etc.), 5- to 7-membered saturated cyclic amino (e.g., pyrrolidin-1-yl, piperidino, piperazin-1-yl, morpholino, thiomorpholino, homo piperazin-1-yl, etc.), formyl, carboxy, C₁₋₆ alkyl-carbonyl (e.g., acetyl, propionyl, etc.), C₃₋₆ cycloalkyl-carbonyl (e.g., cyclopropyl carbonyl, cyclobutyl carbonyl, cyclopentyl carbonyl, cyclohexyl carbonyl, etc.), C₁₋₆ alkoxy-carbonyl (e.g., methoxycarbonyl, ethoxycarbonyl, etc.), C₆₋₁₄ aryl-carbonyl (e.g., benzoyl, etc.), C₇₋₁₆ aralkyl-carbonyl (e.g., phenylacetyl, 3-phenylpropionyl, etc.), C₆₋₁₄ aryloxy-carbonyl (e.g., phenoxycarbonyl, etc.), C₇₋₁₆ aralkyloxy-carbonyl (e.g., benzyloxycarbonyl, etc.), 5- or 6-membered heterocyclic-carbonyl (e.g., nicotinoyl, isonicotinoyl, thenoyl, furoyl, morpholinocarbonyl, thiomorpholinocarbonyl, piperazin-1-ylcarbonyl, pyrrolidin-1-ylcarbonyl, etc.), carbamoyl, mono-C₁₋₆ alkyl-carbamoyl (e.g., methylcarbamoyl, ethylcarbamoyl, etc.), di-C₁₋₆ alkyl-carbamoyl (e.g., dimethylcarbamoyl, diethylcarbamoyl, ethylmethylcarbamoyl, etc.), C₆₋₁₄ aryl-carbamoyl (e.g., phenylcarbamoyl, 1-naphthylcarbamoyl, etc.), C₁₋₆ alkoxy-carbamoyl (e.g., methoxycarbamoyl, ethoxycarbamoyl, etc.), 5- or 6-membered heterocyclic carbamoyl (e.g., 2-pyridylcarbamoyl, 3-pyridylcarbamoyl, 4-pyridylcarbamoyl, 2-thienylcarbamoyl, 3-thienylcarbamoyl, etc.), mercapto, sulfo, C₁₋₆ alkylsulfinyl (e.g., methylsulfinyl, ethylsulfinyl, propylsulfinyl, butylsulfinyl etc.), C₆₋₁₄ arylsulfinyl (e.g., phenylsulfinyl, etc.), C₁₋₆ alkylsulfonyl (e.g., methylsulfonyl, ethylsulfonyl, propylsulfoyl, butylsulfonyl, etc.), C₆₋₁₄ arylsulfinyl (e.g., phenylsulfinyl, etc.), formylamino, C₁₋₆ alkyl-carbonylamino (e.g., acetylamino, etc.), C₆₋₁₄ aryl-carbonylamino (e.g., benzoylamino, etc.), C₁₋₆ alkoxy-carbonylamino (e.g., methoxycarbonylamino, ethoxycarbonylamino, etc.), C₁₋₆ alkylsulfinylamino (e.g., methylsulfinylamino, ethylsulfinylamino, propylsulfinylamino, butylsulfinylamino etc.), C₁₋₆ alkylsulfonylamino (e.g., methylsulfonylamino, ethylsulfonylamino, propylsulfoylamino, butylsulfonylamino, etc.), C₁₋₆ alkyl-carbonyloxy (e.g., acetoxy, propionyloxy, etc.), C₆₋₁₄ aryl-carbonyloxy (e.g., benzoyloxy, etc.), C₁₋₆ alkoxy-carbonyloxy (e.g., methoxycarbonyloxy, ethoxycarbonyloxy, etc.), mono-C₁₋₆ alkyl-carbamoyloxy (e.g., methylcarbamoyloxy, ethylcarbamoyloxy, etc.), di-C₁₋₆ alkyl-carbamoyloxy (e.g., dimethylcarbamoyloxy, diethylcarbamoyloxy, etc.), C₆₋₁₄ aryl-carbamoyloxy (e.g., phenylcarbamoyloxy, etc.), 5- or 6-membered heterocyclic-carbonyloxy (e.g., nicotinoyloxy, isonicotinoyloxy, etc.), oxo, imino, C₁₋₆ alkylimino (e.g., methylimino, ethylimino, etc.), and so forth.

The “benzyloxy” may have 1 to 3, preferably one, substituents as mentioned above at possible positions and, when the number of substituents is two or more, those substituents may be the same as or different from one another.

R² is preferably a hydrogen, or a C₁₋₆ alkyl which may be substituted with 1 to 3 substituents selected from the group consisting of a hydroxy and a di-C₁₋₆ alkylamino.

R³ in the formula (I) is a 5- or 6-membered aromatic group which may be substituted with 1 to 5 substituents selected from the group consisting of (i) halogen, (ii) cyano, (iii) C₁₋₆ alkyl which may be substituted with 1 to 3 substituents selected from the group consisting of (i′) a mono- or di-C₁₋₆ alkylamino, (ii′) a halogen and (iii′) 5- or 6-membered heterocyclic group which may be substituted with 1 to 3 C₁₋₆ alkyl, (iv) amino which may be substituted with a C₁₋₆ alkyl, (v) C₁₋₆ alkoxy which may be substituted with 1 to 3 halogens, (vi) C₁₋₆ alkyl-carbonyl, (vii) carbamoyl, (viii) 5- or 6-membered heterocyclic group which may be substituted with 1 to 3 C₁₋₆ alkyl which may be substituted with 1 to 3 halogens, (ix) C₁₋₆ alkylthio, (x) C₁₋₆ alkylsulfinyl, (xi) C₁₋₆ alkylsulfonyl, (xii) sulfamoyl, and (xiii) tri-C₁₋₆ alkylsilyl, wherein the 5- or 6-membered aromatic group may be fused with a 5- or 6-membered ring which may be substituted with 1 to 3 C₁₋₆ alkyl.

The above-mentioned “5- or 6-membered aromatic group” for R³ includes, for example, phenyl, furyl, thienyl, pyrrolyl, oxazolyl, isoxazolyl, thiazolyl, isothiazolyl, imidazolyl, pyrazolyl, 1,2,3-oxadiazolyl, 1,2,4-oxadiazolyl, 1,3,4-oxadiazolyl, furazanyl, 1,2,3-thiadiazolyl, 1,2,4-thiadiazolyl, 1,3,4-thiadiazolyl, 1,2,3-triazolyl, 1,2,4-triazolyl, tetrazolyl, pyridyl, pyrimidinyl, pyridazinyl, pyrazinyl, triazinyl, etc. Among others, preferred is phenyl, pyridyl, pyrazolyl, etc.

The “5- or 6-membered aromatic group” may have 1 to 5 substituents selected from the group consisting of (i)-(xiii) as mentioned above at possible positions and, when the number of substituents is two or more, those substituents may be the same as or different from one another.

The “5- or 6-membered heterocyclic group” of the above-mentioned item (iii) (iii′) and item (viii) includes, for example, 1- or 3-pyrrolyl, 1-, 2-, 4- or 5-imidazolyl, 1-, 3-, 4- or 5-pyrazolyl, 2- or 3-thienyl, 2- or 3-furyl, 2-, 3- or 4-pyridyl, pyrazinyl, 2- or 4-pyrimidinyl, 3-pyridazinyl, 1,2,3-triazolyl, 2-oxazolyl, 3-thiazolyl, 3-isothiazolyl, 3-isoxazolyl, pyrolynyl, 1-, 2- or 3-piperazinyl, 1-, 2-, 3- or 4-piperidinyl, 1-, 2- or 3-pyrrolidinyl, 2-, 3- or 4-pyrazolydinyl, 2-, 3- or 4-morphonyl or 2-, 3- or 4-thiomorphonyl, 2-, 3-, 4-, 5- or 6-pyranyl, etc.

The “tri-C₁₋₆ alkylsilyl” of the above-mentioned item (xiii) includes, for example, trimethylsilyl, triethylsilyl, tert-butyldimethylsilyl, etc.

The “5- or 6-membered ring” of the “5- or 6-membered aromatic group fused with a 5- or 6-membered ring” which may be substituted with 1 to 3 C₁₋₆ alkyl” for R³ includes, for example, furan, thiophen, pyrrole, pyrazole, pyrazolidine, imidazole, thiazole, oxazole, pyridine, pyrimidine, pyrazine, pyridazine, imidazolidine, piperidine, piperazine, pyrrolidine, pyrroline, oxazolidine, thiazolidine, isoxazole, isoxazolidine, cyclopentane, cyclohexane, cyclopentene, cyclohexene, etc. Among others, preferred is 5-membered non-aromatic ring, etc.

The “5- or 6-membered ring” may have 1 to 3 C₁₋₆ alkyl, preferably one C₁₋₆ alkyl at possible positions and, when the number of substituents is two or more, the C₁₋₆ alkyl may be the same as or different from one another.

Examples of the “5- or 6-membered aromatic group fused with a 5- or 6-membered ring” include 4- or 5-isobenzofuranyl, 5- or 6-chromenyl, indolizinyl, indolinyl, indazolyl, quinolinyl, isoindolyl, 4- or 5-isoindolinyl, 2,4,5,6-tetrahydrocyclopenta[c]pyrazol-3-yl, etc. Among others, preferred is 4-isoindolinyl, etc.

R³ is preferably di- or tri-substituted 5- or 6-membered aromatic group (preferably, phenyl, pyridyl, pyrazolyl, etc.).

More preferably R³ is (i) 2,4,6-trisubstituted phenyl, (ii) 2,4-disubstituted phenyl, (iii) 2,4,6-trisubstituted 3-pyridyl, (iv) 2,6- or 4,6-disubstituted 3-pyridyl, (v) 3,5-disubstituted 2-pyridyl, (vi) 1,4,5-trisubstituted 3-pyrazolyl, etc.

R⁴ in the formula (I) is a hydrogen, a halogen, a hydroxy, a cyano, a C₁₋₆ alkyl or a C₁₋₆ alkoxy. Among others, preferred is a hydrogen, a halogen, a C₁₋₆ alkoxy (e.g., methoxy, ethoxy, propoxy, tert-butoxy, etc.), etc.

Z in the formula (I) is —O—, —S—, —SO—, —SO₂—, or —NR⁵— wherein R⁵ is a hydrogen or a C₁₋₆ alkyl. Among others, preferred is —O—, —NH—, etc.

Preferable examples of the Compound (I) include a compound wherein R¹ is a C₃₋₁₀ alkyl which may be substituted with 1 or 2 substituents selected from the group consisting of an amino and a mono- or di-C₁₋₆ alkylamino;

R² is a hydrogen, or a C₁₋₆ alkyl which may be substituted with 1 to 3 substituents selected from the group consisting of a hydroxy and a di-C₁₋₆ alkylamino;
R³ is (i) 2,4,6-trisubstituted phenyl, (ii) 2,4-disubstituted phenyl, (iii) 2,4,6-trisubstituted 3-pyridyl, (iv) 2,6- or 4,6-disubstituted 3-pyridyl, (v) 3,5-disubstituted 2-pyridyl, (vi) 1,4,5-trisubstituted 3-pyrazolyl;
R⁴ is a hydrogen, a halogen, or a C₁₋₆ alkoxy; and

### Z is —O— or —NH—.

More preferable examples of the Compound (I) include
(1) 2-[2-chloro-4-(trifluoromethoxy)phenoxy]-7-(1-ethylpropyl)-4-methoxy-1-methyl-1H-benzimidazole,
(2) 3,5-dichloro-4-{[7-(1-ethylpropyl)-4-methoxy-1-methyl-1H-benzimidazol-2-yl]oxy}-N,N-dimethylaniline,
(3) 3-chloro-4-{[7-(1-ethylpropyl)-4-methoxy-1-methyl-1H-benzimidazol-2-yl]oxy}-N,N-dimethylaniline,
(4) 5-chloro-2-{[7-(1-ethylpropyl)-4-methoxy-1-methyl-1H-benzimidazol-2-yl]oxy}-N,N-dimethylaniline,
(5) 3-{[7-(1-ethylpropyl)-4-methoxy-1-methyl-1H-benzimidazol-2-yl]oxy}-N,N,6-trimethylpyridin-2-amine,
(6) 1-(4-{[7-(1-ethylpropyl)-4-methoxy-1-methyl-1H-benzimidazol-2-yl]oxy}-3-methylphenyl)ethanone,
(7) 3,5-dichloro-2-{[7-(1-ethylpropyl)-4-methoxy-1-methyl-1H-benzimidazol-2-yl]oxy}-N,N-dimethylaniline,
(8) 2-[(5-bromo-3-methylpyridin-2-yl)oxy]-7-(1-ethylpropyl)-4-methoxy-1-methyl-1H-benzimidazole,
(9) 1-(5-chloro-2-{[7-(1-ethylpropyl)-4-methoxy-1-methyl-1H-benzimidazol-2-yl]oxy}phenyl)-N,N-dimethylmethanamine,
(10) 2-[2-bromo-4-(trifluoromethoxy)phenoxy]-7-(1-ethylpropyl)-4-methoxy-1-methyl-1H-benzimidazole,
(11) 7-(1-ethylpropyl)-4-methoxy-1-methyl-2-{[6-methyl-2-(trifluoromethyl)pyridin-3-yl]oxy}-1H-benzimidazole,
(12) 2-{[7-(1-ethylpropyl)-4-methoxy-1-methyl-1H-benzimidazol-2-yl]oxy}-N,N-dimethyl-5-(trifluoromethyl)aniline,
(13) 1-[2-{[7-(1-ethylpropyl)-4-methoxy-1-methyl-1H-benzimidazol-2-yl]oxy}-5-(trifluoromethoxy)phenyl]-N,N-dimethylmethanamine,
(14) 7-(1-ethylpropyl)-4-methoxy-1-methyl-2-[(1,4,5-trimethyl-1H-pyrazol-3-yl)oxy]-1H-benzimidazole,
(15) 1-[2-{[7-(1-ethylpropyl)-4-methoxy-1-methyl-1H-benzimidazol-2-yl]oxy}-3-methyl-5-(trifluoromethoxy)phenyl]-N,N-dimethylmethanamine,
(16) N-[2-chloro-4-(trifluoromethyl)phenyl]-7-(1-ethylpropyl)-4-methoxy-1-methyl-1H-benzimidazol-2-amine,
(17) N-[4-chloro-2-(trifluoromethyl)phenyl]-7-(1-ethylpropyl)-4-methoxy-1-methyl-1H-benzimidazol-2-amine,
(18) N-[2-chloro-4-(trifluoromethoxy)phenyl]-7-(1-ethylpropyl)-4-methoxy-1-methyl-1H-benzimidazol-2-amine,
(19) N-(2-bromo-4-chlorophenyl)-7-(1-ethylpropyl)-4-methoxy-1-methyl-1H-benzimidazol-2-amine,
(20) N-(2,4-dichlorophenyl)-7-(1-ethylpropyl)-4-methoxy-1-methyl-1H-benzimidazol-2-amine,
(21) 4-chloro-N¹-[7-(1-ethylpropyl)-4-methoxy-1-methyl-1H-benzimidazol-2-yl]-N²,N²-dimethylbenzene-1,2-diamine,
(22) 5-chloro-2-{[7-(1-ethylpropyl)-4-methoxy-1-methyl-1H-benzimidazol-2-yl]oxy}benzenesulfonamide,
(23) 2-(2-bromo-4,6-dichlorophenoxy)-4-chloro-7-(1-ethylpropyl)-1-methyl-1H-benzimidazole,
(24) 3,5-dichloro-2-{[4-chloro-7-(1-ethylpropyl)-1-methyl-1H-benzimidazol-2-yl]oxy}-N,N-dimethylaniline,
(25) 4-chloro-2-(2,4-dichloro-6-morpholin-4-ylphenoxy)-7-(1-ethylpropyl)-1-methyl-1H-benzimidazole,
(26) 4-chloro-2-[2,4-dichloro-6-(4-methylpiperazin-1-yl)phenoxy]-7-(1-ethylpropyl)-1-methyl-1H-benzimidazole,
(27) 4-chloro-2-[2,4-dichloro-6-(1H-imidazol-1-yl)phenoxy]-7-(1-ethylpropyl)-1-methyl-1H-benzimidazole,
(28) 3,5-dichloro-2-{[4-chloro-7-(1-ethylpropyl)-1-methyl-1H-benzimidazol-2-yl]oxy}-N-methylaniline,
(29) 4-chloro-2-(2,4-dichloro-6-pyrrolidin-1-ylphenoxy)-7-(1-ethylpropyl)-1-methyl-1H-benzimidazole,
(30) 2-{[7-(1-ethylpropyl)-4-methoxy-1-methyl-1H-benzimidazol-2-yl]oxy}-N,N-dimethyl-5-(trifluoromethoxy)aniline,
(31) 3,5-dichloro-2-{[4-chloro-7-(1-ethylpropyl)-1-methyl-1H-benzimidazol-2-yl]oxy}benzonitrile,
(32) 3,5-dichloro-2-{[4-chloro-7-(1-ethylpropyl)-1-methyl-1H-benzimidazol-2-yl]oxy}benzenesulfonamide,
(33) 2-(4-bromo-2-chloro-6-methylphenoxy)-4-chloro-7-(1-ethylpropyl)-1-methyl-1H-benzimidazole,
(34) 3-chloro-4-{[4-chloro-7-(1-ethylpropyl)-1-methyl-1H-benzimidazol-2-yl]oxy}-5-methylbenzonitrile,
(35) 1-(3-chloro-4-{[4-chloro-7-(1-ethylpropyl)-1-methyl-1H-benzimidazol-2-yl]oxy}-5-methylphenyl)ethanone,
(36) 4-chloro-2-[2-chloro-6-methyl-4-(methylthio)phenoxy]-7-(1-ethylpropyl)-1-methyl-1H-benzimidazole,
(37) 4-chloro-2-[2-chloro-6-methyl-4-(methylsulfinyl)phenoxy]-7-(1-ethylpropyl)-1-methyl-1H-benzimidazole,
(38) 4-chloro-2-[2-chloro-6-methyl-4-(methylsulfonyl)phenoxy]-7-(1-ethylpropyl)-1-methyl-1H-benzimidazole,
(39) 3-chloro-4-{[4-chloro-7-(1-ethylpropyl)-1-methyl-1H-benzimidazol-2-yl]oxy}-5-methylbenzamide,
(40) 3-chloro-4-{[4-chloro-7-(1-ethylpropyl)-1-methyl-1H-benzimidazol-2-yl]oxy}-5-methylbenzenesulfonamide,
(41) 2-[2-bromo-6-chloro-4-(trifluoromethoxy)phenoxy]-4-chloro-7-(1-ethylpropyl)-1-methyl-1H-benzimidazole,
(42) 4-chloro-2-[2-chloro-6-(methylthio)-4-(trifluoromethoxy)phenoxy]-7-(1-ethylpropyl)-1-methyl-1H-benzimidazole,
(43) 4-chloro-2-[2-chloro-6-(methylsulfinyl)-4-(trifluoromethoxy)phenoxy]-7-(1-ethylpropyl)-1-methyl-1H-benzimidazole,
(44) 4-chloro-2-[2-chloro-6-(methylsulfonyl)-4-(trifluoromethoxy)phenoxy]-7-(1-ethylpropyl)-1-methyl-1H-benzimidazole,
(45) 4-chloro-7-(1-ethylpropyl)-N-[2-methoxy-6-methyl-4-(methylthio)phenyl]-1-methyl-1H-benzimidazol-2-amine,
(46) 4-chloro-7-(1-ethylpropyl)-N-[2-methoxy-6-methyl-4-(methylsulfinyl)phenyl]-1-methyl-1H-benzimidazol-2-amine,
(47) 4-chloro-7-(1-ethylpropyl)-N-[2-methoxy-6-methyl-4-(methylsulfonyl)phenyl]-1-methyl-1H-benzimidazol-2-amine,
(48) 4-{[4-chloro-7-(1-ethylpropyl)-1-methyl-1H-benzimidazol-2-yl]amino}-3-methoxy-5-methylbenzamide,
(49) N-(2-bromo-4-chloro-6-methylphenyl)-4-chloro-7-(1-ethylpropyl)-1-methyl-1H-benzimidazol-2-amine,
(50) 4-chloro-N-[4-chloro-2-methyl-6-(methylthio)phenyl]-7-(1-ethylpropyl)-1-methyl-1H-benzimidazol-2-amine,
(51) 4-chloro-N-[4-chloro-2-methyl-6-(methylsulfinyl)phenyl]-7-(1-ethylpropyl)-1-methyl-1H-benzimidazol-2-amine,
(52) 3-{[4-chloro-7-(1-ethylpropyl)-1-methyl-1H-benzimidazol-2-yl]oxy}-4-methoxy-N,N,6-trimethylpyridin-2-amine,
(53) 4-chloro-7-(1-ethylpropyl)-1-methyl-2-[(2-methyl-2,4,5,6-tetrahydrocyclopenta[c]pyrazol-3-yl)oxy]-1H-benzimidazole,
(54) 4-chloro-7-(1-ethylpropyl)-1-methyl-2-[(2-methyl-2,3-dihydro-1H-isoindol-4-yl)oxy]-1H-benzimidazole,
(55) 4-chloro-2-[(5,7-dichloro-2-methyl-2,3-dihydro-1H-isoindol-4-yl)oxy]-7-(1-ethylpropyl)-1-methyl-1H-benzimidazole,
(56) 4-chloro-7-(1-ethylpropyl)-2-{[4-methoxy-2-methyl-6-(trifluoromethyl)pyridin-3-yl]oxy}-1-methyl-1H-benzimidazole,
(57) 4-chloro-7-(1-ethylpropyl)-N-[2-methoxy-6-methyl-4-(1H-pyrazol-1-yl)phenyl]-1-methyl-1H-benzimidazol-2-amine,
(58) 4-chloro-7-(1-ethylpropyl)-N-{2-methoxy-6-methyl-4-[3-(trifluoromethyl)-1H-pyrazol-1-yl]phenyl}-1-methyl-1H-benzimidazol-2-amine,
(59) 4-chloro-2-[2-chloro-6-methyl-4-(trimethylsilyl)phenoxy]-7-(1-ethylpropyl)-1-methyl-1H-benzimidazole,
(60) 4-chloro-2-[2,4-dichloro-6-(trimethylsilyl)phenoxy]-7-(1-ethylpropyl)-1-methyl-1H-benzimidazole,
(61) N-(4-chloro-2-methoxy-6-methylphenyl)-1-methyl-7-[1-(methylamino)propyl]-1H-benzimidazol-2-amine,
(62) N-(4-chloro-2-methoxy-6-methylphenyl)-7-[1-(dimethylamino)propyl]-1-methyl-1H-benzimidazol-2-amine,
(63) 2-[4-bromo-2-(2,4-dichloro-6-methylphenoxy)-7-(1-ethylpropyl)-1H-benzimidazol-1-yl]-N,N-dimethylethanamine,
(64) 4-chloro-2-(2,4-dichloro-6-methylphenoxy)-7-(1-ethylpropyl)-1H-benzimidazole,
(65) 4-chloro-2-[2,6-dichloro-4-(trifluoromethoxy)phenoxy]-7-(1-ethylpropyl)-1H-benzimidazole,
(66) 4-chloro-N-(2,4-dichloro-6-methylphenyl)-7-(1-ethylpropyl)-1H-benzimidazol-2-amine,
(67) 4-chloro-N-(4-chloro-2-methoxy-6-methylphenyl)-7-(1-ethylpropyl)-1H-benzimidazol-2-amine,
(68) 4-chloro-2-[2,6-dichloro-4-(trifluoromethoxy)phenoxy]-7-(1-ethylpropyl)-1-{2-[(4-methoxybenzyl)oxy]ethyl}-1H-benzimidazole,
(69) 2-[4-chloro-2-[2,6-dichloro-4-(trifluoromethoxy)phenoxy]-7-(1-ethylpropyl)-1H-benzimidazol-1-yl]ethanol,
(70) 4-bromo-2-(2,4-dichloro-6-methylphenoxy)-7-(1-ethylpropyl)-1-methyl-1H-benzimidazole,
(71) 2-(2,4-dichloro-6-methylphenoxy)-4-ethyl-7-(1-ethylpropyl)-1-methyl-1H-benzimidazole,
(72) 2-(2-bromo-4-chlorophenoxy)-4-ethoxy-7-(1-ethylpropyl)-1-methyl-1H-benzimidazole,
(73) 2-(2,4-dichloro-6-methylphenoxy)-4-ethoxy-7-(1-ethylpropyl)-1-methyl-1H-benzimidazole,
(74) 4-chloro-2-[(4,6-dibromo-2-methylpyridin-3-yl)oxy]-7-(1-ethylpropyl)-1-methyl-1H-benzimidazole,
(75) 2-{[4-bromo-2-methyl-6-(trifluoromethyl)pyridin-3-yl]oxy}-4-chloro-7-(1-ethylpropyl)-1-methyl-1H-benzimidazole,
(76) 4-chloro-7-(1-ethylpropyl)-1-methyl-2-{[2-methyl-4,6-bis(trifluoromethyl)pyridin-3-yl]oxy}-1H-benzimidazole,
(77) 1-(3,5-dichloro-2-{[7-(1-ethylpropyl)-4-methoxy-1-methyl-1H-benzimidazol-2-yl]oxy}phenyl)-N,N-dimethylmethanamine,
(78) 4-chloro-2-[2,4-dichloro-6-(pyrrolidin-1-ylmethyl)phenoxy]-7-(1-ethylpropyl)-1-methyl-1H-benzimidazole,
(79) 2-[2,4-dichloro-6-(pyrrolidin-1-ylmethyl)phenoxy]-7-(1-ethylpropyl)-4-methoxy-1-methyl-1H-benzimidazole,
(80) 2-{2,4-dichloro-6-[(2-methylpyrrolidin-1-yl)methyl]phenoxy}-7-(1-ethylpropyl)-4-methoxy-1-methyl-1H-benzimidazole,
(81) 2-(4-bromo-2-chloro-6-fluorophenoxy)-7-(1-ethylpropyl)-4-methoxy-1-methyl-1H-benzimidazole,
(82) 7-(1-ethylpropyl)-2-[2-(1H-imidazol-1-yl)-4-(trifluoromethoxy)phenoxy]-4-methoxy-1-methyl-1H-benzimidazole,
(83) 3-chloro-4-{[4-chloro-7-(1-ethylpropyl)-1-methyl-1H-benzimidazol-2-yl]oxy}-N,N-dimethylaniline,
(84) 1-[2-{[4-chloro-7-(1-ethylpropyl)-1-methyl-1H-benzimidazol-2-yl]oxy}-3-methyl-5-(trifluoromethoxy)phenyl]-N,N-dimethylmethanamine,
(85) 1-(3,5-dichloro-2-{[4-chloro-7-(1-ethylpropyl)-1-methyl-1H-benzimidazol-2-yl]oxy}phenyl)-N,N-dimethylmethanamine,
(86) 3-chloro-4-{[7-(1-ethylpropyl)-4-methoxy-1-methyl-1H-benzimidazol-2-yl]oxy}-N,N-dimethyl-5-(pyrrolidin-1-ylmethyl)aniline,
(87) N-(2,4-dichloro-6-methylphenyl)-7-(1-ethylpropyl)-4-methoxy-1-methyl-1H-benzimidazol-2-amine,
(88) N-[2,6-dichloro-4-(trifluoromethoxy)phenyl]-7-(1-ethylpropyl)-4-methoxy-1-methyl-1H-benzimidazol-2-amine,
(89) 2-[(2,4-dichloro-6-methylphenyl)amino]-7-(1-ethylpropyl)-1-methyl-1H-benzimidazol-4-ol,
(90) 2-(2,4-dichloro-6-methylphenoxy)-7-(1-ethylpropyl)-1-methyl-1H-benzimidazole,
(91) 1-[4-chloro-2-(2,4-dichloro-6-methylphenoxy)-1-methyl-1H-benzimidazol-7-yl]propan-1-one,
(92) 4-chloro-2-(2,4-dichloro-6-methylphenoxy)-1-methyl-7-(1-methylenepropyl)-1H-benzimidazole,
(93) 2-[4-chloro-2-(2,4-dichloro-6-methylphenoxy)-1-methyl-1H-benzimidazol-7-yl]butan-1-ol,
(94) 2-[4-chloro-2-(2,4-dichloro-6-methylphenoxy)-1-methyl-1H-benzimidazol-7-yl]butanal,
(95) 4-chloro-2-(2,4-dichloro-6-methylphenoxy)-7-(1-ethylprop-2-en-1-yl)-1-methyl-1H-benzimidazole,
(96) 3-[4-chloro-2-(2,4-dichloro-6-methylphenoxy)-1-methyl-1H-benzimidazol-7-yl]pentan-1-ol,
(97) 3-[4-chloro-2-(2,4-dichloro-6-methylphenoxy)-1-methyl-1H-benzimidazol-7-yl]pentanal,
(98) 3-[4-chloro-2-(2,4-dichloro-6-methylphenoxy)-1-methyl-1H-benzimidazol-7-yl]pentanoic acid,
(99) 2-[4-chloro-2-[(2,4-dichlorophenyl)amino]-7-(1-ethylpropyl)-1H-benzimidazol-1-yl]ethanol,
(100) 2-[4-chloro-2-({4-chloro-2-[(dimethylamino)methyl]phenyl}amino)-7-(1-ethylpropyl)-1H-benzimidazol-1-yl]ethanol and
(101) 1-{2-[2,6-dichloro-4-(trifluoromethoxy)phenoxy]-1-methyl-1H-benzimidazol-7-yl}-N,N-dimethylpropan-1-amine.

Much more preferable examples of the Compound (I) include
(1) 2-[2-chloro-4-(trifluoromethoxy)phenoxy]-7-(1-ethylpropyl)-4-methoxy-1-methyl-1H-benzimidazole,
(2) 3,5-dichloro-4-{[7-(1-ethylpropyl)-4-methoxy-1-methyl-1H-benzimidazol-2-yl]oxy}-N,N-dimethylaniline,
(3) 3-chloro-4-{[7-(1-ethylpropyl)-4-methoxy-1-methyl-1H-benzimidazol-2-yl]oxy}-N,N-dimethylaniline,
(4) 5-chloro-2-{[7-(1-ethylpropyl)-4-methoxy-1-methyl-1H-benzimidazol-2-yl]oxy}-N,N-dimethylaniline,
(5) 3-{[7-(1-ethylpropyl)-4-methoxy-1-methyl-1H-benzimidazol-2-yl]oxy}-N,N,6-trimethylpyridin-2-amine,
(6) 1-(4-{[7-(1-ethylpropyl)-4-methoxy-1-methyl-1H-benzimidazol-2-yl]oxy}-3-methylphenyl)ethanone,
(7) 3,5-dichloro-2-{[7-(1-ethylpropyl)-4-methoxy-1-methyl-1H-benzimidazol-2-yl]oxy}-N,N-dimethylaniline,
(8) 2-[(5-bromo-3-methylpyridin-2-yl)oxy]-7-(1-ethylpropyl)-4-methoxy-1-methyl-1H-benzimidazole,
(9) 1-(5-chloro-2-{[7-(1-ethylpropyl)-4-methoxy-1-methyl-1H-benzimidazol-2-yl]oxy}phenyl)-N,N-dimethylmethanamine,
(10) 2-[2-bromo-4-(trifluoromethoxy)phenoxy]-7-(1-ethylpropyl)-4-methoxy-1-methyl-1H-benzimidazole,
(11) 7-(1-ethylpropyl)-4-methoxy-1-methyl-2-{[6-methyl-2-(trifluoromethyl)pyridin-3-yl]oxy}-1H-benzimidazole,
(12) 2-{[7-(1-ethylpropyl)-4-methoxy-1-methyl-1H-benzimidazol-2-yl]oxy}-N,N-dimethyl-5-(trifluoromethyl)aniline,
(13) 1-[2-{[7-(1-ethylpropyl)-4-methoxy-1-methyl-1H-benzimidazol-2-yl]oxy}-5-(trifluoromethoxy)phenyl]-N,N-dimethylmethanamine,
(14) 7-(1-ethylpropyl)-4-methoxy-1-methyl-2-[(1,4,5-trimethyl-1H-pyrazol-3-yl)oxy]-1H-benzimidazole,
(15) 1-[2-{[7-(1-ethylpropyl)-4-methoxy-1-methyl-1H-benzimidazol-2-yl]oxy}-3-methyl-5-(trifluoromethoxy)phenyl]-N,N-dimethylmethanamine,
(16) N-[2-chloro-4-(trifluoromethyl)phenyl]-7-(1-ethylpropyl)-4-methoxy-1-methyl-1H-benzimidazol-2-amine,
(17) N-[4-chloro-2-(trifluoromethyl)phenyl]-7-(1-ethylpropyl)-4-methoxy-1-methyl-1H-benzimidazol-2-amine,
(18) N-[2-chloro-4-(trifluoromethoxy)phenyl]-7-(1-ethylpropyl)-4-methoxy-1-methyl-1H-benzimidazol-2-amine,
(19) N-(2-bromo-4-chlorophenyl)-7-(1-ethylpropyl)-4-methoxy-1-methyl-1H-benzimidazol-2-amine,
(20) N-(2,4-dichlorophenyl)-7-(1-ethylpropyl)-4-methoxy-1-methyl-1H-benzimidazol-2-amine,
(21) 4-chloro-N′-[7-(1-ethylpropyl)-4-methoxy-1-methyl-1H-benzimidazol-2-yl]-N²,N²-dimethylbenzene-1,2-diamine,
(22) 5-chloro-2-{[7-(1-ethylpropyl)-4-methoxy-1-methyl-1H-benzimidazol-2-yl]oxy}benzenesulfonamide
(23) 2-(2-bromo-4,6-dichlorophenoxy)-4-chloro-7-(1-ethylpropyl)-1-methyl-1H-benzimidazole,
(24) 3,5-dichloro-2-{[4-chloro-7-(1-ethylpropyl)-1-methyl-1H-benzimidazol-2-yl]oxy}-N,N-dimethylaniline,
(25) 4-chloro-2-(2,4-dichloro-6-morpholin-4-ylphenoxy)-7-(1-ethylpropyl)-1-methyl-1H-benzimidazole,
(26) 4-chloro-2-[2,4-dichloro-6-(4-methylpiperazin-1-yl)phenoxy]-7-(1-ethylpropyl)-1-methyl-1H-benzimidazole,
(27) 4-chloro-2-[2,4-dichloro-6-(1H-imidazol-1-yl)phenoxy]-7-(1-ethylpropyl)-1-methyl-1H-benzimidazole,
(28) 3,5-dichloro-2-{[4-chloro-7-(1-ethylpropyl)-1-methyl-1H-benzimidazol-2-yl]oxy}-N-methylaniline,
(29) 4-chloro-2-(2,4-dichloro-6-pyrrolidin-1-ylphenoxy)-7-(1-ethylpropyl)-1-methyl-1H-benzimidazole,
(30) 2-{[7-(1-ethylpropyl)-4-methoxy-1-methyl-1H-benzimidazol-2-yl]oxy}-N,N-dimethyl-5-(trifluoromethoxy)aniline,
(31) 3,5-dichloro-2-{[4-chloro-7-(1-ethylpropyl)-1-methyl-1H-benzimidazol-2-yl]oxy}benzonitrile,
(32) 3,5-dichloro-2-{[4-chloro-7-(1-ethylpropyl)-1-methyl-1H-benzimidazol-2-yl]oxy}benzenesulfonamide,
(33) 2-(4-bromo-2-chloro-6-methylphenoxy)-4-chloro-7-(1-ethylpropyl)-1-methyl-1H-benzimidazole,
(34) 3-chloro-4-{[4-chloro-7-(1-ethylpropyl)-1-methyl-1H-benzimidazol-2-yl]oxy}-5-methylbenzonitrile,
(35) 1-(3-chloro-4-{[4-chloro-7-(1-ethylpropyl)-1-methyl-1H-benzimidazol-2-yl]oxy}-5-methylphenyl)ethanone,
(36) 4-chloro-2-[2-chloro-6-methyl-4-(methylthio)phenoxy]-7-(1-ethylpropyl)-1-methyl-1H-benzimidazole,
(37) 4-chloro-2-[2-chloro-6-methyl-4-(methylsulfinyl)phenoxy]-7-(1-ethylpropyl)-1-methyl-1H-benzimidazole,
(38) 4-chloro-2-[2-chloro-6-methyl-4-(methylsulfonyl)phenoxy]-7-(1-ethylpropyl)-1-methyl-1H-benzimidazole,
(39) 3-chloro-4-{[4-chloro-7-(1-ethylpropyl)-1-methyl-1H-benzimidazol-2-yl]oxy}-5-methylbenzamide,
(40) 3-chloro-4-{[4-chloro-7-(1-ethylpropyl)-1-methyl-1H-benzimidazol-2-yl]oxy}-5-methylbenzenesulfonamide,
(41) 2-[2-bromo-6-chloro-4-(trifluoromethoxy)phenoxy]-4-chloro-7-(1-ethylpropyl)-1-methyl-1H-benzimidazole,
(42) 4-chloro-2-[2-chloro-6-(methylthio)-4-(trifluoromethoxy)phenoxy]-7-(1-ethylpropyl)-1-methyl-1H-benzimidazole,
(43) 4-chloro-2-[2-chloro-6-(methylsulfinyl)-4-(trifluoromethoxy)phenoxy]-7-(1-ethylpropyl)-1-methyl-1H-benzimidazole,
(44) 4-chloro-2-[2-chloro-6-(methylsulfonyl)-4-(trifluoromethoxy)phenoxy]-7-(1-ethylpropyl)-1-methyl-1H-benzimidazole,
(45) 4-chloro-7-(1-ethylpropyl)-N-[2-methoxy-6-methyl-4-(methylthio)phenyl]-1-methyl-1H-benzimidazol-2-amine,
(46) 4-chloro-7-(1-ethylpropyl)-N-[2-methoxy-6-methyl-4-(methylsulfinyl)phenyl]-1-methyl-1H-benzimidazol-2-amine,
(47) 4-chloro-7-(1-ethylpropyl)-N-[2-methoxy-6-methyl-4-(methylsulfonyl)phenyl]-1-methyl-1H-benzimidazol-2-amine,
(48) 4-{[4-chloro-7-(1-ethylpropyl)-1-methyl-1H-benzimidazol-2-yl]amino}-3-methoxy-5-methylbenzamide,
(49) N-(2-bromo-4-chloro-6-methylphenyl)-4-chloro-7-(1-ethylpropyl)-1-methyl-1H-benzimidazol-2-amine,
(50) 4-chloro-N-[4-chloro-2-methyl-6-(methylthio)phenyl]-7-(1-ethylpropyl)-1-methyl-1H-benzimidazol-2-amine,
(51) 4-chloro-N-[4-chloro-2-methyl-6-(methylsulfinyl)phenyl]-7-(1-ethylpropyl)-1-methyl-1H-benzimidazol-2-amine,
(52) 3-{[4-chloro-7-(1-ethylpropyl)-1-methyl-1H-benzimidazol-2-yl]oxy}-4-methoxy-N,N,6-trimethylpyridin-2-amine,
(53) 4-chloro-7-(1-ethylpropyl)-1-methyl-2-[(2-dimethyl-2,4,5,6-tetrahydrocyclopenta[c]pyrazol-3-yl)oxy]-1H-benzimidazole,
(54) 4-chloro-7-(1-ethylpropyl)-1-methyl-2-[(2-methyl-2,3-dihydro-1H-isoindol-4-yl)oxy]-1H-benzimidazole,
(55) 4-chloro-2-[(5,7-dichloro-2-methyl-2,3-dihydro-1H-isoindol-4-yl)oxy]-7-(1-ethylpropyl)-1-methyl-1H-benzimidazole,
(56) 4-chloro-7-(1-ethylpropyl)-2-{[4-methoxy-2-methyl-6-(trifluoromethyl)pyridin-3-yl]oxy}-1-methyl-1H-benzimidazole,
(57) 4-chloro-7-(1-ethylpropyl)-N-[2-methoxy-6-methyl-4-(1H-pyrazol-1-yl)phenyl]-1-methyl-1H-benzimidazol-2-amine,
(58) 4-chloro-7-(1-ethylpropyl)-N-{2-methoxy-6-methyl-4-[3-(trifluoromethyl)-1H-pyrazol-1-yl]phenyl}-1-methyl-1H-benzimidazol-2-amine,
(59) 4-chloro-2-[2-chloro-6-methyl-4-(trimethylsilyl)phenoxy]-7-(1-ethylpropyl)-1-methyl-1H-benzimidazole,
(60) 4-chloro-2-[2,4-dichloro-6-(trimethylsilyl)phenoxy]-7-(1-ethylpropyl)-1-methyl-1H-benzimidazole,
(61) N-(4-chloro-2-methoxy-6-methylphenyl)-1-meth-yl-7-[1-(methylamino)propyl]-1H-benzimidazol-2-amine,
(62) N-(4-chloro-2-methoxy-6-methylphenyl)-7-[1-(dimethylamino)propyl]-1-methyl-1H-benzimidazol-2-amine,
(63) 2-[4-bromo-2-(2,4-dichloro-6-methylphenoxy)-7-(1-ethylpropyl)-1H-benzimidazol-1-yl]-N,N-dimethylethanamine,
(64) 4-chloro-2-(2,4-dichloro-6-methylphenoxy)-7-(1-ethylpropyl)-1H-benzimidazole,
(65) 4-chloro-2-[2,6-dichloro-4-(trifluoromethoxy)phenoxy]-7-(1-ethylpropyl)-1H-benzimidazole,
(66) 4-chloro-N-(2,4-dichloro-6-methylphenyl)-7-(1-ethylpropyl)-1H-benzimidazol-2-amine,
(67) 4-chloro-N-(4-chloro-2-methoxy-6-methylphenyl)-7-(1-ethylpropyl)-1H-benzimidazol-2-amine,
(68) 4-chloro-2-[2,6-dichloro-4-(trifluoromethoxy)phenoxy]-7-(1-ethylpropyl)-1-{2-[(4-methoxybenzyl)oxy]ethyl}-1H-benzimidazole or
(69) 2-[4-chloro-2-[2,6-dichloro-4-(trifluoromethoxy)phenoxy]-7-(1-ethylpropyl)-1H-benzimidazol-1-yl]ethanol.

A salt of a compound of the formula (I) includes, for example, a metal salt, an ammonium salt or a salt with an organic base, a salt with an inorganic acid, an organic acid, or a basic or acidic amino acid. Preferable examples of a metal salt include alkali metal salts such as a sodium salt or a potassium salt; alkaline earth metal salts such as a calcium salt, a magnesium salt or a barium salt; and an aluminum salt. Preferable examples of a salt with an organic base include salts with trimethylamine, triethylamine, pyridine, picoline, ethanolamine, diethanolamine, triethanolamine, dicyclohexylamine, and N,N-dibenzylethylenediamine. Preferable examples of a salt with an inorganic acid include salts with hydrochloric acid, hydrobromic acid, nitric acid, sulfuric acid and phospholic acid. Preferable examples of a salt with an organic acid include salts with formic acid, acetic acid, trifluoroacetic acid, fumaric acid, oxalic acid, tartaric acid, maleic acid, citric acid, succinic acid, malic acid, methanesulfonic acid, benzenesulfonic acid, and p-toluenesulfonic acid. Preferable examples of a salt with basic amino acid include salts with arginine, lysine and ornithine. Preferable examples of a salt with an acidic amino acid include salts with aspartic acid and glutamic acid.

Among them, pharmaceutically acceptable salts are preferable. Examples thereof include inorganic salts such as alkali metal salts (e.g. sodium salt, potassium salt etc.) or alkaline earth metal salts (e.g. calcium salt, magnesium salt, barium salt etc.) and an ammonium salt when the compound has an acidic functional group, and inorganic salts such as hydrochloride, sulfate, phosphate or hydrobromide and organic salts such as acetate, maleate, fumarate, succinate, methanesulfonate, p-toluenesulfonate, citrate or tartrate when the compound has a basic functional group.

Compound (I) may be a hydrate or a non-hydrate. The hydrate is exemplified by semihydrate, monohydrate, sesquihydrate and dihydrate. When Compound (I) is present as a configuration isomer, diastereomer, conformer and the like, then it can be isolated if desired by an ordinary separation or purification procedure. When Compound (I) is present as a racemate, it can be resolved into S form and R form by an ordinary optical resolution method.

When Compound (I) has its stereoisomers, then individual isomers or a mixture thereof may also encompassed in this invention.

Compound (I) may be in the form of a prodrug thereof. The prodrug of compound (I) refers' to a compound that is converted into compound (I) by a reaction with an enzyme, gastric acid, or the like under a physiological condition in the living body, namely, (i) a compound that is converted into compound (I) by an enzymatic oxidation, reduction, hydrolysis, or the like, and (ii) a compound that is converted into compound (I) by hydrolysis with gastric acid or the like. Examples of a prodrug of compound (I) to be used include a compound or its salt wherein hydroxy in compound (I) is acylated, alkylated, phosphorylated, or converted into borate (e.g., a compound or its salt wherein hydroxy in compound (I) is converted into acetyloxy, palmitoyloxy, propanoyloxy, pivaloyloxy, succinyloxy, fumaryloxy, alanyloxy, dimethylaminomethylcarbonyloxy, etc.), a compound or its salt wherein carboxy in compound (I) is esterified or amidated (e.g., a compound or its salt wherein carboxy in compound (I) is subjected to ethyl esterification, phenyl esterification, dimethylaminomethyl esterification, pivaloyloxymethyl esterification, ethoxycarbonyloxyethyl esterification, phthalidyl esterification, (5-methyl-2-oxo-1,3-dioxolan-4-yl)methyl esterification, cyclohexyloxycarbonyl esterification, or conversion into the methyl amide, etc.), or the like. These prodrugs can be produced according to a per se known method or its modified method.

Further, a prodrug of compound (I) may be a compound or its salt that is converted into compound (I) under physiological conditions as described in “Development of Drugs”, Volume 7, Molecular Design, Hirokawa Shoten, 1990; pages 163-198.

### General Synthetic Method

Production of a compound of formula (I) or a salt thereof of the present invention is discussed below. The following examples are given to illustrate the invention and are not intended to be inclusive in any manner. Alternative methods may be employed by one skilled in the art, and substituents of compound (I) or a salt thereof may be converted to other substituents by known arts.

A process for preparing Compound (I) of the present invention is shown in the following methods. In the scheme, the compound may contain salt forms, and examples of the salts are the same as salts of Compound (I), and the like. The sequence of each step for preparing compound (I) may be changed. Each of the materials in the scheme can be used as it is when it can be commercially available, and it can be produced in accordance with the known methods per se or analogous methods thereof.

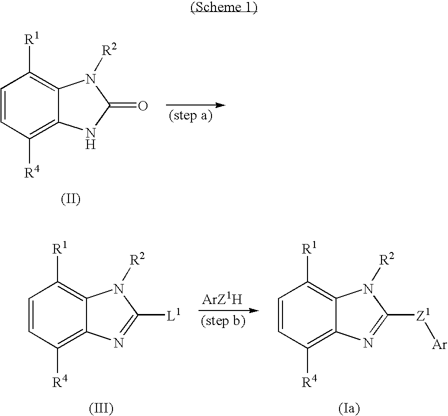

wherein L¹ is a leaving group including a halogen atom such as chlorine, bromine and iodine, sulfonyloxy group such as p-toluenesulfonyloxy group, methanesulfonyloxy group and trifluoromethanesulfonyloxy group, etc. and acyloxy group such as acetyloxy group and benzoyloxy group, etc., Ar is an optionally substituted aryl, Z¹ is —O—, —S—, or —NR⁵— wherein R⁵ is a hydrogen or a C₁₋₆ alkyl, and each of other symbols has a meaning defined above.

Compound (II) or the salt thereof can be prepared by the methods described in Schemes 6 and 7.

Compound (III) or a salt thereof can be prepared by halogenation, sulfonylation or acylation of compound (II) or a salt thereof with a halogenation agent, a sulfonylation agent or an acylation agent, respectively.

Examples of a halogenation agent include phosphorous oxychloride, phosphorous oxybromide, phosphorous trichloride, phosphorous tribromide, phosphorous pentachloride, chlorine, bromine, sulfuryl chloride and thionyl chloride. The halogenation agent is employed in an amount of 1 mole to excess per 1 mole of compound (II) or as a solvent.

Examples of the solvents having no adverse effect on the reaction include aromatic hydrocarbons such as benzene, toluene and xylene, ethers such as diethyl ether, dioxane and tetrahydrofuran, esters such as ethyl acetate, nitriles such as acetonitrile, halogenated hydrocarbon such as 1,2-dichloroethane, chloroform and dichloromethane, amides such as N,N-dimethylformamide, N,N-dimethylacetamide and 1-methyl-2-pirrolidinone, ketones such as acetone and 2-butanone and sulfoxides such as dimethylsulfoxide. These solvents may be used by mixing at an appropriate ratio.

While the reaction temperature may vary depending on compound (II) or a salt thereof employed as well as other conditions, it is 0 to 200° C., preferably 20 to 150° C. The reaction time is 10 minutes to 180 hours, preferably 30 minutes to 96 hours.

The thus obtained compound (III) can be isolated and purified by the known isolating and purifying methods, for example, concentration under reduced pressure, extraction with solvents, crystallization, recrystallization, transfer dissolution and chromatography.

When L¹ is a sulfonyloxy or an acyloxy group in compound (III) or a salt thereof, compound (III) or a salt thereof can be prepared by reacting compound (II) with a sulfonylation agent or an acylation agent after base treatment of compound (II).

A base may for example be an alkaline metal hydroxide such as sodium hydroxide and potassium hydroxide, etc., an alkaline metal hydrogen carbonate such as sodium hydrogen carbonate and potassium hydrogen carbonate, etc., an alkaline metal carbonate such as sodium carbonate and potassium carbonate, etc., a cesium salt such as cesium carbonate, etc., an alkaline metal hydride such as sodium hydride and potassium hydride, etc., sodium amide, an alkoxide such as sodium methoxide and sodium ethoxide, etc., an amine such as trimethylamine, triethylamine and diisopropylethylamine, etc. and a cyclic amine such as pyridine, etc.

Examples of a sulfonylation agent include p-toluenesulfonylchloride, methanesulfonylchloride, trifluoromethanesulfonylchloride, etc. The sulfonylation agent is employed in an amount of 1 to 10 moles, preferably 1 to 5 moles per 1 mole of compound (II).

Examples of an acylation agent include acetylchloride and benzoylchloride, etc. The acylation agent is employed in an amount of 1 to 10 moles, preferably 1 to 5 moles per 1 mole of compound (II).

Examples of the solvents having no adverse effect on the reaction include water, alcohols such as methanol and ethanol, ethers such as diethyl ether, dioxane and tetrahydrofuran, aromatic hydrocarbons such as benzene, toluene and xylene, esters such as ethyl acetate, halogenated hydrocarbons such as 1,2-dichloroethane, chloroform and dichloromethane, nitriles such as acetonitrile, amides such as N,N-dimethylformamide and N,N-dimethylacetamide and sulfoxides such as dimethylsulfoxide. These solvents may be used by mixing at an appropriate ratio.

While the reaction temperature may vary depending on compound (II) or a salt thereof as well as other conditions, it is 0 to 200° C., preferably 0 to 150° C. The reaction time is 10 minutes to 24 hours, preferably 30 minutes to 12 hours.

The thus obtained compound (III) can be isolated and purified by the known isolating and purifying methods, for example, concentration under reduced pressure, extraction with solvents, crystallization, recrystallization, transfer dissolution and chromatography.

Compound (Ia), which is encompassed within compound (I) of the invention, can be prepared by reacting compound (III) with ArZ¹H.

In this step, 1 to 20 moles, preferably 1 to 10 moles of a compound represented by ArZ¹H or a salt thereof are employed per 1 mole of compound (III) or a salt thereof.

This reaction may be performed under basic conditions. A base may for example be an alkaline metal hydroxide such as sodium hydroxide and potassium hydroxide, etc., an alkaline metal hydrogen carbonate such as sodium hydrogen carbonate and potassium hydrogen carbonate, etc., an alkaline metal carbonate such as sodium carbonate and potassium carbonate, etc., a cesium salt such as cesium carbonate, etc., an alkaline metal hydride such as sodium hydride and potassium hydride, etc., sodium amide, an alkaline metal alkoxide such as sodium methoxide, sodium ethoxide, sodium tert-butoxide and potassium tert-butoxide, etc., an amine such as trimethylamine, triethylamine and diisopropylethylamine, etc. and a cyclic amine such as pyridine, etc.

In this step, a palladium catalyst and a catalytic phosphine ligand may be employed. Examples of palladium catalysts may, for example, be tetrakis(triphenylphosphine)palladium(0), bis(triphenylphosphine)palladium(II) dichloride, tris(dibenzylidineacetone)dipalladium(0), trans-dichlorobis(tri-o-tolylphosphine)palladium, palladium(II) trifluoroacetate and palladium(II) acetate, etc.

Examples of catalytic phosphine ligands may, for example, be triphenylphosphine, 2,2′-bis(diphenylphosphino)-1,1′-binaphtyl, 2-(di-tert-butylphosphino)biphenyl, 2-(dicyclohexylphosphino)biphenyl, 2-(dicyclohexylphosphino)-2′,6′-dimethoxy-1,1′-biphenyl, 2-(dicyclohexylphosphino)-2′-(N,N-dimethylamino)biphenyl, 1,1′-bis(diphenylphosphino)ferrocene, tri-tert-butylphosphine and tricyclohexylphosphine, etc.

Examples of the solvents having no adverse effect on the reaction include water, alcohols such as methanol and ethanol, ethers such as diethyl ether, dioxane and tetrahydrofuran, aromatic hydrocarbons such as benzene, toluene and xylene, esters such as ethyl acetate, halogenated hydrocarbons such as 1,2-dichloroethane, chloroform and dichloromethane, nitrites such as acetonitrile, amides such as N,N-dimethylformamide, N,N-dimethylacetamide and 1-methyl-2-pyrrolidinone, ketones such as acetone and 2-butanone and sulfoxides such as dimethylsulfoxide. These solvents may be used by mixing at an appropriate ratio, or may not be used.

While the reaction temperature may vary depending on compound (III) or a salt thereof as well as other reaction conditions, it is 0 to 200° C., preferably 20 to 150° C., or the reaction may be heated by microwave irradiation. The reaction time is 5 minutes to 180 hours, preferably 5 minutes to 96 hours.

The thus obtained compound (Ia) can be isolated and purified by the known isolating and purifying methods, for example, concentration, concentration under reduced pressure, extraction with solvents, crystallization, recrystallization, transfer dissolution and chromatography.

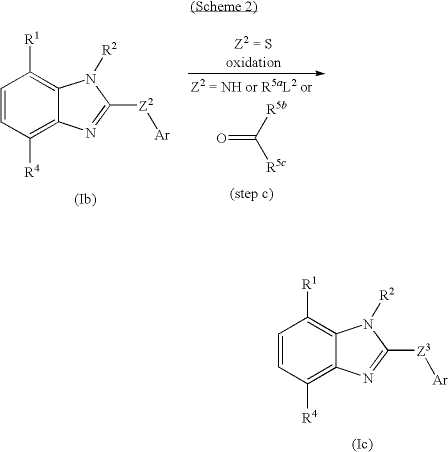

wherein Z² is —S— or —NH—, Z³ is —SO—, —SO₂— or —NR^{5a}— wherein R^{5a} is a C₁₋₆ alkyl, R^{5b} is a C₁₋₅ alkyl, R^{5c} is a hydrogen or a C₁₋₄ alkyl, L² is a halogen atom such as chlorine, bromine and iodine, and each of other symbols has a meaning defined above.

Compound (Ic), which is encompassed within compound (I) of the invention, can be prepared from compound (Ib) by oxidation, alkylation or reductive alkylation of compound (Ib) with an oxidation agent, R^{5a}L² or R^{5b}R^{5c}C═O.

When Z² is —S— in compound (Ib), an amount of 1 to 10 moles, preferably 1 to 5 moles of an oxidation agent, preferably hydrogen peroxide, organic peroxides such as 3-chloroperoxybenzoic acid and peroxyacetic acid, etc., manganese (IV) oxide and sodium metaperiodate, etc., are employed per 1 mole of compound (Ib).

This reaction may be performed under acidic conditions. An acid employed in this oxidation may, for example, be an inorganic acid such as hydrochloric acid, sulfuric acid and nitric acid, etc., and an ordinary organic acid such as formic acid, acetic acid, trifluoroacetic acid and methanesulfonic acid, etc. as well as a Lewis acid.

A reaction solvent may, for example, be water, alcohols such as methanol and ethanol, etc., ethers such as diethyl ether, dioxane and tetrahydrofuran, etc., aromatic hydrocarbons such as benzene, toluene and xylene, etc., esters such as ethyl acetate, etc., halogenated hydrocarbons such as 1,2-dichloroethane, chloroform and dichloromethane, etc., nitriles such as acetonitrile, etc., amides such as N,N-dimethylformamide and N,N-dimethylacetamide, etc. and sulfoxides such as dimethylsulfoxide, etc. These solvents may be used by mixing at an appropriate ratio.

While the reaction temperature may vary depending on compound (Ib) as well as other conditions, it is −20 to 200° C., preferably 0 to 100° C. The reaction time is usually 5 minutes to 24 hours, preferably 5 minutes to 10 hours.

When Z² is NH in compound (Ib), 1 to 10 moles, preferably 1 to 5 moles of a compound represented by R^{5a}L² or a salt thereof and 1 to 10 moles, preferably 1 to 3 moles of a base are employed per 1 mole of compound (Ib).

A base may for example be an alkaline metal hydroxide such as sodium hydroxide and potassium hydroxide, etc., an alkaline metal hydrogen carbonate such as sodium hydrogen carbonate and potassium hydrogen carbonate, etc., an alkaline metal carbonate such as sodium carbonate and potassium carbonate, etc., a cesium salt such as cesium carbonate, etc., an alkaline metal hydride such as sodium hydride and potassium hydride, etc., sodium amide, an alkoxide such as sodium methoxide and sodium ethoxide, etc., an amine such as trimethylamine, triethylamine and diisopropylethylamine, etc. and a cyclic amine such as pyridine, etc.

Examples of solvents having no adverse effect on the reaction include alcohols such as methanol and ethanol, ethers such as diethyl ether, dioxane and tetrahydrofuran, aromatic hydrocarbons such as benzene, toluene and xylene, esters such as ethyl acetate, halogenated hydrocarbons such as 1,2-dichloroethane, chloroform and dichloromethane, nitriles such as acetonitrile, amides such as N,N-dimethylformamide and N,N-dimethylacetamide, and sulfoxides such as dimethylsulfoxide. These solvents may be used by mixing at an appropriate ratio.

While the reaction temperature may vary depending on compound (Ib) as well as other reaction conditions, it is −20 to 200° C., preferably 0 to 150° C. The reaction time is 5 minutes to 48 hours, preferably 5 minutes to 24 hours.

When Z² is NH, compound (Ic) can be also prepared by reacting compound (Ib) with R^{5b}R^{5c}C═O by production of an imine which is then reduced by an appropriate reducing agent or hydrogenation in the presence of a hydrogen catalyst.

In this step, 1 to excess, preferably 1 to 20 moles of a compound represented by R^{5b}R^{5c}C═O or a salt thereof are employed per 1 mole of compound (Ib).

A reducing agent is preferably sodium borohydride, lithium borohydride, sodium cyanoborohydride and sodium triacetoxyborohydride, and 1 to 20 moles, preferably 1 to 10 moles of a reducing agent is employed per 1 mole of compound (Ib).

A hydrogenation catalyst is preferably a palladium catalyst such as palladium black, palladium oxide, palladium barium sulfate, palladium on carbon, palladium hydroxide, a platinum catalyst such as platinum black, platinum oxide and platinum on carbon, or nickel catalyst such as reduced nickel, oxidized nickel or Raney nickel.

Examples of the solvents having no adverse effect on the reaction include water, alcohols such as methanol and ethanol, ethers such as diethyl ether, dioxane and tetrahydrofuran, aromatic hydrocarbons such as benzene, toluene and xylene, esters such as ethyl acetate, halogenated hydrocarbons such as 1,2-dichloroethane, chloroform and dichloromethane, nitriles such as acetonitrile, amides such as N,N-dimethylformamide, N,N-dimethylacetamide and 1-methyl-2-pyrrolidinone, ketones such as acetone and 2-butanone and sulfoxides such as dimethylsulfoxide. These solvents may be used by mixing at an appropriate ratio, or may not be used.

When producing an imine, use of molecular sieves or addition of an acid such as acetic acid and trifluoroacetic acid, etc., or a Lewis acid such as trihalogenated boron (e.g. boron trichloride and boron trifluoride), tetrahalogenated titanium (e.g. titanium tetrachloride, titanium tetrabromide and titanium(IV) isopropoxide) and halogenated aluminium (e.g. aluminium chloride and aluminium bromide) serves to promote the reaction.

While the reaction temperature in this imine production may vary depending on compound (III) or a salt thereof as well as other conditions, it is −50 to 150° C., preferably 0 to 100° C. The reaction time is 30 minutes to 48 hours, preferably 1 hour to 24 hours.

The thus obtained compound (Ic) can be isolated and purified by the known isolating and purifying methods, for example, concentration, concentration under reduced pressure, extraction with solvents, crystallization, recrystallization, transfer dissolution and chromatography.

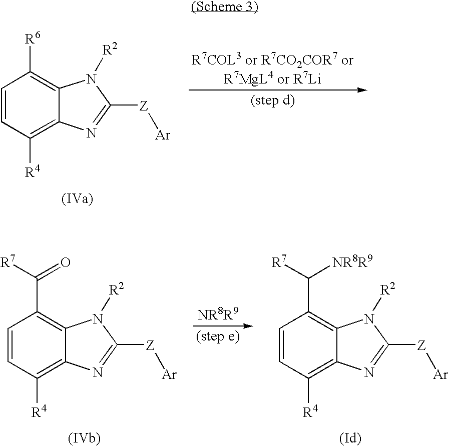

wherein R⁶ is a halogen atom such as chlorine, bromine and iodine, a cyano or derivatives of carboxylic acid such as carboxylic acid, acid halide, ester and amide, etc., R⁷ is an optionally substituted C₁₋₅ alkyl, R⁸ and R⁹ are independently hydrogen or optionally substituted C₁₋₆ alkyl, L³ is a halogen atom such as fluorine, chlorine, bromine and iodine, L⁴ is a halogen atom such as chlorine, bromine and iodine, and each of other symbols has a meaning defined above.

Compound (IVa) or the salt thereof can be prepared by the similar method described in Scheme 5, and R⁶ may be converted to other R⁶ in compound (IVa) or the salt thereof.

When R⁶ is a halogen atom in compound (IVa) or a salt thereof, compound (IVb) can be prepared by reacting compound (IVa) or a salt thereof with an acid halide represented by R⁷COL₃ or a salt thereof or an acid anhydride represented by R⁷CO₂COR⁷ after treating by an organic metal reagent.

An organic metal reagent may be alkyl lithium, such as n-butyl lithium, sec-butyl lithium and tert-butyl lithium, etc. and is employed in an amount of 1 to 10 moles, preferably 1 to 5 moles per 1 mole of compound (IVa) or a salt thereof.

Examples of the solvents having no adverse effect on the reaction include aromatic hydrocarbons such as benzene, toluene and xylene, ethers such as diethyl ether, dioxane and tetrahydrofuran, and halogenated hydrocarbon such as 1,2-dichloroethane, chloroform and dichloromethane. These solvents may be used by mixing at an appropriate ratio.

While the reaction temperature may vary depending on compound (IVa) or a salt thereof as well as other conditions, it is −100 to 100° C., preferably −80 to 50° C. The reaction time is 10 minutes to 24 hours, preferably 30 minutes to 12 hours.

When R⁶ is a cyano or derivatives of carboxylic acid such as carboxylic acid, acid halide, ester and amide, etc. in compound (IVa) or a salt thereof, compound (IVb) or a salt thereof can be prepared by Grignard reaction of compound (IVa) or a salt thereof with R⁷MgL⁴ or by alkylation with R⁷Li.

When R⁷MgL⁴ is employed, 1 to 20 moles, preferably 1 to 10 moles of a compound represented by R⁷MgL⁴ or a salt thereof are employed per 1 mole of compound (IVa) or a salt thereof.

Examples of the solvents having no adverse effect on the reaction include ethers such as diethyl ether, dioxane and tetrahydrofuran, aromatic hydrocarbons such as benzene, toluene and xylene, halogenated hydrocarbons such as 1,2-dichloroethane, chloroform and dichloromethane, ketones such as acetone and 2-butanone and sulfoxides such as dimethylsulfoxide. These solvents may be used by mixing at an appropriate ratio, or may not be used.

While the reaction temperature may vary depending on compound (IVa) or a salt thereof as well as other reaction conditions, it is −20 to 150° C., preferably 0 to 100° C. The reaction time is 5 minutes to 48 hours, preferably 5 minutes to 24 hours.

When R⁷Li is employed, 1 to 20 moles, preferably 1 to 10 moles of a compound represented by R⁷Li or a salt thereof are employed per 1 mole of compound (IVa) or a salt thereof.

Examples of the solvents having no adverse effect on the reaction include ethers such as diethyl ether, dioxane and tetrahydrofuran, aromatic hydrocarbons such as benzene, toluene and xylene, halogenated hydrocarbons such as 1,2-dichloroethane, chloroform and dichloromethane, ketones such as acetone and 2-butanone and sulfoxides such as dimethylsulfoxide. These solvents may be used by mixing at an appropriate ratio, or may not be used.

While the reaction temperature may vary depending on compound (IVa) or a salt thereof as well as other reaction conditions, it is −100 to 150° C., preferably −80 to 100° C. The reaction time is 5 minutes to 48 hours, preferably 5 minutes to 24 hours.

The thus obtained compound (IVb) can be isolated and purified by the known isolating and purifying methods, for example, concentration, concentration under reduced pressure, extraction with solvents, crystallization, recrystallization, transfer dissolution and chromatography.

Compound (Id), which is encompassed within compound (I) of the invention, can be prepared from compound (IVb) and NR⁸R⁹ by reductive amination in the similar method described for reductive alkylation in step c in Scheme 2.

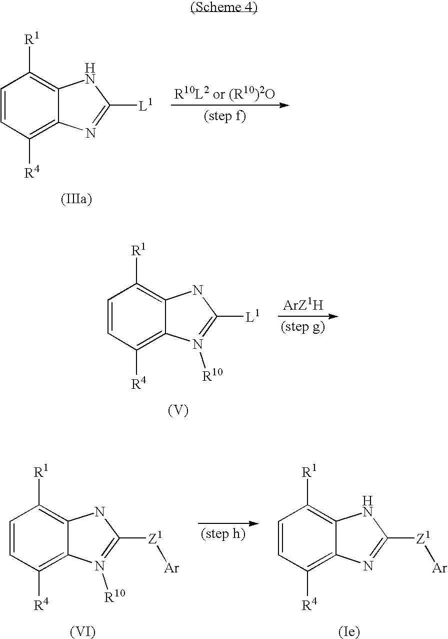

wherein R¹⁰ is an optionally substituted C₁₋₆ alkylcarbonyl such as methylcarbonyl and ethylcarbonyl, etc., phenylcarbonyl, a C₁₋₆ alkyloxycarbonyl such as methoxycarbonyl, ethoxycarbonyl and tert-butoxycarbonyl, etc., phenyloxycarbonyl, benzyloxycarbonyl, etc., C₇₋₁₀ aralkylcarbonyl such as benzylcarbonyl, etc., C₇₋₁₀ aralkyl such as benzyl, etc., trityl, phthaloyl, etc. and each of the groups listed above may be substituted optionally. The substituent on each of the groups listed above may be a halogen atom such as fluorine, chlorine, bromine and iodine, etc., a C₁₋₆ alkoxy such as methoxy, etc., a C₁₋₆ alkylcarbonyl such as methylcarbonyl, ethylcarbonyl and butylcarbonyl, etc. and a nitro, and each of other symbols has a meaning defined above.

Compound (V) or a salt thereof can be prepared by reacting compound (IIIa) with R¹⁰-L² or anhydride (R¹⁰)₂O.

In this step, 1 to 10 moles, preferably 1 to 5 moles of a compound represented by R¹⁰-L² or anhydride (R¹⁰)₂O or a salt thereof are employed per 1 mole of compound (IIIa) or a salt thereof.

This reaction may be performed under basic conditions. A base may for example be an alkaline metal hydroxide such as sodium hydroxide and potassium hydroxide, etc., an alkaline metal hydrogen carbonate such as sodium hydrogen carbonate and potassium hydrogen carbonate, etc., an alkaline metal carbonate such as sodium carbonate and potassium carbonate, cesium carbonate, etc., an alkaline metal hydride such as sodium hydride and potassium hydride, etc., sodium amide, an alkaline metal alkoxide such as sodium methoxide, sodium ethoxide, sodium tert-butoxide and potassium tert-butoxide, etc., an amine such as trimethylamine, triethylamine and diisopropylethylamine, etc., a cyclic amine such as pyridine, 4-dimethylaminopyridine and DBU, etc.

Examples of the solvents having no adverse effect on the reaction include water, alcohols such as methanol and ethanol, ethers such as diethyl ether, dioxane and tetrahydrofuran, aromatic hydrocarbons such as benzene, toluene and xylene, esters such as ethyl acetate, halogenated hydrocarbons such as 1,2-dichloroethane, chloroform and dichloromethane, nitrites such as acetonitrile, amides such as N,N-dimethylformamide, N,N-dimethylacetamide and 1-methyl-2-pyrrolidinone, ketones such as acetone and 2-butanone and sulfoxides such as dimethylsulfoxide. These solvents may be used by mixing at an appropriate ratio, or may not be used.

While the reaction temperature may vary depending on compound (IIIa) or a salt thereof as well as other reaction conditions, it is 0 to 150° C., preferably 0 to 100° C., or the reaction may be heated by microwave irradiation. The reaction time is 5 minutes to 48 hours, preferably 5 minutes to 24 hours.

The thus obtained compound (V) can be isolated and purified by the known isolating and purifying methods, for example, concentration, concentration under reduced pressure, extraction with solvents, crystallization, recrystallization, transfer dissolution and chromatography.

Preparation of Compound (VI) or a Salt Thereof from compound (V) or a salt thereof can be carried out in the similar method described in step b in Scheme I.

Compound (Ie), which is encompassed within compound (I) of the invention, can be prepared by deprotection of compound (VI) with an acid or a base or catalytic hydrogenation.

An acid may for example be an inorganic acid such as hydrochloric acid, sulfuric acid, nitric acid and thionyl chloride, etc. and an ordinary organic acid such as formic acid, acetic acid, trifluoroacetic acid and methanesulfonic acid, etc. as well as a Lewis acid.

A base may, for example, be an alkaline metal hydroxide such as sodium hydroxide and potassium hydroxide, etc., an alkaline metal hydrogen carbonate such as sodium hydrogen carbonate and potassium hydrogen carbonate, etc., an alkaline metal carbonate such as sodium carbonate and potassium carbonate, cesium carbonate, etc., an alkaline metal hydride such as sodium hydride and potassium hydride, etc., sodium amide, an alkaline metal alkoxide such as sodium methoxide, sodium ethoxide, sodium tert-butoxide and potassium tert-butoxide, etc.

Catalytic hydrogenation may be performed in this step. A hydrogenation catalyst is preferably a palladium catalyst such as palladium black, palladium oxide, palladium barium sulfate, palladium on carbon, palladium hydroxide, a platinum catalyst such as platinum black, platinum oxide and platinum on carbon, or nickel catalyst such as reduced nickel, oxidized nickel or Raney nickel.

In this step, 1 mole to excess of an acid or a base is employed per 1 mole of compound (VI) or a salt thereof, or an acid may be employed as a solvent.

Examples of the solvents having no adverse effect on the reaction include water, alcohols such as methanol and ethanol, ethers such as diethyl ether, dioxane and tetrahydrofuran, aromatic hydrocarbons such as benzene, toluene and xylene, esters such as ethyl acetate, halogenated hydrocarbons such as 1,2-dichloroethane, chloroform and dichloromethane, nitrites such as acetonitrile, amides such as N,N-dimethylformamide, N,N-dimethylacetamide and 1-methyl-2-pyrrolidinone, ketones such as acetone and 2-butanone and sulfoxides such as dimethylsulfoxide. These solvents may be used by mixing at an appropriate ratio or may not be used.

While the reaction temperature may vary depending on compound (VI) or a salt thereof as well as other reaction conditions, it is 0 to 200° C., preferably 20 to 150° C. The reaction time is 5 minutes to 48 hours, preferably 5 minutes to 24 hours.

The thus obtained compound (Ie) can be isolated and purified by the known isolating and purifying methods, for example, concentration, concentration under reduced pressure, extraction with solvents, crystallization, recrystallization, transfer dissolution and chromatography.

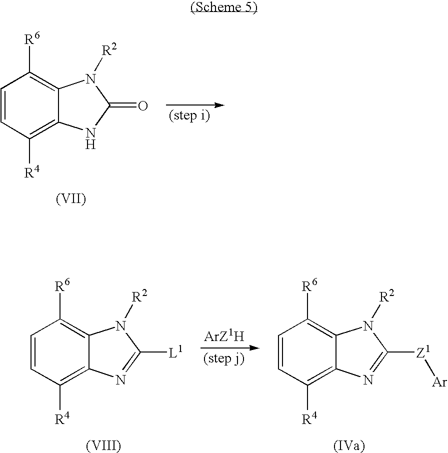

wherein each symbol has a meaning defined above.

Compound (VII) or the salt thereof can be prepared by the methods described in Schemes 7, 8 and 9. Preparation of compound (IVa) or a salt thereof from compound (VII) or a salt thereof via a compound (VIII) or a salt thereof can be carried out in the similar method described in step a and step b in Scheme 1.

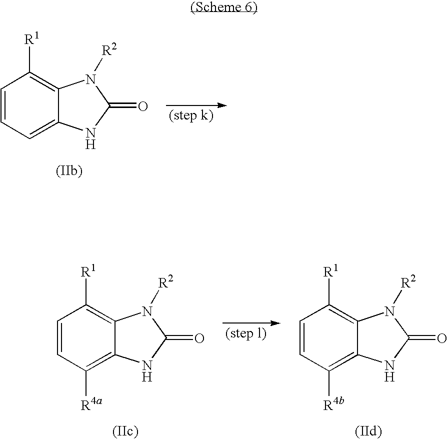

wherein R^{4a} is a halogen atom such as chlorine, bromine and iodine, R^{4b} is a cyano, a C₁₋₆ alkyl, a C₁₋₆ alkoxy, and each of other symbols has a meaning defined above.

Compound (IIc) or a salt thereof can be prepared by reaction of compound (IIb) with a halogenation agent.

A halogenation agent may for example be chlorine, bromine, iodine, thionyl chloride, sulfuryl chloride, N-chlorosuccinimide, N-bromosuccinimide, N-iodosuccinimide, phosphorous oxychloride, phosphorous oxybromide, phosphorous trichloride, phosphorous tribromide, phosphorous pentachloride, potassium bromide, potassium bromate, hydrochloric acid, hydrobromic acid, hydroiodic acid, sodium chloride, sodium bromide, sodium iodide, aluminum chloride and aluminum bromide. The halogenation agent is employed in an amount of 1 mole to 5 moles, preferably 1 mole to 3 moles per 1 mole of compound (IIb).

In this step, catalytic amount to 2 moles, preferably catalytic amount to 1 mole of a radical initiator such as 2,2′-azobis(isobutyronitrile), 2,2′-azobis(4-methoxy-2,4-dimethylvaleronitrile, benzoylperoxide and m-chloroperbenzoic acid may be employed per 1 mole of compound (IIb).

An acid, a base and an additive may also be employed in this step.

An acid may, for example, be an inorganic acid such as hydrochloric acid, sulfuric acid and nitric acid, etc., and an ordinary organic acid such as formic acid, acetic acid, trifluoroacetic acid and methanesulfonic acid, etc. as well as a Lewis acid. The acid is employed in an amount of 0.1 mole to excess, preferably 1 mole to excess per 1 mole of compound (IIb) or as a solvent.

A base may for example be an alkaline metal hydroxide such as sodium hydroxide and potassium hydroxide, etc., an alkaline metal hydrogen carbonate such as sodium hydrogen carbonate and potassium hydrogen carbonate, etc., an alkaline metal carbonate such as sodium carbonate, potassium carbonate, and cesium carbonate, etc., an alkaline metal hydride such as sodium hydride and potassium hydride, etc., sodium amide, an alkoxide such as sodium methoxide and sodium ethoxide, etc., an amine such as trimethylamine, triethylamine and diisopropylethylamine, etc. and a cyclic amine such as pyridine, etc. The base is employed in an amount of 1 mole to 10 moles, preferably 1 mole to 5 moles per 1 mole of compound (IIb) or as a solvent.

An additive such as iron, reductive iron and a Lewis acid may be employed in an appropriate amount.

Examples of the solvents having no adverse effect on the reaction include water, alcohols such as methanol and ethanol, ethers such as diethyl ether, dioxane and tetrahydrofuran, aromatic hydrocarbons such as benzene, chlorobenzene, toluene and xylene, esters such as ethyl acetate, halogenated hydrocarbons such as 1,2-dichloroethane, carbon tetrachloride, chloroform and dichloromethane, nitriles such as acetonitrile, amides such as N,N-dimethylformamide, N,N-dimethylacetamide and 1-methyl-2-pyrrolidinone, ketones such as acetone and 2-butanone, sulfoxides such as dimethylsulfoxide and acids such as acetic acid. These solvents may be used by mixing at an appropriate ratio.

While the reaction temperature may vary depending on compound (IIb) or a salt thereof as well as other reaction conditions, it is −20 to 150° C., preferably 0 to 100° C. The reaction time is 5 minutes to 48 hours, preferably 5 minutes to 24 hours.

The thus obtained compound (IIc) can be isolated and purified by the known isolating and purifying methods, for example, concentration, concentration under reduced pressure, extraction with solvents, crystallization, recrystallization, transfer dissolution and chromatography.

When R^{4b} is a cyano or a C₁₋₆ alkyl, compound (IId) or a salt thereof can be prepared from compound (IIc) or a salt thereof with a cyanation agent or an alkylation agent by a coupling reaction.

A cyanation agent may for example be sodium cyanide, potassium cyanide, copper(I) cyanide, zinc(II) cyanide, palladium(II) cyanide, and is employed in an amount of 1 mole to 10 moles, preferably 1 mole to 5 moles per 1 mole of compound (IIc).

A alkylation agent may for example be a C₁₋₆ alkyl boronic acid such as methylboronic acid, ethylboronic acid and isopropylboronic acid, a C₁₋₆ alkyl borane such as triethylborane, trimethylboroxine, a C₁₋₆ alkyl stannane such as tetramethyltin and tetraethyltin, a C₁₋₆ alkyl halide, such as methyliodide, ethylbromide and ethyliodide and C₁₋₆ alkylmagnesium halide such as methylmagnesium chloride, methylmagnesium bromide, methylmagnesium iodide and ethylmagnesium iodide, and is employed in an amount of 1 mole to 20 moles, preferably 1 mole to 10 moles per 1 mole of compound (IIc).

In this step, a palladium catalyst and a catalytic phosphine ligand may be employed. Examples of palladium catalysts may for example be tetrakis(triphenylphosphine)palladium(0), bis(triphenylphosphine)palladium(II) dichloride, tris(dibenzylidineacetone)dipalladium(0), trans-dichlorobis(tri-o-tolylphosphine)palladium, palladium(II) trifluoroacetate and palladium(II) acetate, etc.

Examples of catalytic phosphine ligands may, for example, be triphenylphosphine, 2,2′-bis(diphenylphosphino)-1,1′-binaphtyl, 2-(di-tert-butylphosphino)biphenyl, 2-(dicyclohexylphosphino)biphenyl, 2-(dicyclohexylphosphino)-2′,6′-dimethoxy-1,1′-biphenyl, 2-(dicyclohexylphosphino)-2′-(N,N-dimethylamino)biphenyl, 1,1′-bis(diphenylphosphino)ferrocene, tri-tert-butylphosphine and tricyclohexylphosphine, etc.

A base may, for example, be an alkaline metal hydroxide such as sodium hydroxide and potassium hydroxide, etc., an alkaline metal hydrogen carbonate such as sodium hydrogen carbonate and potassium hydrogen carbonate, etc., an alkaline metal carbonate such as sodium carbonate, potassium carbonate and cesium carbonate, etc., an alkaline metal hydride such as sodium hydride and potassium hydride, etc., sodium amide, an alkoxide such as sodium methoxide, sodium ethoxide, sodium tert-butoxide and potassium tert-butoxide, etc., an alkaline metal such as lithium, sodium, and potassium, an amine such as trimethylamine, triethylamine and diisopropylethylamine, etc., a cyclic amine such as pyridine, etc.

This reaction may be carried out in the presence of additives. Examples of the additives include copper(I) iodide, copper(II) sulfate, sodium iodide, potassium iodide, zinc(II) bromide, 18-crown-6 and phase transfer catalyst such as tetrabutylammonium bromide and benzyltriethylammonium chloride, etc.

In this step, 0.01 to 0.5 moles, preferably 0.05 to 0.2 moles of a palladium catalyst, 0.01 to 0.5 moles, preferably 0.02 to 0.2 moles of a phosphine ligand, 1.0 to 5.0 moles, preferably 1.2 to 3 moles of a base and 0.01 to 2.0 moles, preferably 0.05 to 1.0 moles of an additive are employed per 1 mole of compound (IIc) or a salt thereof.

Another metal catalysts may be employed. Examples of the other metal catalysts may for example be a copper ate complex, which may be produced from compound (IIc) or the lithium salt of compound (IIc) and C₁₋₆ alkyl lithium with copper bromide in situ. The copper ate complex may be employed in an amount of 1.0 to 5.0 moles, preferably 1.0 to 3 moles per 1 mole of compound (IIc) or a salt thereof.

Examples of solvents having no adverse effect on the reaction include water, alcohols such as methanol, ethanol, ethylene glycol and 2-methoxyethanol, ethers such as diethyl ether, dioxane, tetrahydrofuran and 1,2-dimethoxyethane, aromatic hydrocarbons such as benzene, toluene and xylene, halogenated hydrocarbons such as 1,2-dichloroethane, chloroform and dichloromethane, nitriles such as acetonitrile, amides such as N,N-dimethylformamide, N,N-dimethylacetamide, 1-methyl-2-pyrrolidone and hexamethylphosphoramide, ketones such as acetone and 2-butanone, sulfoxides such as dimethylsulfoxide and pyridine. These solvents may be used by mixing at an appropriate ratio, or may not be used.

While the reaction temperature may vary depending on compound (IIc) or a salt thereof as well as other reaction conditions, it is 0 to 250° C., preferably 50 to 200° C., or the reaction may be heated by microwave irradiation. The reaction time is 5 minutes to 120 hours, preferably 5 minutes to 48 hours.

When R^{4b} is a C₁₋₆ alkoxy, compound (IId) or a salt thereof can be prepared from compound (IIc) or a salt thereof with a C₁₋₆ alkoxide, which may be commercially available or produced in situ from a corresponding alcohol and a base.

The alkoxide is employed in an amount of 1 mole to excess per 1 mole of compound (IIc) or may be employed as a solvent.

In this step, a base may be employed. Examples of bases include an alkaline metal hydroxide such as sodium hydroxide and potassium hydroxide, etc., an alkaline metal hydrogen carbonate such as sodium hydrogen carbonate and potassium hydrogen carbonate, etc., an alkaline metal carbonate such as sodium carbonate, potassium carbonate and cesium carbonate, etc., an alkaline metal hydride such as sodium hydride and potassium hydride, etc., sodium amide, an alkoxide such as sodium methoxide and sodium ethoxide, etc., an alkaline metal such as lithium, sodium and potassium, an amine such as trimethylamine, triethylamine and diisopropylethylamine, etc. and a cyclic amine such as pyridine, etc. The base may be employed in an amount of 1 mole to excess, preferably 1 mole to 20 moles per 1 mole of compound (IIc).

An additive may also be employed in an amount of catalytic amount to 1 mole per 1 mole of compound (IIc). Examples of the additives include copper(I) iodide, copper(I) cyanide, copper(II) chloride, copper(I) bromide, manganese(II) oxide, manganese(IV) oxide, tetrabutylammonium bromide and collidine.

Examples of solvents having no adverse effect on the reaction include water, alcohols such as the corresponding alcohol and ethylene glycol, ethers such as dioxane, tetrahydrofuran and 1,2-dimethoxyethane, aromatic hydrocarbons such as benzene, toluene and xylene, halogenated hydrocarbons such as 1,2-dichloroethane, chloroform and dichloromethane, nitriles such as acetonitrile, amides such as N,N-dimethylformamide, N,N-dimethylacetamide, 1-methyl-2-pyrrolidone and hexamethylphosphoramide, sulfoxides such as dimethylsulfoxide and pyridine. These solvents may be used by mixing at an appropriate ratio.

While the reaction temperature may vary depending on compound (IIc) or a salt thereof as well as other reaction conditions, it is 0 to 250° C., preferably 50 to 200° C., or the reaction may be heated by microwave irradiation. The reaction time is 5 minutes to 120 hours, preferably 5 minutes to 48 hours.

The thus obtained compound (IId) can be isolated and purified by the known isolating and purifying methods, for example, concentration, concentration under reduced pressure, extraction with solvents, crystallization, recrystallization, transfer dissolution and chromatography.

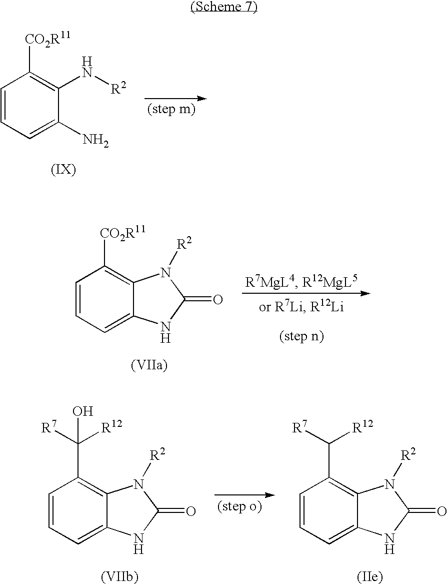

wherein R¹¹ is hydrogen and an optionally substituted C₁₋₆ alkyl, R⁷ and R¹² are independently optionally substituted C₁₋₆ alkyl, L⁴ and L⁵ are independently halogen atoms such as chlorine, bromine and iodine, and R² has a meaning defined above.

Compound (VIIa) or a salt thereof can be prepared by treatment of compound (IX) with 1,1′-carbonyl diimidazole, phosgene, triphosgene, alkyl haloformate such as ethyl chloroformate, phenyl haloformate such as phenyl chloroformate or urea, etc. Compound (IX) or a salt thereof is mainly commercially available or can be prepared from the nitro derivatives corresponded to compound (IX).

In this step, 1 to 5 moles, preferably 1 to 3 moles of an agent or a salt thereof for cyclization are employed per 1 mole of compound (IX) or a salt thereof.

Examples of the solvents having no adverse effect on the reaction include water, alcohols such as methanol and ethanol, ethers such as diethyl ether, dioxane and tetrahydrofuran, aromatic hydrocarbons such as benzene, toluene and xylene, esters such as ethyl acetate, halogenated hydrocarbons such as 1,2-dichloroethane, chloroform and dichloromethane, nitriles such as acetonitrile, amides such as N,N-dimethylformamide, N,N-dimethylacetamide and 1-methyl-2-pyrrolidinone, ketones such as acetone and 2-butanone and sulfoxides such as dimethylsulfoxide. These solvents may be used by mixing at an appropriate ratio, or may not be used.

While the reaction temperature may vary depending on compound (IX) or a salt thereof as well as other reaction conditions, it is 0 to 150° C., preferably 0 to 100° C. The reaction time is 5 minutes to 48 hours, preferably 5 minutes to 24 hours.

The thus obtained compound (VIIa) can be isolated and purified by the known isolating and purifying methods, for example, concentration, concentration under reduced pressure, extraction with solvents, crystallization, recrystallization, transfer dissolution and chromatography.

Compound (VIIb) or a salt thereof can be prepared in the similar method described in step d in Scheme 3. Provided that the Grignard reactions may be performed stepwise by R⁷MgL⁴ and R¹²MgL⁵, when R⁷ is not equal to R¹² in compound (VIIb). The reaction using lithium reagents, R⁷L¹ and R¹²Li, is also carried out stepwise, when R⁷ is not equal to R¹² in compound (VIIb).

Compound (IIe) or a salt thereof can be prepared by dehydration of compound (VIIb) or a salt thereof with an acid, and reduction of the olefine obtained or a salt thereof with an appropriate reducing agent or catalytic hydrogenation.

An acid may, for example, be an inorganic acid such as hydrochloric acid, sulfuric acid, nitric acid and thionyl chloride, etc., and an ordinary organic acid such as formic acid, acetic acid, trifluoroacetic acid and methanesulfonic acid, etc. as well as a Lewis acid.

In dehydration step, 1 mole to excess of an acid is employed per 1 mole of compound (VIIb) or a salt thereof or an acid may be employed as a solvent.

Examples of the solvents having no adverse effect on the reaction include water, alcohols such as methanol and ethanol, ethers such as diethyl ether, dioxane and tetrahydrofuran, aromatic hydrocarbons such as benzene, toluene and xylene, esters such as ethyl acetate, halogenated hydrocarbons such as 1′,2-dichloroethane, chloroform and dichloromethane, nitrites such as acetonitrile, amides such as N,N-dimethylformamide, N,N-dimethylacetamide and 1-methyl-2-pyrrolidinone, ketones such as acetone and 2-butanone and sulfoxides such as dimethylsulfoxide. These solvents may be used by mixing at an appropriate ratio, or may not be used.

While the reaction temperature may vary depending on compound (VIIb) or a salt thereof as well as other reaction conditions, it is −20 to 200° C., preferably −20 to 150° C. The reaction time is 5 minutes to 48 hours, preferably 5 minutes to 24 hours.

The thus obtained olefine can be isolated and purified by the known isolating and purifying methods, for example, concentration, concentration under reduced pressure, extraction with solvents, crystallization, recrystallization, transfer dissolution and chromatography.

In reduction step, a reducing agent is preferably sodium borohydride, lithium borohydride, sodium cyanoborohydride and sodium triacetoxyborohydride.

Catalytic hydrogenation may be performed in this step. A hydrogenation catalyst is preferably a palladium catalyst such as palladium black, palladium oxide, palladium barium sulfate, palladium on carbon, palladium hydroxide, a platinum catalyst such as platinum black, platinum oxide and platinum on carbon, or nickel catalyst such as reduced nickel, oxidized nickel or Raney nickel.

In this step, 1 to 2.0 moles, preferably 1 to 10 moles of a reducing agent are employed per 1 mole of the olefine or a salt thereof.

Examples of the solvents having no adverse effect on the reaction include water, alcohols such as methanol and ethanol, ethers such as diethyl ether, dioxane and tetrahydrofuran, aromatic hydrocarbons such as benzene, toluene and xylene, esters such as ethyl acetate, halogenated hydrocarbons such as 1,2-dichloroethane, chloroform and dichloromethane, nitrites such as acetonitrile, amides such as N,N-dimethylformamide, N,N-dimethylacetamide and 1-methyl-2-pyrrolidinone, ketones such as acetone and 2-butanone and sulfoxides such as dimethylsulfoxide. These solvents may be used by mixing at an appropriate ratio.

While the reaction temperature may vary depending on the olefine or a salt thereof as well as other reaction conditions, it is 0 to 150° C., preferably 0 to 100° C. The reaction time is 5 minutes to 48 hours, preferably 5 minutes to 24 hours.

The thus obtained compound (IIe) can be isolated and purified by the known isolating and purifying methods, for example, concentration, concentration under reduced pressure, extraction with solvents, crystallization, recrystallization, transfer dissolution and chromatography.

These two steps, dehydration and reduction, may be carried out at the same time, which is that an acid and a reducing agent or a catalyst may simultaneously be employed.

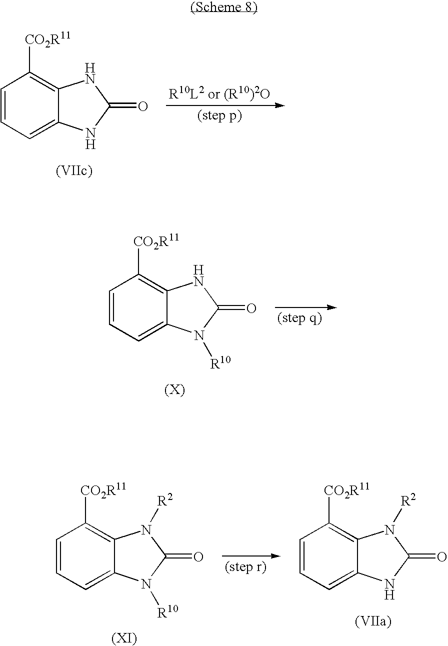

wherein each symbol has a meaning defined above.

Compound (X) or a salt thereof can be prepared from compound (VIIc) or a salt thereof in the similar method described in step f in Scheme 4.

Compound (XI) or a salt thereof can be prepared from compound (X) or a salt thereof in the similar method described in step f in Scheme 4.

Compound (VIIa) or a salt thereof can be prepared from compound (XI) or a salt thereof in the similar method described in step h in Scheme 4.

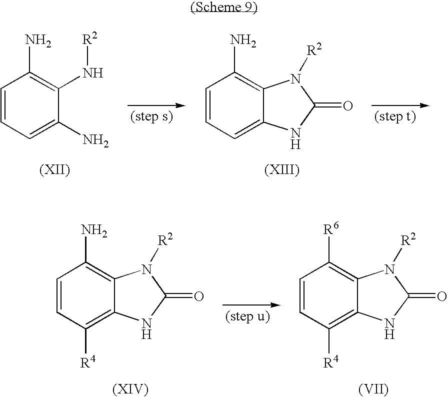

wherein each of symbols has a meaning defined above.

In step s, compound (XIII) or a salt thereof can be prepared by treatment of compound (XII) with 1,1′-carbonyl diimidazole, phosgene, alkyl haloformate such as ethyl chloroformate, phenyl haloformate such as phenyl chloroformate or urea, etc. Compound (XII) or a salt thereof is mainly commercially available or can be prepared from the nitro derivatives corresponded to compound (XII).

Examples of the solvent include ethers such as dioxane and tetrahydrofuran, aromatic hydrocarbons such as benzene, toluene and xylene, esters such as ethyl acetate, halogenated hydrocarbons such as chloroform and dichloromethane, nitrites such as acetonitrile, amides such as N,N-dimethylformamide and N,N-dimethylacetamide, and sulfoxides such as dimethylsulfoxide. These solvents may be used by mixing at an appropriate ratio.

While the reaction temperature may vary depending on the reagent employed as well as other conditions, it is −20 to 200° C., preferably 20 to 100° C. The reaction time is 5 minutes to 48 hours, preferably 30 minutes to 24 hours.

The thus obtained compound (XIII) can be isolated and purified by the known isolating and purifying methods, for example, concentration, concentration under reduced pressure, extraction with solvent, crystallization, recrystallization, transfer dissolution and chromatography.

In step t, compound (XIV) or a salt thereof can be prepared from compound (XIII) or a salt thereof in the similar method described in steps k and l in Scheme 6.

In step u, compound (VII) or a salt thereof can be prepared by halogenation of compound (XIV) or a salt thereof with a halogenation agent.

Examples of a halogenation agent include chlorine, bromine, iodine, thionyl chloride, copper (I) chloride, copper (II) chloride, copper (I) bromide, copper (II) bromide, sodium chloride, sodium bromide, sodium iodide, potassium iodide, etc. The halogenation agent is employed in an amount of 0.5 moles to 10 moles, preferably 0.5 moles to 5 moles, per 1 mole of compound (XIV).

In this step, the diazonium salt type compound of compound (XIV) may be produced before introduction of a halogen atom. Examples of an agent to produce the diazonium salt type compound include sodium nitrite, potassium nitrite and tert-butyl nitrite, etc. The agent is employed in an amount of 1 mole to 10 moles, preferably 1 mole to 5 moles, per 1 mole of compound (XIV).

This reaction can be carried out under an acidic condition. Examples of an acid include an inorganic acid such as hydrochloric acid, hydrobromic acid, hydroiodic acid, sulfuric acid, a nitric acid and copper sulfate, etc., as well as Lewis acid. An acid is employed in an amount of 2 moles to excess per 1 mole of compound (XIV).

Examples of solvent having no adverse effect on the reaction include water, alcohols such as methanol and ethanol, ethers such as diethyl ether, dioxane and tetrahydrofuran, aromatic hydrocarbons such as benzene, toluene and xylene, esters such as ethyl acetate, halogenated hydrocarbons such as chloroform and dichloromethane, nitrites such as acetonitrile, amides such as N,N-dimethylformamide, N,N-dimethylacetamide and 1-methyl-2-pyrrolidinone, ketones such as acetone and 2-butanone and sulfoxides such as dimethylsulfoxide. These solvents may be used by mixing at an appropriate ratio.

While the reaction temperature may vary depending on compound (XIV) or a salt thereof employed as well as other conditions, it is −20 to 150° C., preferably 0 to 100° C. The reaction time is 10 minutes to 24 hours, preferably 30 minutes to 12 hours.

The thus obtained compound (VII) can be isolated and purified by the known isolating and purifying methods, for example, concentration under reduced pressure, extraction with solvent, crystallization, recrystallization, transfer dissolution and chromatography.

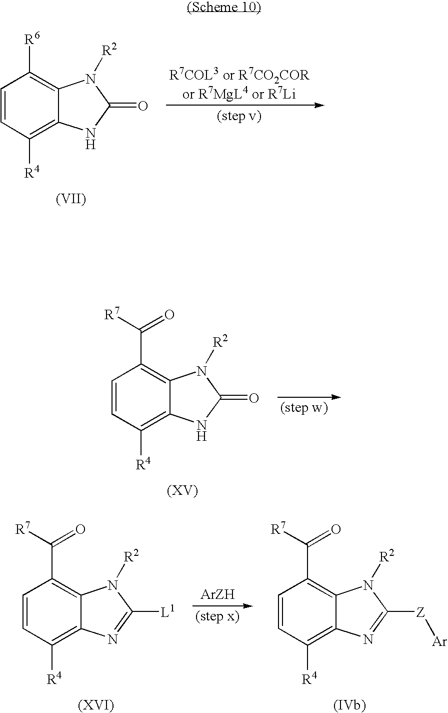

wherein each of symbols has a meaning defined above.

In step v, compound (XV) or a salt thereof can be prepared from compound (VII) or a salt thereof in the similar method described in step d in Scheme 3.

In step w, compound (XVI) or a salt thereof can be prepared from compound (XV) or a salt thereof in the similar method described in step i in Scheme 5.

In step x, compound (IVb) or a salt thereof can be prepared from compound (XVI) or a salt thereof in the similar method described in step j in Scheme 5.

A starting compound for Compound (I) may be in a form of a salt, including a salt with an inorganic acid (for example, hydrochloric acid, phosphoric acid, hydrobromic acid and sulfuric acid, etc.) and a salt with an organic acid (for example, acetic acid, formic acid, propionic acid, fumaric acid, maleic acid, succinic acid, tartaric acid, citric acid, malic acid, oxalic acid, benzoic acid, methanesulfonic acid and benzenesulfonic acid, etc.). When any of these compounds carries an acidic group such as carboxy, etc., a salt with an inorganic base (for example, an alkaline metal or an alkaline earth metal such as sodium, potassium, calcium and magnesium, ammonia, etc.) or with an organic base (for example, tri-C₁₋₃ alkylamine such as triethylamine, etc.) may be formed.

In each of the reactions described above, when a starting compound carries as a substituent an amino group, an amide group, a hydrazine group, a urea group, a carboxyl group or a hydroxyl group, then such group may be derivatized with a protective group employed ordinarily in peptide chemistry, which is cleaved after a reaction if desired to yield an intended compound.

A protective group for an amino, an amide and a urea may for example be an optionally substituted C₁₋₆ alkylcarbonyl (for example, methylcarbonyl and ethylcarbonyl, etc.), phenylcarbonyl, a C₁₋₆ alkyloxycarbonyl (for example, methoxycarbonyl, ethoxycarbonyl and tert-butoxycarbonyl, etc.), phenyloxycarbonyl, benzoxycarbonyl, C₇₋₁₀ aralkylcarbonyl (for example, benzyloxycarbonyl), C₇₋₁₀ aralkyl (for example, benzyl and 4-methoxybenzyl, etc.), trityl, phthaloyl, etc. A substituent on each of the groups listed above may be a halogen atom (for example, fluorine, chlorine, bromine and iodine, etc.), a C₁₋₆ alkylcarbonyl (for example, methylcarbonyl, ethylcarbonyl and butylcarbonyl, etc.) and a nitro group, which may occur 1 to about 3 times.

A protective group for a carboxy may, for example, be an optionally substituted C₁₋₆ alkyl (for example, methyl, ethyl, n-propyl, isopropyl, n-butyl and tert-butyl, etc.), phenyl, trityl and silyl, etc. A substituent on each of the groups listed above may be a halogen atom (for example, fluorine, chlorine, bromine and iodine, etc.), a C₁₋₆ alkylcarbonyl (for example, methylcarbonyl, ethylcarbonyl and butylcarbonyl, etc.) and a nitro group, which may occur 1 to about 3 times.

A protective group for a hydroxy may for example be an optionally substituted C₁₋₆ alkyl (for example, methyl, ethyl, n-propyl, isopropyl, n-butyl and tert-butyl, etc.), phenyl, a C₇₋₁₀ aralkyl (for example, benzyl and benzyl, etc.), a C₁₋₆ alkylcarbonyl (for example, formyl, methylcarbonyl and ethylcarbonyl, etc.), phenyloxycarbonyl (for example, benzoxycarbonyl, etc.), C₇₋₁₀ aralkylcarbonyl (for example, benzyloxycarbonyl, etc.), pyranyl, furanyl, silyl, etc. A substituent on each of the groups listed above may be a halogen atom (for example, fluorine, chlorine, bromine and iodine, etc.), a C₁₋₆ alkyl, a C₁₋₆ alkoxy (for example, methoxy, etc.), phenyl, a C₇₋₁₀ aralkyl, nitro, etc., which may occur 1 to about 4 times.

A method for cleaving a protective group is a method known per se or an analogous method, such as a treatment for example with an acid, a base, a reduction, UV light, hydrazine, phenylhydrazine, sodium N-methyldithiocarbamate, tetrabutylammonium fluoride, palladium acetate, etc.

Compound (I) of the present invention has an excellent corticotropin releasing factor antagonistic activity, and when orally administered, Compound (I) of the present invention shows good pharmacokinetic profiles and exhibits anxiolytic and antidepressive effects to an animal, especially to a mammal (e.g., human, monkey, dog, cat, rabbit, guinea pig, rat, mouse, and the like). Especially, Compound (I) of the present invention shows an excellent solubility profile, an excellent stability in metabolite and an excellent improvement in pharmacokinetics. In addition, Compound (I) of the present invention is a selective antagonist of CRF1 against a wide range of other receptors and has a low toxicity.

On the basis of that, Compound (I) is useful as a safe pharmaceutical and can be used as a pharmaceutical for preventing and/or treating diseases associated with the functions of a CRF receptor or a CRF, such as depression, major depression, bipolar depression, dysthymia, affective disorder (e.g., seasonal affective disorder), recurrent depression, postpartum depression, suppression symptom, mania, anxiety, generalized anxiety disorder, anxiety syndrome, panic disorder, phobia, social phobia, obsessive-compulsive disorder, posttraumatic stress disorder, stress-induced insomnia, post psychic trauma stress disorder, Tourette's syndrome, autism, passion disorder, adjustment disorder, dysthymic disorder, sleep disorder, insomnia, bipolar disorder, circulatory disease, neurosis, schizophrenia, digestive ulcer, irritable bowel syndrome, inflammatory bowel disease, ulcerative colitis, Crohn's disease, stress-induced gastrointestinal disorder, nervous emesis, peptic ulcer, diarrhea, constipation, postoperative ileus, gastrointestine dysfunction and nervous vomiting associated with stress, Alzheimer's disease, Alzheimer's type senile dementia, nervous degenerated disease such as Parkinson's disease and Huntington's disease, multi-infarct dementia, senile dementia, nervous orexia inactivity, eating disorder, anorexia nervosa, hyperphagia and other ingestion disorder, obesity, diabetes, alcohol dependency, pharmacophinia, drug withdrawal, migraine, stress headache, tension headache, ischemic nervous disorder, nervous disorder, cerebral paralysis, progressive supranuclear palsy, amyotrophic lateral sclerosis, multiple sclerosis, muscular convulsion, chronic fatigue syndrome, glaucoma, Meniere syndrome, autonomic imbalance, alopecia, hypertension, cardiovascular disorder, tachycardia, congestive heart attack, hyperplea, bronchial asthma, apnea, infant sudden death syndrome, inflammatory disorder, pain, allergic disorder, impotence, menopausal disorder, fertilization disorder, infertility, cancer, immune function abnormality at HIV infection, immune functional abnormality due to stress, cerebrospinal meningitis, acromegaly, incontinence, metabolic syndrome or osteoporosis. Especially, Compound (I) can be used as a pharmaceutical for preventing and/or treating affective disorder, depression or anxiety.

When Compound (I) and its prodrug are used as a pharmaceutical for preventing and/or treating diseases described above, the administration route may be oral or parenteral in accordance with the known method per se.

The pharmaceutical composition containing compound (I) of the present invention is expected to be useful in the treatment or/and prevention of diseases associated with the functions of a CRF receptor or a CRF, such as depression, major depression, bipolar depression, dysthymia, affective disorder (e.g., seasonal affective disorder), recurrent depression, postpartum depression, suppression symptom, mania, anxiety, generalized anxiety disorder, anxiety syndrome, panic disorder, phobia, social phobia, obsessive-compulsive disorder, posttraumatic stress disorder, stress-induced insomnia, post psychic trauma stress disorder, Tourette's syndrome, autism, passion disorder, adjustment disorder, dysthymic disorder, sleep disorder, insomnia, bipolar disorder, circulatory disease, neurosis, schizophrenia, digestive ulcer, irritable bowel syndrome, inflammatory bowel disease, ulcerative colitis, Crohn's disease, stress-induced gastrointestinal disorder, nervous emesis, peptic ulcer, diarrhea, constipation, postoperative ileus, gastrointestine dysfunction and nervous vomiting associated with stress, Alzheimer's disease, Alzheimer's type senile dementia, nervous degenerated disease such as Parkinson's disease and Huntington's disease, multi-infarct dementia, senile dementia, nervous orexia inactivity, eating disorder, anorexia nervosa, hyperphagia and other ingestion disorder, obesity, diabetes, alcohol dependency, pharmacophinia, drug withdrawal, migraine, stress headache, tension headache, ischemic nervous disorder, nervous disorder, cerebral paralysis, progressive supranuclear palsy, amyotrophic lateral sclerosis, multiple sclerosis, muscular convulsion, chronic fatigue syndrome, glaucoma, Meniere syndrome, autonomic imbalance, alopecia, hypertension, cardiovascular disorder, tachycardia, congestive heart attack, hyperplea, bronchial asthma, apnea, infant sudden death syndrome, inflammatory disorder, pain, allergic disorder, impotence, menopausal disorder, fertilization disorder, infertility, cancer, immune function abnormality at HIV infection, immune functional abnormality due to stress, cerebrospinal meningitis, acromegaly, incontinence, metabolic syndrome or osteoporosis. Especially, pharmaceutical composition containing compound (I) of the present invention can be used as a pharmaceutical for preventing and/or treating affective disorder, depression or anxiety.

Compound (I) of the present invention can be formulated with a pharmaceutically acceptable carrier and can be orally or parenterally administered as solid formulations such as tablets, capsules, granules, powders, or the like; or liquid formulations such as syrups, injections, or the like. Also, there can be prepared formulations for transdermal administration such as patchings, cataplasms, ointments (including creams), plasters, tapes, lotions, liquids and solutions; suspensions, emulsions, sprays, and the like.

As for a pharmaceutically acceptable carrier, a variety of organic or inorganic carrier substances, which have been conventionally employed as formulation materials, is used and compounded as a bulking agent, a lubricant, a binding agent, and a disintegrator in solid formulations; a vehicle, a solubilizing agent, a suspending agent, an isotonicity agent, a buffering agent, and an analgesic in liquid formulations. If necessary, formulation excipients such as a preservative, an antioxidant, a stabilizer, a coloring agent, a sweetening agent, and the like can be used.

Preferred examples of the bulking agent include lactose, sucrose, D-mannitol, starch, crystalline cellulose, light anhydrous silicic acid, and the like. Preferred examples of the lubricant include magnesium stearate, potassium stearate, talc, colloidal silica, and the like. Preferred examples of the binding agent include crystalline cellulose, α-starch, sucrose, D-mannitol, dextrin, hydroxypropyl cellulose, hydroxypropylmethyl cellulose, polyvinyl pyrrolidone, and the like. Preferred examples of the disintegrator include starch, carboxymethyl cellulose, calcium carboxymethyl cellulose, croscarmellose sodium, sodium carboxymethyl starch, low-substituted hydroxypropyl cellulose, and the like. Preferred examples of the vehicle include water for injection, alcohol, propylene glycol, macrogol, sesame oil, corn oil, and the like.

If necessary, for the purpose of taste masking, enteric coating, or prolonged action, oral formulations can be prepared by coating by a per se known method. Examples of this coating agent include hydroxypropylmethyl cellulose, ethyl cellulose, hydroxymethyl cellulose, hydroxypropyl cellulose, polyoxyethylene glycol, Tween 80, Pluronic F68 [polyoxyethylene (160) polyoxypropylene (30) glycol], cellulose acetate phthalate, hydroxypropylmethyl cellulose phthalate, hydroxymethyl cellulose acetate phthalate, Eudragit (manufactured by Rohm Company, methacrylic acid-acrylic acid copolymer), and the like.

Preferred examples of the solubilizing agent include polyethylene glycol, propylene glycol, benzyl benzoate, ethanol, trisamiomethane, cholesterol, triethanolamine, sodium carbonate, sodium citrate, and the like. Preferred examples of the suspending agent include surface active agents such as stearyltriethanolamine, sodium lauryl sulfate, laurylaminopropionic acid, lecithin, benzalkonium chloride, benzethonium chloride, glycerin monostearate, and the like; hydrophilic, high molecular substances such as polyvinyl alcohol, polyvinyl pyrrolidone, sodium carboxymethyl cellulose, methyl cellulose, hydroxymethyl cellulose, hydroxyethyl cellulose, hydroxypropyl cellulose, and the like; and so on. Preferred examples of the isotonicity agent include sodium chloride, glycerin, D-mannitol, and the like. Preferred examples of the buffering agent include buffer solutions of a phosphate, an acetate, a carbonate, a citrate, or the like. Preferable examples of the analgesic include benzyl alcohol and the like. Preferred examples of the preservative include paraoxybenzoic acid esters, chlorobutanol, benzyl alcohol, phenethyl alcohol, dehydroacetic acid, sorbic acid, and the like. Preferred examples of the antioxidant include sulfites, ascorbic acid, and the like.

The content of Compound (I) in the composition of the present invention is, for example, about 0.01 to about 100% by weight of the whole preparation.

The dose varies depending on an administration subject, an administration route, disease and the like. For example, when Compound (I) is orally administered to an adult as an antidepressant, Compound (I) as an active ingredient may be administered in an amount of about 0.1 to about 20 mg/kg body weight, preferably about 0.2 to about 10 mg/kg body weight, further preferably about 0.5 to about 10 mg/kg body weight, preferably about 0.5 to about 5 mg/kg body weight. The dose may be administered in one or several divided portions per day.

When the pharmaceutical composition containing Compound (I) of the present invention is used for treatment or/and prevention of the above-mentioned diseases, Compound (I) may be used in combination with another active ingredient. Examples of such a concomitant active ingredient include, for example, benzodiazepines (chlordiazepoxide, diazepam, clorazepate dipotassium, lorazepam, clonazepam, alprazolam etc.), L-type calcium channel blockers (pregabalin etc.), tricyclic or tetracyclic antidepressants (imipramine hydrochloride, amitriptyline hydrochloride, desipramine hydrochloride, clomipramine hydrochloride, carpipramine), selective serotonin reuptake inhibitors (fluvoxamine maleate, fluoxetine hydrochloride, citalopram hydrobromide, sertraline hydrochloride, paroxetine hydrochloride, escitalopram oxalate etc.), serotonin and norepinephrine reuptake inhibitors (venlafaxine hydrochloride, duloxetine hydrochloride etc.), norepinephrine reuptake inhibitors (reboxetine mesilate etc.), mirtazapine, trazodone hydrochloride, nefazodone hydrochloride, bupropion hydrochloride, setiptiline maleate, 5-HT_{1A} agonists (buspirone hydrochloride, tandospirone citrate, osemozotan hydrochloride etc.), 5-HT₃ antagonists (cyamemazine etc.), noncardioselective beta-blockers (propranolol hydrochloride, oxprenolol hydrochloride etc.), histamine H₁ antagonists (hydroxyzine hydrochloride etc.), antipsychotic agents (chlorpromazine haloperidol, sulpiride, clozapine, trifluoperazine hydrochloride, fluphenazine hydrochloride, olanzapine, quetiapine fumarate, risperidone, aripiprazole etc.), other anxiolytics (meprobamate), tachykinin antagonist (MK-869 etc.), drugs acting on metabotropic glutamate receptors, CCK antagonists, beta3-adrenocepto agonists (amibegron hydrochloride), GAT-1 inhibitors (tiagabine hydrochloride), N-type calcium channel blockers, carbonic anhydrase type II inhibitors, NMDA glycine site agonists, NMDA antagonist (memantine etc.), peripheral benzodiazepine receptor ligands, vasopressin Vlb antagonist, phosphodiesterase II or IV inhibitors, opioid antagonists, uridine, nicotinic receptor agonist, thyroid hormone (T3 T4), TSH, TRH, MAO inhibitors (phenelzine sulfate, tranylcypromine sulfate, moclobemide etc.), 5-HT_{2A} Antagonists, 5-HT_{2A} inverse agonists, a COMT inhibitor (e.g. entacapone etc.), agents for bipolar disorder (lithium carbonate, valproate semisodium, lamotrigine, riluzole, felbamate etc.), cannabinoid CB1 antagonist (rimonabant etc.), sodium channel blockers, anti ADHD drugs (methylphenidate hydrochloride, methamphetamine hydrochloride etc.), agents for alcoholism, agents for autism, agents for chronic fatigue syndrome, agents for fibromyalgia syndrome, agents for agents for epilepsy, agents for insomnia (etizolam, zopiclone, triazolam, zolpidem, ramelteon, indiplon etc.), agents for smoking cessation therapy, agents for narcolepsy, agents for pain, agents for male and female sexual dysfunction, agents for migraine, agents for pathological gambling, agents for restless legs syndrome, agents for substance dependence, agents for irritable bowel syndrome, Alzheimer's disease treating drugs, Parkinson's Disease treating drugs, an amyotrophic lateral sclerosis treating drug (e.g. riluzole etc., neurotrophic factor etc.), a hyperlipidemia treating drug such as a cholesterol lowering drug [statin series (e.g. sodium pravastatin, atorvastatin, simvastatin, rosuvastatin etc.), fibrate (e.g. clofibrate etc.), a squalene synthase inhibitor], agents for treating abnormal behavior or dromomania accompanied with progression of dementia (e.g. sedative, anti-anxiety drug etc.), an apoptosis inhibitor, a nerve differentiation/regeneration promoting agent, a hypotensive drug, a diabetes treating drug, anti-obesity drugs, a non-steroidal anti-inflammatory drug (e.g. meloxicam, tenoxicam, indomethacin, ibuprofen, celecoxib, rofecoxib, aspirin, indomethacin etc.), a disease modifying anti-rheumatoid drug (DMARD), an anti-cytokine drug (e.g. TNF inhibitor, MAP kinase inhibitor etc.), a steroid drug (e.g. dexamethasone, hexestrol, cortisone acetate etc.), sex hormone or a derivative thereof (e.g. progesterone, estradiol, estradiol benzoate etc.), parathyroid hormone (PTH), and a calcium receptor antagonist, anti-cancer drugs.

Such another active ingredient and the Compound (I) of the present invention may be mixed according to a known per se method to be formulated into one pharmaceutical composition (e.g. a tablet, powder, a granule, a capsule (including a soft capsule), liquid, a injection, a suppository, a sustained-release preparation etc.), or they may be formulated into separate compositions and then administered to the same subject simultaneously or at a certain interval.

### EXAMPLES

The following reference examples, examples and experiments describe the manner and process of making and using the present invention and are illustrative rather than limiting. It is to be understood that there may be other embodiments which fall within the spirit and scope of the present invention as defined by the claims appended hereto.

In the following examples, the room temperature is ranged between 0 to 30° C., melting points were determined on a Yanaco micro melting point apparatus and were uncorrected.

Proton nuclear magnetic resonance (¹H-NMR) spectra were recorded on Varian Mercury-300 (300 MHz). Chemical shifts are given in parts per million (ppm) with tetramethylsilane as an internal standard. Abbreviations are used as follows: s=singlet, d=doublet, t=triplet, q=quartet, quin=quintet, m=multiplet, dd=doublets of doublet, brs=broad singlet. Coupling constants (J values) are given in hertz (Hz).

LC-MS (ESI⁺) was performed on a Micromass ZMD, using a CAPCELL PAK UG-120 ODS (Shiseido Co., Ltd.) column (2.0 mm i.d.×50 mm) with aqueous acetonitrile (10-95%) containing 0.05% trifluoroacetic acid, and a HP-1100 (Agilent Technologies) apparatus for monitoring at 220 nm. Reagents and solvents were obtained from commercial sources and used without further purification.

Chromatographic purification was carried out on silica gel columns (Kieselgel 60, 0.063-0.22 mm, Merck) or on Purif-Pack (SI 60 μm or NH 60 μm, Fuji Silysia, Ltd.).

Preparative TLC purification was conducted using TLC plate (silica gel 60, Merck).

Preparative HPLC purification was performed using a Gilson pumping system in conjunction with a photodiode array detector (Hewlett Packard 1100 series) and a Gilson 215 auto sampler. Separations were achieved using an YMC packed column (CombiPrep ODS-A, 5 μm, 50×20 mm) and a linear gradient (90% H₂O for 1.0 min, a linear gradient from 10-100% for 3.70 min, then 100% acetonitrile for 2.7 min. 25 mL/min.).

Preparative HPLC purification was also performed using a Waters Preparative HPLC system. Separations were achieved using an Develosil ODS-UG-10 column (50×100 mm) with a 10-100% acetonitrile/water containing 0.1% trifluoroacetic acid gradient (flow rate: 150 mL/min) or an YMC packed column (CombiPrep ODS-A, 5 μm, 50×20 mm) with a 5-100% acetonitrile/water containing 0.1% trifluoroacetic acid gradient (flow rate: 25 mL/min).

### Reference Example 1

### 2-Amino-4,6-dichlorophenol

To a suspension of 2,4-dichloro-6-nitrophenol (60.0 g, 288 mmol) in ethanol (250 mL) and water (250 mL) was added portionwise sodium hydrosulfite (251 g, 1.44 mmol). The mixture was stirred at 65° C. for 4 h. The mixture was concentrated in vacuo, diluted with saturated aqueous sodium hydrogen carbonate solution (500 mL), extracted with ethyl acetate (200 mL×4) and washed with brine. The organic layer was dried over anhydrous magnesium sulfate, filtered, and concentrated in vacuo. The residue was purified by flash column chromatography on silica gel eluting with a 0-50% ethyl acetate/n-hexane gradient mixture. The filtrate was concentrated in vacuo to give the solid, which was washed with n-hexane to give the title compound (30.6 g, 172 mmol, 60%) as a colorless powder.

¹H NMR (CDCl₃) δ 3.92 (s, 2H), 5.36 (s, 1H), 6.59 (d, J=2.1 Hz, 1H), 6.71 (d, J=2.1 Hz, 1H).

MS Calcd.: 177; MS Found: 178 (M+H).

### Reference Example 2

### 2,4-Dichloro-6-(dimethylamino)phenol

To a suspension of 2-amino-4,6-dichlorophenol (25.0 g, 140 mmol) in acetonitrile (300 mL) were added formaldehyde (36-38% solution in water; 110 mL), sodium cyanoborohydride (26.1 g, 415 mmol) and acetic acid (6.0 mL). The mixture was stirred at 0° C. for 2 h. The mixture was diluted with saturated aqueous sodium hydrogen carbonate solution (400 mL), concentrated in vacuo, and extracted with ethyl acetate (300 mL×3). The combined organic layer was washed with brine, dried over anhydrous magnesium sulfate, filtered and concentrated in vacuo. The residue was purified by flash column chromatography on silica gel eluting with a 5-20% ethyl acetate/n-hexane gradient mixture. The filtrate was concentrated in vacuo to give the solid, which was recrystallized from diisopropyl ether-n-hexane to give the title compound (18.5 g, 89.7 mmol, 64%) as colorless crystals.

¹H NMR (CDCl₃) δ 2.67 (s, 6H), 6.60 (brs, 1H), 6.98 (d, J=2.7 Hz, 1H), 7.09 (d, J=2.7 Hz, 1H).

MS Calcd.: 205; MS Found: 206 (M+H).

### Reference Example 3

### 4-Chloro-2-(dimethylamino)phenol

The title compound was prepared from 2-amino-4-chlorophenol in the similar method described in Reference example 2.

¹H NMR (CDCl₃) δ 2.63 (s, 6H), 5.01 (s, 1H), 6.85 (d, J=8.4 Hz, 1H), 7.00 (dd, J=2.1 Hz, 8.4 Hz, 1H), 7.10 (d, J=2.1 Hz, 1H).

MS Calcd.: 171; MS Found: 172 (M+H).

### Reference Example 4

### 2,6-Dichloro-4-(dimethylamino)phenol

The title compound was prepared from 4-amino-2,6-dichlorophenol in the similar method described in Reference example 2.

¹H NMR (CDCl₃) δ 2.86 (s, 6H), 5.24 (s, 1H), 6.63 (s, 2H).

MS Calcd.: 205; MS Found: 206 (M+H).

### Reference Example 5

### 2-Chloro-4-(dimethylamino)phenol

The title compound was prepared from 4-amino-2-chlorophenol in the similar method described in Reference example 2.

¹H NMR (CDCl₃) δ 2.85 (s, 6H), 5.05 (s, 1H), 6.62 (dd, J=2.7 Hz, J=9.0 Hz, 1H), 6.70 (d, J=2.7 Hz, 1H), 6.90 (d, J=9.0 Hz, 1H).

MS Calcd.: 171; MS Found: 172 (M+H).

### Reference Example 6

### 2-(Dimethylamino)-6-methylpyridin-3-ol

### i) 2-Amino-6-methylpyridin-3-ol

To a solution of 6-methyl-2-nitropyridin-3-ol (25.0 g, 162 mmol) in acetic acid (400 mL) was added 10% palladium on carbon (50% wet, 2.50 g). The reaction mixture was purged with hydrogen and stirred under balloon pressure hydrogen for 13 h. The catalyst was removed by filtration and the filtrate was concentrated in vacuo. The residue was washed with diisopropyl ether to give the title compound (19.1 g, 153 mmol, 95%) as a colorless powder.

¹H NMR (DMSO-d₆) δ 2.14 (s, 3H), 5.31 (s, 2H), 6.20 (d, J=7.5 Hz, 1H), 6.70 (d, J=7.5 Hz, 1H), 9.09 (s, 1H).

### ii) 2-(Dimethylamino)-6-methylpyridin-3-ol

To a solution of 2-amino-6-methylpyridin-3-ol (19.1 g, 153 mmol) in methanol (200 mL) were added formaldehyde (36-38% solution in water; 130 mL), sodium cyanoborohydride (29.0 g, 459 mmol) and acetic acid (2.0 mL). The mixture was stirred at room temperature for 4 h. The mixture was diluted with saturated aqueous sodium hydrogen carbonate solution (400 mL), concentrated in vacuo and extracted with ethyl acetate (400 mL×3). The combined organic layer was washed with brine, dried over anhydrous magnesium sulfate, filtered and concentrated in vacuo. The residue was purified by flash column chromatography on silica gel eluting with a 30-70% ethyl acetate/n-hexane gradient mixture. The filtrate was concentrated in vacuo to give the solid, which was washed with diisopropyl ether to give the title compound (17.9 g, 118 mmol, 77%) as a colorless powder.

¹H NMR (DMSO-d₆) δ 2.23 (s, 3H), 2.83 (s, 6H), 6.47 (d, J=8.4 Hz, 1H), 6.85 (d, J=8.4 Hz, 1H), 9.17 (s, 1H).

MS Calcd.: 152; MS Found: 153 (M+H).

### Reference Example 7

### 4-Chloro-2-[(dimethylamino)methyl]phenol

### i) 5-Chloro-2-hydroxy-N,N-dimethylbenzamide

To a solution of 5-chlorosalicylic acid (15.0 g, 86.9 mmol) in tetrahydrofuran (90 mL) was added thionyl chloride (8.17 mL, 112 mmol). The mixture was stirred at 75° C. for 3 h. To the mixture were added triethylamine and aqueous dimethylamine (ca. 50%; 38.0 mL) with ice bath cooling. The reaction mixture was concentrated in vacuo, extracted with ethyl acetate (200 mL×3) and washed with aqueous sodium hydrogen carbonate solution and brine. The organic layer was dried over anhydrous magnesium sulfate, filtered, and concentrated in vacuo. The residue was recrystallized from ethyl acetate to give the title compound (10.4 g, 52.1 mmol, 60%) as colorless crystals.

¹H NMR (DMSO-d₆) δ 2.81 (s, 3H), 2.94 (s, 3H), 6.86 (d, J=8.7 Hz, 1H), 7.11 (d, J=2.4 Hz, 1H), 7.23 (dd, J=2.4 Hz, 8.7 Hz, 1H), 10.1 (brs, 1H).

MS Calcd.: 199; MS Found: 200 (M+H).

### ii) 4-Chloro-2-[(dimethylamino)methyl]phenol

To a solution of 5-chloro-2-hydroxy-N,N-dimethylbenzamide (10.2 g, 51.0 mmol) in tetrahydrofuran (250 mL) was added lithium aluminum hydride (1.0 M solution in tetrahydrofuran, 55.0 mL, 55.0 mmol). The mixture was stirred at 0° C. for 2 h. The mixture was poured into water with ice bath cooling, extracted with ethyl acetate (200 mL×3) and washed with brine. The organic layer was dried over anhydrous magnesium sulfate, filtered and concentrated in vacuo. The residue was purified by flash column chromatography on silica gel eluting with a 10-60% ethyl acetate/n-hexane gradient mixture. The filtrate was concentrated in vacuo to give the title compound (560 mg, 3.01 mmol, 6%) as a yellow powder.

¹H NMR (DMSO-d₆) δ 2.32 (s, 6H), 3.60 (s, 2H), 6.74 (d, J=8.4 Hz, 1H), 6.93 (d, J=2.7 Hz, 1H), 7.10 (dd, J=2.7 Hz, 8.4 Hz, 1H), 8.25 (brs, 1H).

MS Calcd.: 185; MS Found: 186 (M+H).

### Reference Example 8

### 6-Methyl-2-(trifluoromethyl)pyridin-3-ol

A mixture of copper iodide (10.5 g, 55.3 mmol) and potassium fluoride (3.21 g, 55.3 mmol) was dried at 80° C. for 2 h in vacuo. To the mixture were added 1-methyl-2-pyrrolidone (40 mL), 2-iodo-6-methylpyridin-3-ol (10.0 g, 42.5 mmol) and trifluoromethyl trimethylsilane (6.93 mL, 46.8 mmol). The mixture was stirred at 50° C. for 10 h. The mixture was poured into aqueous ammonia (50 mL), extracted with ethyl acetate (200 mL×3) and washed with brine. The organic layer was dried over anhydrous magnesium sulfate, filtered and concentrated in vacuo. The residue was purified by flash column chromatography on silica gel eluting with a 5-50% ethyl acetate/n-hexane gradient mixture. The filtrate was concentrated in vacuo and washed with n-hexane to give the title compound (3.28 g, 18.5 mmol, 44%) as a colorless powder.

¹H NMR (DMSO-d₆) δ 2.39 (s, 3H), 7.35 (s, 2H), 10.7 (brs, 1H).

MS Calcd.: 177; Found: 178 (M+H).

### Reference Example 9

### 1,4,5-Trimethyl-1H-pyrazol-3-ol

To a solution of ethyl 2-methylacetoacetate (300 mg, 2.08 mmol) in acetic acid (1.0 mL) was added dropwise methylhydrazine (0.12 mL, 2.28 mmol). The mixture was stirred at 60° C. for 16 h. The mixture was diluted with saturated aqueous sodium hydrogen carbonate solution (40 mL), extracted with ethyl acetate (40 mL×4) and washed with brine. The organic layer was dried over anhydrous magnesium sulfate, filtered and concentrated in vacuo. The residue was purified by flash column chromatography on silica gel eluting with a 0-20% ethyl acetate/n-hexane gradient mixture. The filtrate was concentrated in vacuo, and the residue was washed with diisopropyl ether to give a mixture of the title compound and 1,3,4-trimethyl-1H-pyrazol-5-ol (262 mg, 2.08 mmol, 100%) as a colorless powder. The mixture was used for the reaction in example 14 without further purification.

major isomer

¹H NMR (CDCl₃) δ 1.35 (s, 3H), 2.05 (s, 3H), 3.27 (s, 3H).

minor isomer

¹H NMR (CDCl₃) δ 1.32 (s, 3H), 2.08 (s, 3H), 3.32 (s, 3H).

MS Calcd.: 126; Found: 127 (M+H).

### Reference Example 10

### 4-Chloro-N²,N²-dimethylbenzene-1,2-diamine

### i) 5-Chloro-N,N-dimethyl-2-nitroaniline

A solution of dimethylamine in tetrahydrofuran (2M, 28 mL) was added dropwise to a suspension of 4-chloro-2-fluoro-1-nitrobenzene (4.78 g, 27.2 mmol) and potassium carbonate (3.87 g, 28.0 mmol) in tetrahydrofuran (50 mL) at room temperature. The reaction mixture was stirred overnight. The reaction mixture was concentrated in vacuo. The residue was dissolved in water (200 mL) and ethyl acetate (200 mL). The organic layer was separated, and the aqueous layer was extracted with ethyl acetate (200 mL). The organics were dried over anhydrous magnesium sulfate, filtered and concentrated in vacuo. The residue was purified by flash chromatography eluting with a 10% ethyl acetate/n-hexane mixture to give the title compound as an amorphous (612 mg, 11%).

¹H NMR (CDCl₃) δ 2.90 (s, 6H), 6.75 (dd, J=2.1, 8.7 Hz, 1H), 6.78 (d, J=2.1 Hz, 1H), 7.73 (d, J=8.7 Hz, 1H).

### ii) 4-Chloro-N²,N²-dimethylbenzene-1,2-diamine

A solution of 5-Chloro-N,N-dimethyl-2-nitroaniline (612 mg, 3.05 mmol) in a 50% ethanol/water mixture was combined with sodium hydrosulfite (2.66 g, 15.3 mmol). The reaction mixture was stirred at room temperature for 3 h. The reaction mixture was concentrated in vacuo. The residue was dissolved in water (50 mL) and ethyl acetate (75 mL). The organic layer was separated, and the aqueous layer was extracted with ethyl acetate (50 mL). The organics were dried over anhydrous magnesium sulfate, filtered, and concentrated in vacuo. The residue was purified by flash chromatography eluting with a 20% ethyl acetate/n-hexane mixture to give the title compound as an amorphous (500 mg, 96%).

¹H NMR (CDCl₃) δ 2.65 (s, 6H), 3.92 (s, 2H), 6.63 (d, J=358.7 Hz, 1H), 6.86 (dd, J=2.1, 8.7 Hz, 1H), 6.95 (d, J=2.1 Hz, 1H).

MS Calcd.: 170; Found: 171 (M+H).

### Reference Example 11

### 2-Bromo-6-chloro-4-(trifluoromethoxy)phenol

To a solution of 2-chloro-4-(trifluoromethoxy)phenol (10.0 g, 47.0 mmol) in chlorobenzene (50 mL) was added N-bromosuccinimide (9.20 g, 51.7 mmol). The mixture was stirred at 60° C. for 8 h. The reaction mixture was concentrated in vacuo, diluted with saturated aqueous sodium hydrogen carbonate solution (200 mL), extracted with ethyl acetate (100 mL×4) and washed with brine. The organic layer was dried over anhydrous magnesium sulfate, filtered, and concentrated in vacuo. The residue was purified by flash column chromatography on silica gel eluting with a 0-10% ethyl acetate/n-hexane gradient mixture. The filtrate was concentrated in vacuo to give the title compound (7.50 g, 25.8 mmol, 55%) as a red oil.

¹H NMR (CDCl₃) δ 6.01 (brs, 1H), 7.24 (d, J=2.1 Hz, 1H), 7.34 (d, J=2.1 Hz, 1H).

### Reference Example 12

### 2-(Dimethylamino)-4-methoxy-6-methylpyridin-3-ol

### i) 4-Bromo-6-methyl-2-nitropyridin-3-ol

To a solution of 6-methyl-2-nitropyridin-3-ol (18.0 g, 116 mmol) in methanol (350 mL) were added aqueous sodium hydroxide (30 mL, 240 mmol), sodium acetate (9.51 g, 116 mmol) and bromine (12.0 mL, 234 mmol). The mixture was stirred at 100° C. for 2 h. The mixture was diluted with saturated aqueous ammonium chloride solution (150 mL) and extracted with ethyl acetate (300 mL×4). The combined organic layer was washed with brine, dried over anhydrous magnesium sulfate, filtered and concentrated in vacuo. The residue was purified by flash column chromatography on silica gel eluting with a 10-50% ethyl acetate/n-hexane gradient mixture. The filtrate was concentrated in vacuo to give the title compound (5.40 g, 23.1 mmol, 20%) as an orange solid.

¹H NMR (CDCl₃) δ 2.57 (s, 3H), 7.76 (s, 1H), 10.6 (s, 1H).

### ii) 2-Amino-4-methoxy-6-methylpyridin-3-ol

To a solution of 4-bromo-6-methyl-2-nitropyridin-3-ol (5.30 g, 22.7 mmol) in methanol (15 mL) were added sodium methoxide (28% solution in methanol, 25 mL) and copper iodide (4.33 g, 22.7 mmol). The mixture was stirred at 100° C. for 3 h. The mixture was diluted with saturated aqueous ammonium chloride solution (100 mL) and extracted with ethyl acetate (100 mL×4). The combined organic layer was washed with brine, dried over anhydrous magnesium sulfate, filtered and concentrated in vacuo. The residue was purified by flash column chromatography on silica gel eluting with a 10-50% ethyl acetate/n-hexane gradient mixture. The filtrate was concentrated in vacuo to give 4-methoxy-6-methyl-2-nitropyridin-3-ol (560 mg, 3.04 mmol) as an orange solid. To a solution of 4-methoxy-6-methyl-2-nitropyridin-3-ol (560 mg, 3.04 mmol) in methanol (20 mL) was added 10% palladium on carbon (50% wet, 50.0 mg). The reaction mixture was purged with hydrogen and stirred under balloon pressure hydrogen for 4 h. The catalyst was removed by filtration and the filtrate was concentrated in vacuo. The residue was purified by flash column chromatography on silica gel eluting with a 10-60% ethyl acetate/n-hexane gradient mixture. The filtrate was concentrated in vacuo to give the title compound (106 mg, 0.688 mmol, 3%) as a colorless powder.

¹H NMR (CDCl₃) δ 2.26 (s, 3H), 3.93 (s, 3H), 4.03 (brs, 2H), 5.20 (brs, 1H), 6.14 (s, 1H).

MS Calcd.: 154; MS Found: 155 (M+H).

### iii) 2-(Dimethylamino)-4-methoxy-6-methylpyridin-3-ol

To a solution of 2-amino-4-methoxy-6-methylpyridin-3-ol (50.0 mg, 0.324 mmol) in acetonitrile (4.0 mL) were added formaldehyde (36-38% solution in water; 0.26 mL, 3.24 mmol), sodium cyanoborohydride (61.1 mg, 0.972 mmol) and acetic acid (0.20 mL). The mixture was stirred at 0° C. for 45 min. The mixture was diluted with saturated aqueous sodium hydrogen carbonate (20 mL) and extracted with ethyl acetate (10 mL×4). The combined organic layer was washed with brine, dried over anhydrous magnesium sulfate, filtered and concentrated in vacuo. The residue was washed with n-hexane to give the title compound (59.0 mg, 0.324 mmol, 100%) as a colorless powder.

¹H NMR (CDCl₃) δ 2.34 (s, 3H), 2.28 (s, 6H), 3.96 (s, 3H), 5.20 (brs, 1H), 6.29 (s, 1H).

MS Calcd.: 182; MS Found: 183 (M+H).

### Reference Example 13

### 2-Methyl-2,4,5,6-tetrahydrocyclopenta[c]pyrazol-3-ol

To a solution of ethyl 2-oxocyclopentanecarboxylate (4.00 g, 28.2 mmol) in acetic acid (10 mL) was added dropwise methylhydrazine (1.50 mL, 28.2 mmol) with ice bath cooling. The mixture was stirred at 40° C. for 3 days. The mixture was diluted with sodium hydroxide solution (8N solution in water, 20 mL), extracted with ethyl acetate (50 mL×4) and washed with brine. The organic layer was dried over anhydrous magnesium sulfate, filtered and concentrated in vacuo. The residue was purified by flash column chromatography on silica gel eluting with a 10-50% ethyl acetate/n-hexane gradient mixture. The filtrate was concentrated in vacuo to give the title compound (700 mg, 5.07 mmol, 18%) as a colorless powder.

¹H NMR (CDCl₃) δ 1.84-1.90 (m, 2H), 2.50 (t, J=7.5 Hz, 2H), 2.58 (t, J=7.5 Hz, 2H), 3.13 (s, 3H), 8.64 (s, 1H).

MS Calcd.: 138; Found: 139 (M+H).

### Reference Example 14

### 2-Methylisoindolin-4-ol

### i) 4-Hydroxy-2-methyl-1H-isoindole-1,3(2H)-dione

To a solution of 4-hydroxy-2-benzofuran-1,3-dione (4.50 g, 27.4 mmol) in acetic acid (15 mL) were added sodium acetate (2.92 g, 35.6 mmol) and methylamine hydrochloride (2.40 g, 35.6 mmol). The mixture was stirred at 100° C. for 2 h. The mixture was concentrated in vacuo and diluted with saturated aqueous sodium hydrogen carbonate solution (200 mL) and extracted with ethyl acetate (200 mL×3). The combined organic layer was washed with brine, dried over anhydrous magnesium sulfate, filtered and concentrated in vacuo. The residue was washed with diisopropyl ether to give the title compound (3.30 g, 18.6 mmol, 68%) as a yellow powder.

¹H NMR (DMSO-d₆) δ 2.96 (s, 3H), 7.19 (d, J=8.4 Hz, 1H), 7.25 (d, J=8.4 Hz, 1H), 7.57 (d, J=8.4 Hz, 1H), 10.9 (s, 1H).

MS Calcd.: 177; MS Found: 178 (M+H).

### ii) 2-Methylisoindolin-4-ol

To a solution of 4-hydroxy-2-methyl-1H-isoindole-1,3(2H)-dione (3.28 g, 18.5 mmol) in tetrahydrofuran (70 mL) were added lithium aluminum hydride (1.0 M solution in tetrahydrofuran, 74.0 mL, 74.0 mmol). The mixture was stirred at 80° C. for 4 h. The mixture was poured into water with ice bath cooling, extracted with ethyl acetate (100 mL×3) and washed with brine. The organic layer was dried over anhydrous magnesium sulfate, filtered, and concentrated in vacuo. The residue was recrystallized from methanol/ethyl acetate to give the title compound (320 mg, 2.14 mmol, 12%) as a yellow powder.

¹H NMR (DMSO-d₆) δ 2.46 (s, 3H), 3.73 (s, 2H), 3.77 (s, 2H), 6.58-6.65 (m, 2H), 6.95-7.09 (m, 1H), 9.40 (brs, 1H).

MS Calcd.: 149; MS Found: 150 (M+H).

### Reference Example 15

### 5,7-Dichloro-2-methylisoindolin-4-ol

To a solution of 2-methylisoindolin-4-ol (260 mg, 1.74 mmol) in acetic acid (1.5 mL) were added iodine (10.0 mg, 0.0394 mmol) and sulfuryl chloride (0.308 mL, 3.83 mmol). The mixture was stirred at room temperature for 2 days. The mixture was diluted with saturated aqueous sodium hydrogen carbonate solution (30 mL) and extracted with ethyl acetate (30 mL×4). The combined organic layer was washed with brine, dried over anhydrous magnesium sulfate, filtered and concentrated in vacuo. The residue was purified by flash column chromatography on silica gel eluting with a 0-10% methanol/ethyl acetate gradient mixture. The filtrate was concentrated in vacuo to give the solid, which was washed with ethyl acetate to give the title compound (240 mg, 1.10 mmol, 63%) as colorless crystals.

¹H NMR (DMSO-d₆) δ 2.45 (s, 3H), 3.79 (s, 2H), 3.87 (s, 2H), 7.29 (s, 1H), 9.80 (brs, 1H).

MS Calcd.: 217; MS Found: 218 (M+H).

### Reference Example 16

### 4-Methoxy-2-methyl-6-(trifluoromethyl)pyridin-3-ol

### i) 4,6-Dibromo-2-methylpyridin-3-ol

To a solution of 2-methylpyridin-3-ol (15.0 g, 137 mmol) in acetonitrile (750 mL) was added N-bromosuccinimide (53.8 g, 302 mmol). The mixture was stirred at 80° C. for 3 h. The reaction mixture was concentrated in vacuo, diluted with saturated aqueous sodium hydrogen carbonate solution (300 mL), extracted with ethyl acetate (200 mL×3) and washed with brine. The organic layer was dried over anhydrous magnesium sulfate, filtered and concentrated in vacuo. The residue was purified by flash column chromatography on silica gel eluting with a 2-20% ethyl acetate/n-hexane gradient mixture. The filtrate was concentrated in vacuo to give the solid, which was recrystallized from ethyl acetate-n-hexane to give the title compound (11.0 g, 41.2 mmol, 30%) as a red oil.

¹H NMR (CDCl₃) δ 2.52 (s, 3H), 5.63 (brs, 1H), 7.45 (s, 1H).

MS Calcd.: 264; Found: 265 (M+H).

### ii) 4-Methoxy-2-methyl-6-(trifluoromethyl)pyridin-3-ol

A mixture of copper iodide (27.4 g, 144 mmol) and potassium fluoride (8.36 g, 144 mmol) was dried at 80° C. for 2 h in vacuo. To the mixture were added 1-methyl-2-pyrrolidone (50 mL), 4,6-dibromo-2-methylpyridin-3-ol (11.0 g, 41.2 mmol), and trifluoromethyl trimethylsilane (18.0 mL, 123 mmol). The mixture was stirred at 50° C. for 3 days. The mixture was poured into aqueous ammonia (50 mL), extracted with ethyl acetate (200 mL×4) and washed with brine. The organic layer was dried over anhydrous magnesium sulfate, filtered, and concentrated in vacuo. The residue was purified by flash column chromatography on silica gel eluting with a 5-20% ethyl acetate/n-hexane gradient mixture. The filtrate was concentrated in vacuo to give the 4-bromo-2-methyl-6-(trifluoromethyl)pyridin-3-ol (820 mg, 3.20 mmol). To a solution of 4-bromo-2-methyl-6-(trifluoromethyl)pyridin-3-ol (820 mg, 3.20 mmol) in methanol (2.0 mL) were added sodium methoxide (28% solution in methanol, 3.0 mL) and copper iodide (671 mg, 3.52 mmol). The mixture was stirred at 100° C. for 3 h. The mixture was diluted with saturated aqueous ammonium chloride solution (30 mL) and extracted with ethyl acetate (30 mL×4). The combined organic layer was washed with brine, dried over anhydrous magnesium sulfate, filtered and concentrated in vacuo. The residue was purified by flash column chromatography on silica gel eluting with a 50-100% ethyl acetate/n-hexane gradient mixture. The filtrate was concentrated in vacuo to give the title compound and inseparable compounds.

MS Calcd.: 207; Found: 208 (M+H).

### Reference Example 17

### 4-Bromo-2-methoxy-6-methylanilne

Bromine (47.1 mL, 0.92 mol) was added dropwise to a solution of 2-methoxy-6-methylaniline (125 g, 0.91 mol) in methanol (1.0 L) at room temperature. The reaction mixture was stirred for 8 h. The reaction mixture was concentrated in vacuo, and combined with ethyl acetate (500 mL). The precipitate was collected by filtration and washed with ethyl acetate and diethyl ether. The resultant was dissolved in water (400 mL) and ethyl acetate (350 mL), and neutralized with saturated sodium hydrogen carbonate solution. The organic layer was separated, and the aqueous layer was extracted with ethyl acetate (200 mL). The organics were dried over anhydrous magnesium sulfate, filtered, and concentrated in vacuo. The residue was recrystallized from ethyl acetate/n-hexanes mixture (4:1, 500 mL) to give the title compound as purple crystals (87.6 g, 44%).

¹H NMR (CDCl₃) δ 2.14 (s, 3H), 3.72 (s, 2H), 3.83 (s, 3H), 6.79 (d, J=2.1 Hz, 1H), 6.84 (d, J=2.1 Hz, 1H).

MS Calcd.: 215; Found: 216 (M+H).

### Reference Example 18

### 2-Methoxy-6-methyl-4-(1H-pyrazol-1-yl)aniline

To a solution of 4-bromo-2-methoxy-6-methylanilne (2.00 g, 9.25 mmol) in 1,4-dioxane (10 mL) were added potassium carbonate (3.84 g, 27.8 mmol), copper iodide (1.70 g, 9.25 mmol), pyrazole (1.25 g, 18.5 mmol) and N,N′-dimethylethylenediamine (0.68 mL, 6.47 mmol). The mixture was stirred at 105° C. for 19 h. To the mixture were added ethyl acetate (30 mL) and saturated aqueous ammonium chloride (80 mL), and the black precipitate was removed by filtration. The filtrate was extracted with ethyl acetate (30 mL×4). The combined organic layer was washed with brine, dried over anhydrous magnesium sulfate, filtered and concentrated in vacuo to give the title compound (1.79 g, 8.81 mmol, 95%) as a brown solid.

¹H NMR (CDCl₃) δ 2.21 (s, 3H), 3.87 (brs, 2H), 3.88 (s, 3H), 6.41 (t, J=2.1 Hz, 1H), 6.94 (d, J=2.1 Hz, 1H), 7.09 (d, J=2.4 Hz, 1H), 7.68 (d, J=2.1 Hz, 1H), 7.79 (d, J=2.4 Hz, 1H).

MS Calcd.: 203; Found: 204 (M+H).

### Reference Example 19

### 2-Methoxy-6-methyl-4-[3-(trifluoromethyl)-1H-pyrazol-1-yl]aniline

The title compound was prepared from 4-bromo-2-methoxy-6-methylanilne and 3-(trifluoromethyl)-1H-pyrazole in the similar method described in Reference example 18.

¹H NMR (CDCl₃) δ 2.23 (s, 3H), 3.87 (brs, 2H), 3.92 (s, 3H), 6.66 (d, J=2.4 Hz, 1H), 6.94-6.96 (m, 1H), 7.05 (d, J=2.4 Hz, 1H), 7.79-7.81 (m, 1H).

MS Calcd.: 271; Found: 272 (M+H).

### Reference Example 20

### 4-Chloro-2-methoxy-6-methylaniline

### i) 2-Bromo-4-chloro-6-methylaniline

To a solution of 2-chloro-4-methylaniline (98.8 g, 697 mmol) in acetonitrile (400 mL) was added N-bromosuccinimide (137 g, 767 mmol). The mixture was stirred at 0° C. for 90 min. The reaction mixture was concentrated in vacuo, diluted with saturated aqueous sodium hydrogen carbonate solution (300 mL), extracted with ethyl acetate (150 mL×4) and washed with brine. The organic layer was dried over anhydrous magnesium sulfate, filtered and concentrated in vacuo to give the title compound (154 g, 0.697 mmol, 100%) as a brown powder.

¹H NMR (CDCl₃) δ 2.18 (s, 3H), 4.04 (brs, 2H), 6.99 (d, J=−1.2 Hz, 1H), 7.29 (d, J=1.2 Hz, 1H).

MS Calcd.: 219; Found: 220 (M+H).

### ii) 4-Chloro-2-methoxy-6-methylaniline

To a solution of 2-bromo-4-chloro-6-methylaniline (45.0 g, 204 mmol) in methanol (30 mL) were added sodium methoxide (28% solution in methanol, 225 mL) and copper iodide (44.7 g, 234 mmol). The mixture was stirred at 100° C. for 2 h. The mixture was diluted with saturated aqueous ammonium chloride solution (500 mL) and extracted with ethyl acetate (200 mL×4). The combined organic layer was washed with brine, dried over anhydrous magnesium sulfate, filtered and concentrated in vacuo. The residue was purified by flash column chromatography on silica gel eluting with a 0-10% ethyl acetate/n-hexane gradient mixture. The filtrate was concentrated in vacuo to give the title compound (26.4 g, 153 mmol, 75%) as a brown solid.

¹H NMR (CDCl₃) δ 2.13 (s, 3H), 3.70 (brs, 2H), 3.82 (s, 3H), 6.66 (d, J=2.1 Hz, 1H), 6.69 (d, J=2.1 Hz, 1H).

MS Calcd.: 171; Found: 172 (M+H).

### Reference Example 21

### 2,6-Dichloro-4-(trifluoromethoxy)phenol

To a solution of 2-chloro-4-(trifluoromethoxy)phenol (28.2 g, 132 mmol) in chlorobenzene (110 mL) was added N-chlorosuccinimide (23.0 g, 172 mmol). The mixture was stirred at 60° C. for 20 h. The reaction mixture was concentrated in vacuo, diluted with saturated aqueous sodium hydrogen carbonate solution (300 mL), extracted with ethyl acetate (200 mL×4) and washed with brine. The organic layer was dried over anhydrous magnesium sulfate, filtered and concentrated in vacuo. The residue was purified by flash column chromatography on silica gel eluting with a 0-15% ethyl acetate/n-hexane gradient mixture. The filtrate was concentrated in vacuo to give a solid, which was recrystallized from n-hexane to give the title compound (9.66 g, 39.1 mmol, 30%) as colorless crystals.

¹H NMR (CDCl₃) δ 5.88 (s, 1H), 7.20 (s, 2H).

### Reference Example 22

### 2-Chloro-7-(1-ethylpropyl)-4-methoxy-1-methyl-1H-benzimidazole

### i) Methyl 2-chloro-3-nitrobenzoate

A suspension of 2-chloro-3-nitrobenzic acid (20 g, 99 mmol) in dichloromethane (800 mL) was cooled in an ice bath. Dimethylformamide (0.40 mL) was added to the reaction mixture followed by a dropwise addition of oxalyl chloride (13.85 g, 109 mmol). The reaction mixture was allowed to warm to room temperature and stirred for 6 h. Methanol (200 mL) was added dropwise to the reaction mixture and the reaction mixture was stirred overnight. The reaction mixture was concentrated in vacuo, and the residue was dissolved in dichloromethane and passed through a plug of silica eluting with a 50% ethyl acetate/n-hexanes mixture. The filtrate was concentrated in vacuo to give the title compound (21.5 g, 100%).

¹H NMR (CDCl₃) δ 3.98 (s, 3H), 7.48 (t, J=7.8 Hz, 1H), 7.84 (d, J=8.2 Hz, 1H), 7.95 (d, J=7.8 Hz, 1H).

### ii) Methyl 2-methylamino-3-nitrobenzoate

A solution of methyl 2-chloro-3-nitrobenzoate (21.5 g, 99.5 mmol) in tetrahydrofuran (300 mL) was treated with dropwise addition of methylamine (2M solution in THF, 300 mL, 597 mmol) and was stirred overnight at room temperature. The reaction mixture was concentrated in vacuo, dissolved in dichloromethane and washed with aqueous sodium bicarbonate solution and water. The organics were dried over anhydrous sodium sulfate, filtered, and concentrated in vacuo to give the title compound (20.8 g, 100%).

¹H NMR (CDCl₃) δ 2.82 (d, J=5.5 Hz, 3H), 3.9 (s, 3H), 6.65 (t, J=7.8 Hz, 1H), 7.97 (d, J=8.2 Hz, 1H), 8.04 (J=7.8 Hz, 1H), 8.57 (s, 1H).

MS Calcd.: 210; Found: 211 (M+H).

### iii) Methyl 3-amino-2-methylaminobenzoate

A solution of methyl 2-methylamino-3-nitrobenzoate (20.7 g, 98 mmol) in methanol (1200 mL) was inerted with nitrogen. To the solution was added 10% palladium on carbon (50% wet, 5 g). The reaction mixture was purged with hydrogen and stirred under balloon pressure hydrogen for 7 h. The catalyst was removed by filtration and the filtrate was concentrated in vacuo to give the title compound (17.5 g, 99%).

MS Calcd.: 180; Found: 181 (M+H).

### iv) Methyl 3-methyl-2-oxo-2,3-dihydro-1H-benzimidazole-4-carboxylate

To a solution of methyl 3-amino-2-methylaminobenzoate (17.5 g, 97 mmol) in tetrahydrofuran (550 mL) was added 1,1′-carbonyldiimidazole (20.5° g, 146 mmol) and the reaction mixture was stirred overnight at room temperature. The reaction mixture was heated at 50° C. for 2 h and allowed to cool to room temperature overnight. The reaction mixture was concentrated in vacuo and the residue was dissolved in ethyl acetate (1 L) and washed with water (400 mL). The organics were dried over anhydrous sodium sulfate, filtered and concentrated in vacuo. The residue was purified by flash chromatography eluting with 50% ethyl acetate/dichloromethane to give the title compound (7.22 g, 78%).

¹H NMR (CDCl₃) δ 3.59 (s, 3H), 3.95 (s, 3H), 7.08 (t, J=7.8 Hz, 1H), 7.27 (d, J=7.8 Hz, 1H), 7.52 (d, J=8.2 Hz, 1H).

MS Calcd.: 206; Found: 207 (M+H).

### v) 7-(1-Ethyl-1-hydroxypropyl)-1-methyl-1,3-dihydro-2H-benzimidazol-2-one

A solution of ethylmagnesium bromide (3M solution in diethyl ether; 32 mL, 96 mmol) was added dropwise to a suspension of methyl 3-methyl-2-oxo-2,3-dihydro-1H-benzimidazole-4-carboxylate (5.00 g, 24.2 mmol) in tetrahydrofuran (50 mL) at 0° C. The mixture was stirred at 40° C. for 18 h. The reaction mixture was quenched with water and 1N hydrochloric acid and extracted with ethyl acetate. The extract was washed with brine, dried over anhydrous magnesium sulfate, filtered and concentrated in vacuo. The residue was crystallized from ethanol/ethyl ether to afford the title compound as colorless crystals (3.39 g, 70%).

¹H NMR (CDCl₃) δ 0.90 (t, J=7.5 Hz, 6H), 1.90-2.20 (m, 5H), 3.84 (s, 3H), 6.90-7.05 (m, 3H), 9.10-9.30 (m, 1H).

MS Calcd.: 234; MS Found: 235 (M+H).

### vi) 7-(1-Ethylpropyl)-1-methyl-1,3-dihydro-2H-benzimidazol-2-one

A mixture of 7-(1-ethyl-1-hydroxypropyl)-1-methyl-1,3-dihydro-2H-benzimidazol-2-one (6.00 g, 25.6 mmol) and 6N hydrochloric acid (20 mL) in ethanol (100 mL) was stirred at 50° C. for 3 h. The mixture was concentrated in vacuo, and the resulting residue was dissolved in ethyl acetate. The organics were washed with aqueous potassium carbonate solution, dried over anhydrous magnesium sulfate, filtered and concentrated in vacuo to give pale yellow oil which was used for the next reaction without further purification. The crude material (MS Calcd.: 216; Found: 217 (M+H)) was dissolved in ethanol (150 mL). This solution was combined with 10% palladium on carbon (50% wet; 1.00 g), purged with hydrogen and stirred under 5 atoms hydrogen atmosphere for 7 h. The catalyst was removed by filtration and the filtrate was concentrated in vacuo. The residue was crystallized from ethanol/ethyl ether to afford the title compound as colorless crystals (3.02 g, 54%).

¹H NMR (CDCl₃) δ 0.82 (t, J=6.6 Hz, 6H), 1.60-1.85 (m, 4H), 3.15-3.25 (m, 1H), 3.65 (s, 3H), 6.85-6.98 (m, 2H), 7.00-7.10 (m, 1H), 10.2-10.5 (m, 1H).

MS Calcd.: 218; MS Found: 219 (M+H).

### vii) 4-Bromo-7-(1-ethylpropyl)-1-methyl-1,3-dihydro-2H-benzimidazol-2-one

2,2′-Azobis(isobutyronitrile) (601 mg, 3.66 mmol) was added to a mixture of 7-(1-ethylpropyl)-1-methyl-1,3-dihydro-2H-benzimidazol-2-one (8.00 g, 36.6 mmol) and N-bromosuccinimide (7.17 g, 40.3 mmol) in chlorobenzene (250 mL). The mixture was stirred at 75° C. for 5 h and diluted with aqueous saturated sodium hydrogen carbonate solution. The mixture was extracted with ethyl acetate, dried over anhydrous magnesium sulfate and concentrated in vacuo. The residue was crystallized from isopropyl ether/n-hexane (1/1). The resulting crystals were collected by filtration and dried in vacuo to afford the title compound as colorless crystals (5.99 g, 56%).

¹H NMR (CDCl₃) δ 0.81 (t, J=7.5 Hz, 6H), 1.55-1.80 (m, 4H), 3.05-3.20 (m, 1H), 3.63 (s, 3H), 6.82 (d, J=8.4 Hz, 1H), 7.15 (d, J=8.4 Hz, 1H), 8.81 (s, 1H).

MS Calcd.: 296; MS Found: 297 (M+H), 299.

### viii) 7-(1-Ethylpropyl)-4-methoxy-1-methyl-1,3-dihydro-2H-benzimidazol-2-one

A solution of 4-bromo-7-(1-ethylpropyl)-1-methyl-1,3-dihydro-2H-benzimidazol-2-one (5.90 g, 19.9 mmol), anhydrous cuprous iodide (4.65 g, 23.9 mmol) and sodium methoxide (28% solution in methanol; 122 mL) in N,N-dimethylformamide (150 mL) was heated at 100° C. for 1 h. After cooling, the mixture was diluted in water and extracted with ethyl acetate. The extract was dried over anhydrous magnesium sulfate, filtered and concentrated in vacuo. The residue was crystallized from isopropyl ether/n-hexane. The resulting crystals were collected by filtration and dried in vacuo to give the title compound (3.32 g, 13.4 mmol, 67%).

¹H NMR (CDCl₃) δ 0.81 (t, J=7.5 Hz, 6H), 1.50-1.80 (m, 4H), 3.05-3.20 (m, 1H), 3.63 (s, 3H), 3.89 (s, 3H), 6.62 (d, J=8.7 Hz, 1H), 6.84 (d, J=8.7H, 1H), 8.58 (s, 1H).

MS Calcd.: 248; Found: 249 (M+H).

### ix) 2-Chloro-7-(1-ethylpropyl)-4-methoxy-1-methyl-1H-benzimidazole

A mixture of 7-(1-ethylpropyl)-4-methoxy-1-methyl-1,3-dihydro-2H-benzimidazol-2-one (3.40 g, 13.7 mmol) in phosphorous oxychloride (38 ml) was stirred at 110° C. for 4 h. The reaction mixture was allowed to cool to room temperature and concentrated in vacuo. The residue was dissolved in ethyl acetate and washed with aqueous sodium hydrogen carbonate solution and brine. The organic layer was dried over anhydrous magnesium sulfate, filtered and concentrated in vacuo. The residue was purified by flash chromatography on silica gel eluting with a 1-30% ethyl acetate/n-hexane gradient to give the title compound (3.02 g, 82%).

¹H NMR (CDCl₃) δ 0.81 (t, J=7.5 Hz, 6H), 1.60-1.80 (m, 4H), 3.10-3.20 (m, 1H), 3.97 (s, 6H), 6.68 (d, J=8.1 Hz, 1H), 7.00 (d, J=8.1 Hz, 1H).

MS Calcd.: 266; MS Found: 267 (M+H).

### Reference Example 23

### 2,4-Dichloro-7-(1-ethylpropyl)-1-methyl-1H-benzimidazole

### i) 4-Chloro-7-(1-ethylpropyl)-1-methyl-1,3-dihydro-2H-benzimidazol-2-one

2,2′-Azobis(isobutyronitrile) (94 mg, 0.57 mmol) was added to a mixture of 7-(1-ethylpropyl)-1-methyl-1,3-dihydro-2H-benzimidazol-2-one (2.90 g, 13.3 mmol) and N-chlorosuccinimide (1.95 g, 14.6 mmol) in carbon tetrachloride (250 mL). The mixture was stirred at 70° C. for 48 h. The reaction mixture was concentrated in vacuo, extracted with ethyl acetate and washed with brine. The organic layer was dried over anhydrous magnesium sulfate, filtered and concentrated in vacuo. The residue was crystallized from ethanol/2-propanol to afford the title compound as colorless crystals (2.28 g, 68%).

### mp 165-166° C.

¹H NMR (CDCl₃) δ 0.81 (t, J=7.2 Hz, 6H), 1.60-1.85 (m, 4H), 3.10-3.20 (m, 1H), 3.64 (s, 3H), 6.87 (d, J=8.7 Hz, 1H), 7.03 (d, J=8.7 Hz, 1H), 8.55 (s, 1H).

MS Calcd.: 251; MS Found: 252 (M+H).

### ii) 2,4-Dichloro-7-(1-ethylpropyl)-1-methyl-1H-benzimidazole

A mixture of 4-chloro-7-(1-ethylpropyl)-1-methyl-1,3-dihydro-2H-benzimidazol-2-one (1.17 g, 4.63 mmol) in phosphorous oxychloride (28 g) was stirred at 90° C. for 3 h. The reaction mixture was allowed to cool to room temperature and concentrated in vacuo. The residue was dissolved in ethyl acetate and washed with aqueous sodium bicarbonate and brine. The organic layer was dried over anhydrous magnesium sulfate, filtered and concentrated in vacuo. The residue was purified by flash column chromatography on silica gel eluting with a 10-50% ethyl acetate/n-hexane gradient mixture. The filtrate was concentrated in vacuo to give the solid, which was recrystallized from ethyl acetate/n-hexane to afford the title compound as colorless crystals (1.03 g, 82%).

### mp 94-95° C.

¹H NMR (CDCl₃) δ 0.82 (t, J=7.5 Hz, 6H), 1.60-1.90 (m, 4H), 3.20-3.30 (m, 1H), 4.01 (s, 3H), 7.05 (d, J=8.4 Hz, 1H), 7.26 (d, J=8.4 Hz, 1H).

MS Calcd.: 270; MS Found: 271 (M+H).

### Reference Example 24

### 2-[4-Bromo-2-chloro-7-(1-ethylpropyl)-1H-benzimidazol-1-yl]-N,N-dimethylethanamine

### i) 4-(1-Ethylpropyl)-1-(4-methoxybenzyl)-1,3-dihydro-2H-benzimidazol-2-one

To a solution of 4-(1-ethylpropyl)-1,3-dihydro-2H-benzimidazol-2-one (3.02 g, 14.8 mmol) in N,N-dimethylformamide (30 mL) were added 4-methoxybenzylchloride (1.95 mL, 14.8 mmol) and potassium carbonate (2.04 g, 14.8 mmol), and the mixture was stirred at room temperature for 15 hr. The reaction mixture was diluted with water and extracted with ethyl acetate. The organic layer was washed with brine, dried over anhydrous sodium sulfate and concentrated in vacuo. The residue was purified by silica gel column chromatography eluting with a 10-30% ethyl acetate/n-hexane gradient mixture. The fractions containing the title compound were concentrated in vacuo, and the residual solid was washed with toluene to give the title compound (432 mg, 1.33 mmol, 9.0%) as a colorless solid. The filtrate was purified by basic silica gel column chromatography eluting with a 15-35% ethyl acetate/n-hexane gradient mixture to give the title compound (1.31 g, 4.04 mmol, 27% yield) as a colorless solid.

total: 1.74 g, 5.36 mmol, 36%.

¹H NMR (CDCl₃) δ 0.82 (t, J=7.2 Hz, 6H), 1.58-1.87 (m, 4H), 2.55-2.65 (m, 1H), 3.77 (s, 3H), 5.01 (s, 2H), 6.74-7.01 (m, 5H), 7.32 (d, J=8.4 Hz, 2H), 9.78 (s, 1H).

MS Calcd.: 324, MS Found: 325 (M+H).

### ii) 3-[2-(Dimethylamino)ethyl]-4-(1-ethylpropyl)-1-(4-methoxybenzyl)-1,3-dihydro-2H-benzimidazol-2-one

To a solution of 4-(1-ethylpropyl)-1-(4-methoxybenzyl)-1,3-dihydro-2H-benzimidazol-2-one (1.62 g, 4.99 mmol) in N,N-dimethylformamide (15 mL) were added sodium hydride (60% dispersion in oil; 599 mg, 15.0 mmol) and 2-dimethylaminoethyl chloride hydrochloride (1.08 g, 7.49 mmol) at 0° C., and the mixture was stirred at 55° C. for 12 hr. After cooling, the reaction mixture was poured into ice-cold water, and extracted with ethyl acetate (X2). The combined organic layer was washed with brine, dried over anhydrous sodium sulfate and concentrated in vacuo. The residue was purified by basic silica gel column chromatography eluting with a 15-35% ethyl acetate/n-hexane gradient mixture to give the title compound (1.50 g, 3.79 mmol, 76%) as a colorless oil.

¹H NMR (CDCl₃) δ 0.86 (t, J=7.2 Hz, 6H), 1.58-1.84 (m, 4H), 2.35 (s, 6H), 2.58-2.63 (m, 2H), 2.98-3.08 (1H, m), 3.77 (s, 3H), 4.22-4.27 (m, 2H), 4.99 (s, 2H), 6.74-7.00 (m, 5H), 7.24-7.30 (m, 2H).

MS Calcd.: 395, MS Found: 396 (M+H).

### iii) 1-[2-(Dimethylamino)ethyl]-7-(1-ethylpropyl)-1,3-dihydro-2H-benzimidazol-2-one

A mixture of 3-[2-(dimethylamino)ethyl]-4-(1-ethylpropyl)-1-(4-methoxybenzyl)-1,3-dihydro-2H-benzimidazol-2-one (1.49 g, 3.77 mmol) and trifluoroacetic acid (15 mL) was refluxed for 72 hr. After cooling, trifluoroacetic acid was evaporated in vacuo. The reaction mixture was neutralized with saturated aqueous sodium hydrogen carbonate solution and extracted with ethyl acetate (X2). The combined organic layer was washed with brine, dried over anhydrous sodium sulfate and concentrated in vacuo. The residue was purified by basic silica gel column chromatography eluting with a 50-100% ethyl acetate/n-hexane gradient mixture to give the title compound (489 mg, 1.78 mmol, 47%) as a solid.

¹H NMR (CDCl₃) δ 0.87 (t, J=7.2 Hz, 6H), 1.60-1.85 (m, 4H), 2.35 (s, 6H), 2.60 (t, J=7.8 Hz, 2H), 2.98-3.08 (m, 1H), 4.20 (t, J=7.8 Hz, 2H), 6.87-6.94 (m, 2H), 7.00-7.06 (m, 1H), 8.64 (s, 1H).

### iv) 4-Bromo-1-[2-(dimethylamino)ethyl]-7-(1-ethylpropyl)-1,3-dihydro-2H-benzimidazol-2-one

To a solution of 1-[2-(dimethylamino)ethyl]-7-(1-ethylpropyl)-1,3-dihydro-2H-benzimidazol-2-one (43 mg, 0.156 mmol) in acetonitrile (0.7 mL) was added N-bromosuccinimide (28 mg, 0.156 mmol) at 0° C., and the mixture was stirred at room temperature for 1 hr. The reaction mixture was quenched with saturated aqueous sodium hydrogen carbonate solution and extracted with ethyl acetate (X2). The combined organic layer was washed with brine, dried over anhydrous sodium sulfate and concentrated in vacuo. The residue was purified by silica gel column chromatography eluting with a 40-65% ethyl acetate/n-hexane gradient mixture to give the title compound (20 mg, 0.0565 mmol, 36%) as an oil.

¹H NMR (CDCl₃) δ 0.85 (t, J=7.2 Hz, 3H), 1.56-1.85 (m, 4H), 2.34 (s, 6H), 2.60 (t, J=7.5 Hz, 2H), 2.94-3.04 (m, 1H), 4.19 (t, J=7.5 Hz, 2H), 6.82 (d, J=8.5 Hz, 1H), 7.15 (d, J=8.5 Hz, 1H), 9.06 (s, 1H).

MS Calcd.: 353, MS Found: 354 (M+H).

### v) 2-[4-Bromo-2-chloro-7-(1-ethylpropyl)-1H-benzimidazol-1-yl]-N,N-dimethylethanamine

A mixture of 4-bromo-1-[2-(dimethylamino)ethyl]-7-(1-ethylpropyl)-1,3-dihydro-2H-benzimidazol-2-one (19 mg, 0.0536 mmol) and phosphoryl chloride (0.5 mL) was stirred at 70° C. for 30 min and at 90° C. for 4 hr. After cooling, the residue was neutralized with saturated aqueous sodium hydrogen carbonate solution and extracted with ethyl acetate. The organic layer was washed with brine, dried over anhydrous sodium sulfate and concentrated in vacuo. The residue was purified by silica gel column chromatography eluting with a 15-65% ethyl acetate/n-hexane gradient mixture to give the title compound (7.0 mg, 0.0188 mmol, 35%) as a colorless solid.

¹H NMR (CDCl₃) δ 0.85 (t, J=7.2 Hz, 6H), 1.60-1.88 (m, 4H), 2.75-2.90 (m, 1H), 3.00 (s, 6H), 3.48-3.53 (m, 2H), 4.41-4.46 (m, 2H), 6.85 (d, J=8.4 Hz, 1H), 7.33 (d, J=8.4 Hz, 1H).

MS Calcd.: 371, MS Found: 372 (M+H).

### Reference Example 25

### 2,4-Dichloro-7-(1-ethylpropyl)-1H-benzimidazole

### i) tert-Butyl 4-(1-ethylpropyl)-3-(4-methoxybenzyl)-2-oxo-2,3-dihydro-1H-benzimidazole-1-carboxylate

To a solution of tert-butyl 4-(1-ethylpropyl)-2-oxo-2,3-dihydro-1H-benzimidazole-1-carboxylate (2.45 g, 8.05 mmol) in N,N-dimethylformamide (12 mL) were added potassium carbonate (1.22 g, 8.85 mmol) and 4-methoxybenzyl chloride (1.20 mL, 8.85 mmol), and the mixture was stirred at room temperature for 15 hr. The reaction mixture was quenched with saturated aqueous sodium hydrogen carbonate solution and extracted with ethyl acetate (X2). The combined organic layer was washed with water and brine, dried over anhydrous sodium sulfate and concentrated in vacuo. The residue was purified by silica gel column chromatography eluting with a 5-20% ethyl acetate/n-hexane gradient mixture to give the title compound (3.12 g, 7.35 mmol, 91%) as a colorless solid.

¹H NMR (CDCl₃) δ 0.51 (t, J=7.2 Hz, 6H), 1.34-1.63 (m, 4H), 2.79-2.90 (m, 1H), 3.76 (s, 3H), 5.25 (s, 2H), 6.80-6.85 (m, 2H), 6.96-7.13 (m, 4H), 7.80 (dd, J=1.2, 8.1 Hz, 1H).

### ii) 7-(1-Ethylpropyl)-1-(4-methoxybenzyl)-1,3-dihydro-2H-benzimidazol-2-one

To a solution of tert-butyl 4-(1-ethylpropyl)-3-(4-methoxybenzyl)-2-oxo-2,3-dihydro-1H-benzimidazole-1-carboxylate (5.86 g, 13.8-mmol) in ethyl acetate (12 mL) was added 4N hydrogen chloride in ethyl acetate (30 mL) at 0° C., and the mixture was stirred at the same temperature for 30 min. The reaction mixture was neutralized with saturated aqueous sodium hydrogen carbonate solution and extracted with ethyl acetate (X2). The combined organic layer was washed with brine, dried over anhydrous sodium sulfate and concentrated in vacuo to give the title compound (4.32 g, 13.3 mmol, 96%) as a colorless solid.

¹H NMR (CDCl₃) δ 0.53 (t, J=7.2 Hz, 6H), 1.38-1.62 (m, 4H), 2.78-2.90 (m, 1H), 3.77 (s, 3H), 5.28 (s, 2H), 6.82-7.07 (m, 7H), 9.33 (s, 1H).

MS Calcd.: 324; MS Found: 325 (M+H).

### iii) 4-Chloro-7-(1-ethylpropyl)-1-(4-methoxybenzyl)-1,3-dihydro-2H-benzimidazol-2-one

To a solution of 7-(1-ethylpropyl)-1-(4-methoxybenzyl)-1,3-dihydro-2H-benzimidazol-2-one (3.99 g, 12.3 mmol) in chlorobenzene (40 mL) was added 2,2′-azobisisobutyronitrile (3.99 g, 12.3 mmol), and the mixture was heated to 60° C. N-Chlorosuccinimide (1.64 g, 12.3 mmol) was added slowly to the mixture at 60° C., and the mixture was stirred at 70° C. for 15 hr. After cooling, the reaction mixture was neutralized with saturated aqueous sodium hydrogen carbonate solution and extracted with ethyl acetate (X2). The combined organic layer was washed with brine (X2), dried over anhydrous sodium sulfate and concentrated in vacuo. The residue was purified by silica gel column chromatography eluting with a 15-70% ethyl acetate/n-hexane gradient mixture to give the title compound (2.35 g, 6.55 mmol, 53%) as a colorless solid.

¹H NMR (CDCl₃) δ 0.52 (t, J=7.2 Hz, 6H), 1.32-1.64 (m, 4H), 2.76-2.86 (m, 1H), 3.77 (s, 3H), 5.26 (s, 2H), 6.79-6.85 (m, 3H), 7.01-7.09 (m, 3H), 9.04 (s, 1H).

MS Calcd.: 358; MS Found: 359 (M+H).

### iv) 2,4-Dichloro-7-(1-ethylpropyl)-1H-benzimidazole

A mixture of 4-chloro-7-(1-ethylpropyl)-1-(4-methoxybenzyl)-1,3-dihydro-2H-benzimidazol-2-one (2.27 g, 6.33 mmol) and phosphoryl chloride (20 mL) was stirred at 100° C. for 4 days. After cooling, phosphoryl chloride was evaporated in vacuo. The residue was neutralized with saturated aqueous sodium hydrogen carbonate solution and sodium hydroxide solution (2N solution in water) and extracted with ethyl acetate (X2). The combined organic layer was washed with brine (X1), dried over anhydrous sodium sulfate and concentrated in vacuo. The residue was purified by silica gel column chromatography eluting with a 5-15% ethyl acetate/n-hexane gradient mixture to give the title compound (910 mg, 3.54 mmol, 56%) as a solid.

¹H NMR (CDCl₃) δ 0.78 (t, J=7.5 Hz, 6H), 1.64-1.88 (m, 4H), 2.80-3.20 (m, 1H), 7.00 (d, J=8.1 Hz, 1H), 7.23 (d, J=8.1 Hz, 1H), 9.40 (brs, 1H).

MS Calcd.: 256, MS Found: 257 (M+H).

### Reference Example 26

### 2,7-Dichloro-4-(1-ethylpropyl)-1-(4-methoxybenzyl)-1H-benzimidazole

To a solution of 2,4-dichloro-7-(1-ethylpropyl)-1H-benzimidazole (234 mg, 0.910 mmol) in N,N-dimethylformamide (1.0 mL) were added 4-methoxybenzylchloride (0.13 mL, 1.00 mmol) and potassium carbonate (138 mg, 1.00 mmol), and the mixture was stirred at room temperature for 24 hr. The reaction mixture was diluted with water and extracted with ethyl acetate (X2). The combined organic layer was washed with brine (X1), dried over anhydrous sodium sulfate and concentrated in vacuo. The residue was purified by silica gel column chromatography eluting with a 3-10% ethyl acetate/n-hexane gradient mixture to give the title compound (311 mg, 0.824 mmol, 91%) as an oil.

¹H NMR (CDCl₃) δ 0.77 (t, J=7.2 Hz, 6H), 1.65-1.85 (m, 4H), 3.21-3.31 (m, 1H), 3.78 (s, 3H), 5.72 (s, 2H), 6.85 (d, J=8.7 Hz, 2H), 6.98 (d, J=8.1 Hz, 1H), 7.09 (d, J=8.7 Hz, 2H), 7.17 (d, J=8.1 Hz, 1H).

MS Calcd.: 376, MS Found: 377 (M+H).

### Reference Example 27

### 2,4-Dichloro-7-(1-ethylpropyl)-1-{2-[(4-methoxybenzyl)oxy]ethyl}-1H-benzimidazole

### i) tert-Butyl 3-(2-ethoxy-2-oxoethyl)-4-(1-ethylpropyl)-2-oxo-2,3-dihydro-1H-benzimidazole-1-carboxylate

To a solution of tert-butyl 4-(1-ethylpropyl)-2-oxo-2,3-dihydro-1H-benzimidazole-1-carboxylate (4.94 g, 16.2 mmol) in N,N-dimethylformamide (40 mL) were added ethyl bromoacetate (1.98 mL, 17.9 mmol) and potassium carbonate (2.47 g, 17.9 mmol), and the mixture was stirred at room temperature for 3 hr. The reaction mixture was diluted with water and extracted with ethyl acetate (X1). The organic layer was washed with brine (X2), dried over anhydrous sodium sulfate and concentrated in vacuo. The residue was purified by silica gel column chromatography eluting with a 5-20% ethyl acetate/n-hexane gradient mixture to give the title compound (6.17 g, 15.8 mmol, 98%) as a colorless oil.

¹H NMR (CDCl₃) δ 0.78 (t, J=7.2 Hz, 6H), 1.27 (t, J=7.2 Hz, 3H), 1.43-1.76 (m, 4H), 1.67 (s, 9H), 2.58-2.70 (m, 1H), 4.22 (q, J=7.2 Hz, 2H), 7.03 (dd, J=1.2, 8.1 Hz, 1H), 7.14 (t, J=8.1 Hz, 1H), 7.80 (dd, J=1.2, 8.1 Hz, 1H).

### ii) Ethyl [7-(1-ethylpropyl)-2-oxo-2,3-dihydro-1H-benzimidazol-1-yl]acetate

To a solution of tert-butyl 3-(2-ethoxy-2-oxoethyl)-4-(1-ethylpropyl)-2-oxo-2,3-dihydro-1H-benzimidazole-1-carboxylate (6.16 g, 15.8 mmol) in ethyl acetate (15 mL) was added 4N hydrogen chloride in ethyl acetate (15 mL) at 0° C., and the mixture was stirred at the same temperature for 1 hr. The reaction mixture was neutralized with saturated aqueous sodium hydrogen carbonate solution and extracted with ethyl acetate (X1). The organic layer was washed with brine (X1), dried over anhydrous sodium sulfate and concentrated in vacuo. The residual solid was washed with n-hexane to give the title compound (4.28 g, 14.7 mmol, 93%) as colorless crystals.

¹H NMR (CDCl₃) δ 0.78-0.83 (m, 6H), 1.24-1.30 (m, 3H), 1.54-1.80 (m, 4H), 2.63-2.73 (m, 1H), 4.20-4.27 (m, 2H), 4.88 (s, 2H), 6.91-6.97 (m, 2H), 7.03-7.26 (m, 1H), 9.53 (s, 1H).

MS Calcd.: 290, MS Found: 291 (M+H).

### iii) Ethyl [4-chloro-7-(1-ethylpropyl)-2-oxo-2,3-dihydro-1H-benzimidazol-1-yl]acetate

To a solution of ethyl [7-(1-ethylpropyl)-2-oxo-2,3-dihydro-1H-benzimidazol-1-yl]acetate (3.91 g, 13.5 mmol) in chlorobenzene (40 mL) was added 2,2′-azobisisobutyronitrile (222 mg, 1.35 mmol), and the mixture was heated to 60° C. N-Chlorosuccinimide (1.80 g, 13.5 mmol) was added slowly to the mixture, which was stirred at 70° C. for 2 days. After cooling, the reaction mixture was washed with saturated aqueous sodium hydrogen carbonate solution. The aqueous layer was extracted with ethyl acetate (X1). The combined organic layer was washed with brine (X1), dried over anhydrous sodium sulfate and concentrated in vacuo. The residue was purified by silica gel column chromatography eluting with a 15-50% ethyl acetate/n-hexane gradient mixture to give a mixture containing the title compound. The mixture was suspended in diisopropyl ether (5 mL), and the suspension was stirred at 60° C. for 2 hr. After cooling, the resulting solid was collected by filtration and washed with n-hexane to give the title compound (2.77 g, 8.53 mmol, 63%) as a colorless solid.

¹H NMR (CDCl₃) δ 0.79 (t, J=7.5 Hz, 6H), 1.28 (t, J=7.2 Hz, 3H), 1.51-1.78 (m, 4H), 2.58-2.68 (m, 1H), 4.23 (q, J=7.2 Hz, 2H), 4.85 (s, 2H), 6.86 (d, J=8.7 Hz, 1H), 7.04 (d, J=8.7 Hz, 1H), 8.84 (s, 1H).

MS Calcd.: 324, MS Found: 325 (M+H).

### iv) Ethyl [2,4-dichloro-7-(1-ethylpropyl)-1H-benzimidazol-1-yl]acetate

A mixture of ethyl [4-chloro-7-(1-ethylpropyl)-2-oxo-2,3-dihydro-1H-benzimidazol-1-yl]acetate (2.57 g, 7.91 mmol) and phosphoryl chloride (15 mL) was stirred at 90° C. for 3 days. After cooling, phosphoryl chloride was evaporated in vacuo. The residue was neutralized with 2N aqueous sodium hydroxide and extracted with ethyl acetate (X2). The combined organic layer was washed with brine (X1), dried over anhydrous sodium sulfate and concentrated in vacuo. The residue was purified by silica gel column chromatography eluting with a 10-20% ethyl acetate/n-hexane gradient mixture to give the title compound (2.49 g, 7.25 mmol, 92%) as a solid.

¹H NMR (CDCl₃) δ 0.80 (t, J=7.5 Hz, 6H), 1.29 (t, J=7.2 Hz, 3H), 1.59-1.83 (m, 4H), 2.75-2.84 (m, 1H), 4.25 (q, J=7.2 Hz, 2H), 5.12 (s, 2H), 7.05 (d, J=8.1 Hz, 1H), 7.28 (d, J=8.1 Hz, 1H).

MS Calcd.: 342, MS Found: 343 (M+H).

### v) 2-[2,4-Dichloro-7-(1-ethylpropyl)-1H-benzimidazol-1-yl]ethanol

To a solution of ethyl [2,4-dichloro-7-(1-ethylpropyl)-1H-benzimidazol-1-yl]acetate (950 mg, 2.77 mmol) in tetrahydrofuran (10 mL) was added lithium tetrahydroborate (191 mg, 8.77 mmol), and the mixture was stirred at room temperature for 3 hr. The reaction mixture was quenched with ice-cold water and extracted with ethyl acetate (X2). The combined organic layer was washed with brine (X1), dried over anhydrous sodium sulfate and concentrated in vacuo. The residual solid was washed with diisopropyl ether to give the title compound (620 mg, 2.06 mmol, 74%) as a colorless solid. The filtrate concentrated in vacuo was purified by silica gel column chromatography eluting with a 10-50% ethyl acetate/n-hexane gradient mixture to give the title compound (86 mg, 0.286 mmol, 10%) as a colorless solid.

total: 706 mg, 2.34 mmol, 85%.

¹H NMR (CDCl₃) δ 0.84 (t, J=7.5 Hz, 6H), 1.62-1.85 (m, 4H), 2.03-2.07 (m, 1H), 3.10-3.19 (m, 1H), 3.98-4.04 (m, 1H), 4.57 (t, J=6.0 Hz, 2H), 7.05 (d, J=8.1 Hz, 1H), 7.24 (d, J=8.1 Hz, 1H).

MS Calcd.: 300, MS Found: 301 (M+H).

### vi) 2,4-Dichloro-7-(1-ethylpropyl)-1-{2-[(4-methoxybenzyl)oxy]ethyl}-1H-benzimidazole

To a solution of 2-[2,4-dichloro-7-(1-ethylpropyl)-1H-benzimidazol-1-yl]ethanol (93.0 mg, 0.309 mmol) in tetrahydrofuran (1.0 mL) was added triphenylphosphine (97.2 mg, 0.370 mmol), and the mixture was stirred at room temperature for 10 min. An addition of 4-methoxybenzylalcohol (0.046 mL, 0.375 mmol) and diethyl azodicarboxylate (0.059 mL, 0.371 mmol) was followed by stirring at room temperature for 15 hr. Additional 4-methoxybenzylalcohol (0.047 mL, 0.370 mmol) and diethyl azodicarboxylate (0.059 mL, 0.370 mmol) were added to the reaction mixture twice each over two days. The reaction mixture was diluted with water and extracted with ethyl acetate. The organic layer was washed with brine, dried over anhydrous sodium sulfate and concentrated in vacuo. The residue was purified by silica gel column chromatography eluting with a 2-10% ethyl acetate/n-hexane gradient mixture to give the title compound (32.0 mg, 0.0759 mmol, 25%) as an oil.

¹H NMR (CDCl₃) δ 0.79 (t, J=7.5 Hz, 6H), 1.55-1.82 (m, 4H), 3.00-3.07 (m, 1H), 3.70 (t, J=6.3 Hz, 2H), 3.78 (s, 3H), 4.39 (s, 2H), 4.55 (t, J=6.3 Hz, 2H), 6.76-7.08 (m, 4H), 7.23-7.29 (m, 2H).

MS Calcd.: 420, MS Found: 421 (M+H).

### Reference Example 28

### 4-Bromo-2-chloro-7-(1-ethylpropyl)-1-methyl-1H-benzimidazole

A mixture of 4-bromo-7-(1-ethylpropyl)-1-methyl-1,3-dihydro-2H-benzimidazol-2-one (6.69 g, 22.5 mmol) and phosphoryl chloride (20 mL) was stirred at 100° C. for 4 h. After cooling, phosphoryl chloride was evaporated in vacuo. The residue was neutralized with saturated aqueous sodium hydrogen carbonate solution, and the mixture was extracted with ethyl acetate. The extract was washed with brine, dried over anhydrous magnesium sulfate, filtered and concentrated in vacuo. The residue was recrystallized from ethyl acetate/n-hexane (1:1) to give the title compound as colorless crystals (4.70 g, 14.9 mmol, 66%).

¹H NMR (CDCl₃) δ 0.81 (t, J=7.5 Hz, 6H), 1.64-1.81 (m, 4H), 3.20-3.26 (m, 1H), 4.00 (s, 3H), 6.99 (d, J=8.1 Hz, 1H), 7.41 (d, J=8.1 Hz, 1H).

MS Calcd.: 314; MS Found: 315 (M+H).

### Reference Example 29

### 2-Chloro-4-ethoxy-7-(1-ethylpropyl)-1-methyl-1H-benzimidazole

### i) 4-Ethoxy-7-(1-ethylpropyl)-1-methyl-1,3-dihydro-2H-benzimidazol-2-one

To a solution of 4-bromo-7-(1-ethylpropyl)-1-methyl-1,3-dihydro-2H-benzimidazol-2-one (2.20 g, 7.40 mmol) in N,N-dimethylformamide (10 mL) were added sodium ethoxide (21% solution in ethanol; 25 mL) and copper iodide (1.40 g, 7.35 mmol), and the mixture was stirred at 110° C. for 16 h. The mixture was neutralized with aqueous saturated ammonium chloride solution, and extracted with ethyl acetate. The extract was washed with brine, dried over anhydrous magnesium sulfate, filtered and concentrated in vacuo. The residue was purified by flash column chromatography on silica gel eluting with a 10-40% ethyl acetate/n-hexane gradient mixture. The filtrate was concentrated in vacuo to give 4-ethoxy-7-(1-ethylpropyl)-1-methyl-1,3-dihydro-2H-benzimidazol-2-one as a colorless solid (1.53 g, 5.83 mmol, 79%).

¹H NMR (CDCl₃) δ 0.78 (t, J=7.5 Hz, 6H), 1.38 (t, J=7.2 Hz, 3H), 1.46-1.80 (m, 4H), 3.06-3.10 (m, 1H), 3.61 (s, 3H), 4.09 (q, J=7.2 Hz, 2H), 6.61 (d, J=8.4 Hz, 1H), 6.81 (d, J=8.4 Hz, 1H), 8.78 (s, 1H).

MS Calcd.: 262; MS Found: 263 (M+H).

### ii) 2-Chloro-4-ethoxy-7-(1-ethylpropyl)-1-methyl-1H-benzimidazole

A mixture of 4-ethoxy-7-(1-ethylpropyl)-1-methyl-1,3-dihydro-2H-benzimidazol-2-one (1.53 g, 5.83 mmol) and phosphoryl chloride (20 mL) was stirred at 100° C. for 20 h. After cooling, phosphoryl chloride was evaporated in vacuo. The residue was neutralized with saturated aqueous sodium hydrogen carbonate solution, and the mixture was extracted with ethyl acetate. The extract was washed with brine, dried over anhydrous magnesium sulfate, filtered and concentrated in vacuo. The residue was purified by flash column chromatography on silica gel eluting with a 5-30% ethyl acetate/n-hexane gradient mixture. The filtrate was concentrated in vacuo to give the solid, which was washed with n-hexane to give the title compound as a colorless powder (1.23 g, 4.38 mmol, 75%).

¹H NMR (CDCl₃) δ 0.81 (t, J=7.2 Hz, 6H), 1.52 (t, J=6.9 Hz, 3H), 1.63-1.76 (m, 4H), 3.15-3.19 (m, 1H), 3.97 (s, 3H), 4.21 (q, J=6.9 Hz, 2H), 6.67 (d, J=8.7 Hz, 1H), 6.98 (d, J=8.7 Hz, 1H).

MS Calcd.: 280; MS Found: 281 (M+H).

### Reference Example 30

### 4,6-Dibromo-2-methylpyridin-3-ol

To a solution of 2-methylpyridin-3-ol (15.0 g, 137 mmol) in acetonitrile (750 mL) was added N-bromosuccinimide (53.8 g, 302 mmol), and the mixture was stirred at 80° C. for 3 h. After cooling, the mixture was evaporated in vacuo. The residue was neutralized with saturated aqueous sodium hydrogen carbonate solution, and the mixture was extracted with ethyl acetate. The extract was washed with brine, dried over anhydrous magnesium sulfate, filtered and concentrated in vacuo. The residue was purified by flash column chromatography on silica gel eluting with a 2-20% ethyl acetate/n-hexane gradient mixture. The filtrate was concentrated in vacuo to give the solid, which was recrystallized from ethyl acetate/n-hexane to give the title compound as colorless crystals (11.0 g, 41.2 mmol, 30%).

¹H NMR (CDCl₃) δ 2.52 (s, 3H), 5.63 (s, 1H), 7.45 (s, 1H).

MS Calcd.: 265; MS Found: 266 (M+H).

### Reference Example 31

### 2,4-Dichloro-6-[(dimethylamino)methyl]phenol

To a solution of 2,4-dichlorophenol (25.0 g, 153 mmol) and dimethylamine (2.0 M solution in tetrahydrofuran, 21.0 mL, 42.0 mmol) in ethanol (20 mL) was added formaldehyde (36-38% solution in water, 15.5 mL) with ice bath cooling. The mixture was stirred at room temperature for 15 h, diluted with water, and extracted with ethyl acetate. The extract was washed with brine, dried over anhydrous magnesium sulfate, filtered and concentrated in vacuo. The residue was washed with n-hexane to give the title compound as a colorless powder (30.3 g, 138 mmol, 90%).

¹H NMR (CDCl₃) δ 2.35 (s, 6H), 3.63 (s, 2H), 6.85 (d, J=1.5 Hz, 1H), 7.25 (d, J=1.5 Hz, 1H), 9.90 (s, 1H).

MS Calcd.: 219; MS Found: 220 (M+H).

### Reference Example 32

### 2,4-Dichloro-6-(pyrrolidin-1-ylmethyl)phenol

To a solution of 2,4-dichlorophenol (5.00 g, 30.6 mmol) and pyrrolidine (2.83 g, 39.8 mmol) in ethanol (10 mL) was added dropwise formaldehyde (36-38% solution in water, 3.1 mL) with ice bath cooling. The mixture was stirred at room temperature for 4 h, diluted with water and extracted with ethyl acetate. The extract was washed with brine, dried over anhydrous magnesium sulfate, filtered and concentrated in vacuo. The residue was purified by flash column chromatography on silica gel eluting with a 10-50% ethyl acetate/n-hexane gradient mixture. The filtrate was concentrated in vacuo to give the title compound as a colorless oil (6.58 g, 26.7 mmol, 87%).

¹H NMR (CDCl₃) δ 1.83-1.92 (m, 4H), 2.63-2.67 (m, 4H), 3.81 (s, 2H), 6.85 (d, J=2.4 Hz, 1H), 7.23 (d, J=2.4 Hz, 1H), 12.04 (s, 1H).

MS Calcd.: 245; MS Found: 246 (M+H).

### Reference Example 33

### 2,4-Dichloro-6-[(2-methylpyrrolidin-1-yl)methyl]phenol

To a solution of 2,4-dichlorophenol (612 mg, 3.75 mmol) and 2-methylpyrrolidine (640 mg, 7.51 mmol) in ethanol (1.0 mL) was added dropwise formaldehyde (36-38% solution in water, 0.70 mL) with ice bath cooling. The mixture was stirred at room temperature for 4 h, diluted with water and extracted with ethyl acetate. The extract was washed with brine, dried over anhydrous magnesium sulfate, filtered and concentrated in vacuo. The residue was purified by flash column chromatography on silica gel eluting with a 3-50% ethyl acetate/n-hexane gradient mixture. The filtrate was concentrated in vacuo to give the title compound as a colorless oil (640 mg, 2.46 mmol, 66%).

MS Calcd.: 259; MS Found: 260 (M+H).

### Reference Example 34

### 2-Chloro-4-(dimethylamino)-6-(pyrrolidin-1-ylmethyl)phenol

To a solution of 2-chloro-4-dimethylaminophenol (1.00 g, 5.83 mmol) and pyrrolidine (0.633 mL, 7.58 mmol) in ethanol (5.0 mL) was added dropwise formaldehyde (36-38% solution in water, 0.52 mL) with ice bath cooling. The mixture was stirred at room temperature for 18 h, diluted with water, and extracted with ethyl acetate. The extract was washed with brine, dried over anhydrous magnesium sulfate, filtered and concentrated in vacuo. The residue was purified by flash column chromatography on silica gel eluting with a 10-70% ethyl acetate/n-hexane gradient mixture. The filtrate was concentrated in vacuo to give the title compound as a brown oil (314 mg, 1.24 mmol, 21%).

¹H NMR (CDCl₃) δ 1.82-1.87 (m, 4H), 2.61-2.67 (m, 4H), 2.83 (s, 6H), 3.79 (s, 2H), 6.36 (d, J=2.7 Hz, 1H), 6.69 (d, J=2.7 Hz, 1H), 12.04 (s, 1H).

MS Calcd.: 254; MS Found: 255 (M;H).

### Reference Example 35

### 1-(2,4-Dichloro-1-methyl-1H-benzimidazol-7-yl)propan-1-one

### i) Methyl 7-chloro-3-methyl-2-oxo-2,3-dihydro-1H-benzimidazole-4-carboxylate

To a suspension of methyl 3-methyl-2-oxo-2,3-dihydro-1H-benzimidazole-4-carboxylate (13.0 g, 59.5 mmol) in ethyl acetate (360 mL) was added 2,2′-azobis(2-methylpropionitrile (10.7 g, 65.5 mmol), and the mixture was stirred at 75° C. for 40 min. To the mixture was added portionwise N-chlorosuccinimide (8.34 g, 62.4 mmol), and the mixture was stirred at 75° C. for 24 h and diluted with aqueous saturated sodium hydrogen carbonate solution. The mixture was extracted with ethyl acetate, and the extract was washed with brine, dried over anhydrous magnesium sulfate, filtered and concentrated in vacuo. The residue was recrystallized from ethanol to give the title compound as colorless crystals (3.46 g, 14.4 mmol, 24%).

¹H NMR (CDCl₃) δ 3.60 (s, 3H), 3.94 (s, 3H), 7.07 (d, J=8.7 Hz, 1H), 7.48 (d, J=8.7 Hz, 1H), 10.09 (s, 1H).

MS Calcd.: 240; MS Found: 241 (M+H).

### ii) 7-Chloro-3-methyl-2-oxo-2,3-dihydro-1H-benzimidazole-4-carboxamide

To a solution of methyl 7-chloro-3-methyl-2-oxo-2,3-dihydro-1H-benzimidazole-4-carboxylate (4.33 g, 17.9 mmol) in N,N-dimethylformamide (50 mL) were added formamide (2.38 g, 52.8 mmol) and sodium methoxide (28% solution in methanol, 2.1 mL), and the mixture was stirred at 100° C. for 18 h. The mixture was diluted with aqueous saturated ammonium chloride solution, and the precipitate generated was collected, washed with water and ethyl acetate, and dried in vacuo to give the title compound as colorless crystals (3.70 g, 16.4 mmol, 92%).

¹H NMR (DMSO-d₆) δ 3.31 (s, 3H), 7.02 (s, 2H), 7.62-7.64 (m, 1H), 8.01-8.03 (m, 1H), 11.32 (s, 1H).

MS Calcd.: 225; MS Found: 226 (M+H).

### iii) 7-Chloro-3-methyl-2-oxo-2,3-dihydro-1H-benzimidazole-4-carbonitrile

To a solution of 7-chloro-3-methyl-2-oxo-2,3-dihydro-1H-benzimidazole-4-carboxamide (11.2 g, 49.6 mmol) in N,N-dimethylformamide (100 mL) was added thionylchloride (10.8 mL, 148 mmol), and the mixture was stirred at 50° C. for 3 h. The mixture was neutralized with aqueous saturated sodium hydrogen carbonate solution, and the precipitate generated was collected, washed with water and diisopropyl ether, and dried in vacuo to give the title compound as colorless crystals (9.38 g, 45.2 mmol, 91%).

¹H NMR (CDCl₃) δ 3.53 (s, 3H), 7.18 (d, J=8.7 Hz, 1H), 7.40 (d, J=8.7 Hz, 1H), 11.98 (s, 1H).

MS Calcd.: 207; MS Found: 208 (M+H).

### iv) 4-Chloro-1-methyl-7-propionyl-1,3-dihydro-2H-benzimidazol-2-one

To a suspension of methyl 7-chloro-3-methyl-2-oxo-2,3-dihydro-1H-benzimidazole-4-carbonitrile (9.37 g, 41.5 mmol) in tetrahydrofuran (120 mL) was added ethylmagnesium bromide (3.0 M solution in diethyl ether, 41.5 mL, 125 mmol), and the mixture was stirred at 50° C. for 3 h and poured into crashed ice. The mixture was acidified by 1N hydrochloric acid and stirred at 70° C. for 1 h. The mixture was neutralized with aqueous saturated sodium hydrogen carbonate solution, and extracted with ethyl acetate. The extract was washed with brine, dried over anhydrous magnesium sulfate, filtered and concentrated in vacuo. The residue was purified by flash column chromatography on silica gel eluting with a 10-50% ethyl acetate/n-hexane gradient mixture. The filtrate was concentrated in vacuo to give the solid, which was recrystallized from ethanol to give the title compound as colorless crystals (5.99 g, 25.1 mmol, 60%).

¹H NMR (CDCl₃) δ 1.09 (t, J=7.2 Hz, 3H), 3.05 (q, J=7.2 Hz, 2H), 3.16 (s, 3H), 7.13 (d, J=8.7 Hz, 1H), 7.41 (d, J=8.7 Hz, 1H), 11.73 (s, 1H).

MS Calcd.: 238; MS Found: 239 (M+H).

### v) 1-(2,4-Dichloro-1-methyl-1H-benzimidazol-7-yl)propan-1-one

A mixture of 4-chloro-1-methyl-7-propionyl-1,3-dihydro-2H-benzimidazol-2-one (1.50 g, 6.28 mmol) and phosphoryl chloride (20 mL) was stirred at 100° C. for 3 h. After cooling, phosphoryl chloride was evaporated in vacuo. The residue was neutralized with saturated aqueous sodium hydrogen carbonate solution, and the mixture was extracted with ethyl acetate. The extract was washed with brine, dried over anhydrous magnesium sulfate, filtered and concentrated in vacuo. The residue was purified by flash column chromatography on silica gel eluting with a 2-40% ethyl acetate/n-hexane gradient mixture. The filtrate was concentrated in vacuo to give the solid, which was washed with n-heptane to give the title compound as a colorless powder (1.59 g, 6.20 mmol, 99%).

¹H NMR (CDCl₃) δ 1.27 (t, J=6.9 Hz, 3H), 3.06 (q, J=6.9 Hz, 2H), 3.82 (s, 3H), 7.31 (d, J=8.4 Hz, 1H), 7.55 (d, J=8.4 Hz, 1H).

MS Calcd.: 256; MS Found: 257 (M+H).

### Reference Example 36

### Isopropyl [2,4-dichloro-7-(1-ethylpropyl)-1H-benzimidazol-1-yl]acetate

### i) Isopropyl [7-(1-ethylpropyl)-2-oxo-2,3-dihydro-1H-benzimidazol-1-yl]acetate

To a solution of tert-butyl 4-(1-ethylpropyl)-2-oxo-2,3-dihydro-1H-benzimidazole-1-carboxylate (23.2 g, 76.2 mmol) in N,N-dimethylformamide (120 mL) were added isopropyl bromoacetate (10.9 mL, 83.9 mmol) and potassium carbonate (11.6 g, 83.9 mmol), and the mixture was stirred at room temperature for 3 hr. The reaction mixture was diluted with water and extracted with ethyl acetate. The organic layer was washed with water and brine, dried over anhydrous sodium sulfate and concentrated in vacuo. The residue was dissolved in ethyl acetate (20 mL), and 4N hydrogen chloride in ethyl acetate (40 mL) was added to the solution at 0° C. The mixture was stirred at room temperature for 2.5 hr. The reaction mixture was diluted with saturated aqueous sodium hydrogen carbonate solution (100 mL) and extracted with ethyl acetate. The organic layer was washed with water and brine, dried over anhydrous sodium sulfate and concentrated in vacuo. The resulting solid was suspended in hexane (40 mL), and the suspension was stirred at 60° C. for 30 min and at room temperature for 1 hr. The resulting solid was collected by filtration and washed with n-hexane to give the title compound (17.9 g, 58.8 mmol, 77%) as colorless crystals.

¹H NMR (CDCl₃) δ: 0.81 (t, J=7.5 Hz, 6H), 1.26 (d, J=6.3 Hz, 6H), 1.55-1.79 (m, 4H), 2.62-2.73 (m, 1H), 4.82 (s, 2H), 5.05-5.15 (m, 1H), 6.90-6.94 (m, 2H), 7.04 (d, J=7.8 Hz, 1H), 9.12 (s, 1H).

MS Calcd.: 304, MS Found: 305 (M+H).

### ii) Isopropyl [4-chloro-7-(1-ethylpropyl)-2-oxo-2,3-dihydro-1H-benzimidazol-1-yl]acetate

To a solution of isopropyl [7-(1-ethylpropyl)-2-oxo-2,3-dihydro-1H-benzimidazol-1-yl]acetate (17.9 g, 58.8 mmol) in chlorobenzene (170 ml) was added 2,2′-azobisisobutyronitrile (966 mg, 5.88 mmol), and the mixture was heated to 60° C. N-Chlorosuccinimide (7.85 g, 58.8 mmol) was added slowly to the mixture, which was stirred at 70° C. for 3.5 days. After cooling, the reaction mixture was washed with saturated aqueous sodium hydrogen carbonate solution. The aqueous layer was separated and extracted with ethyl acetate. The combined organic layer was washed with aqueous sodium chloride solution and brine, dried over anhydrous sodium sulfate and concentrated in vacuo. The residue was purified by silica gel column chromatography eluting with a 15-25% ethyl acetate/n-hexane gradient mixture to give the title compound (15.0 g, 44.3 mmol, 75%) as a solid.

¹H NMR (CDCl₃) δ: 0.83 (t, J=7.5 Hz, 6H), 1.27 (d, J=6.3 Hz, 6H), 1.57-1.78 (m, 4H), 2.59-2.68 (m, 1H), 4.80 (s, 2H), 5.02-5.14 (m, 1H), 6.86 (d, J=8.7 Hz, 1H), 7.04 (d, J=8.7 Hz, 1H), 8.67 (s, 1H).

MS Calcd.: 338, MS Found: 339 (M+H).

### iii) Isopropyl [2,4-dichloro-7-(1-ethylpropyl)-1H-benzimidazol-1-yl]acetate

A mixture of isopropyl [4-chloro-7-(1-ethylpropyl)-2-oxo-2,3-dihydro-1H-benzimidazol-1-yl]acetate (14.5 g, 42.8 mmol) and phosphoryl chloride (60 mL) was stirred at 100° C. for 3 days. After cooling, phosphoryl chloride was evaporated in vacuo. The residue was poured into ice-cold saturated aqueous sodium hydrogen carbonate solution and extracted with ethyl acetate. The combined organic layer was washed with water and brine, dried over anhydrous sodium sulfate and concentrated in vacuo. The residue was purified by silica gel column chromatography eluting with a 5-15% ethyl acetate/n-hexane gradient mixture to give the title compound (14.1 g, 39.5 mmol, 92%) as an oil.

¹H NMR (CDCl₃) δ: 0.81 (t, J=7.5 Hz, 6H), 1.28 (d, J=6.3 Hz, 6H), 1.62-1.83 (m, 4H), 2.72-2.82 (m, 1H), 5.06-5.21 (m, 1H), 5.08 (s, 2H), 7.05 (d, J=8.0 Hz, 1H), 7.28 (d, J=8.0 Hz, 1H).

MS Calcd.: 356, MS Found: 357 (M+H).

### Reference Example 37

### 7-Bromo-2-chloro-1-methyl-1H-benzimidazole

### i) 7-Amino-1-methyl-1,3-dihydro-2H-benzimidazol-2-one

To a solution of N²-methylbenzene-1,2,3-triamine (9.60 g, 70.0 mmol) in tetrahydrofuran (350 mL) was added N,N′-carbonyldiimidazole (11.3 g, 70 mmol). The reaction mixture was stirred at room temperature for 18 h, and concentrated in vacuo. The crude solid was triturated with dichloromethane and collected by filtration to give 6.94 g (61%) of the title compound as a brown powder.

¹H NMR (DMSO-d₆) δ 3.51 (s, 3H), 4.85 (s, 2H), 6.30 (d, J=7.6 Hz, 1H), 6.35 (d, J=8.0 Hz, 1H), 6.68 (t, J=8.0, 1H), 10.55 (s, 1H).

### ii) 7-Bromo-1-methyl-1,3-dihydro-2H-benzimidazol-2-one

A mixture of 7-amino-1-methyl-1,3-dihydro-2H-benzimidazol-2-one (20.0 g, 122 mmol) and t-butyl nitrite (19.0 g, 184 mmol) in acetonitrile (500 mL) was stirred at room temperature for 30 min. The reaction mixture was cooled at 0° C. To the mixture was added portionwise copper (I) bromide (19.3 g, 135 mmol). The mixture was stirred at room temperature for 18 h. To the reaction mixture was added saturated aqueous ammonium chloride solution (400 mL). The mixture was filtered, and washed with methanol. The filtrate was concentrated in vacuo. The residue was dissolved in water and ethyl acetate. The organic layer was separated and the aqueous part was further extracted with ethyl acetate. The combined organics were dried over anhydrous magnesium sulfate, filtered and concentrated in vacuo. The residue was purified by column chromatography on basic silica gel eluting with a 50% ethyl acetate/hexane mixture to give the title compound (7.30 g, 26%) as a brown amorphous.

¹H NMR (CDCl₃) δ 3.55 (s, 3H), 6.90 (t, J=8.1 Hz, 1H), 6.97 (d, J=8.1 Hz, 1H), 7.14 (d, J=8.1 Hz, 1H), 11.2 (br, 1H).

MS Calcd.: 226; Found: 227 (M+H).

### iii) 7-Bromo-2-chloro-1-methyl-1H-benzimidazole

7-Bromo-1-methyl-1,3-dihydro-2H-benzimidazol-2-one (2.20 g, 9.69 mmol) was dissolved in phosphorous oxychloride (30 mL) and heated at 110° C. for 2 days. The reaction mixture was allowed to cool to room temperature, poured into water with an ice and stirred for 1 h. The mixture was extracted with ethyl acetate, washed with aqueous sodium bicarbonate solution, dried over anhydrous magnesium sulfate, filtered and concentrated in vacuo. The residue was purified by column chromatography eluting with a 50% ethyl acetate/hexane mixture to give the title compound (2.12 g, 89%) as a white amorphous.

¹H NMR (CDCl₃) δ 3.68 (s, 3H), 6.95 (t, J=8.1 Hz, 1H), 7.03 (d, J=8.1 Hz, 1H), 7.18 (d, J=8.1 Hz, 1H).

MS Calcd.: 243; Found: 244 (M+H).

### Example 1

### 2-[2-Chloro-4-(trifluoromethoxy)phenoxy]-7-(1-ethylpropyl)-4-methoxy-1-methyl-1H-benzimidazole

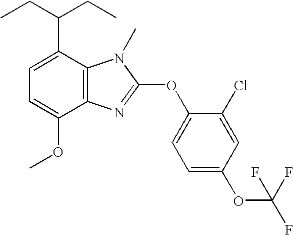

A mixture of 2,4-dichloro-7-(1-ethylpropyl)-4-methoxy-1-methyl-1H-benzimidazole (260 mg, 0.975 mmol), 2-chloro-4-(trifluoromethoxy)phenol (623 mg, 2.93 mmol), potassium carbonate (405 mg, 2.93 mmol) and 1-methyl-2-pyrrolidone (1 mL) was stirred at 120° C. for 18 h under nitrogen atmosphere. The mixture was diluted with water and extracted with ethyl acetate. The extract was washed with brine, dried over anhydrous magnesium sulfate, filtered and concentrated in vacuo. The residue was purified by flash chromatography on silica gel eluting with a 5-20% ethyl acetate/n-hexane gradient mixture. The filtrate was concentrated in vacuo to give the solid, which was recrystallized from methanol to afford the title compound as colorless crystals (196 mg, 45%).

### mp 126-127° C.

¹H NMR (CDCl₃) δ 0.86 (t, J=7.2 Hz, 6H), 1.60-1.85 (m, 4H), 3.10-3.20 (m, 1H), 3.92 (s, 3H), 3.97 (s, 3H), 6.66 (d, J=8.4 Hz, 1H), 6.95 (d, J=8.4 Hz, 1H), 7.19 (dd, J=2.7, 9.0 Hz, 1H), 7.34 (d, J=2.7 Hz, 1H), 7.74 (d, J=9.0 Hz, 1H).

MS Calcd.: 405; MS Found: 406 (M+H).

The compounds of Examples 2-13 were prepared from 2-chloro-7-(1-ethylpropyl)-4-methoxy-1-methyl-1H-benzimidazole and corresponding phenols in the similar method described in Example 1.

### Example 2

### 3,5-Dichloro-4-{[7-(1-ethylpropyl)-4-methoxy-1-methyl-1H-benzimidazol-2-yl]oxy}-N,N-dimethylaniline

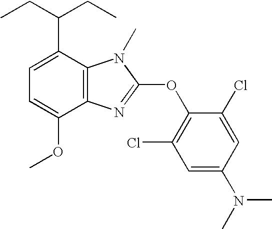

### mp 176-177° C.

¹H NMR (CDCl₃) δ 0.86 (t, J=7.5 Hz, 6H), 1.65-1.85 (m, 4H), 2.94 (s, 6H), 3.15-3.25 (m, 1H), 3.89 (s, 3H), 3.97 (s, 3H), 6.63 (d, J=8.4 Hz, 1H), 6.63 (s, 2H), 6.91 (d, J=8.4 Hz, 1H).

MS Calcd.: 435; MS Found: 436 (M+H), 438.

### Example 3

### 3-Chloro-4-{[7-(1-ethylpropyl)-4-methoxy-1-methyl-1H-benzimidazol-2-yl]oxy}-N,N-dimethylaniline

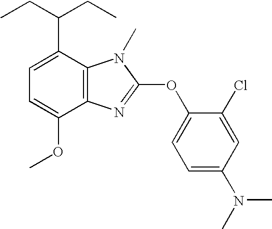

### mp 122-123° C.

¹H NMR (CDCl₃) δ 0.85 (t, J=7.5 Hz, 6H), 1.60-1.85 (m, 4H), 2.94 (s, 6H), 3.15-3.20 (m, 1H), 3.90 (s, 3H), 3.95 (s, 3H), 6.63 (dd, J=3.0, 9.0 Hz, 1H), 6.63 (d, J=9.0 Hz, 1H), 6.72 (d, J=3.0 Hz, 1H), 6.91 (d, J=8.7 Hz, 1H), 7.42 (d, J=8.7 Hz, 1H).

MS Calcd.: 401; MS Found: 402 (M+H), 404.

### Example 4

### 5-Chloro-2-{[7-(1-ethylpropyl)-4-methoxy-1-methyl-1H-benzimidazol-2-yl]oxy}-N,N-dimethylaniline

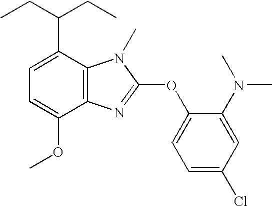

### mp 122-123° C.

¹H NMR (CDCl₃) δ 0.85 (t, J=7.5 Hz, 6H), 1.60-1.85 (m, 4H), 2.82 (s, 6H), 3.18 (m, 1H), 3.91 (s, 3H), 3.93 (s, 3H), 6.65 (d, J=8.1 Hz, 1H), 6.85-6.95 (m, 3H), 7.25 (d, J=8.1 Hz, 1H).

MS Calcd.: 401; MS Found: 402 (M+H), 404.

### Example 5

### 3-{[7-(1-Ethylpropyl)-4-methoxy-1-methyl-1H-benzimidazol-2-yl]oxy}-N,N,6-trimethylpyridin-2-amine

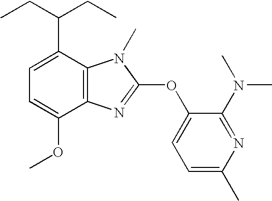

### mp 143-144° C.

¹H NMR (CDCl₃) δ 0.85 (t, J=7.2 Hz, 6H), 1.65-1.85 (m, 4H), 2.41 (s, 3H), 3.00 (s, 6H), 3.10-3.20 (m, 1H), 3.91 (s, 3H), 3.92 (s, 3H), 6.59 (d, J=8.1 Hz, 1H), 6.65 (d, J=8.1 Hz, 1H), 6.93 (d, J=8.1 Hz, 1H), 7.42 (d, J=8.1, 1H).

MS Calcd.: 381; MS Found: 382 (M+H).

### Example 6

### 1-(4-{[7-(1-Ethylpropyl)-4-methoxy-1-methyl-1H-benzimidazol-2-yl]oxy}-3-methylphenyl)ethanone

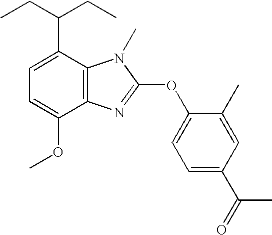

### mp 114-115° C.

¹H NMR (CDCl₃) δ 0.86 (t, J=7.2 Hz, 6H), 1.60-1.85 (m, 4H), 2.38 (s, 3H), 2.59 (s, 3H), 3.10-3.25 (m, 1H), 3.92 (s, 3H), 3.93 (s, 3H), 6.67 (d, J=8.4 Hz, 1H), 6.96 (d, J=8.4 Hz, 1H), 7.46 (d, J=8.4 Hz, 1H), 7.81 (d, J=8.4 Hz, 1H), 7.86 (s, 1H).

MS Calcd.: 380; MS Found: 381 (M+H).

### Example 7

### 3,5-Dichloro-2-{[7-(1-ethylpropyl)-4-methoxy-1-methyl-1H-benzimidazol-2-yl]oxy}-N,N-dimethylaniline

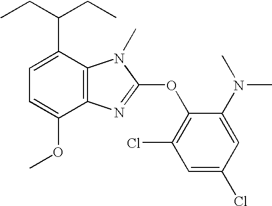

### mp 173-175° C.

¹H NMR (CDCl₃) δ 0.86 (t, J=7.5 Hz, 6H), 1.60-1.85 (m, 4H), 2.84 (s, 6H), 3.15-3.25 (m, 1H), 3.89 (s, 3H), 3.96 (s, 3H), 6.63 (d, J=8.4 Hz, 1H), 6.84 (d, J=2.4 Hz, 1H), 6.92 (d, J=8.4 Hz, 1H), 6.95 (d, J=2.4 Hz, 1H).

MS Calcd.: 435; MS Found: 436 (M+H), 438.

### Example 8

### 2-[(5-Bromo-3-methylpyridin-2-yl)oxy]-7-(1-ethylpropyl)-4-methoxy-1-methyl-1H-benzimidazole

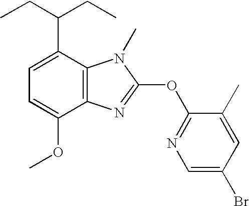

amorphous

¹H NMR (CDCl₃) δ 0.85 (t, J=7.2 Hz, 6H), 1.60-1.85 (m, 4H), 2.38 (s, 3H), 3.10-3.25 (m, 1H), 3.86 (s, 3H), 3.91 (s, 3H), 6.66 (d, J=8.1 Hz, 1H), 6.96 (d, J=8.1 Hz, 1H), 7.70 (d, J=2.1 Hz, 1H), 8.08 (d, J=2.1 Hz, 1H).

MS Calcd.: 417; MS Found: 418 (M+H), 420.

### Example 9

### 1-(5-Chloro-2-{[7-(1-ethylpropyl)-4-methoxy-1-methyl-1H-benzimidazol-2-yl]oxy}phenyl)-N,N-dimethylmethanamine

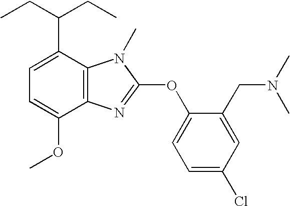

### mp 121-123° C.

¹H NMR (CDCl₃) δ 0.85 (t, J=7.2 Hz, 6H), 1.60-1.75 (m, 4H), 2.25 (s, 6H), 3.10-3.20 (m, 1H), 3.46 (s, 2H), 3.91 (s, 3H), 3.92 (s, 3H), 6.65 (d, J=8.7 Hz, 1H), 6.94 (d, J=8.7 Hz, 1H), 7.20-7.30 (m, 1H), 7.38 (d, J=8.7 Hz, 1H), 7.43 (s, 1H).

MS Calcd.: 415; MS Found: 416 (M+H).

### Example 10

### 2-[2-Bromo-4-(trifluoromethoxy)phenoxy]-7-(1-ethylpropyl)-4-methoxy-1-methyl-1H-benzimidazole

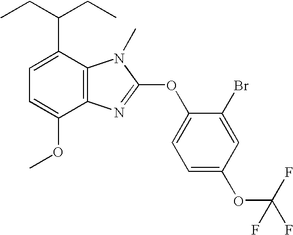

### mp 117-118° C.

¹H NMR (CDCl₃) δ 0.86 (t, J=7.5 Hz, 6H), 1.60-1.85 (m, 4H), 3.10-3.25 (m, 1H), 3.92 (s, 3H), 3.97 (s, 3H), 6.67 (d, J=8.7 Hz, 1H), 6.96 (d, J=8.7 Hz, 1H), 7.20-7.30 (m, 1H), 7.49 (d, J=2.7 Hz, 1H), 7.76 (d, J=8.7 Hz, 1H).

MS Calcd.: 486; MS Found: 487 (M+H), 489.

### Example 11

### 7-(1-Ethylpropyl)-4-methoxy-1-methyl-2-{[6-methyl-2-(trifluoromethyl)pyridin-3-yl]oxy}-1H-benzimidazole

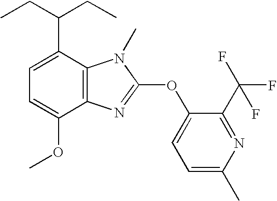

### mp 135-137° C.

¹H NMR (CDCl₃) δ 0.85 (t, J=7.5 Hz, 6H), 1.60-1.85 (m, 4H), 2.62 (s, 3H), 3.10-3.20 (m, 1H), 3.93 (s, 3H), 3.94 (s, 3H), 6.67 (d, J=8.7 Hz, 1H), 6.97 (d, J=8.7 Hz, 1H), 7.42 (d, J=8.7 Hz, 1H), 8.25 (d, J=8.7 Hz, 1H).

MS Calcd.: 407; MS Found: 408 (M+H).

### Example 12

### 2-{[7-(1-Ethylpropyl)-4-methoxy-1-methyl-1H-benzimidazol-2-yl]oxy}-N,N-dimethyl-5-(trifluoromethyl)aniline

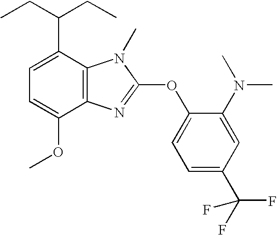

### mp 131-133° C.

¹H NMR (CDCl₃) δ 0.86 (t, J=7.5 Hz, 6H), 1.60-1.85 (m, 4H), 2.07 (s, 6H), 3.10-3.25 (m, 1H), 3.92 (s, 3H), 3.94 (s, 3H), 6.67 (d, J=8.4 Hz, 1H), 6.96 (d, J=8.4 Hz, 1H), 7.19 (d, J=7.8 Hz, 1H), 7.20 (s, 1H), 7.41 (d, J=7.8 Hz, 1H).

MS Calcd.: 435; MS Found: 436 (M+H).

### Example 13

### 1-[2-{[7-(1-Ethylpropyl)-4-methoxy-1-methyl-1H-benzimidazol-2-yl]oxy}-5-(trifluoromethoxy)phenyl]-N,N-dimethylmethanamine

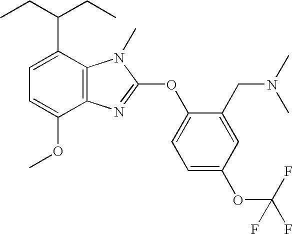

### mp 95-97° C.

¹H NMR (CDCl₃) δ 0.86 (t, J=7.5 Hz, 6H), 1.60-1.85 (m, 4H), 2.43 (s, 6H), 3.15-3.25 (m, 1H), 3.78 (s, 2H), 3.91 (s, 3H), 3.96 (s, 3H), 6.67 (d, J=8.4 Hz, 1H), 6.96 (d, J=8.4 Hz, 1H), 7.20 (d, J=9.0 Hz, 1H), 7.43 (s, 1H), 7.45 (d, J=9.0 Hz, 1H).

MS Calcd.: 465; MS Found: 466 (M+H).

### Example 14

### 7-(1-Ethylpropyl)-4-methoxy-1-methyl-2-[(1,4,5-trimethyl-1H-pyrazol-3-yl)oxy]-1H-benzimidazole

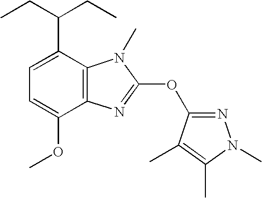

To a mixture of 2-chloro-7-(1-ethylpropyl)-4-methoxy-1-methyl-1H-benzimidazole (70.2 mg, 0.264 mmol), potassium carbonate (109 mg, 0.788 mmol) in N,N-dimethylformamide (0.5 mL) was added a mixture of 1,4,5-trimethyl-1H-pyrazol-3-ol and 1,3,4-trimethyl-1H-pyrazol-5-ol (100 mg, 0.792 mmol). The mixture was stirred at 120° C. for 3 days. The mixture was diluted with water and extracted with ethyl acetate. The extract was washed with brine, dried over anhydrous magnesium sulfate, filtered and concentrated in vacuo. The residue was purified by flash column chromatography on silica gel eluting with a 10-50% ethyl acetate/n-hexane gradient mixture. The filtrate was concentrated in vacuo to give a solid, which was washed with n-hexane to give the title compound as a colorless amorphous (1.6 mg, 0.00449 mmol, 2%).

¹H NMR (CDCl₃) δ 0.84 (t, J=7.4 Hz, 6H), 1.61-1.84 (m, 4H), 1.89 (s, 3H), 2.16 (s, 3H), 3.14-3.17 (m, 1H), 3.66 (s, 3H), 3.91 (s, 3H), 3.89 (s, 3H), 6.63 (d, J=8.6 Hz, 1H), 6.90 (d, J=8.6 Hz, 1H)

MS Calcd.: 356; MS Found: 357 (M+H).

### Example 15

### 1-[2-{[7-(1-Ethylpropyl)-4-methoxy-1-methyl-1H-benzimidazol-2-yl]oxy}-3-methyl-5-(trifluoromethoxy)phenyl]-N,N-dimethylmethanamine Hydrochloride

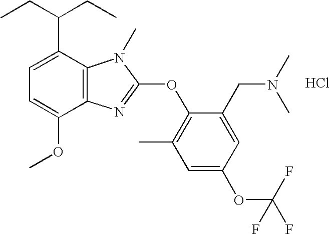

The free base of the title compound, which was prepared from 2-chloro-7-(1-ethylpropyl)-4-methoxy-1-methyl-1H-benzimidazole and 2-[(dimethylamino)methyl]-6-methyl-4-(trifluoromethoxy)phenol in the similar method described in Example 1, was treated with 4N hydrogen chloride in ethyl acetate to give the title compound.

### mp 92-94° C.

¹H NMR (CDCl₃) δ 0.83 (t, J=7.2 Hz, 6H), 1.60-1.80 (m, 4H), 2.10 (s, 3H), 2.76 (s, 3H), 2.77 (s, 3H), 3.20-3.30 (m, 1H), 3.74 (s, 3H), 3.97 (s, 3H), 4.30-4.40 (m, 2H), 6.67 (d, J=8.4 Hz, 1H), 6.92 (d, J=8.4 Hz, 1H), 7.55 (s, 1H), 7.77 (s, 1H), 10.61 (m, 1H).

MS Calcd.: 479; MS Found: 480 (M+H).

### Example 16

### N-[2-Chloro-4-(trifluoromethyl)phenyl]-7-(1-ethylpropyl)-4-methoxy-1-methyl-1H-benzimidazol-2-amine

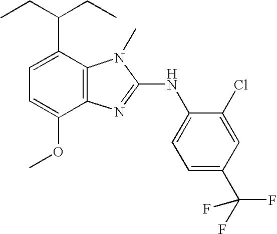

A mixture of 2-chloro-7-(1-ethylpropyl)-4-methoxy-1-methyl-1H-benzimidazole (200 mg, 0.750 mmol) and 2-chloro-4-trifluoromethylaniline (450 mg, 2.25 mmol) in 1-methyl-2-pyrrolidone (0.5 mL) was stirred at 120° C. for 96 h under nitrogen atmosphere. The mixture was diluted with aqueous saturated sodium hydrogen carbonate solution and extracted with ethyl acetate. The extract was washed with brine, dried over anhydrous magnesium sulfate, filtered and concentrated in vacuo. The residue was purified by flash chromatography on silica gel with a 5-30% ethyl acetate/n-hexane gradient mixture. The filtrate was concentrated in vacuo to give the solid, which was recrystallized from methanol to afford the title compound as colorless crystals (103 mg, 32%).

### mp 170-171° C.

¹H NMR (CDCl₃) δ 0.85 (t, J=7.5 Hz, 6H), 1.60-1.85 (m, 4H), 3.15-3.20 (m, 1H), 3.88 (s, 3H), 4.00 (s, 3H), 6.71 (d, J=8.4 Hz, 1H), 6.85 (s, 1H), 6.99 (d, J=7.8 Hz, 1H), 7.50 (d, J=8.4 Hz, 1H), 7.65 (s, 1H), 8.00 (d, J=7.8 Hz, 1H).

MS Calcd.: 425; MS Found: 426 (M+H), 428.

The compounds of Examples 17-21 were prepared from 2-chloro-7-(1-ethylpropyl)-4-methoxy-1-methyl-1H-benzimidazole and corresponding anilines in the similar method described in Example 16.

### Example 17

### N-[4-Chloro-2-(trifluoromethyl)phenyl]-7-(1-ethylpropyl)-4-methoxy-1-methyl-1H-benzimidazol-2-amine

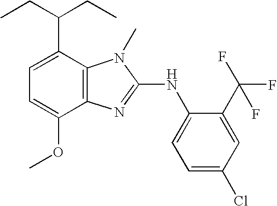

### mp 122-123° C.

¹H NMR (CDCl₃) δ 0.84 (t, J=7.5 Hz, 6H), 1.60-1.85 (m, 4H), 3.10-3.20 (m, 1H), 3.75 (s, 3H), 3.99 (s, 3H), 6.50 (s, 1H), 6.70 (d, J=8.1 Hz, 1H), 6.97 (d, J=8.1 Hz, 1H), 7.40 (dd, J=2.1, 8.7 Hz, 1H), 7.50 (d, J=8.7 Hz, 1H), 7.57 (d, J=2.1 Hz, 1H).

MS Calcd.: 425; MS Found: 426 (M+H), 428.

### Example 18

### N-[2-Chloro-4-(trifluoromethoxy)phenyl]-7-(1-ethylpropyl)-4-methoxy-1-methyl-1H-benzimidazol-2-amine

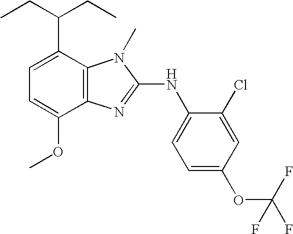

### mp 175-177° C.

¹H NMR (CDCl₃) δ 0.84 (t, J=7.5 Hz, 6H), 1.60-1.85 (m, 4H), 3.10-3.20 (m, 1H), 3.86 (s, 3H), 3.99 (s, 3H), 6.63 (s, 1H), 6.69 (d, J=8.4 Hz, 1H), 6.95 (d, J=8.4 Hz, 1H), 7.14 (dd, J=2.7, 8.7 Hz, 1H), 7.28 (d, J=2.7 Hz, 1H), 7.99 (d, J=8.7 Hz, 1H).

MS Calcd.: 441; MS Found: 442 (M+H), 444.

### Example 19

### N-(2-Bromo-4-chlorophenyl)-7-(1-ethylpropyl)-4-methoxy-1-methyl-1H-benzimidazol-2-amine

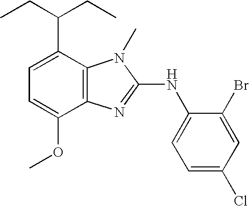

### mp 193-195° C.

¹H NMR (CDCl₃) δ 0.85 (t, J=7.5 Hz, 6H), 1.60-1.85 (m, 4H), 3.10-3.20 (m, 1H), 3.84 (s, 3H), 3.99 (s, 3H), 6.65 (s, 1H), 6.69 (d, J=8.4 Hz, 1H), 6.95 (d, J=8.4 Hz, 1H), 7.25 (m, 1H), 7.53 (s, 1H), 7.88 (d, J=8.4 Hz, 1H).

MS Calcd.: 435; MS Found: 436 (M+H), 438.

### Example 20

### N-(2,4-Dichlorophenyl)-7-(1-ethylpropyl)-4-methoxy-1-methyl-1H-benzimidazol-2-amine

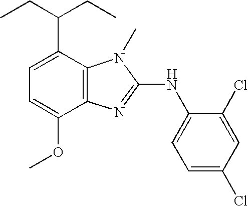

### mp 198-200° C.

¹H NMR (CDCl₃) δ 0.84 (t, J=7.5 Hz, 6H), 1.60-1.85 (m, 4H), 3.10-3.20 (m, 1H), 3.84 (s, 3H), 3.99 (s, 3H), 6.62 (s, 1H), 6.69 (d, J=8.4 Hz, 1H), 6.95 (d, J=8.4 Hz, 1H), 7.21 (dd, J=2.7, 8.7 Hz, 1H), 7.37 (d, J=2.7 Hz, 1H), 7.91 (d, J=8.7 Hz, 1H).

MS Calcd.: 391; MS Found: 392 (M+H), 394.

### Example 21

### 4-Chloro-N¹-[7-(1-ethylpropyl)-4-methoxy-1-methyl-1H-benzimidazol-2-yl]-N²,N²-dimethylbenzene-1,2-diamine

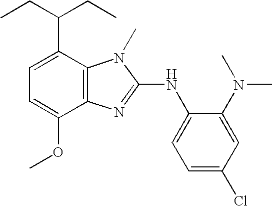

### mp 47-49° C.

¹H NMR (CDCl₃) δ 0.85 (t, J=7.5 Hz, 6H), 1.64-1.83 (m, 4H), 2.71 (s, 6H), 3.13-3.22 (m, 1H), 3.83 (s, 3H), 3.99 (s, 3H), 6.68 (d, J=8.4 Hz, 1H), 6.91 (d, J=8.4 Hz, 1H), 7.07-7.12 (m, 2H), 7.55 (s, 1H), 7.98 (d, J=8.7 Hz, 1H).

MS Calcd.: 400; MS Found: 401 (M+H).

### Example 22

### 5-Chloro-2-{[7-(1-ethylpropyl)-4-methoxy-1-methyl-1H-benzimidazol-2-yl]oxy}benzenesulfonamide

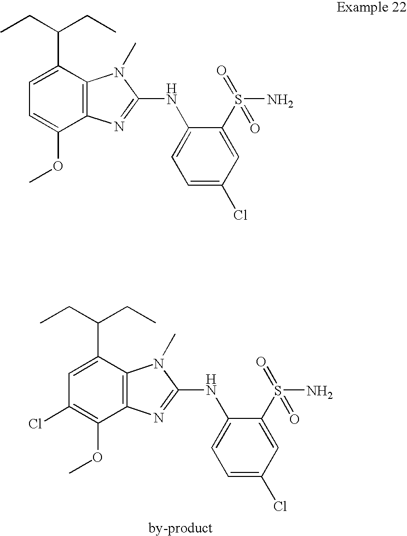

2-(2-Bromo-4-chlorophenoxy)-7-(1-ethylpropyl)-4-methoxy-1-methyl-1H-benzimidazole, which was prepared from 2-chloro-7-(1-ethylpropyl)-4-methoxy-1-methyl-1H-benzimidazole and 2-bromo-4-chlorophenol in the similar method described in Example 1, was dissolved in tetrahydrofuran (4.0 mL). To the solution was added n-butyllithium in n-hexane (1.60 M, 0.31 mL, 0.482 mmol) at −78° C., and the mixture was stirred for 20 min. Sulfurous acid gas was bubbled through the mixture for 5 min, and the mixture was stirred at −78° C. for 20 min. Sulfuryl chloride (73 μL, 0.912 mmol) was added to the mixture, followed by an addition of aqueous ammonia (6.0 mL) after 10 min. The mixture was stirred at room temperature for 1 h, poured into saturated aqueous ammonium chloride solution, and extracted with ethyl acetate (20 mL×3). The combined organic layer was washed with brine, dried over anhydrous magnesium sulfate, filtered and concentrated in vacuo. The residue was purified by flash column chromatography on silica gel eluting with a 1-20% ethyl acetate/n-hexane gradient mixture to give the crude title compound and a by-product. Each compound was recrystallized from ethyl acetate-n-hexane to give the title compound (23.3 mg, 0.0532 mmol, 12%) as colorless crystals and 5-chloro-2-{[5-chloro-7-(1-ethylpropyl)-4-methoxy-1-methyl-1H-benzimidazol-2-yl]oxy}benzenesulfonamide (37.0 mg, 0.0783 mmol, 17%) as colorless crystals.

### mp 148-149° C.

¹H NMR (CDCl₃) δ 0.86 (t, J=7.2 Hz, 6H), 1.54-1.79 (m, 4H), 3.14-3.19 (m, 1H), 3.89 (s, 3H), 3.95 (s, 3H), 5.62 (brs, 2H), 6.68 (d, 1H, J=8.7 Hz), 6.99 (d, J=8.7 Hz, 1H), 7.38 (d, J=8.4 Hz, 1H), 7.53 (dd, J=2.4 Hz, 8.4 Hz, 1H), 8.02 (d, J=2.4 Hz, 1H).

MS Calcd.: 437; MS Found: 438 (M+H).

### 5-Chloro-2-{[5-chloro-7-(1-ethylpropyl)-4-methoxy-1-methyl-1H-benzimidazol-2-yl]oxy}benzenesulfonamide (by-product)

### mp 153-154° C.

¹H NMR (CDCl₃) δ 0.86 (t, J=7.2 Hz, 6H), 1.61-1.81 (m, 4H), 3.12-3.17 (m, 1H), 3.94 (s, 3H), 4.02 (s, 3H), 5.52 (brs, 2H), 7.02 (s, 1H), 7.50 (d, J=9.0 Hz, 1H), 7.57 (dd, J=2.7 Hz, 9.0 Hz, 1H), 8.04 (d, J=2.7 Hz, 1H).

MS Calcd.: 471; MS Found: 472 (M+H).

### Example 23

### 2-(2-Bromo-4,6-dichlorophenoxy)-4-chloro-7-(1-ethylpropyl)-1-methyl-1H-benzimidazole

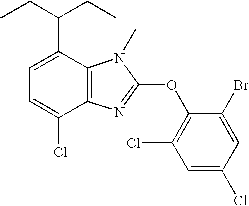

To a solution of 2-bromo-4,6-dichlorophenol (9.50 g, 39.2 mmol) in N,N-dimethylformamide (10 mL) were added potassium carbonate (9.50 g, 39.2 mmol) and 2,4-dichloro-7-(1-ethylpropyl)-1-methyl-1H-benzimidazole (5.00 g, 18.4 mmol). The mixture was heated at 120° C. for 3 days. The mixture was diluted with water (100 mL) and extracted with ethyl acetate (100 mL×3). The combined organic layer was washed with brine, dried over anhydrous magnesium sulfate, filtered and concentrated in vacuo. The residue was purified by flash column chromatography on silica gel eluting with a 1-20% ethyl acetate/n-hexane gradient mixture. The filtrate was concentrated in vacuo to give the solid, which was recrystallized from ethyl acetate-n-hexane to give the title compound (2.06 g, 4.32 mmol, 23%) as white crystals.

### mp 165-167° C.

¹H NMR (CDCl₃) δ 0.86 (t, J=7.2 Hz, 6H), 1.65-1.80 (m, 4H), 3.18-3.24 (m, 1H), 3.99 (s, 3H), 6.94 (d, J=8.1 Hz, 1H), 7.14 (d, J=8.1 Hz, 1H), 7.46 (d, J=2.7 Hz, 1H), 7.56 (d, J=2.7 Hz, 1H).

MS Calcd.: 474; MS Found: 475 (M+H).

### Example 24

### 3,5-Dichloro-2-{[4-chloro-7-(1-ethylpropyl)-1-methyl-1H-benzimidazol-2-yl]oxy}-N,N-dimethylaniline

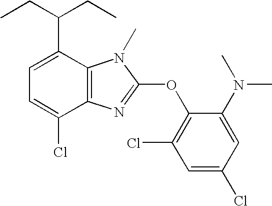

To a solution of sodium tert-butoxide (75.5 mg, 0.786 mmol) in toluene (1.5 mL) were added 2-(di-tert-butylphosphino)biphenyl (46.9 mg, 0.157 mmol), tris(dibenzylideneacetone)dipalladium (143 mg, 0.157 mmol), 2-(2-bromo-4,6-dichlorophenoxy)-4-chloro-7-(1-ethylpropyl)-1-methyl-1H-benzimidazole (250 mg, 0.524 mmol) and dimethylamine (2.0 M solution in tetrahydrofuran, 1.2 mL, 2.4 mmol). The mixture was stirred at 70° C. under nitrogen for 32 h. To the mixture were added ethyl acetate (10 mL) and water (15 mL), and the black precipitation was removed by filtration. The filtrate was extracted with ethyl acetate (15 mL×4). The combined organic layer was washed with brine, dried over anhydrous magnesium sulfate, filtered and concentrated in vacuo. The residue was purified by flash column chromatography on silica gel eluting with a 0-15% ethyl acetate/n-hexane gradient mixture. The filtrate was concentrated in vacuo to give the solid, which was collected by filtration and washed with diisopropyl ether to give the title compound (37.6 mg, 0.0853 mmol, 16%) as a white powder.

### mp 172-173° C.

¹H NMR (CDCl₃) δ 0.86 (t, J=7.2 Hz, 6H), 1.55-1.83 (m, 4H), 2.84 (s, 6H), 3.20-3.24 (m, 1H), 3.98 (s, 3H), 6.88 (d, J=2.4 Hz, 1H), 6.89 (d, J=8.1 Hz, 1H), 7.00 (d, J=2.4 Hz, 1H), 7.13 (d, J=8.1 Hz, 1H).

MS Calcd.: 439; MS Found: 440 (M+H).

The compounds of Examples 25-29 were prepared from 2-(2-bromo-4,6-dichlorophenoxy)-4-chloro-7-(1-ethylpropyl)-1-methyl-1H-benzimidazole and corresponding amines in the similar method described in Example 24.

### Example 25

### 4-Chloro-2-(2,4-dichloro-6-morpholin-4-ylphenoxy)-7-(1-ethylpropyl)-1-methyl-1H-benzimidazole

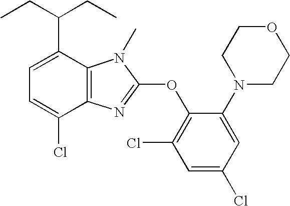

### mp 155-157° C.

¹H NMR (CDCl₃) δ 0.86 (t, J=7.5 Hz, 6H), 1.59-1.85 (m, 4H), 2.97 (t, J=4.8 Hz, 4H), 3.15-3.29 (m, 1H), 3.49 (t, J=4.8 Hz, 4H), 3.96 (s, 3H), 6.93 (d, J=8.4 Hz, 1H), 7.01 (d, J=2.7 Hz, 1H), 7.15 (d, J=8.4 Hz, 1H), 7.19 (d, J=2.7 Hz, 1H).

MS Calcd.: 481; MS Found: 482 (M+H).

### Example 26

### 4-Chloro-2-[2,4-dichloro-6-(4-methylpiperazin-1-yl)phenoxy]-7-(1-ethylpropyl)-1-methyl-1H-benzimidazole

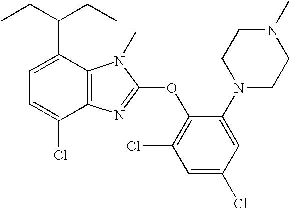

### mp 162-164° C.

¹H NMR (CDCl₃) δ 0.86 (t, J=7.2 Hz, 6H), 1.65-1.85 (m, 4H), 2.11 (s, 3H), 2.18 (t, J=4.8 Hz, 4H), 2.98 (t, J=4.8. Hz, 4H), 3.18-3.28 (m, 1H), 3.97 (s, 3H), 6.93 (d, J=8.7 Hz, 1H), 7.01 (d, J=2.4 Hz, 1H), 7.14 (d, J=8.7 Hz, 1H), 7.17 (d, J=2.4 Hz, 1H).

MS Calcd.: 494; MS Found: 495 (M+H).

### Example 27

### 4-Chloro-2-[2,4-dichloro-6-(1H-imidazol-1-yl)phenoxy]-7-(1-ethylpropyl)-1-methyl-1H-benzimidazole

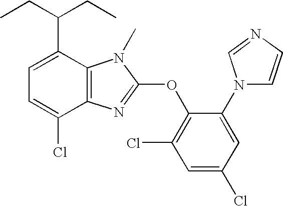

### mp 134-135° C.

¹H NMR (CDCl₃) δ 0.83 (t, J=7.2 Hz, 6H), 1.56-1.80 (m, 4H), 3.10-3.18 (m, 1H), 3.83 (s, 3H), 6.68 (s, 1H), 6.93 (d, J=8.1 Hz, 1H), 7.04 (brs, 1H), 7.15 (d, J=8.1 Hz, 1H), 7.38 (d, J=2.7 Hz, 1H), 7.55 (d, J=2.7 Hz, 1H), 7.77 (brs, 1H).

MS Calcd.: 462; MS Found: 463 (M+H).

### Example 28

### 3,5-Dichloro-2-{[4-chloro-7-(1-ethylpropyl)-1-methyl-1H-benzimidazol-2-yl]oxy}-N-methylaniline

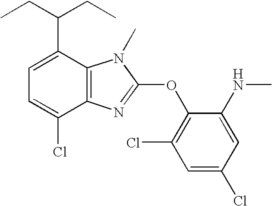

### mp 162-163° C.

¹H NMR (CDCl₃) δ 0.79 (t, J=7.5 Hz, 6H), 1.60-1.78 (m, 4H), 3.08-3.13 (m, 1H), 3.41 (s, 3H), 3.41 (brs, 1H), 3.57 (s, 3H), 6.87 (d, J=2.4 Hz, 1H), 6.95 (d, J=8.1 Hz, 1H), 7.20 (d, J=8.1 Hz, 1H), 7.23 (d, J=2.4 Hz, 1H).

MS Calcd.: 425; MS Found: 426 (M+H).

### Example 29

### 4-Chloro-2-(2,4-dichloro-6-pyrrolidin-1-ylphenoxy)-7-(1-ethylpropyl)-1-methyl-1H-benzimidazole

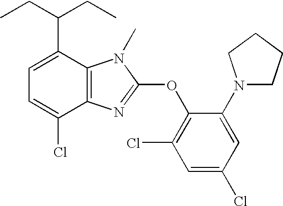

### mp 209-210° C.

¹H NMR (CDCl₃) δ 0.86 (t, J=7.5 Hz, 6H), 1.71-1.79 (m, 4H), 1.88 (t, J=6.6 Hz, 4H), 3.08-3.11 (m, 1H), 3.41 (t, J=6.6 Hz, 4H), 3.95 (s, 3H), 6.61 (d, J=2.4 Hz, 1H), 6.73 (d, J=2.4 Hz, 1H), 6.92 (d, J=7.8 Hz, 1H), 7.13 (d, J=7.8 Hz, 1H).

MS Calcd.: 465; MS Found: 466 (M+H).

### Example 30

### 2-{[7-(1-Ethylpropyl)-4-methoxy-1-methyl-1H-benzimidazol-2-yl]oxy}-N,N-dimethyl-5-(trifluoromethoxy)aniline

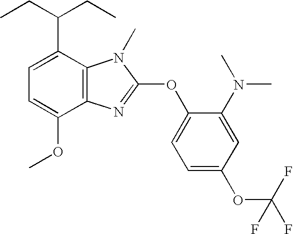

The title compound was prepared from 2-[2-bromo-4-(trifluoromethoxy)phenoxy]-7-(1-ethylpropyl)-4-methoxy-1-methyl-1H-benzimidazole and tetrahydrofuran solution of dimethylamine in the similar method described in Example 24.

### mp 93-95° C.

¹H NMR (CDCl₃) δ 0.86 (t, J=7.5 Hz, 6H), 1.60-1.80 (m, 4H), 2.84 (s, 6H), 3.15-3.25 (m, 1H), 3.91 (s, 3H), 3.94 (s, 3H), 6.66 (d, J=8.7 Hz, 1H), 6.78 (d, J=8.1 Hz, 1H), 6.79 (d, J=1.2 Hz, 1H), 6.94 (d, J=8.7 Hz, 1H), 7.32 (dd, J=1.2, 8.1 Hz, 1H).

MS Calcd.: 451; MS Found: 452 (M+H).

### Example 31

### 3,5-Dichloro-2-{[4-chloro-7-(1-ethylpropyl)-1-methyl-1H-benzimidazol-2-yl]oxy}benzonitrile

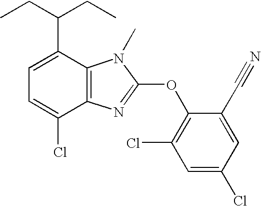

To a solution of 2-(2-bromo-4,6-dichlorophenoxy)-4-chloro-7-(1-ethylpropyl)-1-methyl-1H-benzimidazole (1.40 g, 2.93 mmol) in 1-methyl-2-pyrrolidone (13 mL) was added copper(I) cyanide (840 mg, 4.41 mmol). The mixture was irradiated at 150° C. by a microwave for 20 min. The mixture was diluted with saturated aqueous sodium hydrogen carbonate solution and extracted with ethyl acetate (50 mL×4). The combined organic layer was washed with brine, dried over anhydrous magnesium sulfate, filtered and concentrated in vacuo. The residue was purified by flash column chromatography on silica gel eluting with a 0-15% ethyl acetate/n-hexane gradient mixture. The filtrate was concentrated in vacuo to give the solid, which was collected by filtration and washed with n-hexane to give the title compound (510 mg, 1.21 mmol, 41%) as a white powder.

### mp 155-156° C.

¹H NMR (CDCl₃) δ 0.86 (t, J=7.5 Hz, 6H), 1.69-1.83 (m, 4H), 3.20-3.23 (m, 1H), 4.01 (s, 3H), 6.97 (d, J=8.4 Hz, 1H), 7.17 (d, J=8.4 Hz, 1H), 7.62 (d, J=2.4 Hz, 1H), 7.73 (d, J=2.4 Hz, 1H).

MS Calcd.: 421; MS Found: 422 (M+H).

### Example 32

### 3,5-Dichloro-2-{[4-chloro-7-(1-ethylpropyl)-1-methyl-1H-5 benzimidazol-2-yl]oxy}benzenesulfonamide

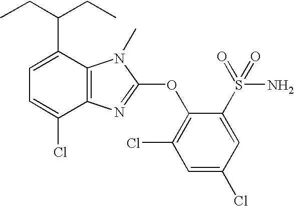

To a solution of 2-(2-bromo-4,6-dichlorophenoxy)-4-chloro-7-(1-ethylpropyl)-1-methyl-1H-benzimidazole (200 mg, 0.419 mmol) in tetrahydrofuran (4.0 mL) was added n-butyllithium (1.60 M solution in n-hexane, 0.30 mL, 0.480 mmol) at −78° C., and the mixture was stirred for 20 min. Sulfurous acid gas was bubbled through the mixture for 5 min, and the mixture was stirred at −78° C. for 20 min. To the mixture was added sulfuryl chloride (0.037 mL, 0.461 mmol), followed by an addition of aqueous ammonia (3.0 mL) after 10 min. The mixture was stirred at room temperature for 1 h, poured into saturated aqueous ammonium chloride solution, and extracted with ethyl acetate (20 mL×3). The combined organic layer was washed with brine, dried over anhydrous magnesium sulfate, filtered and concentrated in vacuo. The residue was purified by flash column chromatography on silica gel eluting with a 1-20% ethyl acetate/n-hexane gradient mixture. The filtrate was concentrated in vacuo to give a solid, which was recrystallized from ethyl acetate/n-hexane to give the title compound (72.6 mg, 0.152 mmol, 36%) as white crystals.

### mp 133-134° C.

¹H NMR (CDCl₃) δ 0.87 (t, J=7.2 Hz, 6H), 1.67-1.83 (m, 4H), 3.20-3.24 (m, 1H), 4.03 (s, 3H), 6.33 (s, 2H), 6.98 (d, J=8.7 Hz, 1H), 7.16 (d, J=8.7 Hz, 1H), 7.69 (d, J=2.4 Hz, 1H), 8.03 (d, J=2.4 Hz, 1H).

MS Calcd.: 475; MS Found: 476 (M+H).

### Example 33

### 2-(4-Bromo-2-chloro-6-methylphenoxy)-4-chloro-7-(1-ethylpropyl)-1-methyl-1H-benzimidazole

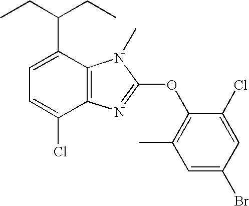

The title compound was prepared from 2,4-dichloro-7-(1-ethylpropyl)-1-methyl-1H-benzimidazole and 4-bromo-2-chloro-6-methylphenol in the similar method described in Example 24.

### mp 163-164° C.

¹H NMR (CDCl₃) δ 0.86 (t, J=7.2 Hz, 6H), 1.68-1.82 (m, 4H), 2.31 (s, 3H), 3.20-3.24 (m, 1H), 3.98 (s, 3H), 6.92 (d, J=8.4 Hz, 1H), 7.13 (d, J=8.4 Hz, 1H), 7.35 (d, J=2.4 Hz, 1H), 7.45 (d, J=2.4 Hz, 1H).

MS Calcd.: 454; MS Found: 455 (M+H).

### Example 34

### 3-Chloro-4-{[4-chloro-7-(1-ethylpropyl)-1-methyl-1H-benzimidazol-2-yl]oxy}-5-methylbenzonitrile

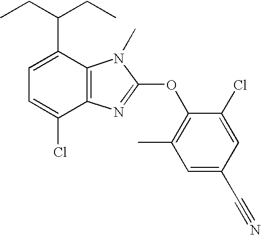

The title compound was prepared from 2-(4-bromo-2-chloro-6-methylphenoxy)-4-chloro-7-(1-ethylpropyl)-1-methyl-1H-benzimidazole in the similar method described in Example 31.

### mp 147-148° C.

¹H NMR (CDCl₃) δ 0.87 (t, J=7.2 Hz, 6H), 1.66-1.83 (m, 4H), 2.38 (s, 3H), 3.20-3.24 (m, 1H), 4.00 (s, 3H), 6.94 (d, J=8.4 Hz, 1H), 7.14 (d, J=8.4 Hz, 1H), 7.52 (d, J=1.8 Hz, 1H), 7.62 (d, J=1.8 Hz, 1H).

MS Calcd.: 401; MS Found: 402 (M+H).

### Example 35

### 1-(3-Chloro-4-{[4-chloro-7-(1-ethylpropyl)-1-methyl-1H-benzimidazol-2-yl]oxy}-5-methylphenyl)ethanone

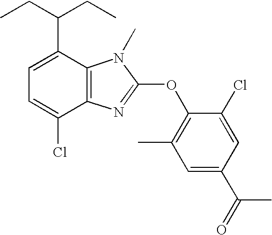

To a solution of 3-chloro-4-{[4-chloro-7-(1-ethylpropyl)-1-methyl-1H-benzimidazol-2-yl]oxy}-5-methylbenzonitrile (185 mg, 0.459 mmol) in tetrahydrofuran (1.0 mL) was added methylmagnesium bromide in tetrahydrofuran (1.4 M, 1.0 mL, 1.4 mmol) at room temperature. The mixture was stirred at 40° C. for 1 h. The mixture was poured into saturated aqueous ammonium chloride solution, and extracted with ethyl acetate (10 mL×4). The combined organic layer was washed with brine, dried over anhydrous magnesium sulfate, filtered and concentrated in vacuo. The residue was purified by flash column chromatography on silica gel eluting with a 0-15% ethyl acetate/n-hexane gradient mixture. The filtrate was concentrated in vacuo to give a solid, which was collected by filtration and washed with n-hexane to give the title compound (126 mg, 0.300 mmol, 66%) as a white powder.

### mp 160-161° C.

¹H NMR (CDCl₃) δ 0.87 (t, J=6.9 Hz, 6H), 1.69-1.83 (m, 4H), 2.39 (s, 3H), 2.61 (s, 3H), 3.20-3.24 (m, 1H), 4.01 (s, 3H), 6.93 (d, J=8.4 Hz, 1H), 7.14 (d, J=8.4 Hz, 1H), 7.80 (d, J=1.8 Hz, 1H), 7.89 (d, J=1.8 Hz, 1H).

MS Calcd.: 418; MS Found: 419 (M+H).

### Example 36

### 4-Chloro-2-[2-chloro-6-methyl-4-(methylthio)phenoxy]-7-(1-ethylpropyl)-1-methyl-1H-benzimidazole

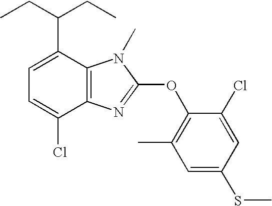

To a solution of 2-(4-bromo-2-chloro-6-methylphenoxy)-4-chloro-7-(1-ethylpropyl)-1-methyl-1H-benzimidazole (2.50 g, 5.48 mmol) in tetrahydrofuran (55 mL) was added n-butyllithium (1.60 M solution in n-hexane, 3.76 mL, 6.02 mmol) at −78° C., and the mixture was stirred for 20 min. Dimethyldisulfide (3.7 mL, 41.2 mmol) was added to the mixture, which was stirred at room temperature for 1 h. The mixture was poured into saturated aqueous ammonium chloride solution and extracted with ethyl acetate (100 mL×4). The combined organic layer was washed with brine, dried over anhydrous magnesium sulfate, filtered and concentrated in vacuo. The residue was recrystallized from methanol to give the title compound (2.02 g, 4.77 mmol, 87%) as white crystals.

### mp 141-143° C.

¹H NMR (CDCl₃) δ 0.86 (t, J=7.5 Hz, 6H), 1.68-1.79 (m, 4H), 2.30 (s, 3H), 2.49 (s, 3H), 3.21-3.25 (m, 1H), 3.98 (s, 3H), 6.91 (d, J=8.4 Hz, 1H), 7.06 (d, J=2.1 Hz, 1H), 7.12 (d, J=8.4 Hz, 1H), 7.15 (d, J=2.1 Hz, 1H).

MS Calcd.: 422; MS Found: 423 (M+H).

### Example 37

### 4-Chloro-2-[2-chloro-6-methyl-4-(methylsulfinyl)phenoxy]-7-(1-ethylpropyl)-1-methyl-1H-benzimidazole

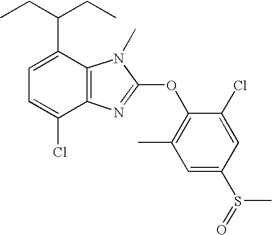

To a solution of 4-chloro-2-[2-chloro-6-methyl-4-(methylthio)phenoxy]-7-(1-ethylpropyl)-1-methyl-1H-benzimidazole (200 mg, 0.472 mmol) in dichloromethane (3.0 mL) was added m-chloroperbenzoic acid (89.0 mg, 0.519 mmol), and the mixture was stirred at room temperature for 1 h. The mixture was diluted with saturated aqueous sodium hydrogen carbonate solution (15 mL) and extracted with ethyl acetate (15 mL×4). The combined organic layer was washed with brine, dried over anhydrous magnesium sulfate, filtered and concentrated in vacuo. The residue was purified by flash column chromatography on silica gel eluting with a 10-60% ethyl acetate/n-hexane gradient mixture. The filtrate was concentrated in vacuo to give the solid, which was recrystallized from ethyl acetate-n-hexane to give the title compound (98.1 mg, 0.223 mmol, 47%) as white crystals.

### mp 182-184° C.

¹H NMR (CDCl₃) δ 0.87 (t, J=7.5 Hz, 6H), 1.69-1.83 (m, 4H), 2.41 (s, 3H), 2.80 (s, 3H), 3.21-3.25 (m, 1H), 4.01 (s, 3H), 6.94 (d, J=8.4 Hz, 1H), 7.15 (d, J=8.4 Hz, 1H), 7.48 (d, J=2.1 Hz, 1H), 7.59 (d, J=2.1 Hz, 1H).

MS Calcd.: 438; MS Found: 439 (M+H).

### Example 38

### 4-Chloro-2-[2-chloro-6-methyl-4-(methylsulfonyl)phenoxy]-7-(1-ethylpropyl)-1-methyl-1H-benzimidazole

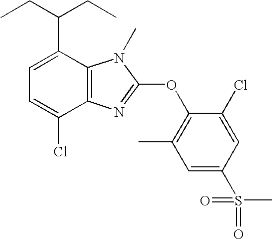

To a solution of 4-chloro-2-[2-chloro-6-methyl-4-(methylthio)phenoxy]-7-(1-ethylpropyl)-1-methyl-1H-benzimidazole (200 mg, 0.472 mmol) in dichloromethane (2.0 mL) was added m-chloroperbenzoic acid (244 mg, 1.41 mmol), and the mixture was stirred at room temperature for 2 h. The mixture was diluted with saturated aqueous sodium hydrogen carbonate solution (20 mL) and extracted with ethyl acetate (20 mL×3). The combined organic layer was washed with brine, dried over anhydrous magnesium sulfate, filtered and concentrated in vacuo. The residue was purified by flash column chromatography on silica gel eluting with a 5-50% ethyl acetate/n-hexane gradient mixture. The filtrate was concentrated in vacuo to give the solid, which was washed with diisopropyl ether to give the title compound (163 mg, 0.358 mmol, 76%) as a white powder.

### mp 199-200° C.

¹H NMR (CDCl₃) δ 0.87 (t, J=7.5 Hz, 6H), 1.69-1.85 (m, 4H), 2.43 (s, 3H), 3.13 (s, 3H), 3.20-3.25 (m, 1H), 4.02 (s, 3H), 6.96 (d, J=8.4 Hz, 1H), 7.15 (d, J=8.4 Hz, 1H), 7.79 (d, J=2.4 Hz, 1H), 7.90 (d, J=2.4 Hz, 1H).

MS Calcd.: 454; MS Found: 455 (M+H).

### Example 39

### 3-Chloro-4-{[4-chloro-7-(1-ethylpropyl)-1-methyl-1H-benzimidazol-2-yl]oxy}-5-methylbenzamide

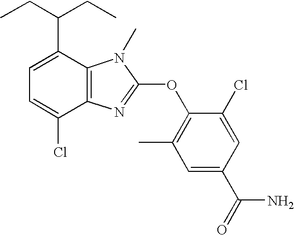

To a solution of 2-(4-bromo-2-chloro-6-methylphenoxy)-4-chloro-7-(1-ethylpropyl)-1-methyl-1H-benzimidazole (300 mg, 0.657 mmol) in tetrahydrofuran (6.0 mL) was added n-butyllithium (1.60 M in n-hexane, 0.45 mL, 0.720 mmol) at −78° C., and the mixture was stirred for 15 min. To the mixture was added dry ice, and the mixture was stirred at room temperature for 1 h. The mixture was poured into saturated aqueous ammonium chloride solution and extracted with ethyl acetate (20 mL×3). The combined organic layer was washed with brine, dried over anhydrous magnesium sulfate, filtered and concentrated in vacuo. The residue was recrystallized from methanol to give 3-chloro-4-{[4-chloro-7-(1-ethylpropyl)-1-methyl-1H-benzimidazol-2-yl]oxy}-5-methylbenzoic acid (80.0 mg). To a solution of the benzoic acid (80.0 mg, 0.189 mmol) in N,N-dimethylformamide (1.5 mL) were added 1-hydroxy-1H-benzotriazole ammonium salt (41.7 mg, 0.245 mmol) and 1-ethyl-3-(3-dimethylaminopropyl)carbodiimide hydrochloride (39.8 mg, 0.208 mmol). The mixture was stirred at room temperature for 11 hr. The mixture was diluted with saturated aqueous sodium hydrogen carbonate solution (20 mL), and extracted with ethyl acetate (20 mL×3). The combined organic layer was washed with brine, dried over anhydrous magnesium sulfate, filtered and concentrated in vacuo. The residue was purified by flash column chromatography on silica gel eluting with a 10-100% ethyl acetate/n-hexane gradient mixture. The filtrate was concentrated in vacuo to give the solid, which was washed with diisopropyl ether to give the title compound (63.2 mg, 0.150 mmol, 23%) as a white powder.

### mp 294-295° C.

¹H NMR (DMSO-d₆) δ 0.82 (t, J=7.5 Hz, 6H), 1.66-1.76 (m, 4H), 2.29 (s, 3H), 3.28-3.33 (m, 1H), 4.00 (s, 3H), 7.02 (d, J=8.4 Hz, 1H), 7.17 (d, J=8.4 Hz, 1H), 7.88 (d, J=2.1 Hz, 1H), 7.93 (d, J=2.1 Hz, 1H), 7.56 (brs, 1H), 8.11 (brs, 1H).

MS Calcd.: 419; MS Found: 420 (M+H).

### Example 40

### 3-Chloro-4-{[4-chloro-7-(1-ethylpropyl)-1-methyl-1H-benzimidazol-2-yl]oxy}-5-methylbenzenesulfonamide

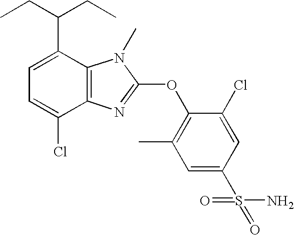

The title compound was prepared from 2-(4-bromo-2-chloro-6-methylphenoxy)-4-chloro-7-(1-ethylpropyl)-1-methyl-1H-benzimidazole in the similar method described in Example 32.

### mp 293-294° C.

¹H NMR (DMSO-d₆) δ 0.82 (t, J=7.2 Hz, 6H), 1.64-1.76 (m, 4H), 2.33 (s, 3H), 3.30-3.36 (m, 1H), 4.00 (s, 3H), 6.71 (brs, 1H), 7.03 (d, J=8.4 Hz, 1H), 7.17 (d, J=8.4 Hz, 1H), 7.55 (brs, 1H), 7.83 (brs, 1H), 7.87 (brs, 1H).

MS Calcd.: 455; MS Found: 456 (M+H).

### Example 41

### 2-[2-Bromo-6-chloro-4-(trifluoromethoxy)phenoxy]-4-chloro-7-(1-ethylpropyl)-1-methyl-1H-benzimidazole

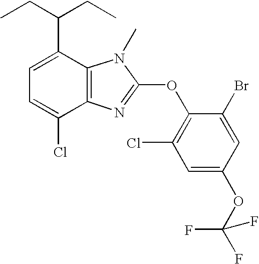

The title compound was prepared from 2,4-dichloro-7-(1-ethylpropyl)-1-methyl-1H-benzimidazole and 2-bromo-6-chloro-4-(trifluoromethoxy)phenol in the similar method described in Example 23.

### mp 149-151° C.

¹H NMR (CDCl₃) δ 0.87 (t, J=7.2 Hz, 6H), 1.68-1.84 (m, 4H), 3.20-3.24 (m, 1H), 4.00 (s, 3H), 6.94 (d, J=8.1 Hz, 1H), 7.15 (d, J=8.1 Hz, 1H), 7.36 (d, J=2.1 Hz, 1H), 7.46 (d, J=2.1 Hz, 1H).

MS Calcd.: 524; MS Found: 525 (M+H).

### Example 42

### 4-Chloro-2-[2-chloro-6-(methylthio)-4-(trifluoromethoxy)phenoxy]-7-(1-ethylpropyl)-1-methyl-1H-benzimidazole

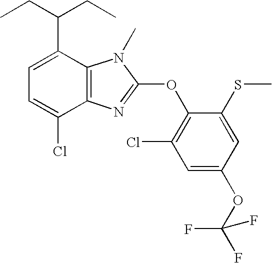

The title compound was prepared from 2-[2-bromo-6-chloro-4-(trifluoromethoxy)phenoxy]-4-chloro-7-(1-ethylpropyl)-1-methyl-1H-benzimidazole in the similar method described in Example 36.

### mp 138-139° C.

¹H NMR (CDCl₃) δ 0.86 (t, J=7.5 Hz, 6H), 1.66-1.82 (m, 4H), 2.44 (s, 3H), 3.20-3.24 (m, 1H), 3.99 (s, 3H), 6.93 (d, J=8.7 Hz, 1H), 7.02 (d, J=2.1 Hz, 1H), 7.13 (d, J=8.7 Hz, 1H), 7.15 (d, J=2.1 Hz, 1H).

MS Calcd.: 492; MS Found: 493 (M+H).

### Example 43

### 4-Chloro-2-[2-chloro-6-(methylsulfinyl)-4-(trifluoromethoxy)phenoxy]-7-(1-ethylpropyl)-1-methyl-1H-benzimidazole

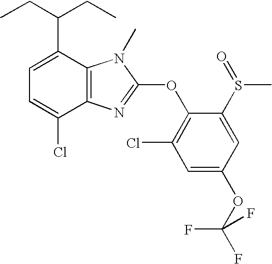

The title compound was prepared from 4-chloro-2-[2-chloro-6-(methylthio)-4-(trifluoromethoxy)phenoxy]-7-(1-ethylpropyl)-1-methyl-1H-benzimidazole in the similar method described in Example 37.

### mp 150-151° C.

¹H NMR (CDCl₃) δ 0.87 (t, J=7.5 Hz, 6H), 1.68-1.84 (m, 4H), 3.05 (s, 3H), 3.19-3.23 (m, 1H), 4.00 (s, 3H), 6.98 (d, J=8.4 Hz, 1H), 7.18 (d, J=8.4 Hz, 1H), 7.47 (d, J=2.4 Hz, 1H), 7.84 (d, J=2.4 Hz, 1H).

MS Calcd.: 508; MS Found: 509 (M+H).

### Example 44

### 4-Chloro-2-[2-chloro-6-(methylsulfonyl)-4-(trifluoromethoxy)phenoxy]-7-(1-ethylpropyl)-1-methyl-1H-benzimidazole

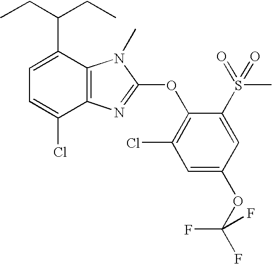

The title compound was prepared from 4-chloro-2-[2-chloro-6-(methylthio)-4-(trifluoromethoxy)phenoxy]-7-(1-ethylpropyl)-1-methyl-1H-benzimidazole in the similar method described in Example 38.

### mp 164-165° C.

¹H NMR (CDCl₃) δ 0.87 (t, J=7.5 Hz, 6H), 1.70-1.84 (m, 4H), 3.20-3.26 (m, 1H), 3.26 (s, 3H), 4.01 (s, 3H), 6.97 (d, J=8.1 Hz, 1H), 7.17 (d, J=8.1 Hz, 1H), 7.64 (d, J=2.4 Hz, 1H), 7.87 (d, J=2.4 Hz, 1H).

MS Calcd.: 524; MS Found: 525 (M+H).

### Example 45

### 4-Chloro-7-(1-ethylpropyl)-N-[2-methoxy-6-methyl-4-(methylthio)phenyl]-1-methyl-1H-benzimidazol-2-amine

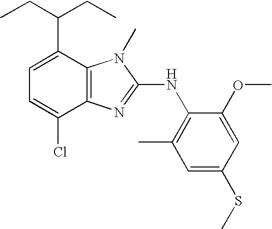

The title compound was prepared from N-(4-bromo-2-methoxy-6-methylphenyl)-4-chloro-7-(1-ethylpropyl)-1-methyl-1H-benzimidazol-2-amine in the similar method described in Example 36.

### mp 223-225° C.

¹H NMR (CDCl₃) δ 0.82 (t, J=6.9 Hz, 6H), 1.66-1.80 (m, 4H), 2.17 (s, 3H), 2.50 (s, 3H), 3.14-3.17 (m, 1H), 3.71 (s, 3H), 3.77 (s, 3H), 6.09 (s, 1H), 6.72 (brs, 1H), 6.78 (brs, 1H), 6.83 (d, J=8.1 Hz, 1H), 7.10 (d, J=8.1 Hz, 1H).

MS Calcd.: 417; MS Found: 418 (M+H).

### Example 46

### 4-Chloro-7-(1-ethylpropyl)-N-[2-methoxy-6-methyl-4-(methylsulfinyl)phenyl]-1-methyl-1H-benzimidazol-2-amine

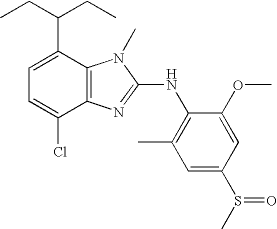

The title compound was prepared from 4-chloro-7-(1-ethylpropyl)-N-[2-methoxy-6-methyl-4-(methylthio)phenyl]-1-methyl-1H-benzimidazol-2-amine in the similar method described in Example 37.

### mp 222-223° C.

¹H NMR (CDCl₃) δ 0.84 (t, J=7.2 Hz, 6H), 1.67-1.82 (m, 4H), 2.19 (s, 3H), 2.75 (s, 3H), 3.16-3.24 (m, 1H), 3.84 (s, 3H), 3.89 (s, 3H), 6.35 (brs, 1H), 6.89 (d, J=8.4 Hz, 1H), 7.02 (d, J=1.8 Hz, 1H), 7.12 (d, J=8.4 Hz, 1H), 7.19 (d, J=1.8 Hz, 1H).

MS Calcd.: 433; MS Found: 434 (M+H).

### Example 47

### 4-Chloro-7-(1-ethylpropyl)-N-[2-methoxy-6-methyl-4-(methylsulfonyl)phenyl]-1-methyl-1H-benzimidazol-2-amine

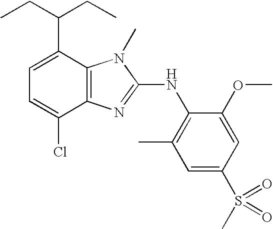

The title compound was prepared from 4-chloro-7-(1-ethylpropyl)-N-[2-methoxy-6-methyl-4-(methylthio)phenyl]-1-methyl-1H-benzimidazol-2-amine in the similar method described in Example 38.

### mp 234-236° C.

¹H NMR (CDCl₃) δ 0.85 (t, J=7.2 Hz, 6H)—, 1.65-1.83 (m, 4H), 2.16 (s, 3H), 3.08 (s, 3H), 3.18-3.24 (m, 1H), 3.87 (s, 3H), 3.91 (s, 3H), 6.46 (brs, 1H), 6.92 (d, J=8.4 Hz, 1H), 7.12 (d, J=1.8 Hz, 1H), 7.31 (brs, 1H), 7.45 (brs, 1H).

MS Calcd.: 449; MS Found: 450 (M+H).

### Example 48

### 4-{[4-Chloro-7-(1-ethylpropyl)-1-methyl-1H-benzimidazol-2-yl]amino}-3-methoxy-5-methylbenzamide

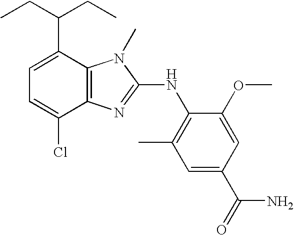

The title compound was prepared from N-(4-bromo-2-methoxy-6-methylphenyl)-4-chloro-7-(1-ethylpropyl)-1-methyl-1H-benzimidazol-2-amine in the similar method described in Example 39.

### mp 298-299° C.

¹H NMR (DMSO-d₆) δ 0.79 (t, J=7.5 Hz, 6H), 1.61-1.75 (m, 4H), 2.11 (s, 3H), 3.30-3.35 (m, 1H), 3.79 (s, 3H), 3.84 (s, 3H), 6.81 (d, J=8.4 Hz, 1H), 7.01 (d, J=8.4 Hz, 1H), 7.31 (brs, 1H), 7.42 (s, 2H), 7.95 (brs, 1H), 8.21 (s, 1H).

MS Calcd.: 414; MS Found: 415 (M+H).

### Example 49

### N-(2-Bromo-4-chloro-6-methylphenyl)-4-chloro-7-(1-ethylpropyl)-1-methyl-1H-benzimidazol-2-amine

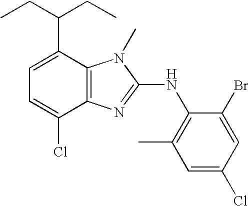

The title compound was prepared from 2,4-dichloro-7-(1-ethylpropyl)-1-methyl-1H-benzimidazole and 2-bromo-4-chloro-6-methylaniline in the similar method described in Example 16.

### mp 232-233° C.

¹H NMR (CDCl₃) δ 0.86 (t, J=7.2 Hz, 6H), 1.60-1.80 (m, 4H), 2.18 (s, 3H), 3.17-3.21 (m, 1H), 3.77 (s, 3H), 6.15 (brs, 1H), 6.85 (d, J=8.4 Hz, 1H), 7.02 (d, J=8.4 Hz, 1H), 7.18 (d, J=2.7 Hz, 1H), 7.46 (d, J=2.7 Hz, 1H).

MS Calcd.: 453; MS Found: 454 (M+H).

### Example 50

### 4-Chloro-N-[4-chloro-2-methyl-6-(methylthio)phenyl]-7-(1-ethylpropyl)-1-methyl-1H-benzimidazol-2-amine

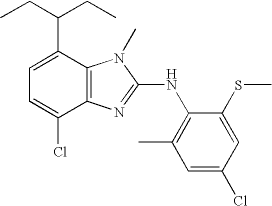

The title compound was prepared from N-(2-bromo-4-chloro-6-methylphenyl)-4-chloro-7-(1-ethylpropyl)-1-methyl-1H-benzimidazol-2-amine in the similar method described in Example 36.

### mp 167-169° C.

¹H NMR (CDCl₃) δ 0.85 (t, J=7.2 Hz, 6H), 1.57-1.81 (m, 4H), 2.09 (s, 3H), 2.40 (s, 3H), 3.10-3.18 (m, 1H), 3.67 (s, 3H), 6.18 (brs, 1H), 6.81-6.86 (m, 2H), 7.01-7.04 (m, 2H).

MS Calcd.: 421; MS Found: 422 (M+H).

### Example 51

### 4-Chloro-N-[4-chloro-2-methyl-6-(methylsulfinyl)phenyl]-7-(1-ethylpropyl)-1-methyl-1H-benzimidazol-2-amine

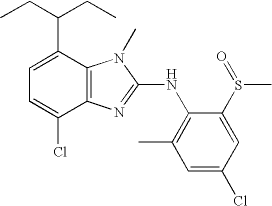

The title compound was prepared from 4-chloro-N-[4-chloro-2-methyl-6-(methylthio)phenyl]-7-(1-ethylpropyl)-1-methyl-1H-benzimidazol-2-amine in the similar method described in Example 37.

### mp 212-213° C.

¹H NMR (CDCl₃) δ 0.89 (t, J=7.2 Hz, 6H), 1.61-1.77 (m, 4H), 2.17 (s, 3H), 2.96 (s, 3H), 3.15-3.22 (m, 1H), 3.58 (s, 3H), 6.84 (d, J=8.7 Hz, 1H), 7.03 (brs, 1H), 7.31 (brs, 1H), 7.48 (brs, 1H), 8.55 (brs, 1H).

MS Calcd.: 437; MS Found: 438 (M+H).

The compounds of Examples 52-56 were prepared from 2,4-dichloro-7-(1-ethylpropyl)-1-methyl-1H-benzimidazole and corresponding phenols in the similar method described in Example 23.

### Example 52

### 3-{[4-Chloro-7-(1-ethylpropyl)-1-methyl-1H-benzimidazol-2-yl]oxy}-4-methoxy-N,N,6-trimethylpyridin-2-amine

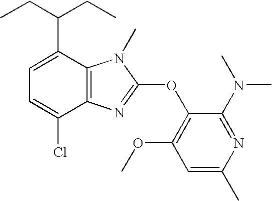

### mp 202-203° C.

¹H NMR (CDCl₃) δ 0.87 (t, J=6.6 Hz, 6H), 1.70-2.05 (m, 4H), 2.37 (s, 3H), 3.00 (s, 6H), 3.21-3.25 (m, 1H), 3.76 (s, 3H), 3.94 (s, 3H), 6.28 (s, 1H), 6.91 (d, J=8.4 Hz, 1H), 7.12 (d, J=8.4 Hz, 1H).

MS Calcd.: 416; MS Found: 417 (M+H).

### Example 53

### 4-Chloro-7-(1-ethylpropyl)-1-methyl-2-[(2-methyl-2,4,5,6-tetrahydrocyclopenta[c]pyrazol-3-yl)oxy]-1H-benzimidazole

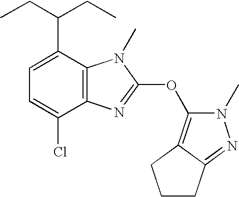

amorphous.

¹H NMR (CDCl₃) δ 0.84 (t, J=7.2 Hz, 6H), 1.66-1.84 (m, 4H), 2.35 (quin, J=7.2 Hz, 2H), 2.67-2.77 (m, 4H), 3.17-3.21 (m, 1H), 3.77 (s, 3H), 3.93 (s, 3H), 6.97 (d, J=8.4 Hz, 1H), 7.20 (d, J=8.4 Hz, 1H).

MS Calcd.: 372; MS Found: 373 (M+H).

### Example 54

### 4-Chloro-7-(1-ethylpropyl)-1-methyl-2-[(2-methyl-2,3-dihydro-1H-isoindol-4-yl)oxy]-1H-benzimidazole

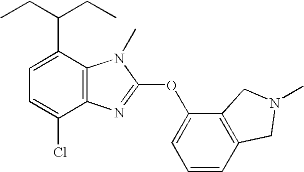

### mp 99-100° C.

¹H NMR (CDCl₃) δ 0.87 (t, J=7.2 Hz, 6H), 1.65-1.83 (m, 4H), 2.57 (s, 3H), 3.20-3.23 (m, 1H), 3.90 (s, 3H), 3.92 (s, 2H), 3.97 (s, 2H), 6.94 (d, J=8.4 Hz, 1H), 7.06 (d, J=6.9 Hz, 1H), 7.17 (d, J=8.4 Hz, 1H), 7.18-7.28 (m, 2H).

MS Calcd.: 383; MS Found: 384 (M+H).

### Example 55

### 4-Chloro-2-[(5,7-dichloro-2-methyl-2,3-dihydro-1H-isoindol-4-yl)oxy]-7-(1-ethylpropyl)-1-methyl-1H-benzimidazole

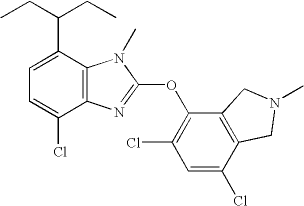

### mp 148-149° C.

¹H NMR (CDCl₃) δ 0.87 (t, J=7.2 Hz, 6H), 1.67-1.82 (m, 4H), 2.54 (s, 3H), 3.19-3.24 (m, 1H), 3.96 (s, 3H), 3.99 (s, 2H), 4.00 (s, 2H), 6.94 (d, J=8.4 Hz, 1H), 7.15 (d, J=8.4 Hz, 1H), 7.30 (s, 1H).

MS Calcd.: 451; MS Found: 452 (M+H).

### Example 56

### 4-Chloro-7-(1-ethylpropyl)-2-{[4-methoxy-2-methyl-6-(trifluoromethyl)pyridin-3-yl]oxy}-1-methyl-1H-benzimidazole

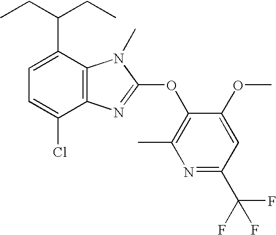

### mp 178-179° C.

¹H NMR (CDCl₃) δ 0.86 (t, J=7.2 Hz, 6H), 1.65-1.83 (m, 4H), 2.56 (s, 3H), 3.21-3.25 (m, 1H), 3.86 (s, 3H), 3.98 (s, 3H), 6.94 (d, J=8.4 Hz, 1H), 7.15 (d, J=8.4 Hz, 1H), 7.19 (s, 1H).

MS Calcd.: 441; MS Found: 442 (M+H).

The compounds of Examples 57-58 were prepared from 2,4-dichloro-7-(1-ethylpropyl)-1-methyl-1H-benzimidazole in the similar method described in Example 17.

### Example 57

### 4-Chloro-7-(1-ethylpropyl)-N-[2-methoxy-6-methyl-4-(1H-pyrazol-1-yl)phenyl]-1-methyl-1H-benzimidazol-2-amine

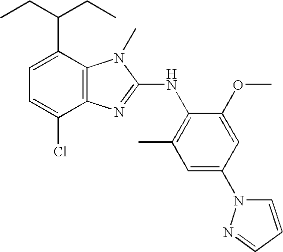

### mp 237-239° C.

¹H NMR (CDCl₃) δ 0.84 (t, J=7.5 Hz, 6H), 1.65-1.83 (m, 4H), 2.25 (s, 3H), 3.15-3.19 (m, 1H), 3.78 (s, 3H), 3.86 (s, 3H), 6.14 (brs, 1H), 6.44 (dd, J=1.8 Hz, 2.1 Hz, 1H), 6.86 (d, J=8.1 Hz, 1H), 7.07 (d, J=2.1 Hz, 1H), 7.12 (d, J=8.1 Hz, 1H), 7.23 (d, J=2.1 Hz, 1H), 7.70 (d, J=1.8 Hz, 1H), 7.88 (d, J=2.1 Hz, 1H).

MS Calcd.: 437; MS Found: 438 (M+H).

### Example 58

### 4-Chloro-7-(1-ethylpropyl)-N-{2-methoxy-6-methyl-4-[3-(trifluoromethyl)-1H-pyrazol-1-yl]phenyl}-1-methyl-1H-benzimidazol-2-amine

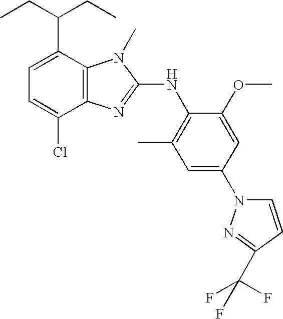

### mp 249-251° C.

¹H NMR (CDCl₃) δ 0.86 (t, J=6.9 Hz, 6H), 1.65-1.83 (m, 4H), 2.25 (s, 3H), 3.16-3.19 (m, 1H), 3.81 (s, 3H), 3.90 (s, 3H), 6.19 (s, 1H), 6.70 (d, J=2.4 Hz, 1H), 6.87 (d, J=8.1 Hz, 1H), 7.10 (d, J=2.1 Hz, 1H), 7.11 (d, J=8.1 Hz, 1H), 7.21 (d, J=2.4 Hz, 1H), 7.90 (d, J=2.1 Hz, 1H).

MS Calcd.: 505; MS Found: 506 (M+H).

### Example 59

### 4-Chloro-2-[2-chloro-6-methyl-4-(trimethylsilyl)phenoxy]-7-(1-ethylpropyl)-1-methyl-1H-benzimidazole

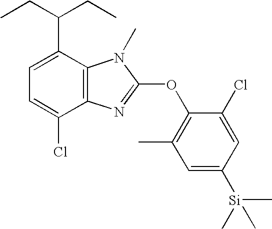

To a solution of 2-(4-bromo-2-chloro-6-methylphenoxy)-4-chloro-7-(1-ethylpropyl)-1-methyl-1H-benzimidazole (250 mg, 0.548 mmol) in tetrahydrofuran (5.0 mL) was added n-butyllithium (1.60 M solution in n-hexane, 0.37 mL, 0.602 mmol) at −78° C., and the mixture was stirred for 15 min. To the mixture was added trimethylsilyl isocyanate (126 mg, 1.09 mmol). The mixture was stirred at room temperature for 1 hr, and poured into water. The mixture was extracted with ethyl acetate (20 mL×4). The combined organic layer was washed with brine, dried over anhydrous magnesium sulfate, filtered and concentrated in vacuo. The residue was purified by flash column chromatography on silica gel eluting with a 0-15% ethyl acetate/n-hexane gradient mixture. The filtrate was concentrated in vacuo to give the solid, which was washed with n-hexane to give the title compound (75.4 mg, 0.167 mmol, 31%) as a white powder.

### mp 146-148° C.

¹H NMR (CDCl₃) δ 0.28 (s, 9H), 0.87 (t, J=7.2 Hz, 6H), 1.66-1.84 (m, 4H), 2.33 (s, 3H), 3.20-3.26 (m, 1H), 3.99 (s, 3H), 6.91 (d, J=8.4 Hz, 1H), 7.13 (d, J=8.4 Hz, 1H), 7.28 (d, J=0.9 Hz, 1H), 7.37 (d, J=0.9 Hz, 1H).

MS Calcd.: 448; MS Found: 449 (M+H).

### Example 60

### 4-Chloro-2-[2,4-dichloro-6-(trimethylsilyl)phenoxy]-7-(1-ethylpropyl)-1-methyl-1H-benzimidazole

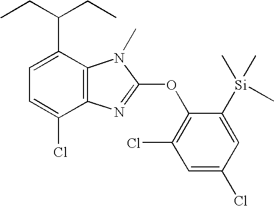

The title compound was prepared from 2-(2,4-dichloro-6-methylphenoxy)-4-chloro-7-(1-ethylpropyl)-1-methyl-1H-benzimidazole in the similar method described in Example 59.

amorphous.

¹H NMR (CDCl₃) δ 0.25 (s, 9H), 0.85 (t, J=7.5 Hz, 6H), 1.67-1.83 (m, 4H), 3.20-3.24 (m, 1H), 3.96 (s, 3H), 6.92 (d, J=8.4 Hz, 1H), 7.13 (d, J=8.4 Hz, 1H), 7.35 (d, J=2.4 Hz, 1H), 7.45 (d, J=2.4 Hz, 1H).

MS Calcd.: 468; MS Found: 469 (M+H).

### Example 61

### N-(4-Chloro-2-methoxy-6-methylphenyl)-1-methyl-7-[1-(methylamino)propyl]-1H-benzimidazol-2-amine

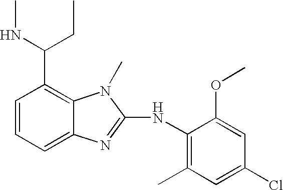

### i) 2-[(4-Chloro-2-methoxy-6-methylphenyl)amino]-1-methyl-1H-benzimidazole-7-carbonitrile

A solution of 7-bromo-N-(4-chloro-2-methoxy-6-methylphenyl)-1-methyl-1H-benzimidazol-2-amine (100 mg, 0.26 mmol) and copper cyanide (28.2 mg, 0.32 mmol) in 1-methyl-2-pyrrolidone (2 mL) was irradiated by a microwave (150 W) at 180° C. for 0.5 hr. To the mixture was added dropwise water and the reaction mixture was stirred for 1 hr. The mixture was dissolved in ethyl acetate (20 mL) and washed with water (20 mL). The organics were dried over anhydrous sodium sulfate, filtered, and concentrated in vacuo. The residue was purified by flash column chromatography eluting with a 50% ethyl acetate/n-hexanes mixture to give the title compound (60.0 mg, 70%) as an amorphous.

¹H NMR (CDCl₃) δ 2.21 (s, 3H), 3.83 (s, 3H), 4.03 (s, 3H), 5.97 (s, 1H), 6.81 (d, J=2.1 Hz, 1H), 6.92 (d, J=2.1 Hz, 1H), 7.14 (t, J=7.8 Hz, 1H), 7.37 (d, J=7.8 Hz, 1H), 7.67 (d, J=7.8 Hz, 1H).

MS Calcd.: 326; Found: 327 (M+H).

### ii) 1-{2-[(4-Chloro-2-methoxy-6-methylphenyl)amino]-1-methyl-1H-benzimidazol-7-yl}propan-1-one

A solution of ethylmagnesium bromide (3M solution in diethyl ether; 1.0 mL, 3.0 mmol) was added dropwise to a solution of 2-[(4-chloro-2-methoxy-6-methylphenyl)amino]-1-methyl-1H-benzimidazole-7-carbonitrile (60.0 mg, 0.18 mmol) in tetrahydrofuran (5 mL) at room temperature. The mixture was refluxed for 18 hr. After cooling, the reaction mixture was quenched with 1N hydrochloric acid and extracted with ethyl acetate. The extract was washed with brine, dried over anhydrous magnesium sulfate, filtered, and concentrated in vacuo. The residue was purified by flash column chromatography eluting with a 50% ethyl acetate/n-hexanes mixture to give the title compound (52.0 mg, 79%).

¹H NMR (CDCl₃) δ 1.29 (t, J=7.5 Hz, 3H), 2.19 (s, 3H), 3.11 (q, J=7.5 Hz, 2H), 3.63 (s, 3H), 3.81 (s, 3H), 6.80 (d, J=2.1 Hz, 1H), 6.89 (d, J=2.1 Hz, 1H), 7.16 (t, J=7.5 Hz, 1H), 7.47 (d, J=7.5 Hz, 1H), 7.65 (d, J=7.5 Hz, 1H).

MS Calcd.: 357; Found: 358 (M+H).

### iii) N-(4-Chloro-2-methoxy-6-methylphenyl)-1-methyl-7-[1-(methylamino)propyl]-1H-benzimidazol-2-amine

Titanium(IV) isopropoxide (0.5 mL, 1.65 mmol) was added to a solution of methylamine in methanol (2M, 1.88 mL) followed by an addition of 1-{2-[(4-chloro-2-methoxy-6-methylphenyl)amino]-1-methyl-1H-benzimidazol-7-yl}propan-1-one (52.0 mg, 0.145 mmol). The reaction mixture was stirred at room temperature for 5 hr and then sodium borohydride (50 mg, 1.25 mmol) was added thereto. The resulting mixture was further stirred for 1 h. The reaction was quenched by an addition of water (1 mL), the resulting inorganic precipitate was removed by filtration and washed with ethyl acetate. The organic layer was separated and the aqueous part was further extracted with ethyl acetate. The combined extracts were dried over anhydrous magnesium sulfate, filtered, and concentrated in vacuo. The residue was purified by preparative HPLC to give the title compound as the trifluoroacetic acid salt. The salt was washed with aqueous sodium hydrogen carbonate solution and extracted with ethyl acetate. The extract was washed with brine, dried over anhydrous magnesium sulfate, filtered and concentrated in vacuo to give the title compound (4.0 mg, 7%) as an amorphous.

¹H NMR (CDCl₃) δ 0.90 (t, J=7.5 Hz, 3H), 1.85-1.95 (m, 2H), 2.18 (s, 3H), 2.35 (s, 3H), 3.80 (s, 3H), 3.91 (s, 3H), 4.23-4.28 (m, 1H), 6.78 (d, J=2.4 Hz, 1H), 6.88 (d, J=2.4 Hz, 1H), 7.11-7.13 (m, 2H), 7.39 (t, J=7.5 Hz, 1H).

MS Calcd.: 372; Found: 373 (M+H).

### Example 62

### N-(4-Chloro-2-methoxy-6-methylphenyl)-7-[1-(dimethylamino)propyl]-1-methyl-1H-benzimidazol-2-amine

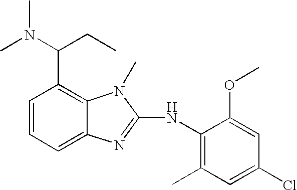

A solution of formaldehyde (36-38% in water; 1.0 mL) was added dropwise to a solution of N-(4-chloro-2-methoxy-6-methylphenyl)-1-methyl-7-[1-(methylamino)propyl]-1H-benzimidazol-2-amine (20.0 mg, 0.054 mmol) in acetonitrile (0.5 mL) at room temperature. The mixture was stirred at room temperature for 1 hr, after which sodium cyanoborohydride (31.4 mg, 0.50 mmol) was added to the mixture and the resulting mixture was further stirred for 1 h. The reaction was quenched by an addition of water (1 mL). The resulting inorganic precipitate was filtered and washed with ethyl acetate. The organic layer was separated and the aqueous part was further extracted with ethyl acetate. The combined extracts were dried over anhydrous magnesium sulfate, filtered, and concentrated in vacuo. The residue was purified by preparative HPLC to give the title compound as the trifluoroacetic acid salt. The salt was washed with aqueous sodium hydrogen carbonate solution and extracted with ethyl acetate. The extract was washed with brine, dried over anhydrous magnesium sulfate, filtered and concentrated in vacuo to give the title compound (2.1 mg, 10%) as an oil.

¹H NMR (CDCl₃) δ 0.91 (t, J=7.5 Hz, 3H), 1.83-1.94 (m, 2H), 2.18 (s, 3H), 2.40 (s, 6H), 3.79 (s, 3H), 3.90 (s, 3H), 4.22-4.27 (m, 1H), 6.78 (d, J=2.4 Hz, 1H), 6.88 (d, J=2.4 Hz, 1H), 7.10-7.13 (m, 2H), 7.41 (t, J=7.5 Hz, 1H).

MS Calcd.: 386; Found: 387 (M+H).

### Example 63

### 2-[4-Bromo-2-(2,4-dichloro-6-methylphenoxy)-7-(1-ethylpropyl)-1H-benzimidazol-1-yl]-N,N-dimethylethanamine

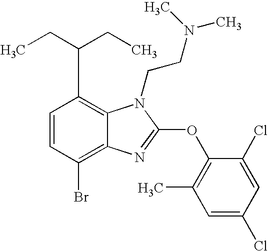

To a solution of 2-[4-bromo-2-chloro-7-(1-ethylpropyl)-1H-benzimidazol-1-yl]-N,N-dimethylethanamine (7.4 mg, 0.0199 mmol) in 1-methyl-2-pyrrolidone (0.5 mL) were added 2,4-dichloro-6-methylphenol (11 mg, 0.060 mmol) and potassium carbonate (8.2 mg, 0.0560 mmol), and the mixture was stirred at 110° C. for 30 min. The reaction mixture was neutralized with saturated aqueous sodium hydrogen carbonate solution and extracted with ethyl acetate (X2). The combined organic layer was washed with brine, dried over anhydrous sodium sulfate and concentrated in vacuo. The residue was purified by preparative TLC eluting with a 25% ethyl acetate/n-hexane gradient mixture to give the title compound (7.0 mg, 0.0136 mmol, 69%) as an oil.

¹H NMR (CDCl₃) δ 0.90 (t, J=7.2 Hz, 6H), 1.65-1.87 (m, 4H), 2.30 (s, 3H), 2.37 (s, 6H), 2.79 (t, J=7.8 Hz, 2H), 3.04-3.13 (m, 1H), 4.47 (t, J=7.8 Hz, 2H), 6.89 (d, J=8.4 Hz, 1H), 7.20-7.33 (m, 3H).

MS Calcd.: 511, MS Found: 512 (M+H).

### Example 64

### 4-Chloro-2-(2,4-dichloro-6-methylphenoxy)-7-(1-ethylpropyl)-1H-benzimidazole

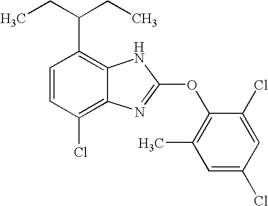

### i) 7-Chloro-2-(2,4-dichloro-6-methylphenoxy)-4-(1-ethylpropyl)-1-(4-methoxybenzyl)-1H-benzimidazole

To a solution of 2,7-dichloro-4-(1-ethylpropyl)-1-(4-methoxybenzyl)-1H-benzimidazole (205 mg, 0.543 mmol) in 1-methyl-2-pyrrolidone (2.0 mL, 20.8 mmol) were added 2,4-dichloro-6-methylphenol (289 mg, 1.63 mmol) and potassium carbonate (225 mg, 1.630 mmol), and the mixture was stirred at 100° C. for 4 days. The reaction mixture was diluted with water and extracted with ethyl acetate (X2). The combined organic layer was washed with brine, dried over anhydrous sodium sulfate and concentrated in vacuo. The residue was purified by silica gel column chromatography eluting with a 0-3% ethyl acetate/n-hexane gradient mixture. The fractions containing the title compound were concentrated in vacuo, and the residual solid was washed with water. The solid was recrystallized from n-hexane to give the title compound (261 mg, 0.504 mmol, 93%) as a colorless solid.

¹H NMR (CDCl₃) δ 0.69 (t, J=7.2 Hz, 6H), 1.54-1.70 (m, 4H), 2.08 (s, 3H), 2.93-3.04 (m, 1H), 3.79 (s, 3H), 5.69 (s, 2H), 6.83-6.87 (m, 3H), 7.05 (d, J=8.1 Hz, 1H), 7.16 (d, J=1.8 Hz, 1H), 7.24-7.31 (m, 3H).

MS Calcd.: 516, MS Found: 517 (M+H).

### ii) 4-Chloro-2-(2,4-dichloro-6-methylphenoxy)-7-(1-ethylpropyl)-1H-benzimidazole

A mixture of 7-chloro-2-(2,4-dichloro-6-methylphenoxy)-4-(1-ethylpropyl)-1-(4-methoxybenzyl)-1H-benzimidazole (19 mg, 0.0367 mmol) and trifluoroacetic acid (1.0 mL) was refluxed for 4 days. After cooling, trifluoroacetic acid was evaporated in vacuo, and the residue was neutralized with saturated aqueous sodium hydrogen carbonate solution and extracted with ethyl acetate. The organic layer was washed with brine, dried over anhydrous sodium sulfate and concentrated in vacuo. The residue was purified by silica gel column chromatography eluting with a 3-10% ethyl acetate/n-hexane gradient mixture to give the title compound (12 mg, 0.0302 mmol, 82%) as an oil.

¹H NMR (CDCl₃) δ 0.69 (t, J=7.2 Hz, 4H), 0.84 (t, J=7.2 Hz, 2H), 1.55-1.88 (m, 4H), 2.26 (s, 2H), 2.30 (s, 1H), 2.50-2.62 (m, 0.33H), 2.88-2.98 (m, 0.66H), 6.89 (d, J=8.4 Hz, 1H), 6.91-7.33 (m, 3H), 8.58 (s, 0.33H), 8.66 (s, 0.66H).

MS Calcd.: 396, MS Found: 397 (M+H).

### Example 65

### 4-Chloro-2-[2,6-dichloro-4-(trifluoromethoxy)phenoxy]-7-(1-ethylpropyl)-1H-benzimidazole

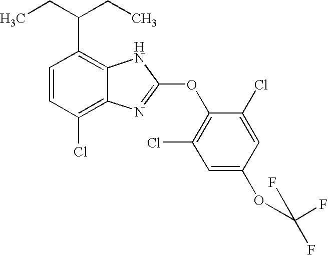

The title compound was prepared from 2,7-dichloro-4-(1-ethylpropyl)-1-(4-methoxybenzyl)-1H-benzimidazole and 2,6-dichloro-4-(trifluoromethoxy)phenol in the similar method described in Example 64.

¹H NMR (CDCl₃) δ 0.69 (t, J=7.2 Hz, 3.6H), 0.85 (t, J=7.2 Hz, 2.4H), 1.54-1.87 (m, 4H), 2.51-2.59 (m, 0.4H), 2.88-2.98 (m, 0.6H), 6.89-6.93 (m, 1H), 7.07-7.18 (m, 1H), 7.32-7.33 (m, 2H), 8.68 (s, 0.4H), 8.77 (s, 0.6H).

MS Calcd.: 466, MS Found: 467 (M+H).

### Example 66

### 4-Chloro-N-(2,4-dichloro-6-methylphenyl)-7-(1-ethylpropyl)-1H-benzimidazol-2-amine

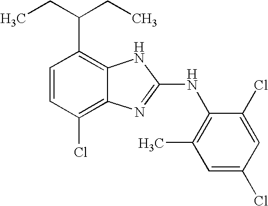

A mixture of 2,4-dichloro-7-(1-ethylpropyl)-1H-benzimidazole (286 mg, 1.11 mmol), 2,4-dichloro-6-methylaniline (587 mg, 3.34 mmol) and 1-methyl-2-pyrrolidone (0.2 mL) was stirred at 110° C. for 4 days. After cooling, the reaction mixture was diluted with water and extracted with ethyl acetate (X2). The combined organic layer was washed with brine, dried over anhydrous sodium sulfate and concentrated in vacuo. The residue was crystallized from diisopropyl ether to give the title compound (25 mg, 0.0630 mmol, 5.7%) as a pale purple solid. The filtrate was concentrated in vacuo and purified by basic silica gel column chromatography eluting with a 2-15% ethyl acetate/n-hexane gradient mixture. The desired fractions were concentrated in vacuo, and the residual solid was washed with diisopropyl ether to give the title compound (55 mg, 0.134 mmol, 12% yield) as a colorless solid.

total: 80 mg, 0.202 mmol, 18%

¹H NMR (DMSO-d₆) δ 0.70 (t, J=7.2 Hz, 6H), 1.57-1.69 (m, 4H), 2.23 (s, 3H), 2.78-2.90 (m, 1H), 6.71 (d, J=7.5 Hz, 1H), 6.94 (d, J=7.5 Hz, 1H), 7.41 (s, 1H), 7.54 (s, 1H), 8.59 (s, 1H), 11.22 (s, 1H).

MS Calcd.: 395, MS Found: 396 (M+H).

### Example 67

### 4-Chloro-N-(4-chloro-2-methoxy-6-methylphenyl)-7-(1-ethylpropyl)-1H-benzimidazol-2-amine

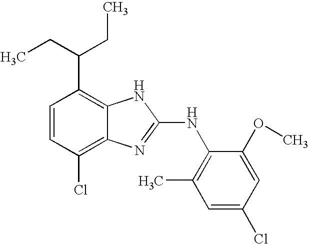

The title compound was prepared from 2,4-dichloro-7-(1-ethylpropyl)-1H-benzimidazole and 4-chloro-2-methoxy-6-methylaniline in the similar method described in Example 66.

¹H NMR (CDCl₃-DMSO-d₆ 3 drops) δ 0.67 (t, J=7.2 Hz, 6H), 1.45-1.62 (m, 4H), 2.20 (s, 3H), 2.60-2.75 (m, 1H), 3.69 (s, 3H), 6.64 (d, J=7.8 Hz, 1H), 6.70 (d, J=2.1 Hz, 1H), 6.80 (d, J=2.1 Hz, 1H), 6.87 (d, J=7.8 Hz, 1H).

MS Calcd.: 391, MS Found: 392 (M+H).

### Example 68

### 4-Chloro-2-[2,6-dichloro-4-(trifluoromethoxy)phenoxy]-7-(1-ethylpropyl)-1-{2-[(4-methoxybenzyl)oxy]ethyl}-1H-benzimidazole

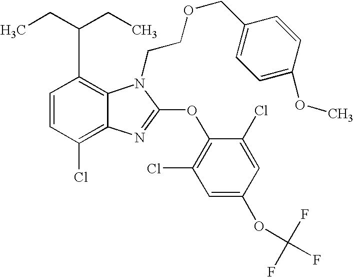

To a solution of 2,4-dichloro-7-(1-ethylpropyl)-1-{2-[(4-methoxybenzyl)oxy]ethyl}-1H-benzimidazole (31 mg, 0.0731 mmol) in N,N-dimethylformamide (1.0 mL) were added 2,6-dichloro-4-trifluoromethoxyphenol (55 mg, 0.221 mmol) and potassium carbonate (31 mg, 0.221 mmol), and the mixture was stirred at 130° C. for 48 hr. After cooling, the reaction mixture was quenched with water and extracted with ethyl acetate (X2). The combined organic layer was washed with brine, dried over anhydrous sodium sulfate and concentrated in vacuo. The residue was purified by silica gel column chromatography eluting with a 2-10% ethyl acetate/n-hexane gradient mixture to give the title compound (8.8 mg, 0.0139 mmol, 19%) as an oil.

¹H NMR (CDCl₃) δ 0.81 (t, J=7.5 Hz, 6H), 1.55-1.80 (m, 4H), 3.05-3.15 (m, 1H), 3.77 (s, 3H), 3.83 (t, J=6.6 Hz, 2H), 4.47 (s, 2H), 4.54 (t, J=6.6 Hz, 2H), 6.76-6.83 (m, 2H), 6.93 (d, J=8.4 Hz, 1H), 7.10-7.18 (m, 3H), 7.28-7.31 (m, 2H).

MS Calcd.: 630, MS Found: 631 (M+H).

### Example 69

### 2-[4-Chloro-2-[2,6-dichloro-4-(trifluoromethoxy)phenoxy]-7-(1-ethylpropyl)-1H-benzimidazol-1-yl]ethanol

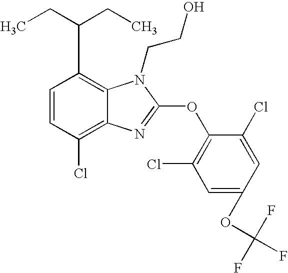

A mixture of 4-chloro-2-[2,6-dichloro-4-(trifluoromethoxy)phenoxy]-7-(1-ethylpropyl)-1-{2-[(4-methoxybenzyl)oxy]ethyl}-1H-benzimidazole (9.7 mg, 0.0154 mmol) and trifluoroacetic acid (0.5 mL) was stirred at 0° C. for 10 min. After trifluoroacetic acid was evaporated in vacuo, the residue was diluted with saturated aqueous sodium hydrogen carbonate solution and extracted with ethyl acetate. The organic layer was washed with brine, dried over anhydrous sodium sulfate and concentrated in vacuo. The residue was purified by preparative TLC eluting with a 25% ethyl acetate/n-hexane mixture to give the title compound (6.8 mg, 0.0133 mmol, 87%) as a colorless solid.

¹H NMR (CDCl₃) δ 0.92 (t, J=7.2 Hz, 6H), 1.70-1.86 (m, 4H), 3.13-3.21 (m, 1H), 3.75-3.80 (m, 1H), 4.11 (q, J=6.0 Hz, 2H), 4.56 (2H, t, J=6.0 Hz), 6.97 (1H, d, J=8.4 Hz), 7.17 (d, J=8.4 Hz, 1H), 7.33 (s, 2H).

MS Calcd.: 512, MS Found: 513 (M+H).

### Example 70

### 4-Bromo-2-(2,4-dichloro-6-methylphenoxy)-7-(1-ethylpropyl)-1-methyl-1H-benzimidazole

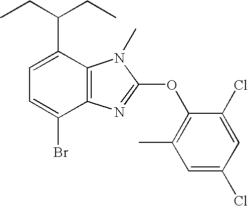

A mixture of 4-bromo-2-chloro-7-(1-ethylpropyl)-1-methyl-1H-benzimidazole (4.15 g, 13.1 mmol), 2,4-dichloro-6-methylphenol (6.97 g, 39.4 mmol), potassium carbonate (5.45 g, 39.4 mmol) and N,N-dimethylformamide (10 mL) was stirred at 120° C. for 20 hr. The mixture was diluted with water and extracted with ethyl acetate. The extract was washed with brine, dried over anhydrous magnesium sulfate, filtered and concentrated in vacuo. The residue was purified by flash column chromatography on silica gel eluting with a 0-10% ethyl acetate/n-hexane gradient mixture. The filtrate was concentrated in vacuo to give a solid, which was recrystallized from ethyl acetate/n-hexane to give the title compound as colorless crystals (4.48 g, 9.82 mmol, 75%).

### mp 146-147° C.

¹H NMR (CDCl₃) δ 0.86 (t, J=7.5 Hz, 6H), 1.68-1.79 (m, 4H), 2.31 (s, 3H), 3.20-3.24 (m, 1H), 3.98 (s, 3H), 6.88 (d, J=8.7 Hz, 1H), 7.20 (d, J=2.4 Hz, 1H), 7.26 (d, J=8.7 Hz, 1H), 7.30 (d, J=2.4 Hz, 1H).

MS Calcd.: 454; MS Found: 455 (M+H).

### Example 71

### 2-(2,4-Dichloro-6-methylphenoxy)-4-ethyl-7-(1-ethylpropyl)-1-methyl-1H-benzimidazole

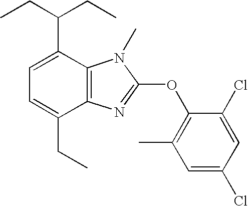

To a mixture of 4-bromo-2-(2,4-dichloro-6-methylphenoxy)-7-(1-ethylpropyl)-1-methyl-1H-benzimidazole (400 mg, 0.876 mmol) and tetraethyltin (411 mg, 1.75 mmol) in toluene (8.0 mL) was added tetrakis(triphenylphosphine)palladium (303 mg, 0.328 mmol), and the mixture was stirred at 120° C. under nitrogen atmosphere for 17 hr. The mixture was diluted with aqueous saturated potassium fluoride and extracted with ethyl acetate. The extract was washed with brine, dried over anhydrous magnesium sulfate, filtered and concentrated in vacuo. The residue was purified by flash column chromatography on silica gel eluting with a 0-15% ethyl acetate/n-hexane gradient mixture. The filtrate was concentrated in vacuo to give the solid, which was recrystallized from methanol to give the title compound as colorless crystals (44.9 mg, 0.111 mmol, 13%).

### mp 96-97° C.

¹H NMR (CDCl₃) δ 0.88 (t, J=7.5 Hz, 6H), 1.21 (t, J=7.2 Hz, 3H), 1.70-1.81 (m, 4H), 2.29 (s, 3H), 2.79 (q, J=7.2 Hz, 2H), 3.19-3.23 (m, 1H), 3.96 (s, 3H), 6.90-6.97 (m, 2H), 7.19 (d, J=2.4 Hz, 1H), 7.30 (d, J=2.4 Hz, 1H).

MS Calcd.: 404; MS Found: 405 (M+H).

### Example 72

### 2-(2-Bromo-4-chlorophenoxy)-4-ethoxy-7-(1-ethylpropyl)-1-methyl-1H-benzimidazole

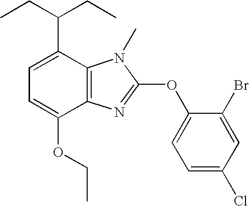

A mixture of 2-chloro-4-ethoxy-7-(1-ethylpropyl)-1-methyl-1H-benzimidazole (509 mg, 1.81 mmol), 2-bromo-4-chlorophenol (1.12 g, 5.43 mmol), potassium carbonate (749 mg, 5.43 mmol) and N,N-dimethylformamide (2.0 mL) was stirred at 120° C. for 18 hr. The mixture was diluted with water and extracted with ethyl acetate. The extract was washed with brine, dried over anhydrous magnesium sulfate, filtered and concentrated in vacuo. The residue was purified by flash column chromatography on silica gel eluting with a 0-10% ethyl acetate/n-hexane gradient mixture. The filtrate was concentrated in vacuo to give the solid, which was recrystallized from methanol to give the title compound as colorless crystals (26.4 mg, 0.0584 mmol, 3%).

### mp 93-94° C.

¹H NMR (CDCl₃) δ 0.85 (t, J=6.9 Hz, 6H), 1.46 (t, J=7.2 Hz, 3H), 1.66-1.80 (m, 4H), 3.15-3.19 (m, 1H), 3.95 (s, 3H), 4.20 (q, J=7.2 Hz, 2H), 6.66 (d, J=8.4 Hz, 1H), 6.93 (d, J=8.4 Hz, 1H), 7.32 (dd, J=2.7 Hz, 9.0 Hz, 1H), 7.60 (d, J=2.7 Hz, 1H), 7.72 (d, J=9.0 Hz, 1H).

MS Calcd.: 450; MS Found: 451 (M+H).

### Example 73

### 2-(2,4-Dichloro-6-methylphenoxy)-4-ethoxy-7-(1-ethylpropyl)-1-methyl-1H-benzimidazole

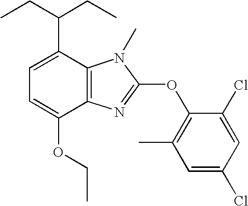

A mixture of 2-chloro-4-ethoxy-7-(1-ethylpropyl)-1-methyl-1H-benzimidazole (609 mg, 2.16 mmol), 2,4-dichloro-6-methylphenol (1.15 g, 6.50 mmol), potassium carbonate (898 mg, 6.50 mmol) and N,N-dimethylformamide (2.0 mL) was stirred at 120° C. for 19 hr. The mixture was diluted with water and extracted with ethyl acetate. The extract was washed with brine, dried over anhydrous magnesium sulfate, filtered and concentrated in vacuo. The residue was purified by flash column chromatography on silica gel eluting with a 0-10% ethyl acetate/n-hexane gradient mixture. The filtrate was concentrated in vacuo to give the solid, which was recrystallized from ethyl acetate/n-hexane to give the title compound as colorless crystals (97.1 mg, 0.230 mmol, 11%).

### mp 116-117° C.

¹H NMR (CDCl₃) δ 0.86 (t, J=7.5 Hz, 6H), 1.39 (t, J=6.9 Hz, 3H), 1.60-1.78 (m, 4H), 2.30 (s, 3H), 3.16-3.20 (m, 1H), 3.96 (s, 3H), 4.19 (q, J=6.9 Hz, 2H), 6.63 (d, J=8.4 Hz, 1H), 6.89 (d, J=8.4 Hz, 1H), 7.17 (d, J=2.4 Hz, 1H), 7.27 (d, J=2.4 Hz, 1H).

MS Calcd.: 420; MS Found: 421 (M+H).

### Example 74

### 4-Chloro-2-[(4,6-dibromo-2-methylpyridin-3-yl)oxy]-7-(1-ethylpropyl)-1-methyl-1H-benzimidazole

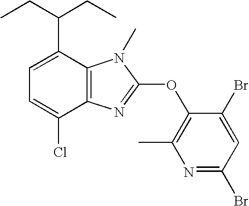

A mixture of 2,4-dichloro-7-(1-ethylpropyl)-1-methyl-1H-benzimidazole (1.50 g, 5.53 mmol), 4,6-dibromo-2-methylpyridin-3-ol (4.43 g, 16.6 mmol), potassium carbonate (2.28 g, 16.5 mmol) and N,N-dimethylformamide (8.0 mL) was stirred at 120° C. for 48 hr. The mixture was diluted with water and extracted with ethyl acetate. The extract was washed with brine, dried over anhydrous magnesium sulfate, filtered and concentrated in vacuo. The residue was purified by flash column chromatography on silica gel eluting with a 5-30% ethyl acetate/n-hexane gradient mixture. The filtrate was concentrated in vacuo to give the solid, which was recrystallized from ethyl acetate/n-hexane to give the title compound as colorless crystals (1.03 g, 2.05 mmol, 37%).

### mp 202-203° C.

¹H NMR (CDCl₃) δ 0.86 (t, J=7.5 Hz, 6H), 1.68-1.83 (m, 4H), 2.55 (s, 3H), 3.20-3.24 (m, 1H), 3.99 (s, 3H), 6.95 (d, J=8.7 Hz, 1H), 7.16 (d, J=8.7 Hz, 1H), 7.63 (s, 1H).

MS Calcd.: 499; MS Found: 500 (M+H).

### Example 75

### 2-{[4-Bromo-2-methyl-6-(trifluoromethyl)pyridin-3-yl]oxy}-4-chloro-7-(1-ethylpropyl)-1-methyl-1H-benzimidazole

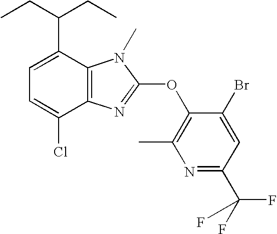

and

### Example 76

### 4-Chloro-7-(1-ethylpropyl)-1-methyl-2-{[2-methyl-4,6-bis(trifluoromethyl)pyridin-3-yl]oxy}-1H-benzimidazole

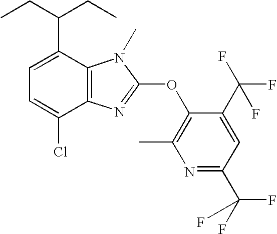

To a mixture of 4-chloro-2-[(4,6-dibromo-2-methylpyridin-3-yl)oxy]-7-(1-ethylpropyl)-1-methyl-1H-benzimidazole (927 mg, 1.85 mmol), copper iodide (1.05 g, 5.54 mmol) and potassium fluoride (321 mg, 5.54 mmol) in 1-methyl-2-pyrrolidone (8.0 mL) was added dropwise trimethyl(trifluoromethyl)silane (658 mg, 4.63 mmol), and the mixture was stirred at 50° C. under nitrogen atmosphere for 6 hr. To the mixture were added copper iodide (500 mg, 263 mmol), potassium fluoride (160 mg, 2.75 mmol) and trimethyl(trifluoromethyl)silane (150 mg, 1.05 mmol), and the mixture was stirred at 50° C. under nitrogen atmosphere for 12 hr. The mixture was neutralized with aqueous saturated sodium hydrogen carbonate solution, and extracted with ethyl acetate. The extract was washed with brine, dried over anhydrous magnesium sulfate, filtered and concentrated in vacuo. The residue was purified by flash column chromatography on silica gel eluting with a 0-20% ethyl acetate/n-hexane gradient mixture. The filtrate was concentrated in vacuo to give the 2-{[4-bromo-2-methyl-6-(trifluoromethyl)pyridin-3-yl]oxy}-4-chloro-7-(1-ethylpropyl)-1-methyl-1H-benzimidazole (26.3 mg, 0.0536 mmol, 3%, Example 75) and 4-chloro-7-(1-ethylpropyl)-1-methyl-2-{[2-methyl-4,6-bis(trifluoromethyl)pyridin-3-yl]oxy}-1H-benzimidazole (34.3 mg, 0.0715 mmol, 4%, Example 76) as a colorless amorphous.

### Example 75

¹H NMR (CDCl₃) δ 0.86 (t, J=7.5 Hz, 6H), 1.68-1.82 (m, 4H), 2.55 (s, 3H), 3.19-3.23 (m, 1H), 3.96 (s, 3H), 6.95 (d, J=8.1 Hz, 1H), 7.15 (d, J=8.1 Hz, 1H), 7.63 (s, 1H).

MS Calcd.: 489; MS Found: 490 (M+H).

### Example 76

¹H NMR (CDCl₃) δ 0.87 (t, J=7.2 Hz, 6H), 1.69-1.81 (m, 4H), 2.63 (s, 3H), 3.20-3.24 (m, 1H), 3.99 (s, 3H), 6.98 (d, J=8.7 Hz, 1H), 7.17 (d, J=8.7 Hz, 1H), 7.83 (s, 1H).

MS Calcd.: 479; MS Found: 480 (M+H).

### Example 77

### 1-(3,5-Dichloro-2-{[7-(1-ethylpropyl)-4-methoxy-1-methyl-1H-benzimidazol-2-yl]oxy}phenyl)-N,N-dimethylmethanamine

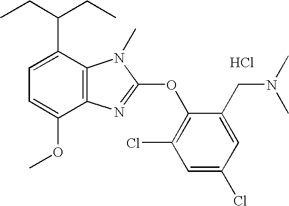

A mixture of 2-chloro-7-(1-ethylpropyl)-4-methoxy-1-methyl-1H-benzimidazole (1.00 g, 3.74 mmol), 2,4-dichloro-6-[(dimethylamino)methyl]phenol (2.47 g, 11.2 mmol), potassium carbonate (1.55 g, 11.2 mmol) and N,N-dimethylformamide (5.0 mL) was stirred at 110° C. for 19 h. The mixture was diluted with water and extracted with ethyl acetate. The extract was washed with brine, dried over anhydrous magnesium sulfate, filtered and concentrated in vacuo. The residue was purified by flash column chromatography on silica gel eluting with a 0-15% ethyl acetate/n-hexane gradient mixture. The filtrate was concentrated in vacuo to give the oil, which was dissolved in methanol. To the solution was added 4.0 M hydrochloric acid in ethyl acetate (0.20 mL), and the mixture was stirred with ice bath cooling for 5 min and concentrated in vacuo. The residue was recrystallized from ethyl acetate/methanol to give the title compound as colorless crystals (359 mg, 0.737 mmol, 20%).

### mp 169-170° C.

¹H NMR (DMSO-d₆) δ 0.82 (t, J=7.2 Hz, 6H), 1.63-1.73 (m, 4H), 2.76 (s, 6H), 3.24-3.28 (m, 1H), 3.74 (s, 3H), 3.96 (s, 3H), 4.39 (s, 2H), 6.68 (d, J=8.7 Hz, 1H), 6.93 (d, J=8.4 Hz, 1H), 7.98 (d, J=2.7 Hz, 1H), 8.07 (d, J=2.7 Hz, 1H).

MS Calcd.: 449; MS Found: 450 (M+H).

### Example 78

### 4-Chloro-2-[2,4-dichloro-6-(pyrrolidin-1-ylmethyl)phenoxy]-7-(1-ethylpropyl)-1-methyl-1H-benzimidazole

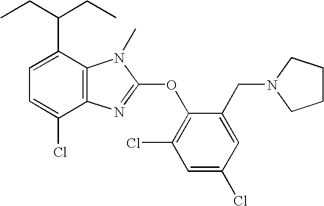

A mixture of 2,4-dichloro-7-(1-ethylpropyl)-1-methyl-1H-benzimidazole (300 mg, 1.07 mmol), 2,4-Dichloro-6-(pyrrolidin-1-ylmethyl)phenol (790 mg, 3.21 mmol), potassium carbonate (443 mg, 3.21 mmol) and N,N-dimethylformamide (2.0 mL) was stirred at 105° C. for 20 hr. The mixture was diluted with water and extracted with ethyl acetate. The extract was washed with brine, dried over anhydrous magnesium sulfate, filtered and concentrated in vacuo. The residue was purified by flash column chromatography on silica gel eluting with a 0-20% ethyl acetate/n-hexane gradient mixture. The filtrate was concentrated in vacuo to give the solid, which was recrystallized from methanol to give the title compound as colorless crystals (66.1 mg, 0.137 mmol, 13%).

### mp 122-123° C.

¹H NMR (CDCl₃) δ 0.86 (t, J=7.5 Hz, 6H), 1.65-1.70 (m, 4H), 1.70-1.82 (m, 4H), 2.44-2.48 (m, 4H), 3.20-3.24 (m, 1H), 3.64 (s, 2H), 3.97 (s, 3H), 6.91 (d, J=8.4 Hz, 1H), 7.13 (d, J=8.4 Hz, 1H), 7.36 (d, J=2.4 Hz, 1H), 7.47 (d, J=2.4 Hz, 1H).

MS Calcd.: 479; MS Found: 480 (M+H).

### Example 79

### 2-[2,4-Dichloro-6-(pyrrolidin-1-ylmethyl)phenoxy]-7-(1-ethylpropyl)-4-methoxy-1-methyl-1H-benzimidazole

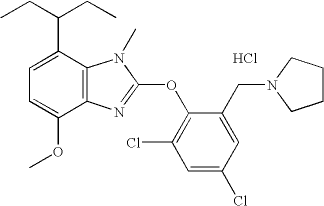

A mixture of 2-chloro-7-(1-ethylpropyl)-4-methoxy-1-methyl-1H-benzimidazole (300 g, 1.12 mmol), 2,4-dichloro-6-(pyrrolidin-1-ylmethyl)phenol (830 mg, 3.36 mmol), potassium carbonate (464 mg, 3.36 mmol) and N,N-dimethylformamide (2.0 mL) was stirred at 105° C. for 2 days. The mixture was diluted with water and extracted with ethyl acetate. The extract was washed with brine, dried over anhydrous magnesium sulfate, filtered and concentrated in vacuo. The residue was purified by flash column chromatography on silica gel eluting with a 0-20% ethyl acetate/n-hexane gradient mixture. The filtrate was concentrated in vacuo to give the oil, which was dissolved in methanol. To the solution was added 4.0 M hydrochloric acid in ethyl acetate (0.20 mL), and the mixture was stirred with ice bath cooling for 5 min and concentrated in vacuo. The residue was recrystallized from ethyl acetate/methanol to give the title compound as colorless crystals (214 mg, 0.417 mmol, 37%).

### mp 183-185° C.

¹H NMR (DMSO-d₆) δ 0.82 (t, J=7.5 Hz, 6H), 1.63-1.70 (m, 4H), 1.88-1.99 (m, 4H), 3.22-3.25 (m, 1H), 3.44-3.48 (m, 4H), 3.74 (s, 3H), 3.97 (s, 3H), 4.46 (s, 2H), 6.69 (d, J=8.7 Hz, 1H), 6.93 (d, J=8.7 Hz, 1H), 7.97 (d, J=2.7 Hz, 1H), 8.11 (d, J=2.7 Hz, 1H).

MS Calcd.: 475; MS Found: 476 (M+H).

### Example 80

### 2-{2,4-Dichloro-6-[(2-methylpyrrolidin-1-yl)methyl]phenoxy}-7-(1-ethylpropyl)-4-methoxy-1-methyl-1H-benzimidazole

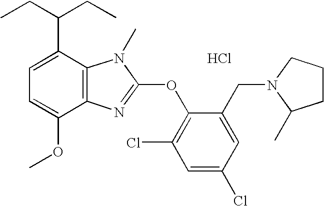

A mixture of 2-chloro-7-(1-ethylpropyl)-4-methoxy-1-methyl-1H-benzimidazole (437 mg, 1.64 mmol), 2,4-dichloro-6-[(2-methylpyrrolidin-1-yl)methyl]phenol (640 mg, 2.46 mmol), potassium carbonate (339 mg, 2.46 mmol) and N,N-dimethylformamide (1.0 mL) was stirred at 110° C. for 16 h. The mixture was diluted with water and extracted with ethyl acetate. The extract was washed with brine, dried over anhydrous magnesium sulfate, filtered and concentrated in vacuo. The residue was purified by flash column chromatography on silica gel eluting with a 5-20% ethyl acetate/n-hexane gradient mixture. The filtrate was concentrated in vacuo to give the oil, which was dissolved in methanol. To the solution was added 4.0 M hydrochloric acid in ethyl acetate (0.10 mL), and the mixture was stirred with ice bath cooling for 5 min, and concentrated in vacuo. The residue was recrystallized from ethyl acetate/methanol to give the title compound as colorless crystals (70.7 mg, 0.134 mmol, 8%).

### mp 171-173° C.

¹H NMR (DMSO-d₆) δ 0.81 (t, J=7.2 Hz, 6H), 1.35 (d, J=6.3 Hz, 3H) 1.65-1.72 (m, 4H), 1.90-1.95 (m, 2H), 2.18-2.20 (m, 2H), 3.20-3.26 (m, 2H), 3.40-3.45 (m, 1H), 3.56-3.63 (m, 1H), 3.75 (s, 3H), 3.97 (s, 3H), 4.28-4.33 (m, 1H), 4.54-4.59 (m, 1H), 6.70 (d, J=8.4 Hz, 1H), 6.95 (d, J=8.4 Hz, 1H), 7.99 (s, 1H), 8.07 (s, 1H).

MS Calcd.: 489; MS Found: 490 (M+H).

### Example 81

### 2-(4-Bromo-2-chloro-6-fluorophenoxy)-7-(1-ethylpropyl)-4-methoxy-1-methyl-1H-benzimidazole

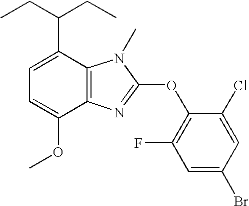

A mixture of 2-chloro-7-(1-ethylpropyl)-4-methoxy-1-methyl-1H-benzimidazole (300 mg, 1.12 mmol), 4-bromo-2-chloro-6-fluorophenol (861 mg, 3.37 mmol), potassium carbonate (465 mg, 3.37 mmol) and N,N-dimethylformamide (1.0 mL) was stirred at 130° C. for 4 days. The mixture was diluted with water and extracted with ethyl acetate. The extract was washed with brine, dried over anhydrous magnesium sulfate, filtered and concentrated in vacuo. The residue was purified by flash column chromatography on silica gel eluting with a 0-15% ethyl acetate/n-hexane gradient mixture. The filtrate was concentrated in vacuo to give the solid, which was recrystallized from ethyl acetate/n-hexane to give the title compound as a colorless crystal (79.4 mg, 0.174 mmol, 16%).

### mp 129-131° C.

¹H NMR (CDCl₃) δ 0.85 (t, J=7.2 Hz, 6H), 1.67-1.80 (m, 4H), 3.15-3.19 (m, 1H), 3.89 (s, 3H), 3.97 (s, 3H), 6.64 (d, J=8.4 Hz, 1H), 6.94 (d, J=8.4 Hz, 1H), 7.25 (d, J=2.1 Hz, 1H), 7.41 (d, J=2.1 Hz, 1H).

MS Calcd.: 454; MS Found: 455 (M+H).

### Example 82

### 7-(1-Ethylpropyl)-2-[2-(1H-imidazol-1-yl)-4-(trifluoromethoxy)phenoxy]-4-methoxy-1-methyl-1H-benzimidazole

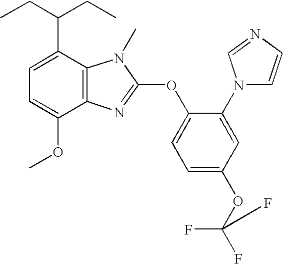

A mixture of 2-[2-bromo-4-(trifluoromethoxy)phenoxy]-7-(1-ethylpropyl)-4-methoxy-1-methyl-1H-benzimidazole (2.19 g, 4.49 mmol), imidazole (1.53 g, 22.5 mmol), potassium carbonate (1.86 g, 13.5 mmol), copper iodide (855 mmol, 4.49 mmol) and N,N′-dimethylethylenediamine (39.5 mg, 0.448 mmol) in N,N-dimethylformamide (10 mL) was stirred at 120° C. under nitrogen atmosphere for 18 hr. The mixture was diluted with aqueous saturated ammonium chloride solution, and extracted with ethyl acetate. The extract was washed with brine, dried over anhydrous magnesium sulfate, filtered and concentrated in vacuo. The residue was purified by flash column chromatography on silica gel eluting with a 0-30% ethyl acetate/n-hexane gradient mixture. The filtrate was concentrated in vacuo to give the solid, which was recrystallized from ethyl acetate/n-hexane to give the title compound as white crystals (110 mg, 0.232 mmol, 5%).

### mp 168-169° C.

¹H NMR (CDCl₃) δ 0.83 (t, J=7.5 Hz, 6H), 1.65-1.79 (m, 4H), 3.08-3.12 (m, 1H), 3.73 (s, 3H), 3.93 (s, 3H), 6.67 (d, J=8.4 Hz, 1H), 6.95 (d, J=8.4 Hz, 1H), 7.13 (d, J=1.8 Hz, 2H), 7.27-7.35 (m, 2H), 7.74 (s, 1H), 7.80 (d, J=9.0 Hz, 1H).

MS Calcd.: 474; MS Found: 475 (M+H).

### Example 83

### 3-Chloro-4-{[4-chloro-7-(1-ethylpropyl)-1-methyl-1H-benzimidazol-2-yl]oxy}-N,N-dimethylaniline

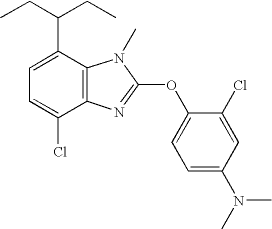

A mixture of 2,4-dichloro-7-(1-ethylpropyl)-1-methyl-1H-benzimidazole (300 mg, 1.11 mmol), 2,6-dichloro-4-dimethylaminophenol (686 mg, 3.33 mmol), potassium carbonate (460 mg, 3.33 mmol) and 1-methyl-2-pyrrolidone (0.5 mL) was stirred at 110° C. for 5 days. The mixture was diluted with water and extracted with ethyl acetate. The extract was washed with brine, dried over anhydrous magnesium sulfate, filtered and concentrated in vacuo. The residue was purified by preparative HPLC. The more polar fraction was neutralized with saturated aqueous sodium hydrogen carbonate solution, and the mixture was extracted with ethyl acetate. The extract was washed with brine, dried over anhydrous magnesium sulfate, filtered and concentrated in vacuo to give the solid, which was recrystallized from methanol to give the title compound as colorless crystals (26 mg, 0.064 mmol, 6%).

### mp 148-149° C.

¹H NMR (CDCl₃) δ 0.84 (t, J=7.5 Hz, 6H), 1.60-1.85 (m, 4H), 2.96 (s, 6H), 3.15-3.25 (m, 1H), 3.96 (s, 3H), 6.65 (dd, J=3.0, 9.0 Hz, 1H), 6.74 (d, J=3.0 Hz, 1H), 6.91 (d, J=8.4 Hz, 1H), 7.13 (d, J=8.4 Hz, 1H), 7.49 (d, J=9.0 Hz, 1H).

MS Calcd.: 405; MS Found: 406 (M+H).

### Example 84 and 85

### 1-[2-{[4-Chloro-7-(1-ethylpropyl)-1-methyl-1H-benzimidazol-2-yl]oxy}-3-methyl-5-(trifluoromethoxy)phenyl]-N,N-dimethylmethanamine and its Hydrochloride

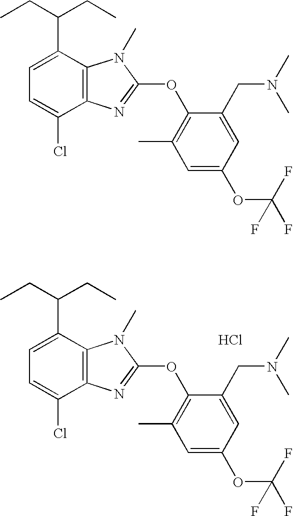

A mixture of 2,4-dichloro-7-(1-ethylpropyl)-1-methyl-1H-benzimidazole (250 mg, 0.92 mmol), 2-[(dimethylamino)methyl]-6-methyl-4-(trifluoromethoxy)phenol (690 mg, 2.77 mmol), potassium carbonate (383 mg, 2.77 mmol) and 1-methyl-2-pyrrolidone (0.5 mL) was stirred at 110° C. for 120 h. The mixture was diluted with saturated aqueous sodium hydrogen carbonate solution and extracted with ethyl acetate. The extract was washed with brine, dried over anhydrous magnesium sulfate, filtered and concentrated in vacuo. The residue was purified by preparative HPLC. The filtrate was neutralized with saturated aqueous sodium hydrogen carbonate, and the mixture was extracted with ethyl acetate. The extract was washed with brine, dried over anhydrous magnesium sulfate, filtered and concentrated in vacuo to give the solid, which was recrystallized from methanol to give the free base of the title compound as colorless crystals (30 mg, 0.062 mmol, 7%, Example 84). The filtrate was concentrated in vacuo to give a solid, which was dissolved in ethyl acetate. To the solution was added 4.0 M hydrochloric acid in ethyl acetate (0.10 mL), and the mixture was stirred with ice bath cooling for 5 min, and concentrated in vacuo to give the hydrochloride of the title compound as a white solid (59 mg, 0.113 mmol, 12%, Example 85).

### Example 84

### Free Base

¹H NMR (CDCl₃) δ 0.87 (t, J=7.2 Hz, 6H), 1.60-1.85 (m, 4H), 2.15 (s, 6H), 2.25 (s, 3H), 3.15-3.30 (m, 1H), 3.34 (s, 2H), 3.97 (s, 3H), 6.92 (d, J=8.4 Hz, 1H), 7.04 (s, 1H), 7.13 (d, J=8.4 Hz, 1H), 7.21 (s, 1H).

MS Calcd.: 483; MS Found: 484 (M+H).

### Example 85

### Hydrochloride

¹H NMR (DMSO-d₆) δ 0.84 (t, J=7.2 Hz, 6H), 1.60-1.80 (m, 4H), 2.13 (s, 3H), 2.20-2.25 (m, 1H), 2.77 (2, 6H), 3.95-4.00 (m, 2H), 3.97 (s, 3H), 7.02 (d, J=8.1 Hz, 1H), 7.17 (d, J=8.1 Hz, 1H), 7.52 (s, 1H), 7.78 (s, 1H).

MS Calcd.: 483; MS Found: 484 (M+H).

### Example 86

### 1-(3,5-Dichloro-2-{[4-chloro-7-(1-ethylpropyl)-1-methyl-1H-1-benzimidazol-2-yl]oxy}phenyl)-N,N-dimethylmethanamine

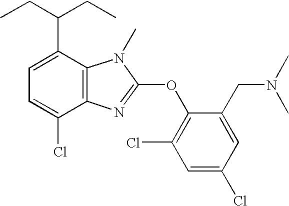

A mixture of 2,4-dichloro-7-(1-ethylpropyl)-1-methyl-1H-benzimidazole (250 mg, 0.922 mmol), 2,4-dichloro-6-[(dimethylamino)methyl]phenol (609 mg, 2.77 mmol), potassium carbonate (382 mg, 2.77 mmol) and 1-methyl-2-pyrrolidone (1 mL) was stirred at 120° C. for 18 hr. The mixture was diluted with saturated aqueous sodium hydrogen carbonate solution and extracted with ethyl acetate. The extract was washed with brine, dried over anhydrous magnesium sulfate, filtered and concentrated in vacuo. The residue was purified by flash column chromatography on silica gel eluting with a 10-50% ethyl acetate/n-hexane gradient mixture. The resulting product was purified by preparative HPLC. The filtrate was neutralized with saturated aqueous sodium hydrogen carbonate solution, and the mixture was extracted with ethyl acetate. The extract was washed with brine, dried over anhydrous magnesium sulfate, filtered and concentrated in vacuo to give the solid, which was recrystallized from methanol to give the title compound (72 mg, 0.158 mmol, 17%) as colorless crystals.

### mp 149-151° C.

¹H NMR (CDCl₃) δ 0.86 (t, J=7.5 Hz, 6H), 1.65-1.85 (m, 4H), 2.22 (s, 6H), 3.20-3.30 (m, 1H), 3.45 (s, 2H), 3.98 (s, 3H), 6.93 (d, J=8.4 Hz, 1H), 7.13 (d, J=8.4 Hz, 1H), 7.37 (d, J=2.7 Hz, 1H), 7.49 (d, J=2.7 Hz, 1H).

MS Calcd.: 453; MS Found: 454 (M+H), 456.

### Example 87

### 3-Chloro-4-{[7-(1-ethylpropyl)-4-methoxy-1-methyl-1H-benzimidazol-2-yl]oxy}-N,N-dimethyl-5-(pyrrolidin-1-ylmethyl)aniline

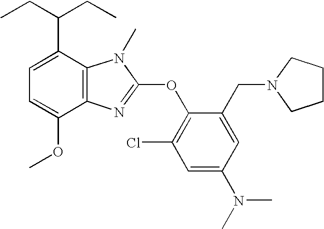

A mixture of 2-chloro-7-(1-ethylpropyl)-4-methoxy-1-methyl-1H-benzimidazole (50 mg, 0.189 mmol), 2-chloro-4-(dimethylamino)-6-(pyrrolidin-1-ylmethyl)phenol (144 mg, 0.567 mmol), potassium carbonate (78 mg, 0.567 mmol) and 1-methyl-2-pyrrolidone (0.5 mL) was stirred at 115° C. for 40 hr. The mixture was diluted with saturated aqueous sodium hydrogen carbonate solution and extracted with ethyl acetate. The extract was washed with brine, dried over anhydrous magnesium sulfate, filtered and concentrated in vacuo. The residue was purified by flash column chromatography on basic silica gel eluting with a 10-50% ethyl acetate/n-hexane gradient mixture. The resulting product was purified by preparative HPLC eluting with a 5-90% acetonitrile/water gradient mixture containing 0.1% trifluoroacetic acid. The filtrate was neutralized with saturated aqueous sodium hydrogen carbonate solution, and the mixture was extracted with ethyl acetate. The extract was washed with brine, dried over anhydrous magnesium sulfate, filtered and concentrated in vacuo to give the title compound (10 mg, 0.0206 mmol, 11%) as a white solid.

¹H NMR (CDCl₃) δ 0.87 (t, J=7.5 Hz, 6H), 1.65-1.85 (m, 8H), 2.45-2 2.60 (m, 4H), 2.93 (s, 6H), 3.15-3.25 (m, 1H), 3.64 (s, 2H), 3.87 (s, 3H), 3.96 (s, 3H), 6.61 (d, J=8.4 Hz, 1H), 6.63 (d, J=3.0 Hz, 1H), 6.747 (d, J=3.0 Hz, 1H), 6.89 (d, J=8.4 Hz, 1H).

MS Calcd.: 484; MS Found: 485 (M+H).

### Example 88

### N-(2,4-Dichloro-6-methylphenyl)-7-(1-ethylpropyl)-4-methoxy-1-methyl-1H-benzimidazol-2-amine

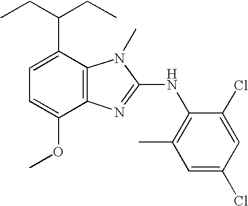

To a solution of 2,4-dichloro-6-methylaniline (990 mg, 5.62 mmol) in 1-methyl-2-pyrrolidone (0.1 mL) were added sodium hydride (225 mg, 5.62 mmol) and 2-chloro-7-(1-ethylpropyl)-4-methoxy-1-methyl-1H-benzimidazole (500 mg, 1.87 mmol). The reaction mixture was irradiated at 180° C. by a microwave for 30 min. The reaction mixture was neutralized with saturated aqueous sodium hydrogen carbonate solution and extracted with ethyl acetate (X3). The combined organic layer was washed with brine, dried over anhydrous magnesium sulfate, filtered and concentrated in vacuo. The residue was purified by basic silica gel column chromatography eluting with a 15-25% ethyl acetate/n-hexane gradient mixture. The resulting solid was recrystallized from ethyl acetate/n-hexane to give the title compound as a colorless powder (221 mg, 0.544 mmol, 29%).

### mp 201-203° C.

¹H NMR (CDCl₃) δ: 0.86 (t, J=7.5 Hz, 6H), 1.63-1.81 (m, 4H), 2.13 (s, 3H), 3.12-3.21 (m, 1H), 3.80 (s, 3H), 3.84 (s, 3H), 6.58 (d, J=8.4 Hz, 1H), 6.85 (d, J=8.4 Hz, 1H), 7.09 (d, J=2.7 Hz, 1H), 7.26 (d, J=2.7 Hz, 1H).

MS Calcd.: 405, MS Found: 406 (M+H).

### Example 89

### N-[2,6-Dichloro-4-(trifluoromethoxy)phenyl]-7-(1-ethylpropyl)-4-methoxy-1-methyl-1H-benzimidazol-2-amine

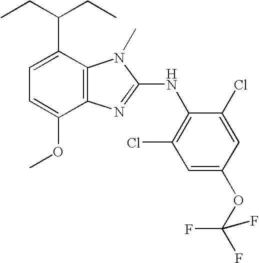

To a solution of 2-chloro-7-(1-ethylpropyl)-4-methoxy-1-methyl-1H-benzimidazole (500 mg, 1.87 mmol) in 1-methyl-2-pyrrolidone (0.1 mL) was added 2,6-dichloro-4-(trifluoromethoxy)aniline (1.38 g, 5.62 mmol). The reaction mixture was heated at 150° C. for 3 h, and heated to 180° C. for 11 days. The reaction mixture was diluted with water and extracted with ethyl acetate (X3). The combined organic layer was washed with brine, dried over anhydrous magnesium sulfate, filtered and concentrated in vacuo. The residue was purified by preparative HPLC to give the title compound as the trifluoroacetic acid salt. The salt was washed with aqueous sodium hydrogen carbonate solution and extracted with ethyl acetate. The extract was washed with brine, dried over anhydrous magnesium sulfate, filtered and concentrated in vacuo. The residue was purified by preparative TLC eluting with a 25% ethyl acetate/n-hexane gradient mixture to give the title compound as a colorless powder (15.9 mg, 0.0334 mmol, 1.8%).

¹H NMR (CDCl₃) δ: 0.87 (t, J=7.5 Hz, 6H), 1.61-1.81 (m, 4H), 3.17-3.21 (m, 1H), 3.83 (s, 3H), 3.86 (s, 3H), 6.60 (d, J=8.7 Hz, 1H), 6.86 (d, J=8.7 Hz, 1H), 7.25-7.26 (m, 2H).

MS Calcd.: 475, MS Found: 476 (M+H).

### Example 90

### 2-[(2,4-Dichloro-6-methylphenyl)amino]-7-(1-ethylpropyl)-1-methyl-1H-benzimidazol-4-ol

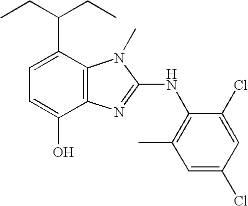

To a solution of 2-chloro-7-(1-ethylpropyl)-4-methoxy-1-methyl-1H-benzimidazole (500 mg, 1.87 mmol) in 1-methyl-2-pyrrolidone (0.4 mL) was added 2,4-dichloro-6-methylaniline (990 mg, 5.62 mmol). The reaction mixture was stirred at 110° C. for 66 h, heated to 150° C. for 7 h, and heated to 180° C. for 2 days. The reaction mixture was diluted with water and extracted with ethyl acetate (X3). The combined organic layer was washed with brine, dried over anhydrous magnesium sulfate, filtered and concentrated in vacuo. The residue was purified by silica gel column chromatography eluting with a 5-25% ethyl acetate/n-hexane gradient mixture to give a mixture contained the title compound. The mixture was purified by preparative HPLC to give the title compound as the trifluoroacetic acid salt. The salt was washed with aqueous sodium hydrogen carbonate solution and extracted with ethyl acetate. The extract was washed with brine, dried over anhydrous magnesium sulfate, filtered and concentrated in vacuo. The residue was purified by preparative TLC eluting with a 25% ethyl acetate/n-hexane gradient mixture to give the title compound as a colorless powder (19.4 mg, 0.0494 mmol, 2.6%).

¹H NMR (CDCl₃) δ: 0.85 (t, J=7.2 Hz, 6H), 1.61-1.80 (m, 4H), 2.07 (s, 3H), 3.05-3.14 (m, 1H), 3.77 (s, 3H), 6.58 (d, J=8.4 Hz, 1H), 6.82 (d, J=8.4 Hz, 1H), 7.00 (s, 1H), 7.21 (d, J=2.4 Hz, 1H).

MS Calcd.: 391, MS Found: 392 (M+H).

### Example 91

### 2-(2,4-Dichloro-6-methylphenoxy)-7-(1-ethylpropyl)-1-methyl-1H-benzimidazole

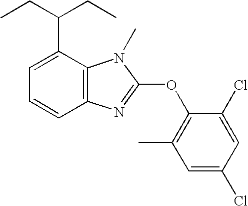

A mixture of 2-chloro-7-(1-ethylpropyl)-1-methyl-1H-benzimidazole (382 mg, 1.61 mmol), 2,4-dichloro-6-methylphenol (852 mg, 4.84 mmol), potassium carbonate (669 mg, 4.84 mmol) and N,N-dimethylformamide (0.5 mL) was stirred at 110° C. for 4 days. After cooling, the reaction mixture was neutralized with aqueous saturated sodium hydrogen carbonate and extracted with ethyl acetate (X3). The combined organic layer was washed with 1N aqueous sodium hydroxide (X2), washed with brine, dried over anhydrous magnesium sulfate, filtered and concentrated in vacuo. The residue was purified by basic silica gel column chromatography eluting with a 10-50% ethyl acetate/n-hexane gradient mixture. The resulting solid was recrystallized from ethyl acetate/n-hexane to give the title compound as a colorless powder (169.9 mg, 0.450 mmol, 28%).

### mp 91-92° C.

¹H NMR (CDCl₃) δ: 0.88 (t, J=7.5 Hz, 6H), 1.72-1.84 (m, 4H), 2.30 (s, 3H), 3.23-3.28 (m, 1H), 4.00 (s, 3H), 7.00-7.20 (m, 3H), 7.31-7.37 (m, 2H).

MS Calcd.: 376, MS Found: 377 (M+H).

### Example 92

### 1-[4-Chloro-2-(2,4-dichloro-6-methylphenoxy)-1-methyl-1H-benzimidazol-7-yl]propan-1-one

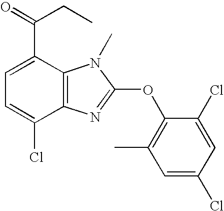

A mixture of 1-(2,4-dichloro-1-methyl-1H-benzimidazol-7-yl)propan-1-one (3.51 g, 13.7 mmol), 2,4-dichloro-6-methylphenol (7.25 g, 40.9 mmol), potassium carbonate (5.65 g, 40.9 mmol) and N,N-dimethylformamide (10 mL) was stirred at 110° C. for 13 hr. The mixture was diluted with water and extracted with ethyl acetate. The extract was washed with 1N aqueous sodium hydroxide, washed with brine, dried over anhydrous magnesium sulfate, filtered and concentrated in vacuo. The residue was washed with ethyl acetate to give the title compound as a colorless powder (3.09 mg, 7.77 mmol, 57%).

¹H NMR (CDCl₃) δ 1.47 (t, J=7.2 Hz, 3H), 2.25 (s, 3H), 3.13 (q, J=7.2 Hz, 2H), 3.72 (s, 3H), 7.32 (d, J=8.1 Hz, 1H), 7.55 (d, J=2.7 Hz, 1H), 7.66 (d, J=8.1 Hz, 1H), 7.69 (d, J=2.7 Hz, 1H).

MS Calcd.: 396; MS Found: 397 (M+H).

### Example 93

### 4-Chloro-2-(2,4-dichloro-6-methylphenoxy)-1-methyl-7-(1-methylenepropyl)-1H-benzimidazole

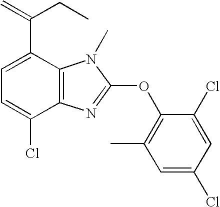

To a solution of 1-[4-chloro-2-(2,4-dichloro-6-methylphenoxy)-1-methyl-1H-benzimidazol-7-yl]propan-1-one (2.50 g, 6.29 mmol) in toluene (50 mL) was added Tebbe reagent (0.5 M solution in toluene, 39.0 mL, 19.5 mmol), and the mixture was stirred at 50° C. for 1 hr under nitrogen atmosphere. The mixture was neutralized with aqueous saturated sodium hydrogen carbonate solution, and extracted with ethyl acetate. The extract was washed with brine, dried over anhydrous magnesium sulfate, filtered and concentrated in vacuo. The residue was purified by flash column chromatography on silica gel eluting with a 0-20% ethyl acetate/n-hexane gradient mixture. The filtrate was concentrated in vacuo to give a solid, which was washed with n-hexane to give the title compound as a colorless powder (1.01 g, 2.55 mmol, 41%).

¹H NMR (CDCl₃) δ 1.17 (t, J=7.5 Hz, 3H), 2.30 (s, 3H), 2.46 (q, J=7.5 Hz, 2H), 3.79 (s, 3H), 5.07 (d, J=1.8 Hz, 1H), 5.38 (d, J=1.8 Hz, 1H), 6.82 (d, J=8.1 Hz, 1H), 7.11 (d, J=8.1 Hz, 1H), 7.19 (d, J=2.4 Hz, 1H), 7.30 (d, J=2.4 Hz, 1H).

MS Calcd.: 394; MS Found: 395 (M+H).

### Example 94

### 2-[4-Chloro-2-(2,4-dichloro-6-methylphenoxy)-1-methyl-1H-benzimidazol-7-yl]butan-1-ol

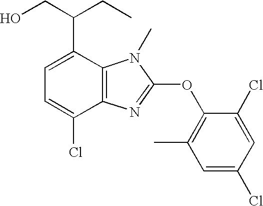

To a solution of 4-chloro-2-(2,4-dichloro-6-methylphenoxy)-1-methyl-7-(1-methylenepropyl)-1H-benzimidazole (70.0 mg, 0.176 mmol) in tetrahydrofuran (1.0 mL) was added borane-tetrahydrofuran complex (1.0 M solution in tetrahydrofuran, 0.19 mL, 0.19 mmol), and the mixture was stirred at room temperature for 90 min under nitrogen atmosphere. To the mixture were added ethanol (1.1 mL), 1N aqueous sodium hydroxide (0.20 mL, 0.20 mmol) and 30% aqueous hydrogen peroxide solution (0.10 mL), and the mixture was stirred at room temperature for 90 min under nitrogen atmosphere. The mixture was diluted with aqueous saturated sodium thiosulfate solution, and extracted with ethyl acetate. The extract was washed with brine, dried over anhydrous magnesium sulfate, filtered and concentrated in vacuo. The residue was purified by flash column chromatography on silica gel eluting with a 3-35% ethyl acetate/n-hexane gradient mixture. The filtrate was concentrated in vacuo to give the solid, which was recrystallized from methanol to give the title compound as colorless crystals (28.0 mg, 0.0677 mmol, 38%).

### mp 208-209° C.

¹H NMR (CDCl₃) δ 0.91 (t, J=7.5 Hz, 3H), 1.69-1.82 (m, 2H), 2.29 (s, 3H), 3.52-3.59 (m, 2H), 3.71-3.82 (m, 2H), 4.02 (s, 3H), 6.94 (d, J=8.1 Hz, 1H), 7.18 (d, J=8.1 Hz, 1H), 7.19 (d, J=2.4 Hz, 1H), 7.29 (d, J=2.4 Hz, 1H).

MS Calcd.: 412; MS Found: 413 (M+H).

### Example 95

### 2-[4-Chloro-2-(2,4-dichloro-6-methylphenoxy)-1-methyl-1H-benzimidazol-7-yl]butanal

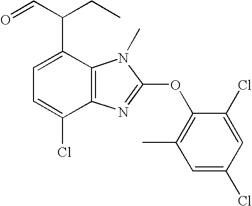

To a solution of 2-[4-chloro-2-(2,4-dichloro-6-methylphenoxy)-1-methyl-1H-benzimidazol-7-yl]butan-1-ol (635 mg, 1.53 mmol) in dichloromethane (15 mL) was added Dess-Martin reagent (716 mg, 1.68 mmol), and the mixture was stirred with ice bath cooling for 45 min. The mixture was neutralized with aqueous saturated sodium hydrogen carbonate, and extracted with ethyl acetate. The extract was washed with brine, dried over anhydrous magnesium sulfate, filtered and concentrated in vacuo. The residue was purified by flash column chromatography on silica gel eluting with a 0-20% ethyl acetate/n-hexane gradient mixture. The filtrate was concentrated in vacuo to give the title compound as a colorless solid (507 mg, 1.23 mmol, 80%).

¹H NMR (CDCl₃) δ 1.04 (t, J=7.5 Hz, 3H), 1.93-1.94 (m, 1H), 2.19-2.24 (m, 1H), 2.30 (s, 3H), 4.03 (s, 3H), 4.10-4.15 (m, 1H), 6.89 (d, J=8.1 Hz, 1H), 7.19 (d, J=8.1 Hz, 1H), 7.20 (d, J=1.8 Hz, 1H), 7.31 (d, J=2.4 Hz, 1H), 9.64 (d, J=2.7 Hz, 1H).

MS Calcd.: 410; MS Found: 411 (M+H).

### Example 96

### 4-Chloro-2-(2,4-dichloro-6-methylphenoxy)-7-(1-ethylprop-2-en-1-yl)-1-methyl-1H-benzimidazole

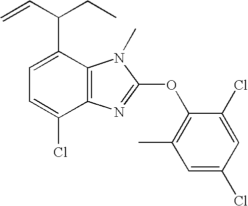

To a solution of 2-[4-chloro-2-(2,4-dichloro-6-methylphenoxy)-1-methyl-1H-benzimidazol-7-yl]butanal (500 mg, 1.21 mmol) in toluene (35 mL) was added Tebbe reagent (0.5 M solution in toluene, 4.9 mL, 2.45 mmol), and the mixture was stirred at 50° C. for 13 hr under nitrogen atmosphere. The mixture was neutralized with aqueous saturated sodium hydrogen carbonate solution, and extracted with ethyl acetate. The extract was washed with brine, dried over anhydrous magnesium sulfate, filtered and concentrated in vacuo. The residue was purified by flash column chromatography on silica gel eluting with a 0-20% ethyl acetate/n-hexane gradient mixture. The filtrate was concentrated in vacuo to give the title compound as colorless amorphous (233 mg, 0.569 mmol, 47%).

¹H NMR (CDCl₃) δ 0.97 (t, J=7.8 Hz, 3H), 1.82-1.90 (m, 2H), 2.29 (s, 3H), 3.87-3.94 (m, 1H), 3.98 (s, 3H), 4.92-4.96 (m, 1H), 5.10-5.14 (m, 1H), 6.01-6.12 (m, 1H), 6.93 (d, J=8.1 Hz, 1H), 7.13 (d, J=8.1 Hz, 1H), 7.18 (d, J=2.4 Hz, 1H), 7.29 (d, J=2.4 Hz, 1H).

MS Calcd.: 408; MS Found: 409 (M+H).

### Example 97

### 3-[4-Chloro-2-(2,4-dichloro-6-methylphenoxy)-1-methyl-1H-benzimidazol-7-yl]pentan-1-ol

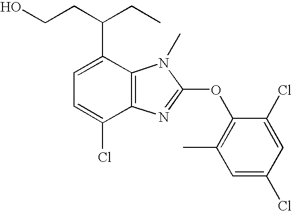

To a solution of 4-chloro-2-(2,4-dichloro-6-methylphenoxy)-7-(1-ethylprop-2-en-1-yl)-1-methyl-1H-benzimidazole (230 mg, 0.561 mmol) in tetrahydrofuran (6.0 mL) was added tetrahydrofuran-tetrahydrofuran complex (1.0 M solution in tetrahydrofuran, 1.12 mL, 1.12 mmol), and the mixture was stirred at room temperature for 90 min under nitrogen atmosphere. To the mixture were added ethanol (2.0 mL), 1N aqueous sodium hydroxide (0.85 mL, 0.85 mmol) and 30% aqueous hydrogen peroxide solution (0.32 mL), and the mixture was stirred at room temperature for 1 hr under nitrogen atmosphere. The mixture was diluted with aqueous saturated sodium thiosulfate solution, and extracted with ethyl acetate. The extract was washed with brine, dried over anhydrous magnesium sulfate, filtered and concentrated in vacuo. The residue was purified by flash column chromatography on silica gel eluting with a 5-40% ethyl acetate/n-hexane gradient mixture. The filtrate was concentrated in vacuo to give a solid, which was recrystallized from methanol to give the title compound as colorless crystals (127 mg, 0.297 mmol, 53%).

### mp 165-167° C.

¹H NMR (CDCl₃) δ 0.86 (t, J=7.5 Hz, 3H), 1.72-2.04 (m, 4H), 2.31 (s, 3H), 3.41-3.47 (m, 1H), 3.61-3.68 (m, 2H), 4.04 (s, 3H), 6.93 (d, J=8.4 Hz, 1H), 7.13 (d, J=8.4 Hz, 1H), 7.19 (d, J=2.1 Hz, 1H), 7.30 (d, J=2.1 Hz, 1H).

MS Calcd.: 426; MS Found: 427 (M+H).

### Example 98

### 3-[4-Chloro-2-(2,4-dichloro-6-methylphenoxy)-1-methyl-1H-benzimidazol-7-yl]pentanal

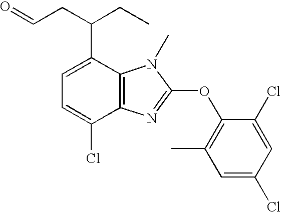

To a solution of 3-[4-chloro-2-(2,4-dichloro-6-methylphenoxy)-1-methyl-1H-benzimidazol-7-yl]pentan-1-ol (113 mg, 0.264 mmol) in dichloromethane (2.0 mL) was added Dess-Martin reagent (123 mg, 0.291 mmol), and the mixture was stirred with ice bath cooling for 1 hr. The mixture was neutralized with aqueous saturated sodium hydrogen carbonate solution, and extracted with ethyl acetate. The extract was washed with brine, dried over anhydrous magnesium sulfate, filtered and concentrated in vacuo. The residue was purified by flash column chromatography on silica gel eluting with a 10-50% ethyl acetate/n-hexane gradient mixture. The filtrate was concentrated in vacuo to give a solid, which was washed with ethyl acetate-diisopropyl ether to give the title compound as a colorless powder (106 mg, 0.297 mmol, 53%).

### mp 157-158° C.

¹H NMR (CDCl₃) δ 0.87 (t, J=7.2 Hz, 3H), 1.73-1.83 (m, 2H), 2.31 (s, 3H), 2.83-3.03 (m, 2H), 3.95-3.99 (m, 1H), 4.09 (s, 3H), 6.90 (d, J=8.4 Hz, 1H), 7.13 (d, J=8.4 Hz, 1H), 7.19 (d, J=1.8 Hz, 1H), 7.30 (d, J=1.8 Hz, 1H), 9.74 (s, 1H).

MS Calcd.: 424; MS Found: 425 (M+H).

### Example 99

### 3-[4-Chloro-2-(2,4-dichloro-6-methylphenoxy)-1-methyl-1H-benzimidazol-7-yl]pentanoic acid

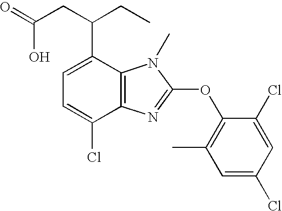

To a suspension of 3-[4-chloro-2-(2,4-dichloro-6-methylphenoxy)-1-methyl-1H-benzimidazol-7-yl]pentanal (91.7 mg, 0.215 mmol) in 2-propanol (2.0 mL), 2-methyl-2-butene (0.20 mL) and water (0.60 mL) were added sodium dihydrogen phosphate (28.3 mg, 0.236 mmol) and sodium chlorite (77.9 mg, 0.861 mmol), and the mixture was stirred at room temperature for 1 hr. The mixture was diluted with aqueous saturated sodium thiosulfate solution, acidified by 1N hydrochloric acid, and extracted with ethyl acetate. The extract was washed with brine, dried over anhydrous magnesium sulfate, filtered and concentrated in vacuo. The residue was purified by flash column chromatography on silica gel eluting with a 10-60% ethyl acetate/n-hexane gradient mixture. The filtrate was concentrated in vacuo to give a solid, which was recrystallized from ethyl acetate/diisopropyl ether to give the title compound as colorless crystals (24.4 mg, 0.0552 mmol, 26%).

### mp 201-202° C.

¹H NMR (DMSO-d₆) δ 0.79 (t, J=7.5 Hz, 3H), 1.67-1.74 (m, 2H), 2.24 (s, 3H), 2.68-2.72 (m, 2H), 3.77-3.82 (m, 1H), 4.06 (s, 3H), 7.08 (d, J=8.4 Hz, 1H), 7.15 (d, J=8.4 Hz, 1H), 7.54 (d, J=2.4 Hz, 1H), 7.68 (d, J=2.4 Hz, 1H), 12.18 (s, 1H).

MS Calcd.: 440; MS Found: 441 (M+H).

### Example 100

### 2-[4-Chloro-2-[(2,4-dichlorophenyl)amino]-7-(1-ethylpropyl)-1H-benzimidazol-1-yl]ethanol

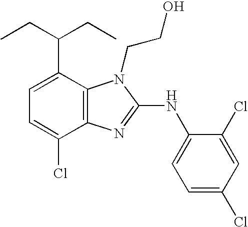

### i) Ethyl [4-chloro-2-[(2,4-dichlorophenyl)amino]-7-(1-ethylpropyl)-1H-benzimidazol-1-yl]acetate

A mixture of ethyl [2,4-dichloro-7-(1-ethylpropyl)-1H-benzimidazol-1-yl]acetate (708 mg, 2.06 mmol), 2,4-dichloroaniline (1.00 g, 6.19 mmol) and 1-methyl-2-pyrrolidone (0.5 mL) was stirred at 110° C. for 3.5 days. The reaction mixture was neutralized with saturated aqueous sodium hydrogen carbonate solution and extracted with ethyl acetate (X2). The combined organic layer was washed with brine, dried over anhydrous sodium sulfate and concentrated in vacuo. The residue was purified by basic silica gel column chromatography eluting with a 3-15% ethyl acetate/n-hexane gradient mixture. The resulting solid was washed with n-hexane to give the title compound (115 mg, 0.245 mmol, 12%).

¹H NMR (CDCl₃) δ: 0.82 (t, J=7.5 Hz, 6H), 1.34 (t, J=7.2 Hz, 3H), 1.63-1.86 (m, 4H), 2.90-3.05 (m, 1H), 4.35 (q, J=7.2 Hz, 2H), 4.93 (s, 2H), 6.96 (d, J=8.4 Hz, 1H), 7.21-7.44 (m, 4H), 8.28 (d, J=8.4 Hz, 1H).

MS Calcd.: 467, MS Found: 468 (M+H).

### ii) 2-[4-Chloro-2-[(2,4-dichlorophenyl)amino]-7-(1-ethylpropyl)-1H-benzimidazol-1-yl]ethanol

To a solution of ethyl [4-chloro-2-[(2,4-dichlorophenyl)amino]-7-(1-ethylpropyl)-1H-benzimidazol-1-yl]acetate (99 mg, 0.211 mmol) in tetrahydrofuran (1.5 mL) were added lithium tetrahydroborate (14 mg, 0.634 mmol), and the mixture was stirred at 65° C. for 1.5 hr. After cooling, the reaction was quenched with water and extracted with ethyl acetate. The organic layer was washed with brine, dried over anhydrous sodium sulfate and concentrated in vacuo. The resulting solid was washed with n-hexane to give the title compound (86 mg, 0.202 mmol, 96%) as a colorless solid.

### mp 192-193° C.

¹H NMR (CDCl₃) δ: 0.77 (t, J=7.5 Hz, 6H), 1.47-1.78 (m, 4H), 2.65-2.75 (m, 1H), 4.15 (t, J=4.2 Hz, 2H), 4.29 (t, J=4.2 Hz, 2H), 5.03 (br s, 1H), 6.79 (d, J=8.4 Hz, 1H), 7.07 (d, J=8.4 Hz, 1H), 7.20-7.30 (m, 2H), 8.43 (d, J=9.0 Hz, 1H), 8.82 (br s, 1H).

MS Calcd.: 425, MS Found: 426 (M+H).

### Example 101

### 2-[4-Chloro-2-({4-chloro-2-[(dimethylamino)methyl]phenyl}amino)-7-(1-ethylpropyl)-1H-benzimidazol-1-yl]ethanol

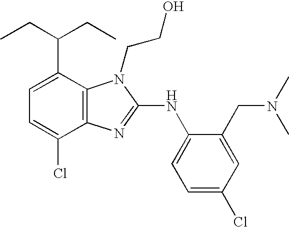

To a solution of 2-[2,4-dichloro-7-(1-ethylpropyl)-1H-benzimidazol-1-yl]ethanol (222 mg, 0.737 mmol) in 1-butylalcohol (2 mL) were added 4-chloro-2-[(dimethylamino)methyl]aniline (408 mg, 2.21 mmol) and p-toluenesulfonic acid monohydrate (140 mg, 0.737 mmol), and the mixture was stirred at 120° C. for 36 hr. The reaction mixture was diluted with saturated aqueous sodium hydrogen carbonate solution and extracted with ethyl acetate. The organic layer was washed with brine, dried over anhydrous sodium sulfate and concentrated in vacuo. The residue was purified by basic silica gel column chromatography eluting with a 15-50% ethyl acetate/n-hexane gradient mixture and preparative HPLC. The filtrate was neutralized with saturated aqueous sodium hydrogen carbonate solution, and the mixture was extracted with ethyl acetate. The extract was washed with brine, dried over anhydrous magnesium sulfate, filtered and concentrated in vacuo to give the title compound (13 mg, 0.0289 mmol, 3.9%).

¹H NMR (CDCl₃) δ: 0.84 (t, J=7.2 Hz, 6H), 1.57-2.83 (m, 4H), 2.14, (s, 6H), 2.90-2.99 (m, 1H), 3.32 (s, 2H), 3.45 (t, J=6.9 Hz, 2H), 4.32 (t, J=6.9 Hz, 2H), 6.42 (br s, 1H), 6.59 (d, J=8.7 Hz, 1H), 6.87 (d, J=8.7 Hz, 1H), 6.93 (d, J=2.7 Hz, 1H), 7.04 (d, J=8.7 Hz, 1H), 7.10 (dd, J=8.7, 2.7 Hz, 1H), 9.14 (br s, 1H).

MS Calcd.: 448, MS Found: 449 (M+H).

### Example 102

### 1-{2-[2,6-Dichloro-4-(trifluoromethoxy)phenoxy]-1-methyl-1H-benzimidazol-7-yl}-N,N-dimethylpropan-1-amine

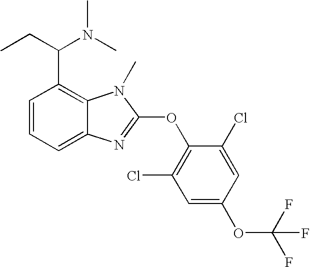

### i) 2-[2,6-Dichloro-4-(trifluoromethoxy)phenoxy]-1-methyl-1H-benzimidazole-7-carbonitrile

To a solution of 7-bromo-2-chloro-1-methyl-1H-benzimidazole (500 mg, 2.04 mmol) and 2,6-dichloro-4-trifluoromethoxyphenol (1.51 g, 6.12 mmol) in acetonitrile (5 mL) was added potassium carbonate (846 mg, 6.12 mmol). The mixture was heated at 110° C. for 3 days. After cooling to room temperature, the reaction mixture was dissolved in ethyl acetate and water. The mixture was extracted with ethyl acetate (X3). The combined organics were dried over anhydrous magnesium sulfate, filtered, and concentrated in vacuo. The residue was washed with ethyl acetate/hexane (1:5) to give a brown amorphous. The amorphous was dissolved in 1-methyl-2-pyrrolidone (2 mL). To the solution was added copper cyanide (269 mg, 3.00 mmol). The mixture was irradiated by a microwave (150 W) at 180° C. for 0.5 hr. After cooling, ethyl acetate (20 mL) and water (20 mL) was added dropwise to the reaction mixture. The mixture was stirred for 1 hr, and extracted with ethyl acetate (X2). The organics were dried over anhydrous sodium sulfate, filtered, and concentrated in vacuo. The residue was purified by column chromatography on silica gel eluting with a 50% ethyl acetate/hexane mixture to give the title compound (156 mg, 0.388 mmol) as a white amorphous.

¹H NMR (CDCl₃) δ 3.83 (s, 3H), 7.14 (t, J=7.8 Hz, 1H), 7.25-7.26 (m, 2H), 7.37 (d, J=7.8 Hz, 1H), 7.67 (d, J=7.8 Hz, 1H).

MS Calcd.: 401; Found: 402 (M+H).

### ii) 1-{2-[2,6-dichloro-4-(trifluoromethoxy)phenoxy]-1-methyl-1H-benzimidazol-7-yl}propan-1-one

A solution of ethylmagnesium bromide (3M solution in diethyl ether; 2.0 mL, 6.0 mmol) was added dropwise to a solution of 2-[2,6-dichloro-4-(trifluoromethoxy)phenoxy]-1-methyl-1H-benzimidazole-7-carbonitrile (156 mg, 0.388 mmol) in tetrahydrofuran (5 mL) at room temperature. The mixture was refluxed for 18 hr. After cooling, the reaction mixture was quenched with 1N hydrochloric acid and extracted with ethyl acetate. The extract was washed with brine, dried over anhydrous magnesium sulfate, filtered and concentrated in vacuo to give a mixture contained the title compound. The mixture was used for the next step without further purification.

### iii) 1-{2-[2,6-dichloro-4-(trifluoromethoxy)phenoxy]-1-methyl-1H-benzimidazol-7-yl}-N-methylpropan-1-amine

The mixture described above was dissolved in a solution of methylamine (2M solution in methanol, 2.3 mL). To the mixture was added titanium (IV) isopropoxide (2.0 mL, 4.60 mmol). The mixture was stirred at room temperature for 5 hr, after which sodium borohydride (50 mg, 1.25 mmol) was added and the resulting mixture was further stirred for 1 hr. The reaction was quenched by the addition of water (1 mL), and dissolved in ethyl acetate. The organic layer was separated and the aqueous part was further extracted with ethyl acetate. The combined organics were dried over anhydrous magnesium sulfate, filtered and concentrated in vacuo. The residue was purified by preparative HPLC to give a mixture contained an intermediate of the title compound as the trifluoroacetic acid salt. The mixture was washed with aqueous sodium hydrogen carbonate solution and extracted with ethyl acetate. The extract was washed with brine, dried over anhydrous magnesium sulfate, filtered and concentrated in vacuo to give a mixture containing the title compound. The mixture was used for the next step without further purification.

### iv) 1-{2-[2,6-Dichloro-4-(trifluoromethoxy)phenoxy]-1-methyl-1H-benzimidazol-7-yl}-N,N-dimethylpropan-1-amine

The mixture described above was dissolved in acetonitrile (0.5 mL). To the solution was added dropwise a solution of formaldehyde (37% solution in water, 5.0 mL) at room temperature. The mixture was stirred at room temperature for 1 hr. After sodium cyanoborohydride (62.8 mg, 1.00 mmol) was added to the mixture, the mixture was further stirred for 2 hr. The reaction was quenched by the addition of water (3 mL). The resulting inorganic precipitate was filtered and washed with ethyl acetate. The organic layer was separated and the aqueous part was further extracted with ethyl acetate. The combined organics were dried over anhydrous magnesium sulfate, filtered and concentrated in vacuo. The residue was purified by preparative HPLC to give the title compound as the trifluoroacetic acid salt. The salt was washed with aqueous sodium hydrogen carbonate and extracted with ethyl acetate. The extract was washed with brine, dried over anhydrous magnesium sulfate, filtered and concentrated in vacuo to give the title compound (16.1 mg, 9%) as an oil.

¹H NMR (CDCl₃) δ 0.92 (t, J=7.5 Hz, 3H), 1.82-1.94 (m, 2H), 2.40 (s, 6H), 3.80 (s, 3H), 4.20-4.25 (m, 1H), 7.14 (t, J=7.8 Hz, 1H), 7.24-7.26 (m, 2H), 7.37 (d, J=7.8 Hz, 1H), 7.66 (d, J=7.8 Hz, 1H).

MS Calcd.: 461; Found: 462 (M+H).

### Experiment 1

### Measurement of CRF Binding Inhibitory Rate

A receptor binding experiment was carried out using a human CRF receptor expressing CHO cellular membrane fraction and ovine CRF, [¹²⁵I]-tyr⁰(¹²⁵I-CRF). 1000 nM of a test compound was incubated with 1 μg of human CRF receptor expressing CHO cellular membrane fraction and 50 pM of ¹²⁵I-CRF in a binding assay buffer (50 mM Tris-HCl, 5 mM EDTA, 10 mM MgCl₂, 0.05% CHAPS, 0.1% BSA, 0.5 mM PMSF, 0.1 μg/mL pepstatin, 20 μg/mL leupeptin, pH 7.5). In addition, for measuring nonspecific binding (NSB), 0.1 μM unlabelled human Urocortin was incubated with 5 μg of human CRF receptor expressing CHO cellular membrane fraction and 50 pM of ¹²⁵I-CRF in a binding assay buffer. After a binding reaction was carried out at room temperature for 1.5 hr, the membrane was entrapped on a glass filter (UniFilter plate GF-C/Perkin Elmer) by suction filtration using a cell harvester (Perkin Elmer), and washed with ice-cooled 50 mM Tris-HCl (pH 7.5). After drying the glass filter, a liquid scintillation cocktail (Microscinti 0, Perkin Elmer) was added, and the radioactivity of ¹²⁵I-CRF remaining on a glass filter was measured using Topcount (Perkin Elmer).

(TB-SB)/(TB-NSB)×100 (SB: radioactivity when a compound is added, TB: maximum binding radioactivity, NSB: nonspecific binding radioactivity) were calculated to obtain binding inhibitory rates under the range of the presence from 0.05 nM to 10 μM of each compounds. The IC₅₀ values were calculated by using GraphPad Prism software.

The compounds of Examples 1, 2, 3, 4, 5, 6, 7, 8, 10, 12, 13, 14, 15, 19, 20, 24, 25, 26, 27, 29, 30, 31, 32, 34, 35, 36, 37, 38, 39, 40, 42, 43, 44, 45, 50, 51, 52, 55, 56, 60, 62, 67, 70, 74, 75, 76, 77, 78, 79, 80, 81, 83, 84, 86, 88, 90, 91, 94, 97 and 98 have been tested in this assay and found to exhibit IC₅₀ values of less than 1 μM.

### Preparation Example 1

| | | |
|---|---|---|
| (1) | Compound of Example 1 | 50 mg |
| (2) | Lactose | 34 mg |
| (3) | Corn starch | 10.6 mg |
| (4) | Corn starch (paste) | 5 mg |
| (5) | Magnesium stearate | 0.4 mg |
| (6) | Carboxymethylcellulose calcium | 20 mg |
| | Total | 120 mg |

According to a conventional method, the above (1) to (6) are mixed and compressed into a tablet with a tableting machine.

### INDUSTRIAL APPLICABILITY

Compound (I) of the present invention has an excellent CRF antagonistic activity, etc. and therefore useful as pharmaceuticals for treating or preventing affective disorder, depression, anxiety, and the like.

1. A compound represented by the formula (I): 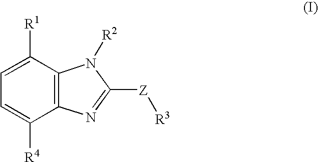
wherein R¹ is an optionally substituted C₁₋₁₀ alkyl;
R² is a hydrogen, or a C₁₋₆ alkyl which may be substituted with 1 to 3 substituents selected from the group consisting of a hydroxy, a cyano, an amino, a mono- or di-C₁₋₆ alkylamino, a C₁₋₆ alkoxy and an optionally substituted benzyloxy;
R³ is a 5- or 6-membered aromatic group which may be substituted with 1 to 5 substituents selected from the group consisting of (i) halogen, (ii) cyano, (iii) C₁₋₆ alkyl which may be substituted with 1 to 3 substituents selected from the group consisting of (i′) a mono- or di-C₁₋₆ alkylamino, (ii′) a halogen and (iii′) 5- or 6-membered heterocyclic group which may be substituted with 1 to 3 C₁₋₆ alkyls, (iv) amino which may be substituted with a C₁₋₆ alkyl, (v) C₁₋₆ alkoxy which may be substituted with 1 to 3 halogens, (vi) C₁₋₆ alkyl-carbonyl, (vii) carbamoyl, (viii) 5- or 6-membered heterocyclic group which may be substituted with 1 to 3 C₁₋₆ alkyls which may be substituted with 1 to 3 halogens, (ix) C₁₋₆ alkylthio, (x) C₁₋₆ alkylsulfinyl, (xi) C₁₋₆ alkylsulfonyl, (xii) sulfamoyl, and (xiii) tri-C₁₋₆ alkylsilyl, wherein the 5- or 6-membered aromatic group may be fused with a 5- or 6-membered ring which may be substituted with 1 to 3 C₁₋₆ alkyls;
R⁴ is a hydrogen, a halogen, a hydroxy, a cyano, a C₁₋₆ alkyl or a C₁₋₆ alkoxy;
Z is —O—, —S—, —SO—, —SO₂—, or —NR⁵— wherein R⁵ is a hydrogen or a C₁₋₆ alkyl; or a salt thereof.

2. A prodrug of the compound according to claim 1.

3. The compound according to claim 1 wherein R¹ is a C₃₋₁₀ alkyl which may be substituted with 1 or 2 substituents selected from the group consisting of an amino and a mono- or di-C₁₋₆ alkylamino.

4. The compound according to claim 1 wherein R² is a hydrogen, or a C₁₋₆ alkyl which may be substituted with 1 to 3 substituents selected from the group consisting of a hydroxy and di-C₁₋₆ alkylamino.

5. The compound according to claim 1 wherein R³ is (i) 2,4,6-trisubstituted phenyl, (ii) 2,4-disubstituted phenyl, (iii) 2,4,6-trisubstituted 3-pyridyl, (iv) 2,6- or 4,6-disubstituted 3-pyridyl, (v) 3,5-disubstituted 2-pyridyl, or (vi) 1,4,5-trisubstituted 3-pyrazolyl.

6. The compound according to claim 1 wherein R⁴ is a hydrogen, a halogen, or a C₁₋₆ alkoxy.

7. The compound according to claim 1 wherein Z is —O— or —NH—.

8. The compound according to claim 1 wherein the compound is
(1) 2-[2-chloro-4-(trifluoromethoxy)phenoxy]-7-(1-ethylpropyl)-4-methoxy-1-methyl-1H-benzimidazole,
(2) 3,5-dichloro-4-{[7-(1-ethylpropyl)-4-methoxy-1-methyl-1H-benzimidazol-2-yl]oxy}-N,N-dimethylaniline,
(3) 3-chloro-4-{[7-(1-ethylpropyl)-4-methoxy-1-methyl-1H-benzimidazol-2-yl]oxy}-N,N-dimethylaniline,
(4) 5-chloro-2-{[7-(1-ethylpropyl)-4-methoxy-1-methyl-1H-benzimidazol-2-yl]oxy}-N,N-dimethylaniline,
(5) 3-{[7-(1-ethylpropyl)-4-methoxy-1-methyl-1H-benzimidazol-2-yl]oxy}-N,N,6-trimethylpyridin-2-amine,
(6) 1-(4-{[7-(1-ethylpropyl)-4-methoxy-1-methyl-1H-benzimidazol-2-yl]oxy}-3-methylphenyl)ethanone,
(7) 3,5-dichloro-2-{[7-(1-ethylpropyl)-4-methoxy-1-methyl-1H-benzimidazol-2-yl]oxy}-N,N-dimethylaniline,
(8) 2-[(5-bromo-3-methylpyridin-2-yl)oxy]-7-(1-ethylpropyl)-4-methoxy-1-methyl-1H-benzimidazole,
(9) 1-(5-chloro-2-{[7-(1-ethylpropyl)-4-methoxy-1-methyl-1H-benzimidazol-2-yl]oxy}phenyl)-N,N-dimethylmethanamine,
(10) 2-[2-bromo-4-(trifluoromethoxy)phenoxy]-7-(1-ethylpropyl)-4-methoxy-1-methyl-1H-benzimidazole,
(11) 7-(1-ethylpropyl)-4-methoxy-1-methyl-2-{[6-methyl-2-(trifluoromethyl)pyridin-3-yl]oxy}-1H-benzimidazole,
(12) 2-{[7-(1-ethylpropyl)-4-methoxy-1-methyl-1H-benzimidazol-2-yl]oxy}-N,N-dimethyl-5-(trifluoromethyl)aniline,
(13) 1-[2-{[7-(1-ethylpropyl)-4-methoxy-1-methyl-1H-benzimidazol-2-yl]oxy}-5-(trifluoromethoxy)phenyl]-N,N-dimethylmethanamine,
(14) 7-(1-ethylpropyl)-4-methoxy-1-methyl-2-[(1,4,5-trimethyl-1H-pyrazol-3-yl)oxy]-1H-benzimidazole,
(15) 1-[2-{[7-(1-ethylpropyl)-4-methoxy-1-methyl-1H-benzimidazol-2-yl]oxy}-3-methyl-5-(trifluoromethoxy)phenyl]-N,N-dimethylmethanamine,
(16) N-[2-chloro-4-(trifluoromethyl)phenyl]-7-(1-ethylpropyl)-4-methoxy-1-methyl-1H-benzimidazol-2-amine,
(17) N-[4-chloro-2-(trifluoromethyl)phenyl]-7-(1-ethylpropyl)-4-methoxy-1-methyl-1H-benzimidazol-2-amine,
(18) N-[2-chloro-4-(trifluoromethoxy)phenyl]-7-(1-ethylpropyl)-4-methoxy-1-methyl-1H-benzimidazol-2-amine,
(19) N-(2-bromo-4-chlorophenyl)-7-(1-ethylpropyl)-4-methoxy-1-methyl-1H-benzimidazol-2-amine,
(20) N-(2,4-dichlorophenyl)-7-(1-ethylpropyl)-4-methoxy-1-methyl-1H-benzimidazol-2-amine,
(21) 4-chloro-N¹-[7-(1-ethylpropyl)-4-methoxy-1-methyl-1H-benzimidazol-2-yl]-N²,N²-dimethylbenzene-1,2-diamine,
(22) 5-chloro-2-{[7-(1-ethylpropyl)-4-methoxy-1-methyl-1H-benzimidazol-2-yl]oxy}benzenesulfonamide,
(23) 2-(2-bromo-4,6-dichlorophenoxy)-4-chloro-7-(1-ethylpropyl)-1-methyl-1H-benzimidazole,
(24) 3,5-dichloro-2-{[4-chloro-7-(1-ethylpropyl)-1-methyl-1H-benzimidazol-2-yl]oxy}-N,N-dimethylaniline,
(25) 4-chloro-2-(2,4-dichloro-6-morpholin-4-ylphenoxy)-7-(1-ethylpropyl)-1-methyl-1H-benzimidazole,
(26) 4-chloro-2-[2,4-dichloro-6-(4-methylpiperazin-1-yl)phenoxy]-7-(1-ethylpropyl)-1-methyl-1H-benzimidazole,
(27) 4-chloro-2-[2,4-dichloro-6-(1H-imidazol-1-yl)phenoxy]-7-(1-ethylpropyl)-1-methyl-1H-benzimidazole,
(28) 3,5-dichloro-2-{[4-chloro-7-(1-ethylpropyl)-1-methyl-1H-benzimidazol-2-yl]oxy}-N-methylaniline,
(29) 4-chloro-2-(2,4-dichloro-6-pyrrolidin-1-ylphenoxy)-7-(1-ethylpropyl)-1-methyl-1H-benzimidazole,
(30) 2-{[7-(1-ethylpropyl)-4-methoxy-1-methyl-1H-benzimidazol-2-yl]oxy}-N,N-dimethyl-5-(trifluoromethoxy)aniline,
(31) 3,5-dichloro-2-{[4-chloro-7-(1-ethylpropyl)-1-methyl-1H-benzimidazol-2-yl]oxy}benzonitrile,
(32) 3,5-dichloro-2-{[4-chloro-7-(1-ethylpropyl)-1-methyl-1H-benzimidazol-2-yl]oxy}benzenesulfonamide,
(33) 2-(4-bromo-2-chloro-6-methylphenoxy)-4-chloro-7-(1-ethylpropyl)-1-methyl-1H-benzimidazole,
(34) 3-chloro-4-{[4-chloro-7-(1-ethylpropyl)-1-methyl-1H-benzimidazol-2-yl]oxy}-5-methylbenzonitrile,
(35) 1-(3-chloro-4-{[4-chloro-7-(1-ethylpropyl)-1-methyl-1H-benzimidazol-2-yl]oxy}-5-methylphenyl)ethanone,
(36) 4-chloro-2-[2-chloro-6-methyl-4-(methylthio)phenoxy]-7-(1-ethylpropyl)-1-methyl-1H-benzimidazole,
(37) 4-chloro-2-[2-chloro-6-methyl-4-(methylsulfinyl)phenoxy]-7-(1-ethylpropyl)-1-methyl-1H-benzimidazole,
(38) 4-chloro-2-[2-chloro-6-methyl-4-(methylsulfonyl)phenoxy]-7-(1-ethylpropyl)-1-methyl-1H-benzimidazole,
(39) 3-chloro-4-{[4-chloro-7-(1-ethylpropyl)-1-methyl-1H-benzimidazol-2-yl]oxy}-5-methylbenzamide,
(40) 3-chloro-4-{[4-chloro-7-(1-ethylpropyl)-1-methyl-1H-benzimidazol-2-yl]oxy}-5-methylbenzenesulfonamide,
(41) 2-[2-bromo-6-chloro-4-(trifluoromethoxy)phenoxy]-4-chloro-7-(1-ethylpropyl)-1-methyl-1H-benzimidazole,
(42) 4-chloro-2-[2-chloro-6-(methylthio)-4-(trifluoromethoxy)phenoxy]-7-(1-ethylpropyl)-1-methyl-1H-benzimidazole,
(43) 4-chloro-2-[2-chloro-6-(methylsulfinyl)-4-(trifluoromethoxy)phenoxy]-7-(1-ethylpropyl)-1-methyl-1H-benzimidazole,
(44) 4-chloro-2-[2-chloro-6-(methylsulfonyl)-4-(trifluoromethoxy)phenoxy]-7-(1-ethylpropyl)-1-methyl-1H-benzimidazole,
(45) 4-chloro-7-(1-ethylpropyl)-N-[2-methoxy-6-methyl-4-(methylthio)phenyl]-1-methyl-1H-benzimidazol-2-amine,
(46) 4-chloro-7-(1-ethylpropyl)-N-[2-methoxy-6-methyl-4-(methylsulfinyl)phenyl]-1-methyl-1H-benzimidazol-2-amine,
(47) 4-chloro-7-(1-ethylpropyl)-N-[2-methoxy-6-methyl-4-(methylsulfonyl)phenyl]-1-methyl-1H-benzimidazol-2-amine,
(48) 4-{[4-chloro-7-(1-ethylpropyl)-1-methyl-1H-benzimidazol-2-yl]amino}-3-methoxy-5-methylbenzamide,
(49) N-(2-bromo-4-chloro-6-methylphenyl)-4-chloro-7-(1-ethylpropyl)-1-methyl-1H-benzimidazol-2-amine,
(50) 4-chloro-N-[4-chloro-2-methyl-6-(methylthio)phenyl]-7-(1-ethylpropyl)-1-methyl-1H-benzimidazol-2-amine,
(51) 4-chloro-N-[4-chloro-2-methyl-6-(methylsulfinyl)phenyl]-7-(1-ethylpropyl)-1-methyl-1H-benzimidazol-2-amine,
(52) 3-{[4-chloro-7-(1-ethylpropyl)-1-methyl-1H-benzimidazol-2-yl]oxy}-4-methoxy-N,N,6-trimethylpyridin-2-amine,
(53) 4-chloro-7-(1-ethylpropyl)-1-methyl-2-[(2-methyl-2,4,5,6-tetrahydrocyclopenta[c]pyrazol-3-yl)oxy]-1H-benzimidazole,
(54) 4-chloro-7-(1-ethylpropyl)-1-methyl-2-[(2-methyl-2,3-dihydro-1H-isoindol-4-yl)oxy]-1H-benzimidazole,
(55) 4-chloro-2-[(5,7-dichloro-2-methyl-2,3-dihydro-1H-isoindol-4-yl)oxy]-7-(1-ethylpropyl)-1-methyl-1H-benzimidazole,
(56) 4-chloro-7-(1-ethylpropyl)-2-{[4-methoxy-2-methyl-6-(trifluoromethyl)pyridin-3-yl]oxy}-1-methyl-1H-benzimidazole,
(57) 4-chloro-7-(1-ethylpropyl)-N-[2-methoxy-6-methyl-4-(1H-pyrazol-1-yl)phenyl]-1-methyl-1H-benzimidazol-2-amine,
(58) 4-chloro-7-(1-ethylpropyl)-N-{2-methoxy-6-methyl-4-[3-(trifluoromethyl)-1H-pyrazol-1-yl]phenyl}-1-methyl-1H-benzimidazol-2-amine,
(59) 4-chloro-2-[2-chloro-6-methyl-4-(trimethylsilyl)phenoxy]-7-(1-ethylpropyl)-1-methyl-1H-benzimidazole,
(60) 4-chloro-2-[2,4-dichloro-6-(trimethylsilyl)phenoxy]-7-(1-ethylpropyl)-1-methyl-1H-benzimidazole,
(61) N-(4-chloro-2-methoxy-6-methylphenyl)-1-methyl-7-[1-(methylamino)propyl]-1H-benzimidazol-2-amine,
(62) N-(4-chloro-2-methoxy-6-methylphenyl)-7-[1-(dimethylamino)propyl]-1-methyl-1H-benzimidazol-2-amine,
(63) 2-[4-bromo-2-(2,4-dichloro-6-methylphenoxy)-7-(1-ethylpropyl)-1H-benzimidazol-1-yl]-N,N-dimethylethanamine,
(64) 4-chloro-2-(2,4-dichloro-6-methylphenoxy)-7-(1-ethylpropyl)-1H-benzimidazole,
(65) 4-chloro-2-[2,6-dichloro-4-(trifluoromethoxy)phenoxy]-7-(1-ethylpropyl)-1H-benzimidazole,
(66) 4-chloro-N-(2,4-dichloro-6-methylphenyl)-7-(1-ethylpropyl)-1H-benzimidazol-2-amine,
(67) 4-chloro-N-(4-chloro-2-methoxy-6-methylphenyl)-7-(1-ethylpropyl)-1H-benzimidazol-2-amine,
(68) 4-chloro-2-[2,6-dichloro-4-(trifluoromethoxy)-phenoxy]-7-(1-ethylpropyl)-1-{2-[(4-methoxybenzyl)oxy]ethyl}-1H-benzimidazole,
(69) 2-[4-chloro-2-[2,6-dichloro-4-(trifluoromethoxy)phenoxy]-7-(1-ethylpropyl)-1H-benzimidazol-1-yl]ethanol,
(70) 4-bromo-2-(2,4-dichloro-6-methylphenoxy)-7-(1-ethylpropyl)-1-methyl-1H-benzimidazole,
(71) 2-(2,4-dichloro-6-methylphenoxy)-4-ethyl-7-(1-ethylpropyl)-1-methyl-1H-benzimidazole,
(72) 2-(2-bromo-4-chlorophenoxy)-4-ethoxy-7-(1-ethylpropyl)-1-methyl-1H-benzimidazole,
(73) 2-(2,4-dichloro-6-methylphenoxy)-4-ethoxy-7-(1-ethylpropyl)-1-methyl-1H-benzimidazole,
(74) 4-chloro-2-[(4,6-dibromo-2-methylpyridin-3-yl)oxy]-7-(1-ethylpropyl)-1-methyl-1H-benzimidazole,
(75) 2-{[4-bromo-2-methyl-6-(trifluoromethyl)pyridin-3-yl]oxy}-4-chloro-7-(1-ethylpropyl)-1-methyl-1H-benzimidazole,
(76) 4-chloro-7-(1-ethylpropyl)-1-methyl-2-{[2-methyl-4,6-bis(trifluoromethyl)pyridin-3-yl]oxy}-1H-benzimidazole,
(77) 1-(3,5-dichloro-2-{[7-(1-ethylpropyl)-4-methoxy-1-methyl-1H-benzimidazol-2-yl]oxy}phenyl)-N,N-dimethylmethanamine,
(78) 4-chloro-2-[2,4-dichloro-6-(pyrrolidin-1-ylmethyl)phenoxy]-7-(1-ethylpropyl)-1-methyl-1H-benzimidazole,
(79) 2-[2,4-dichloro-6-(pyrrolidin-1-ylmethyl)phenoxy]-7-(1-ethylpropyl)-4-methoxy-1-methyl-1H-benzimidazole,
(80) 2-{2,4-dichloro-6-[(2-methylpyrrolidin-1-yl)methyl]phenoxy}-7-(1-ethylpropyl)-4-methoxy-1-methyl-1H-benzimidazole,
(81) 2-(4-bromo-2-chloro-6-fluorophenoxy)-7-(1-ethylpropyl)-4-methoxy-1-methyl-1H-benzimidazole,
(82) 7-(1-ethylpropyl)-2-[2-(1H-imidazol-1-yl)-4-(trifluoromethoxy)phenoxy]-4-methoxy-1-methyl-1H-benzimidazole,
(83) 3-chloro-4-{[4-chloro-7-(1-ethylpropyl)-1-methyl-1H-benzimidazol-2-yl]oxy}-N,N-dimethylaniline,
(84) 1-[2-{[4-chloro-7-(1-ethylpropyl)-1-methyl-1H-benzimidazol-2-yl]oxy}-3-methyl-5-(trifluoromethoxy)phenyl]-N,N-dimethylmethanamine,
(85) 1-(3,5-dichloro-2-{[4-chloro-7-(1-ethylpropyl)-1-methyl-1H-benzimidazol-2-yl]oxy}phenyl)-N,N-dimethylmethanamine,
(86) 3-chloro-4-{[7-(1-ethylpropyl)-4-methoxy-1-methyl-1H-benzimidazol-2-yl]oxy}-N,N-dimethyl-5-(pyrrolidin-1-ylmethyl)aniline,
(87) N-(2,4-dichloro-6-methylphenyl)-7-(1-ethylpropyl)-4-methoxy-1-methyl-1H-benzimidazol-2-amine,
(88) N-[2,6-dichloro-4-(trifluoromethoxy)phenyl]-7-(1-ethylpropyl)-4-methoxy-1-methyl-1H-benzimidazol-2-amine,
(89) 2-[(2,4-dichloro-6-methylphenyl)amino]-7-(1-ethylpropyl)-1-methyl-1H-benzimidazol-4-ol,
(90) 2-(2,4-dichloro-6-methylphenoxy)-7-(1-ethylpropyl)-1-methyl-1H-benzimidazole,
(91) 1-[4-chloro-2-(2,4-dichloro-6-methylphenoxy)-1-methyl-1H-benzimidazol-7-yl]propan-1-one,
(92) 4-chloro-2-(2,4-dichloro-6-methylphenoxy)-1-methyl-7-(1-methylenepropyl)-1H-benzimidazole,
(93) 2-[4-chloro-2-(2,4-dichloro-6-methylphenoxy)-1-methyl-1H-benzimidazol-7-yl]butan-1-ol,
(94) 2-[4-chloro-2-(2,4-dichloro-6-methylphenoxy)-1-methyl-1H-benzimidazol-7-yl]butanal,
(95) 4-chloro-2-(2,4-dichloro-6-methylphenoxy)-7-(1-ethylprop-2-en-1-yl)-1-methyl-1H-benzimidazole,
(96) 3-[4-chloro-2-(2,4-dichloro-6-methylphenoxy)-1-methyl-1H-benzimidazol-7-yl]pentan-1-ol,
(97) 3-[4-chloro-2-(2,4-dichloro-6-methylphenoxy)-1-methyl-1H-benzimidazol-7-yl]pentanal,
(98) 3-[4-chloro-2-(2,4-dichloro-6-methylphenoxy)-1-methyl-1H-benzimidazol-7-yl]pentanoic acid,
(99) 2-[4-chloro-2-[(2,4-dichlorophenyl)amino]-7-(1-ethylpropyl)-1H-benzimidazol-1-yl]ethanol,
(100) 2-[4-chloro-2-({4-chloro-2-[(dimethylamino)methyl]phenyl}amino)-7-(1-ethylpropyl)-1H-benzimidazol-1-yl]ethanol or
(101) 1-{2-[2,6-dichloro-4-(trifluoromethoxy)phenoxy]-1-methyl-1H-benzimidazol-7-yl}-N,N-dimethylpropan-1-amine.

9. A pharmaceutical which comprises the compound according to claim 1 or its prodrug.

10. The pharmaceutical according to claim 9 which is for treating or preventing a disease associated with the functions of a CRF receptor.

11. The pharmaceutical according to claim 10 wherein the disease is affective disorder, depression or anxiety.

12. A method for treating or preventing a disease associated with the functions of a CRF receptor, which comprises administering to a subject in need thereof an effective amount of the compound according to claim 1 or its prodrug.

13. The method according to claim 12 wherein the disease is affective disorder, depression or anxiety.

14. Use of the compound according to claim 1 or its prodrug for manufacturing an agent of treating or preventing a disease associated with the functions of a CRF receptor.

15. The use according to claim 14 wherein the disease is affective disorder, depression or anxiety.
